(19) United States
(12) Patent Application Publication
(10) Pub. No.: US 2018/0271849 A1
(43) Pub. Date: Sep. 27, 2018

(54) PHARMACEUTICAL COMPOSITION AND APPLICATION THEREOF

(71) Applicant: KANGPU BIOPHARMACEUTICALS, LTD., Shanghai (CN)

(72) Inventors: Chuansheng GE, Shanghai (CN); Baisong LIAO, Shanghai (CN); Wen-Cherng LEE, Shanghai (CN)

(21) Appl. No.: 15/763,883

(22) PCT Filed: Sep. 28, 2016
(86) PCT No.: PCT/CN2016/100642

§ 371 Date: Mar. 28, 2018

(30) Foreign Application Priority Data
Sep. 29, 2015 (CN) 201510631654.9

(51) Int. Cl. A61K 31/454; A61P 35/04

(52) U.S. Cl. CPC A61K 31/454; A61K 31/4035; A61P 35/04

(57) ABSTRACT

Disclosed are a pharmaceutical composition and an application thereof. The pharmaceutical composition includes one or more of a benzoheterocyclic compound as shown in formula (I), a pharmaceutically acceptable salt thereof, a solvate thereof, a crystalline form thereof, a co-crystal thereof, a stereoisomer thereof, an isotope compound thereof, a metabolite thereof and a pro-drug thereof, and an androgen receptor pathway modulator. The pharmaceutical composition better inhibits prostate cancer cell growth.

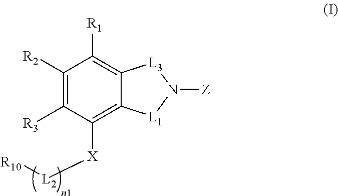

The present application claims the benefit of Chinese Patent Application No. CN201510631654.9 filed on Sep. 29, 2015, the contents of which are incorporated herein by reference in their entireties.

### FIELD OF INVENTION

The present invention relates to a pharmaceutical composition and application thereof.

### PRIOR ARTS

Prostate cancer is a common malignancy in the male reproductive system. The statistics which was made by the International Agency for Research on Cancer of World Health Organization in 2012 showed that the number of newly diagnosed prostate cancer patients in the world was 1.1 million in that year, accounting for about 15% of the total number of new cancer cases, making it the second most common cancer in men worldwide. In the United States, the incidence of prostate cancer ranks first in all malignancies, with the second highest mortality rate, second only to lung cancer. Although the incidence of prostate cancer in China is much lower than that in western countries, it has shown a significant growth trend in recent years and ranks first among male urological tumors, and most of prostate cancer were diagnosed in the terminal stage.

The growth of the prostate cancer cells requires the supporting of androgens including testosterone. Therefore, the targeted treatment strategies for prostate cancer mainly focus on the synthesis of androgen and the binding to the androgen receptor thereof. For example, Enzalutamide, a prostate cancer drug marketed by the U.S. FDA in August 2012, is a small molecule androgen receptor antagonist, which finally inhibits the androgen receptor pathway by competitive inhibition of the binding of androgen to its receptor, thereby achieving the effect of treating castration-resistant prostate cancer.

Enzalutamide also shows some side effects in clinical studies, such as weakness or fatigue, lumbago, diarrhea, joint pain, hot flashes, tissue swelling, musculoskeletal pain, headache, upper respiratory tract infection, dizziness, spinal cord compression and cauda equina syndrome, muscle weakness, dyscoimesis, lower respiratory tract infection, hematuria, tingling, anxiety and hypertension and so on.

For the treatment of cancer, the drug combination is often used in the clinical practice to improve the treatment effect, for example, the combination of docetaxel and prednisone for use in the treatment of prostate cancer. However, people have met great setbacks when exploring new combination regimens. One of the typical examples is that although the combination of docetaxel and prednisone can treat prostate cancer (Tannock et al. N. Eng. J. Med. (2004), 351, 1502-1512), the combination regimen of docetaxel, prednisone and lenalidomide failed in a Phase III clinical trial involving more than 1000 prostate cancer patients (Petrylak et al. Lancet Oncol. (2015) 16-4, 417-425). It should also be noted that, the results of several phase II clinical studies also indicated that the clinical efficacy of lenalidomide alone in the treatment of prostate cancer was not satisfying (Xing et al. Asian Pac. J. Cancer Prev. (2015) 16-9, 3969-3972). Therefore, it has become an urgent technical problem to be solved in the art to explore combination regimens of anti-prostate cancer drugs (including Enzalutamide etc.) to improve the efficacy and reduce the toxic and side effect.

### CONTENT OF THE PRESENT INVENTION

The technical problem to be solved in the present invention is to improve the efficacy of the present anti-prostate cancer drugs (including Enzalutamide etc.).

In one aspect of the present invention, it provides a pharmaceutical composition, comprising a benzoheterocyclic compound as shown in formula (I), a pharmaceutically acceptable salt thereof, a solvate thereof, a polymorph thereof, a co-crystal thereof, a stereoisomer thereof, an isotope compound thereof, a metabolite thereof or a prodrug thereof, and an androgen receptor pathway modulator;

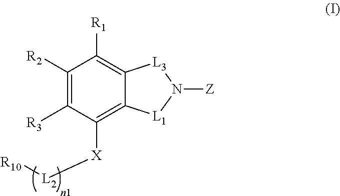

in formula (I), n1 is 0 or 1;

L₁ and L₃ are

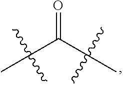 [SMILES: *C(*)=O]

independently CH₂, CHD, CD₂,

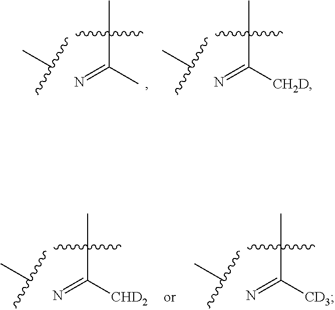

L₂ is CD₂, CHD or CH₂;

X is NH, ND or O;

R₁, R₂ and R₃ are independently H or D;

Z is

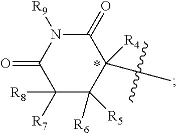

wherein, R₄ is H, D, CH₃, CH₂D, CHD₂ or CD₃; R₅, R₆, R₇, R₈ and R₉ are independently H or D; the carbon marked with * is an asymmetric center;

R₁₀ is H, D or

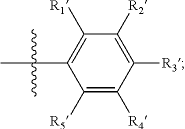

wherein, R₁′, R₂′, R₃′, R₄′ and R₅′ are independently selected form H, D, or substituted or unsubstituted (C₁-C₁₂) alkyl;

the substituent in the substituted (C₁-C₁₂) alkyl is selected from one or more of the following groups: D, (C₂-C₂₀) heterocycloalkyl, deuterated C₂-C₂₀ heterocycloalkyl, (C₂-C₂₀) heterocycloalkyl substituted by (C₁-C₁₂) alkyl and (C₂-C₂₀) heterocycloalkyl substituted by deuterated (C₁-C₁₂) alkyl;

when there are a plurality of substituents in the substituted (C₁-C₁₂) alkyl, the substituents may be the same or different;

in each of the above groups, the heteroatom in the (C₂-C₂₀) heterocycloalkyl which is referred in the (C₂-C₂₀) heterocycloalkyl, deuterated (C₂-C₂₀) heterocycloalkyl, (C₂-C₂₀) heterocycloalkyl substituted by (C₁-C₁₂) alkyl and (C₂-C₂₀) heterocycloalkyl substituted by deuterated (C₁-C₁₂) alkyl is selected from one or more of O, N and S;

when n1 is 0, X is NH or ND, L₁ is

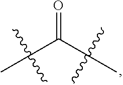 [SMILES: *C(*)=O]

CH₂, CHD or CD₂; L₃ is

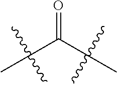

in formula (I), R₁₀ is H or D;

when n1 is 0, X is NH or ND, L₁ is

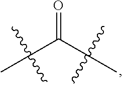 [SMILES: *C(*)=O]

### and L₃ is

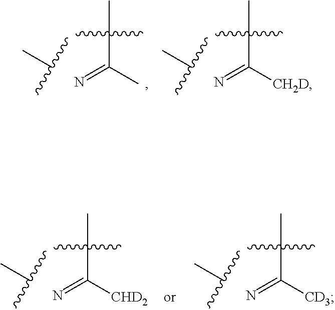

in formula (I), R₁₀ is H or D;

when n1 is 1, L₁ is CH₂, CHD or CD₂, L₃ is

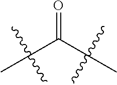

in formula (I), R₁₀ is the

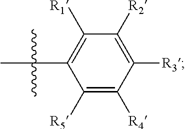

D represents deuterium-enriched hydrogen, and H represents non-deuterium-enriched hydrogen;

the androgen receptor pathway modulator is one or more of the group consisting of Enzalutamide, ARN-509, ODM-201, VT-464, Orteronel, EPI-001, Andarine, RD162, BMS-641988, CH5137291, Flutamide, Hydroxy flutamide, RU58642, LG120907, LG105, Galeterone, Spironolactone, MK-2866, AZD3514, Cyproterone acetate, ORM-15341, Bicalutamide, Nilutamide, Degarelix, Goserelin acetate, Leuprolide acetat, Abiraterone and Abiraterone Acetate;

when the androgen receptor pathway modulator is selected from one of the above compounds, the androgen receptor pathway modulator is not Bicalutamide, Nilutamide, Leuprolide acetate, Abiraterone or Abiraterone Acetate.

In the formula (I) of the present invention, the asymmetric center preferably refers to achiral carbon, (S)-configured carbon, (R)-configured carbon or racemate.

In the formula (I) of the present invention, the Z is preferably any one of the following structures:

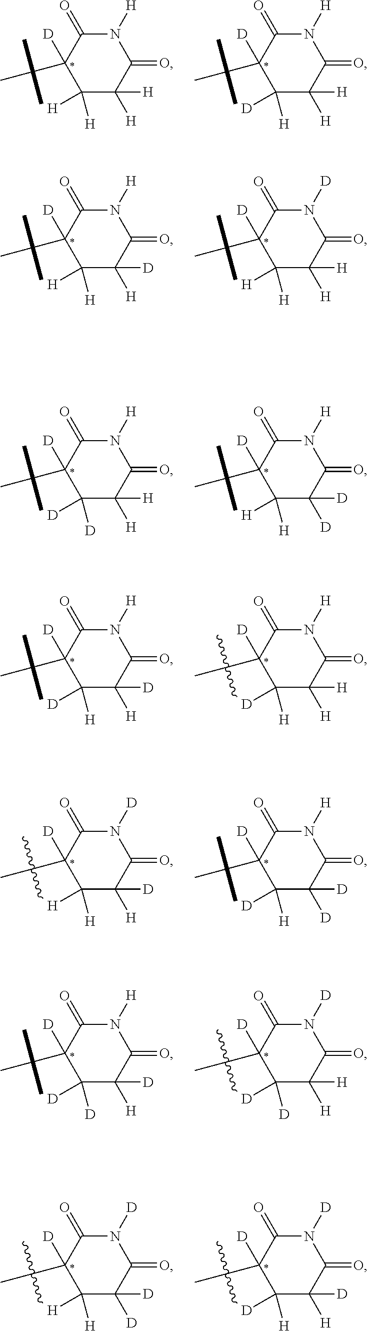

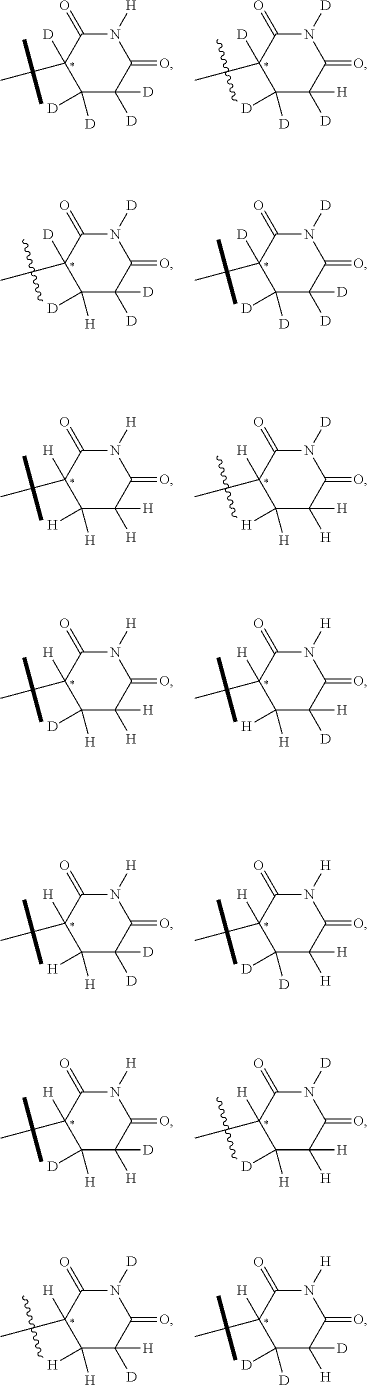

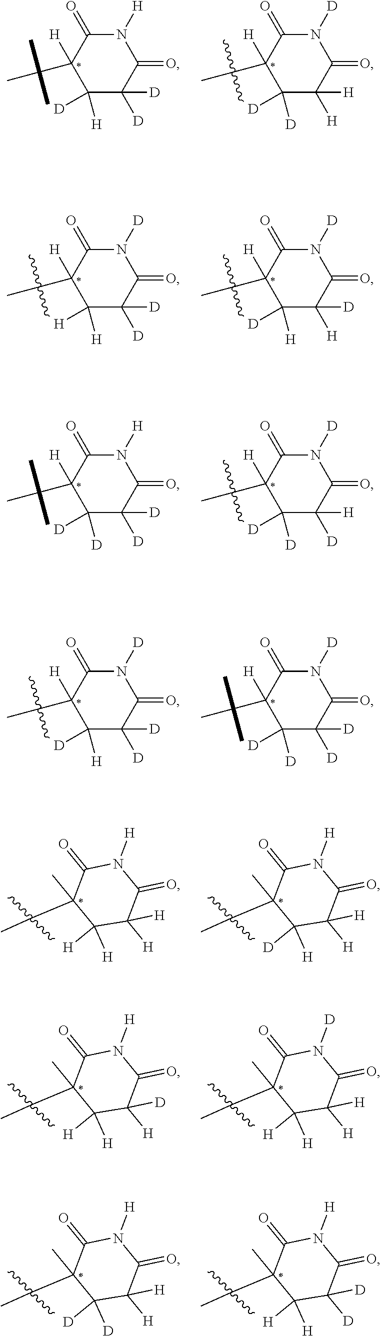

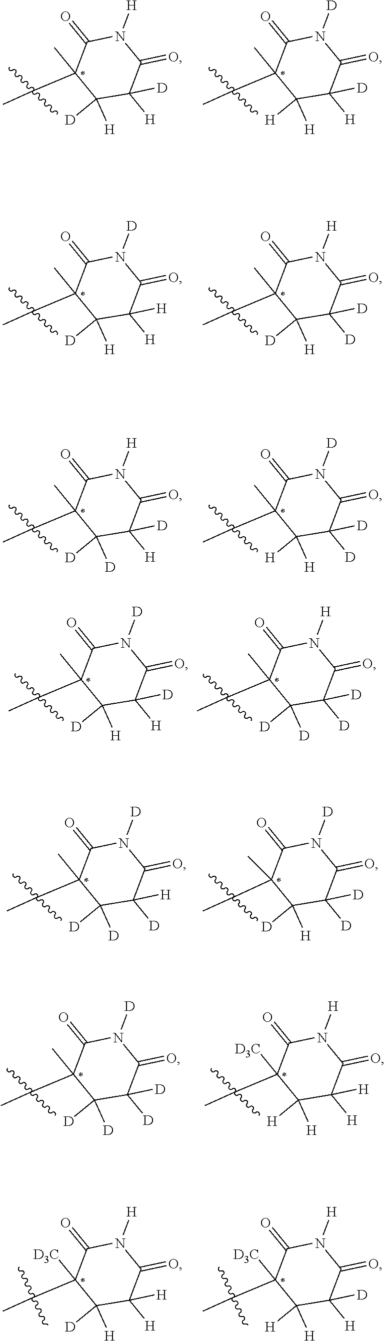

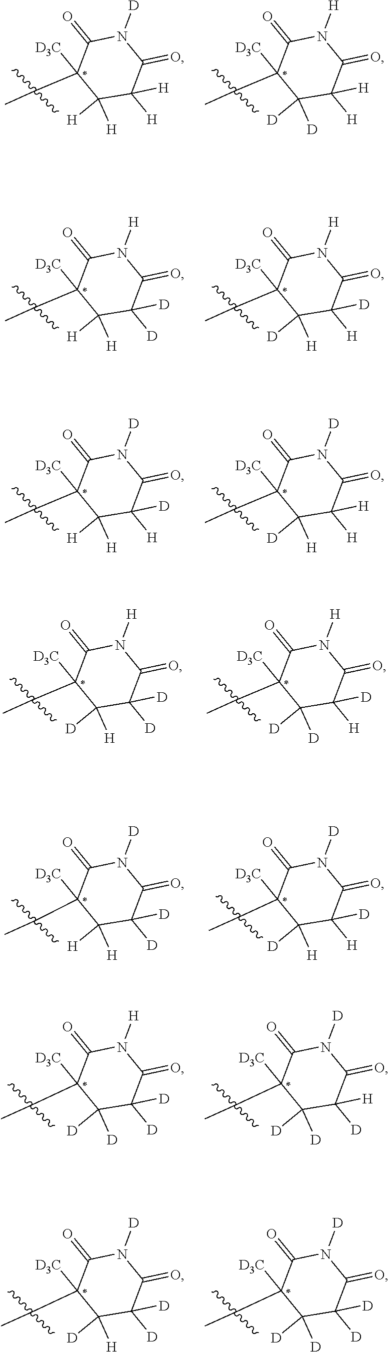

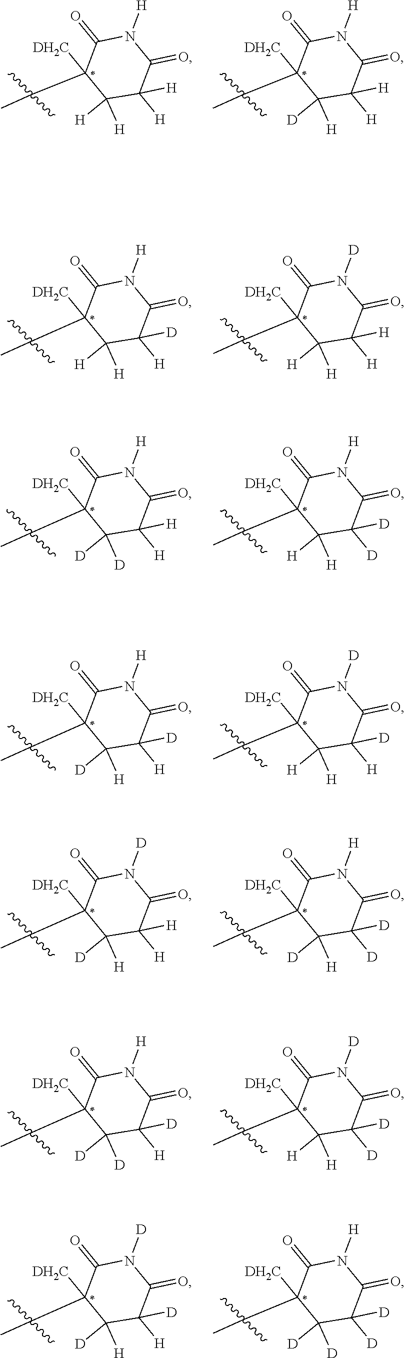

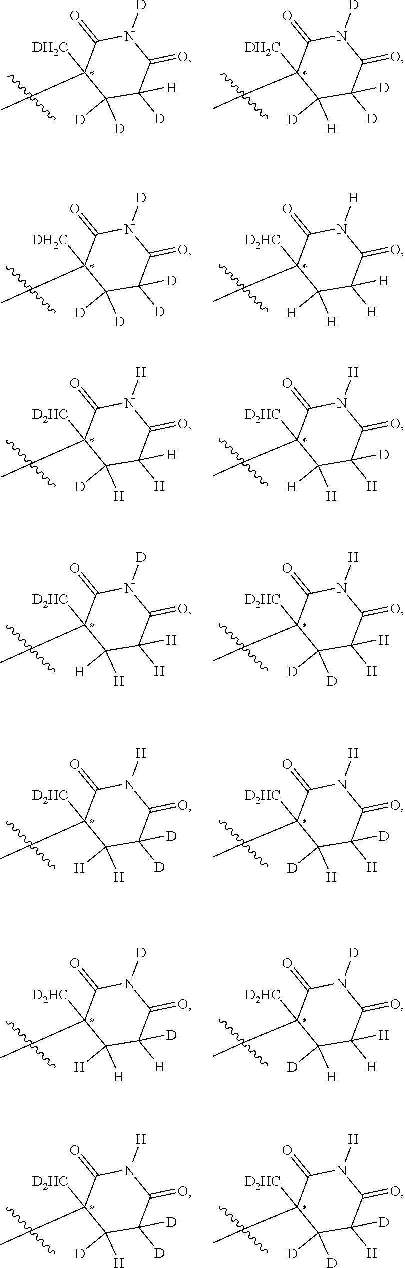

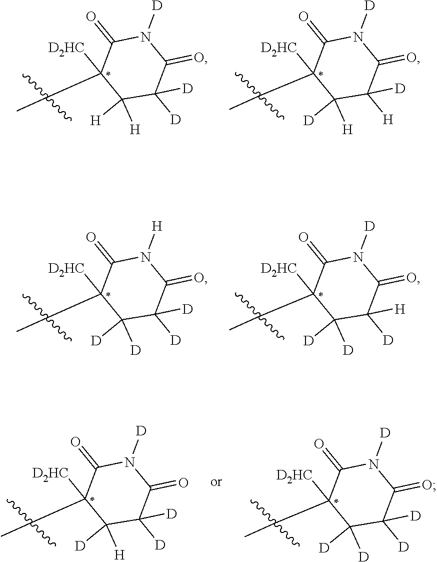

Z is more preferably any one of the following structures:

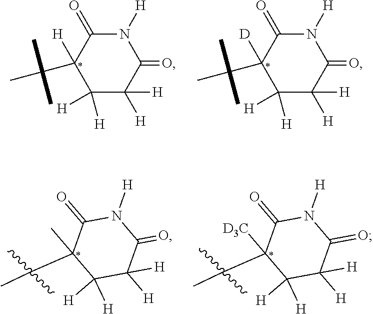

wherein, the carbon marked with * is an asymmetric center, and the asymmetric center, H and D are defined as described above.

In the formula (I) of the present invention, the (C₂-C₂₀) heterocycloalkyl of the (C₂-C₂₀) heterocycloalkyl, the deuterated (C₂-C₂₀) heterocycloalkyl, the (C₂-C₂₀) heterocycloalkyl substituted by (C₁-C₁₂) alkyl or the (C₂-C₂₀) heterocycloalkyl substituted by deuterated (C₁-C₁₂) alkyl preferably refers to (C₂-C₆) heterocycloalkyl wherein the heteroatom is N or O and the number of heteroatoms is 1-2. The (C₂-C₆) heterocycloalkyl is preferably a morpholinyl

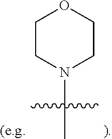 [SMILES: *N1CCOCC1]

The (C₁-C₁₂) alkyl of the (C₂-C₂₀) heterocycloalkyl substituted by (C₁-C₁₂) alkyl or the (C₂-C₂₀) heterocycloalkyl substituted by deuterated (C₁-C₁₂) alkyl is preferably (C₁-C₄) alkyl. The (C₁-C₄) alkyl is preferably methyl, ethyl, n-propyl, isopropyl, n-butyl, isobutyl or tertiary-butyl.

The substituted or unsubstituted (C₁-C₁₂) alkyl in the formula (I) of the present invention is preferably substituted or unsubstituted (C₁-C₄) alkyl. The substituted or unsubstituted (C₁-C₄) alkyl is preferably substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, or substituted or unsubstituted tertiary-butyl. The substituted (C₁-C₄) alkyl is preferably

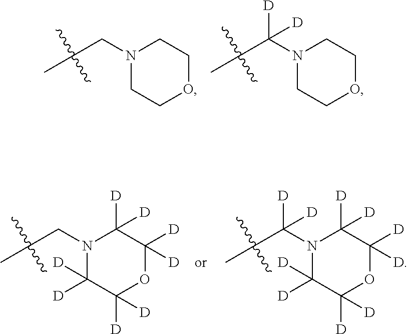

In the formula (I), the

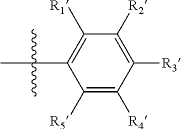

is preferably

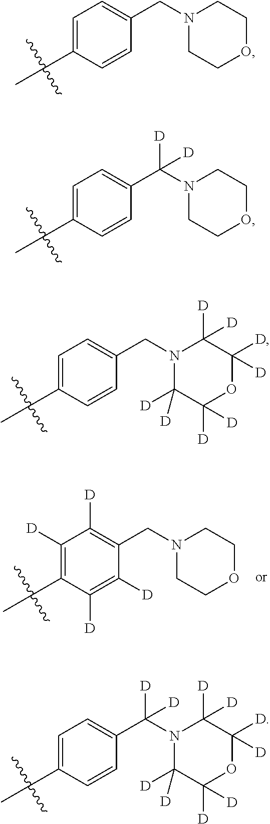

The benzoheterocyclic compound as shown in formula (I) of the present invention is preferably any one of the following compounds:

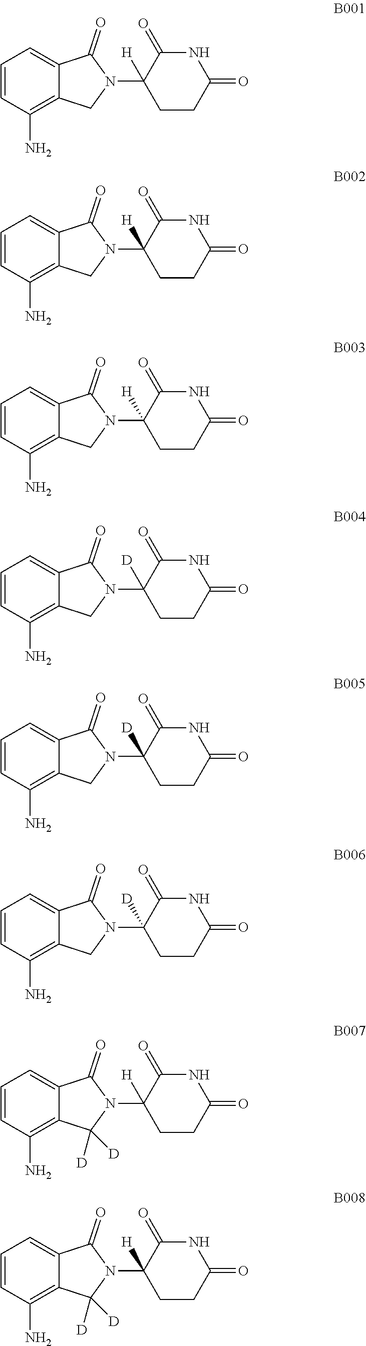

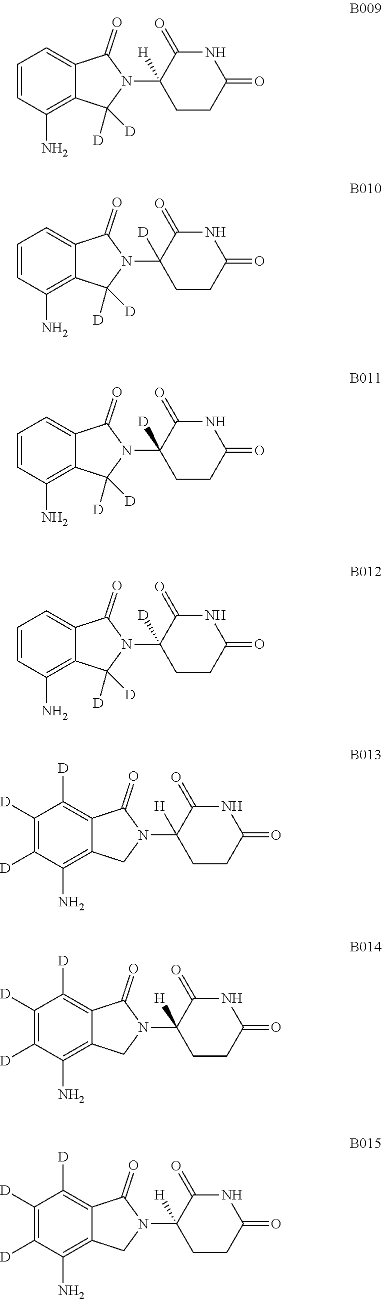

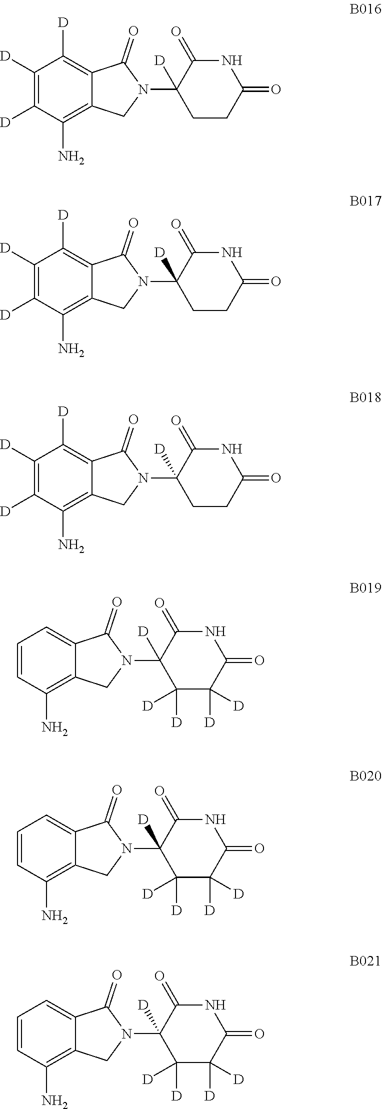 [SMILES: [2H]c1c([2H])c(N)c2c(c1[2H])C(=O)N(C1([2H])CCC(=O)NC1=O)C2 ; [2H]c1c([2H])c(N)c2c(c1[2H])C(=O)N([C@@]1([2H])CCC(=O)NC1=O)C2 ; [2H]c1c([2H])c(N)c2c(c1[2H])C(=O)N([C@]1([2H])CCC(=O)NC1=O)C2 ; [2H]C1([2H])C(=O)NC(=O)C([2H])(N2Cc3c(N)cccc3C2=O)C1([2H])[2H] ; [2H]C1([2H])C(=O)NC(=O)[C@@]([2H])(N2Cc3c(N)cccc3C2=O)C1([2H])[2H] ; [2H]C1([2H])C(=O)NC(=O)[C@]([2H])(N2Cc3c(N)cccc3C2=O)C1([2H])[2H]]

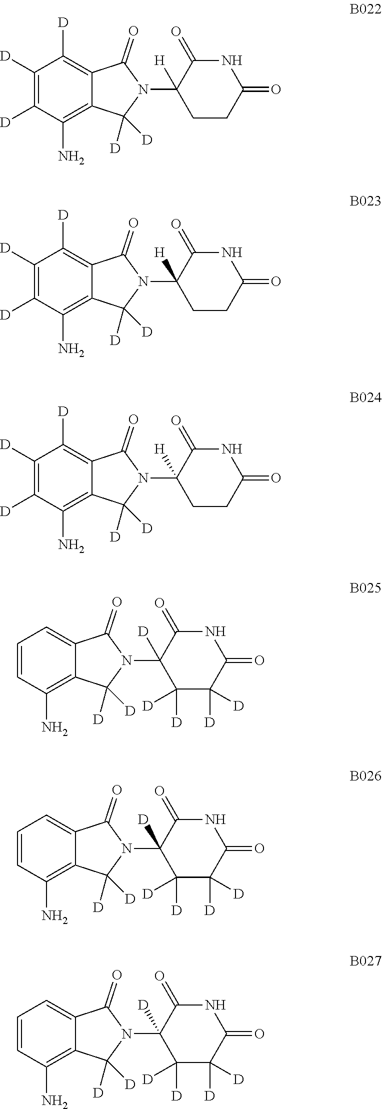 [SMILES: [2H]c1c([2H])c(N)c2c(c1[2H])C(=O)N(C1CCC(=O)NC1=O)C2([2H])[2H] ; [2H]c1c([2H])c(N)c2c(c1[2H])C(=O)N([C@H]1CCC(=O)NC1=O)C2([2H])[2H] ; [2H]c1c([2H])c(N)c2c(c1[2H])C(=O)N([C@@H]1CCC(=O)NC1=O)C2([2H])[2H] ; [2H]C1([2H])c2c(N)cccc2C(=O)N1C1([2H])C(=O)NC(=O)C([2H])([2H])C1([2H])[2H] ; [2H]C1([2H])c2c(N)cccc2C(=O)N1[C@]1([2H])C(=O)NC(=O)C([2H])([2H])C1([2H])[2H] ; [2H]C1([2H])c2c(N)cccc2C(=O)N1[C@@]1([2H])C(=O)NC(=O)C([2H])([2H])C1([2H])[2H]]

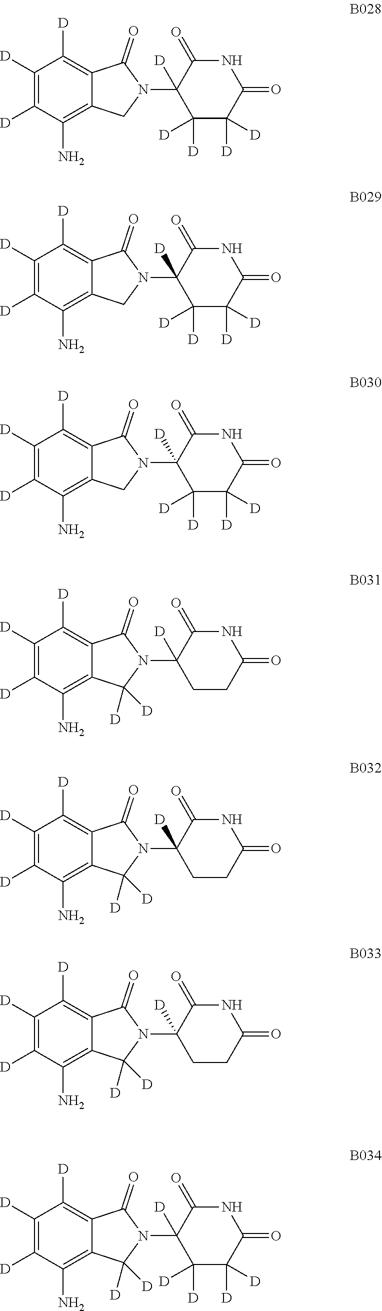

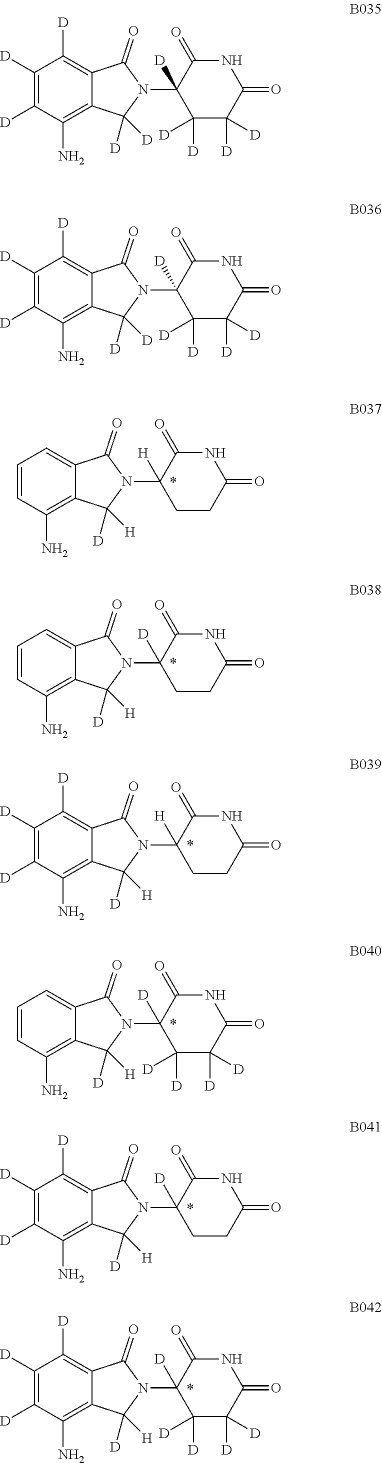

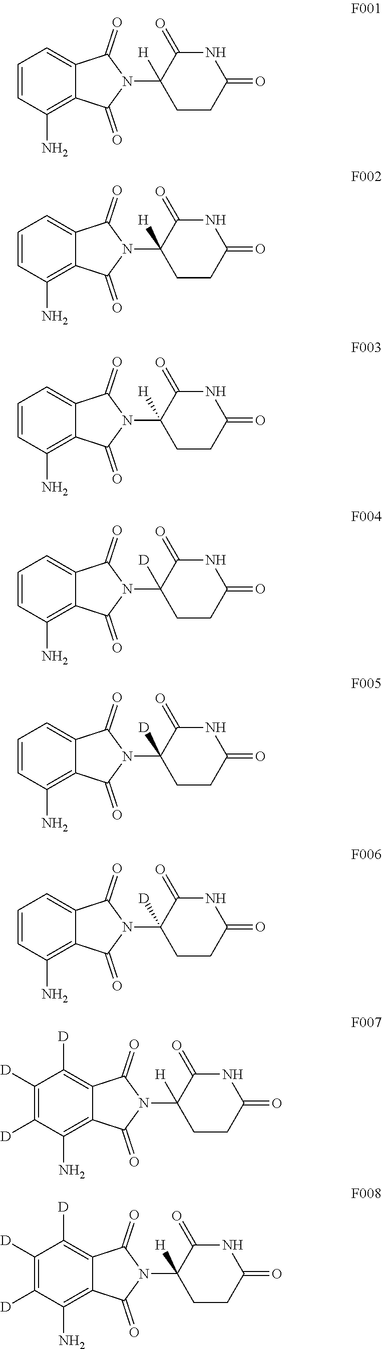

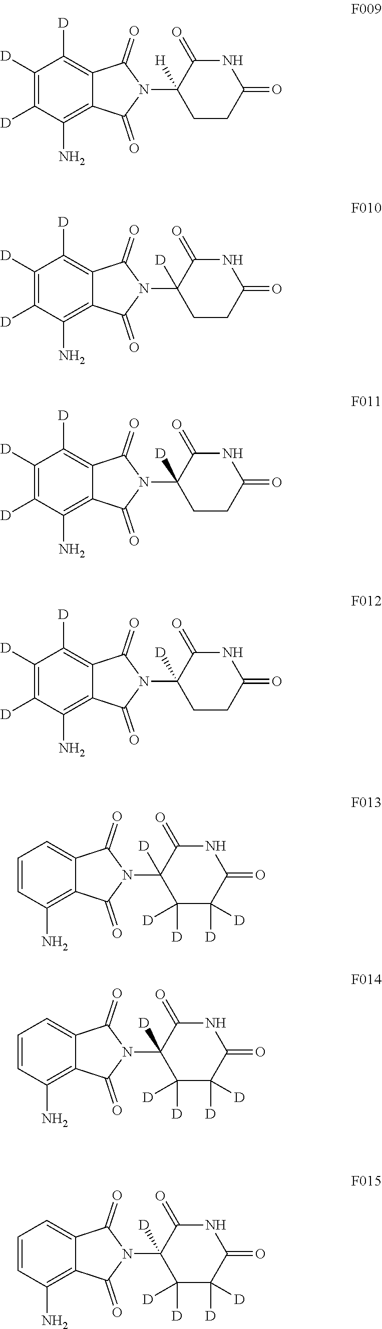

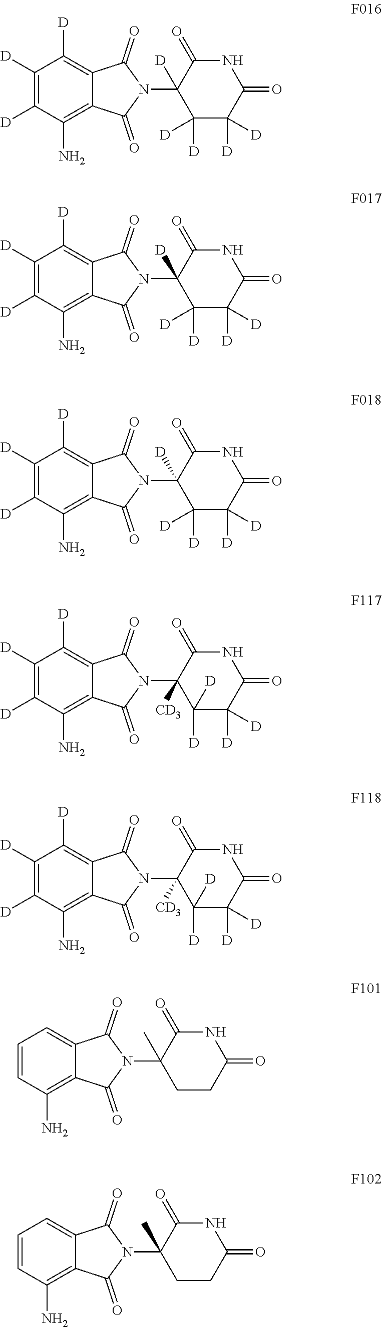

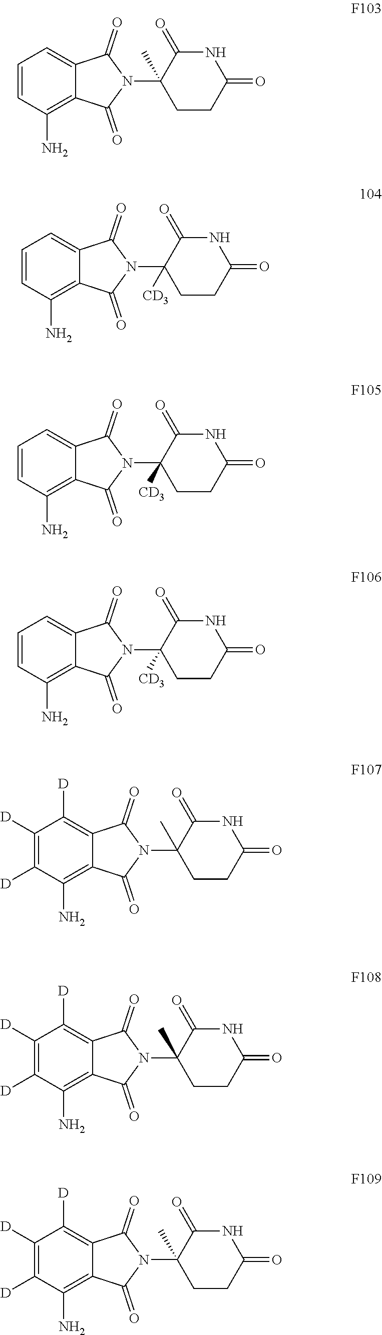

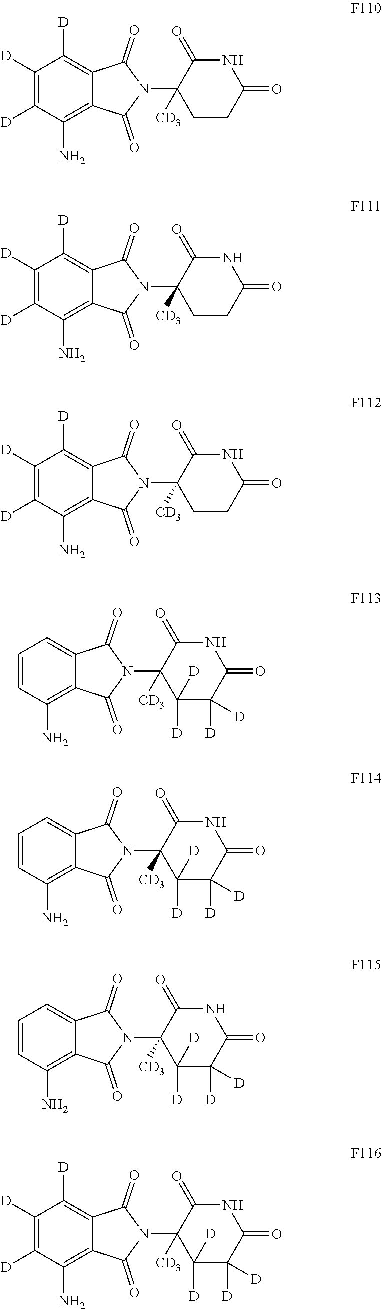

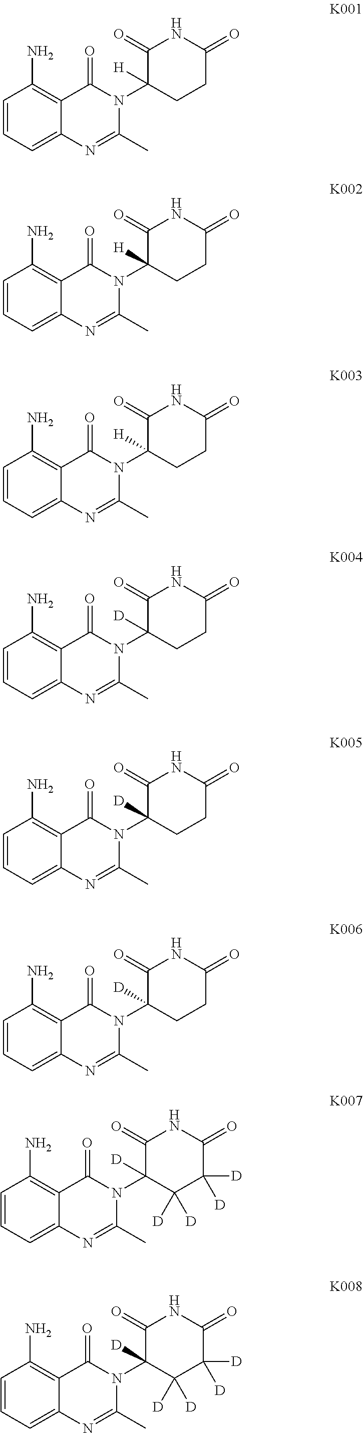

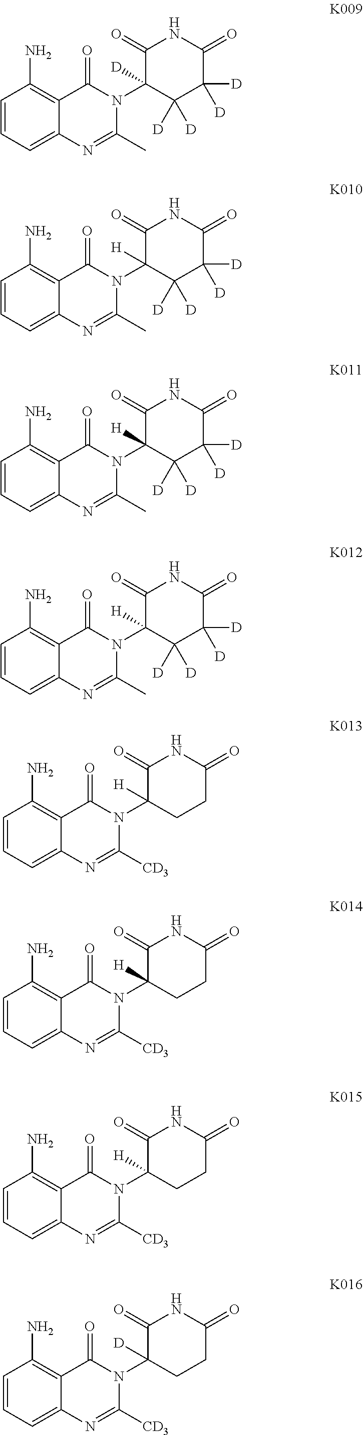

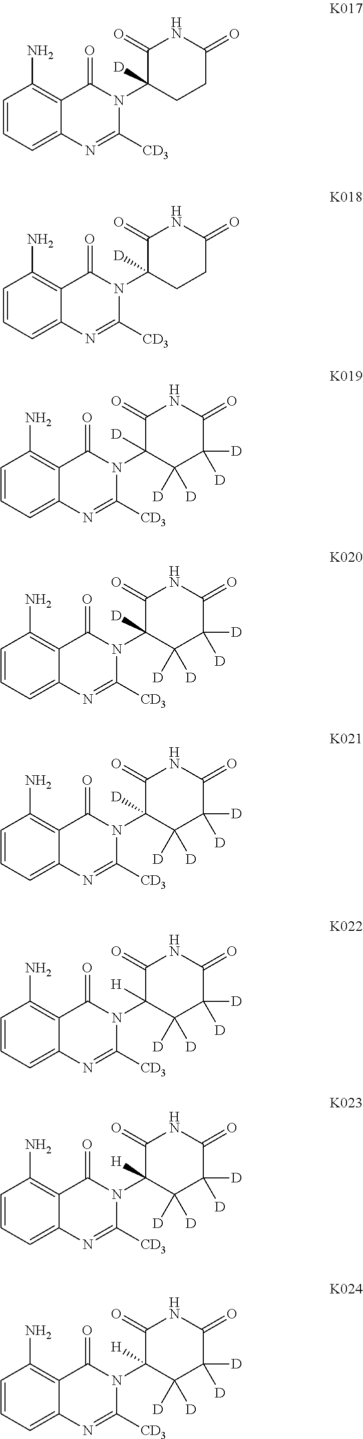

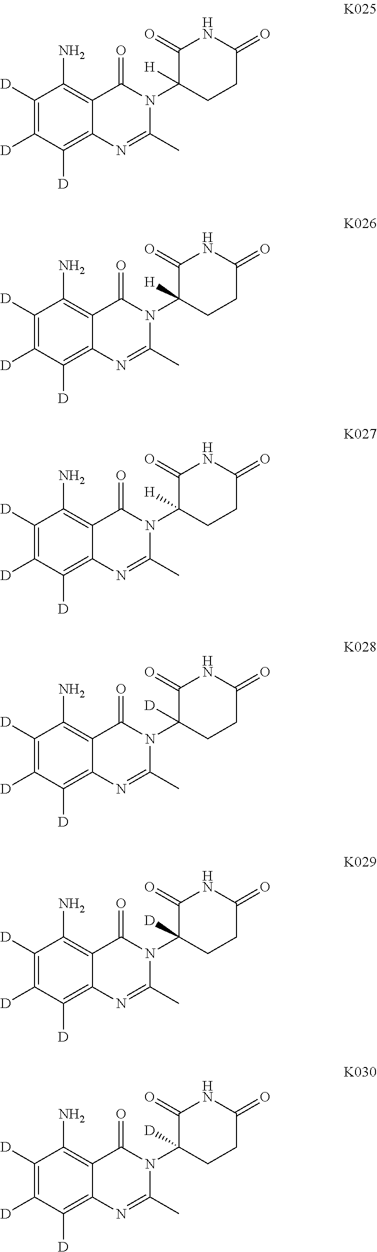 [SMILES: [2H]c1c([2H])c(N)c2c(=O)n(C3CCC(=O)NC3=O)c(C)nc2c1[2H] ; [2H]c1c([2H])c(N)c2c(=O)n([C@H]3CCC(=O)NC3=O)c(C)nc2c1[2H] ; [2H]c1c([2H])c(N)c2c(=O)n([C@@H]3CCC(=O)NC3=O)c(C)nc2c1[2H] ; [2H]c1c([2H])c(N)c2c(=O)n(C3([2H])CCC(=O)NC3=O)c(C)nc2c1[2H] ; [2H]c1c([2H])c(N)c2c(=O)n([C@@]3([2H])CCC(=O)NC3=O)c(C)nc2c1[2H] ; [2H]c1c([2H])c(N)c2c(=O)n([C@]3([2H])CCC(=O)NC3=O)c(C)nc2c1[2H]]

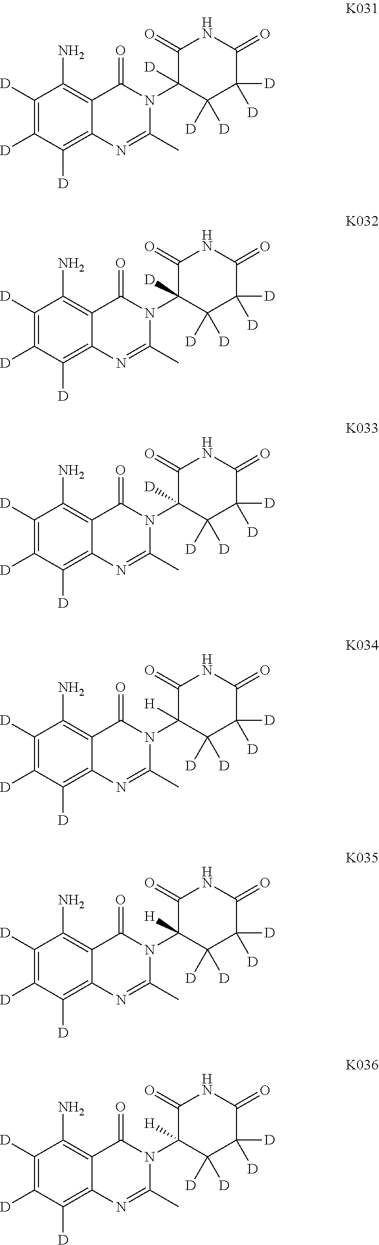

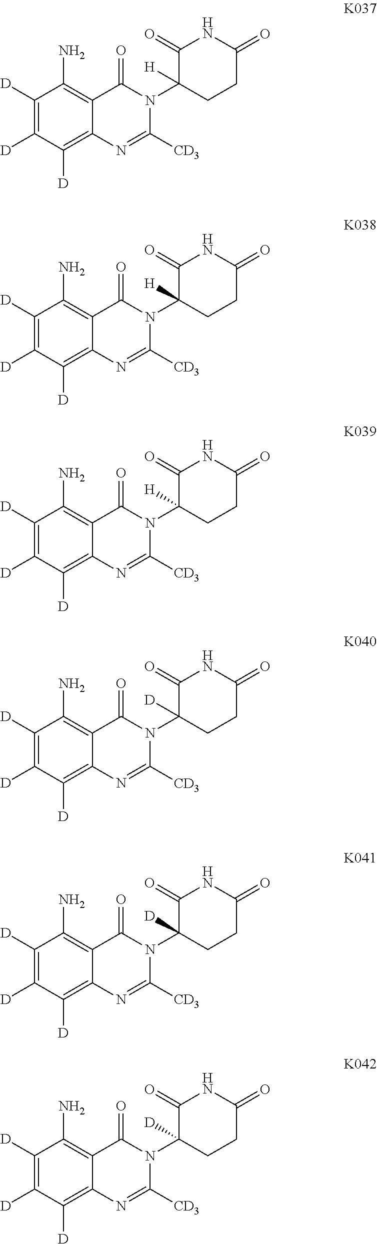 [SMILES: [2H]c1c([2H])c(N)c2c(=O)n(C3CCC(=O)NC3=O)c(C([2H])([2H])[2H])nc2c1[2H] ; [2H]c1c([2H])c(N)c2c(=O)n([C@H]3CCC(=O)NC3=O)c(C([2H])([2H])[2H])nc2c1[2H] ; [2H]c1c([2H])c(N)c2c(=O)n([C@@H]3CCC(=O)NC3=O)c(C([2H])([2H])[2H])nc2c1[2H] ; [2H]c1c([2H])c(N)c2c(=O)n(C3([2H])CCC(=O)NC3=O)c(C([2H])([2H])[2H])nc2c1[2H] ; [2H]c1c([2H])c(N)c2c(=O)n([C@@]3([2H])CCC(=O)NC3=O)c(C([2H])([2H])[2H])nc2c1[2H] ; [2H]c1c([2H])c(N)c2c(=O)n([C@]3([2H])CCC(=O)NC3=O)c(C([2H])([2H])[2H])nc2c1[2H]]

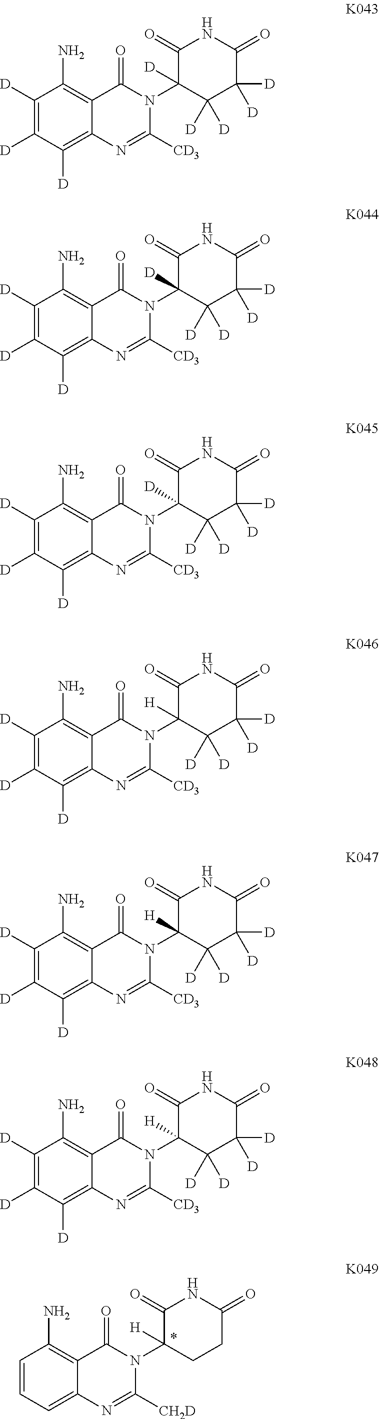

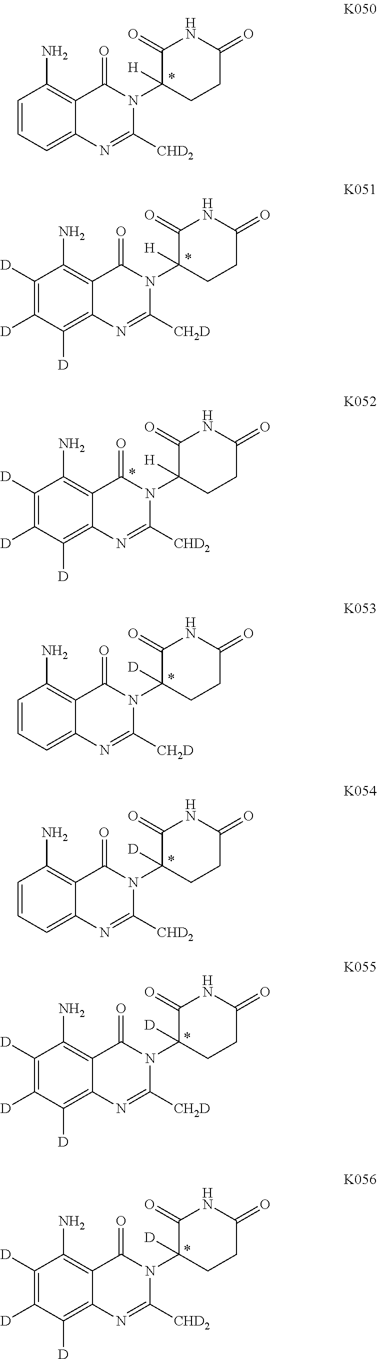

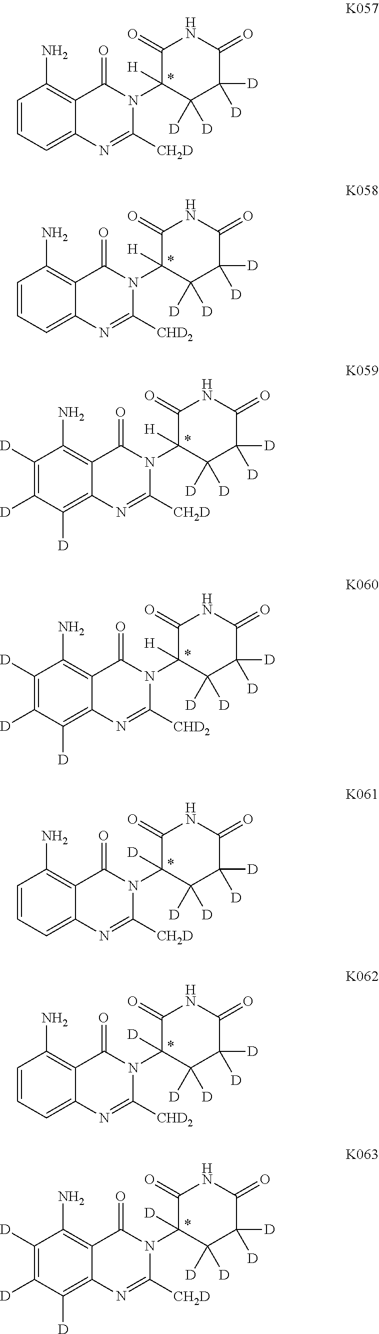

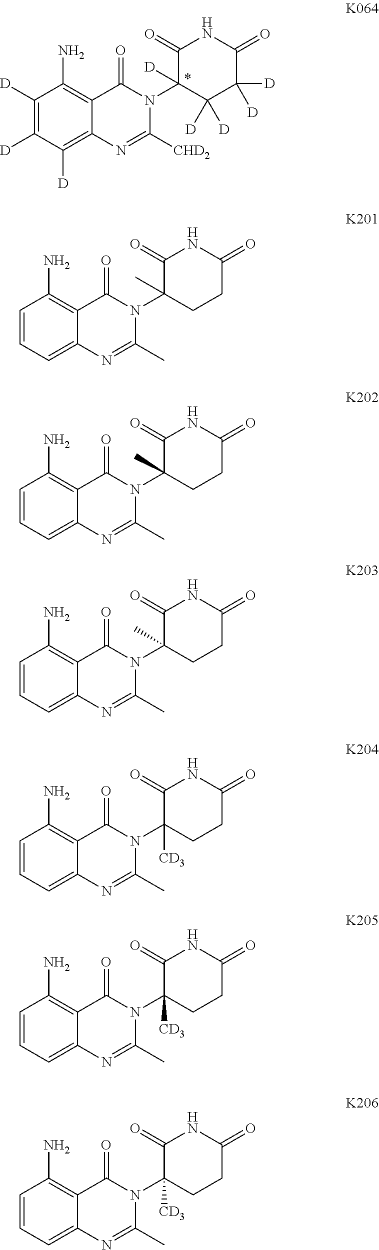

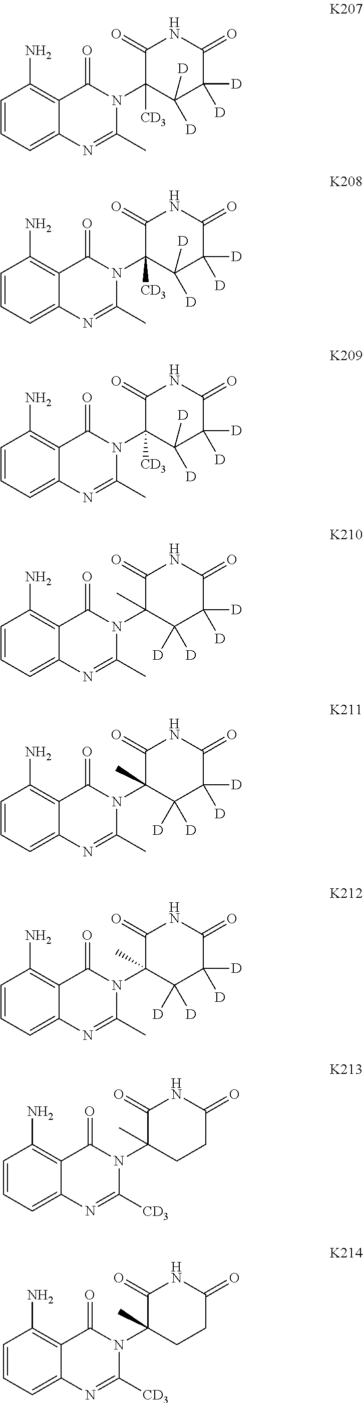

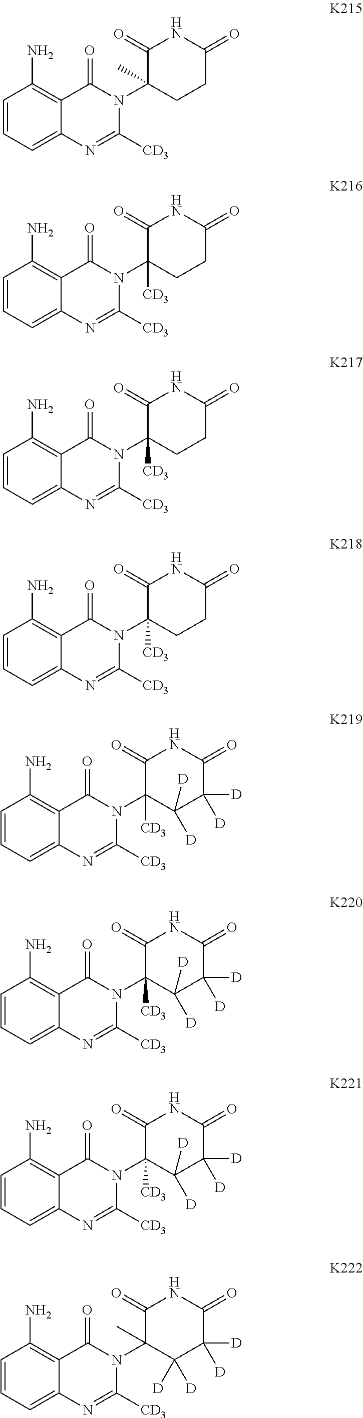

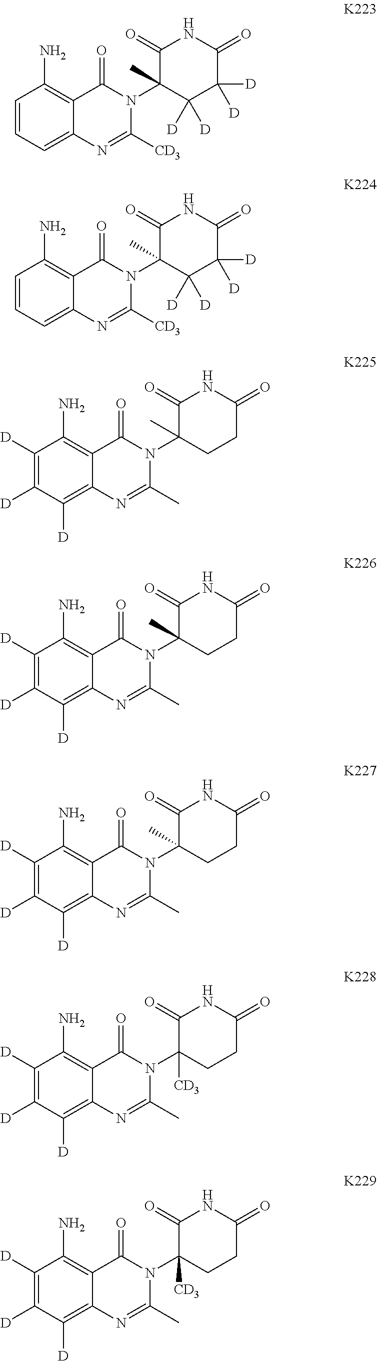

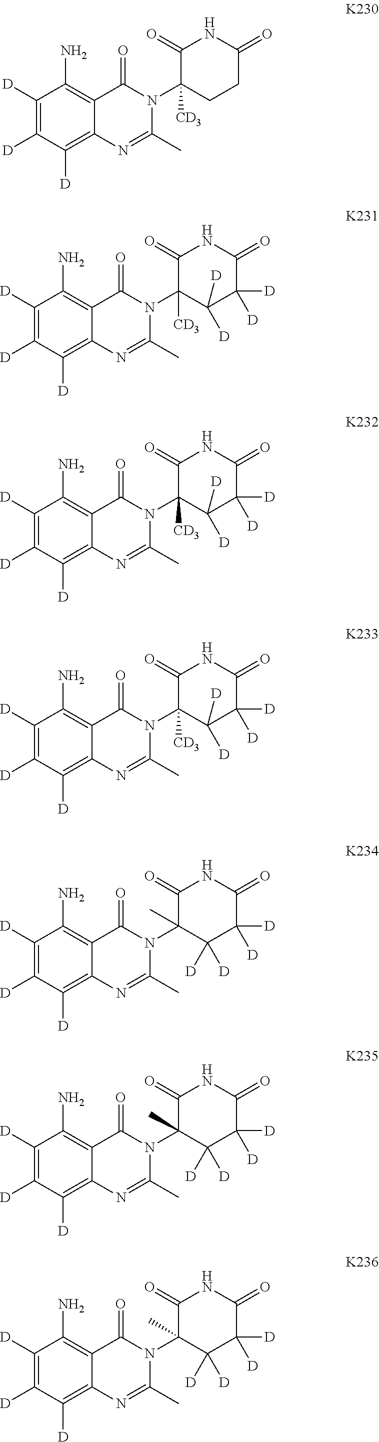

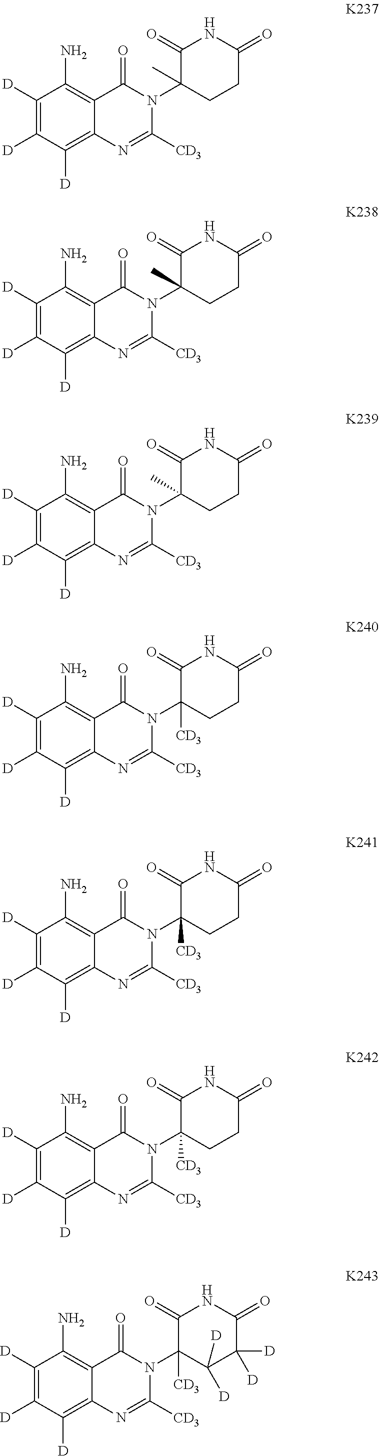

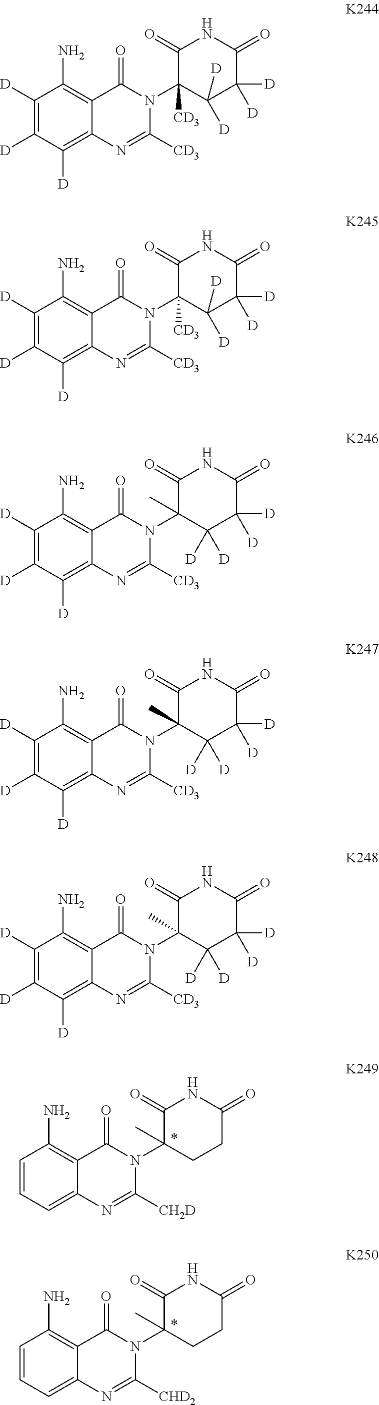

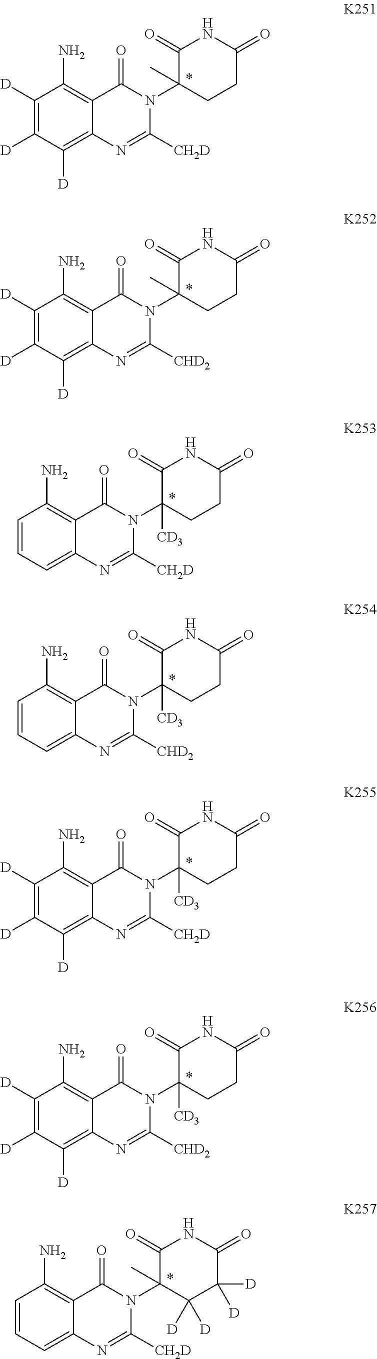

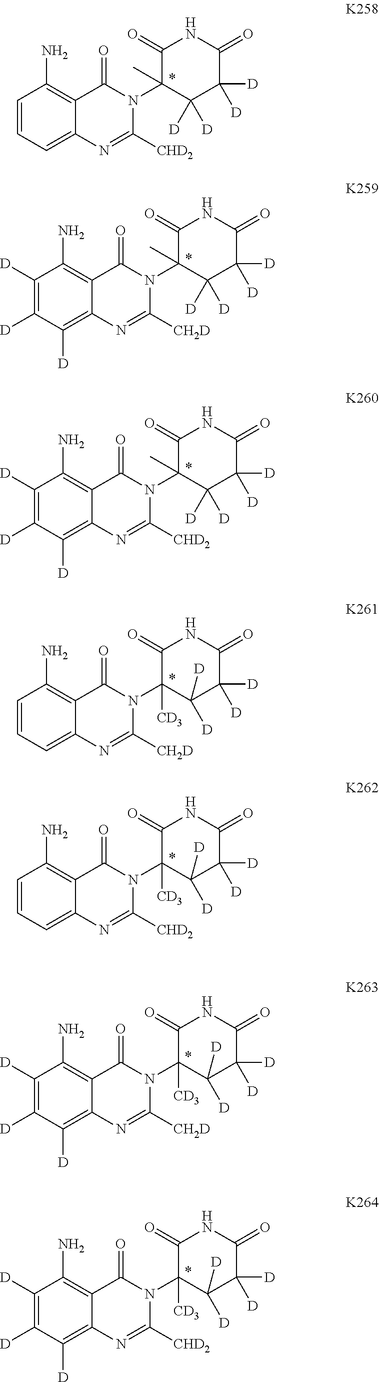

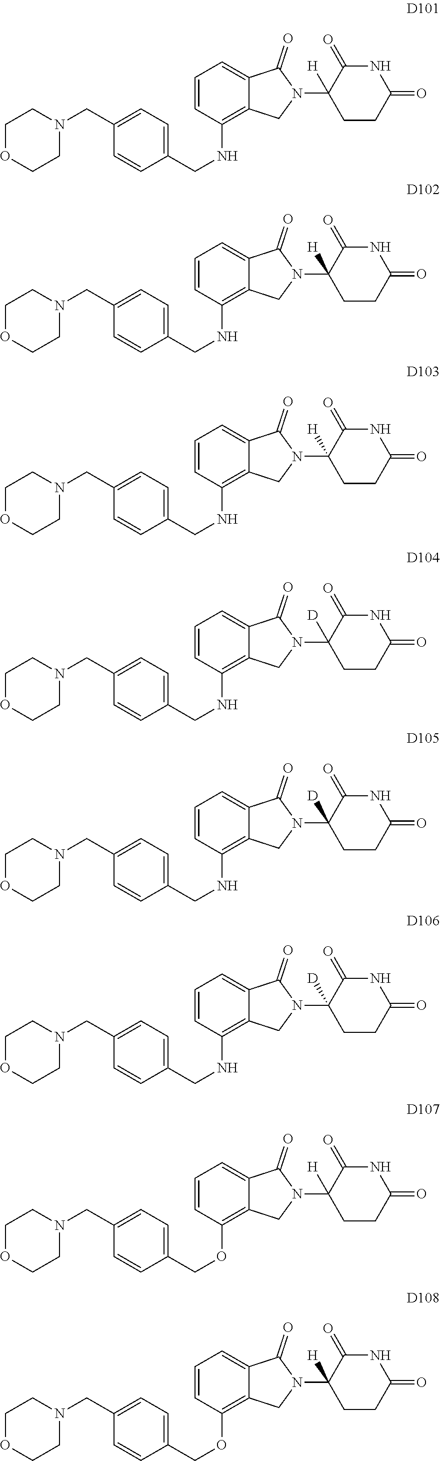

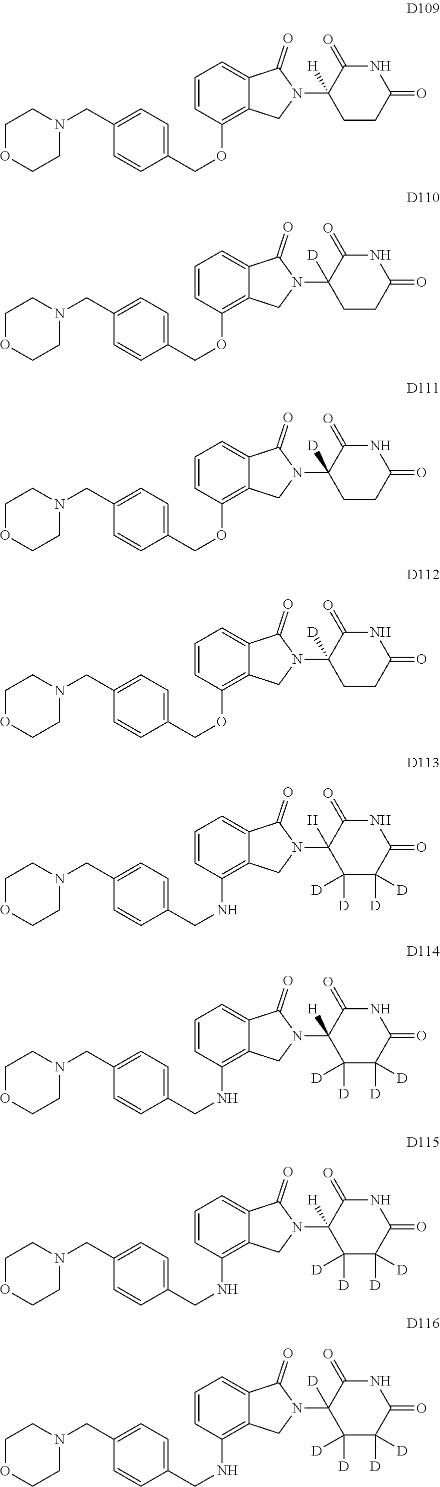

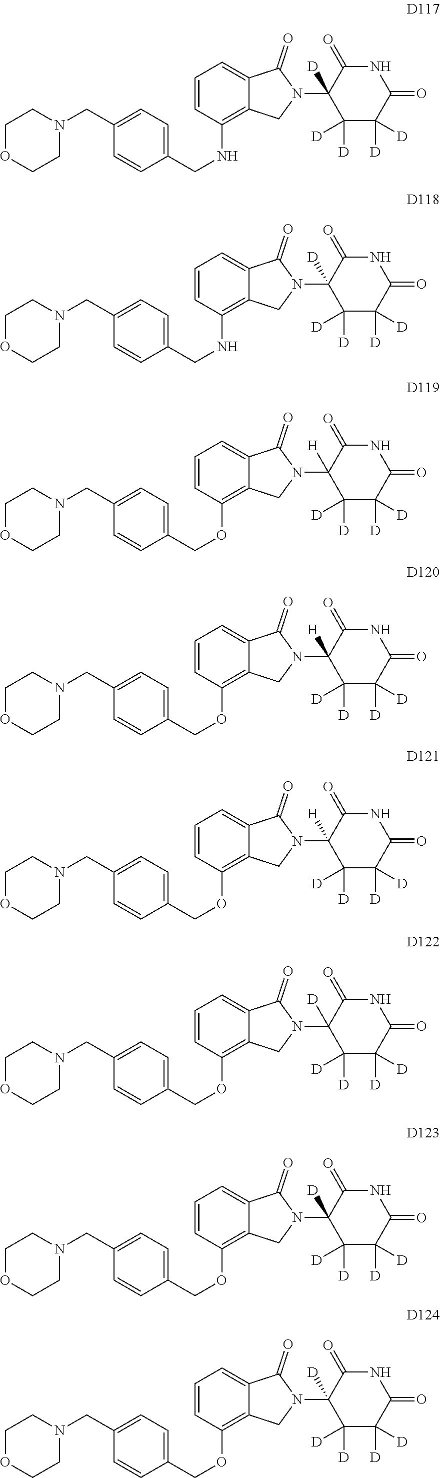

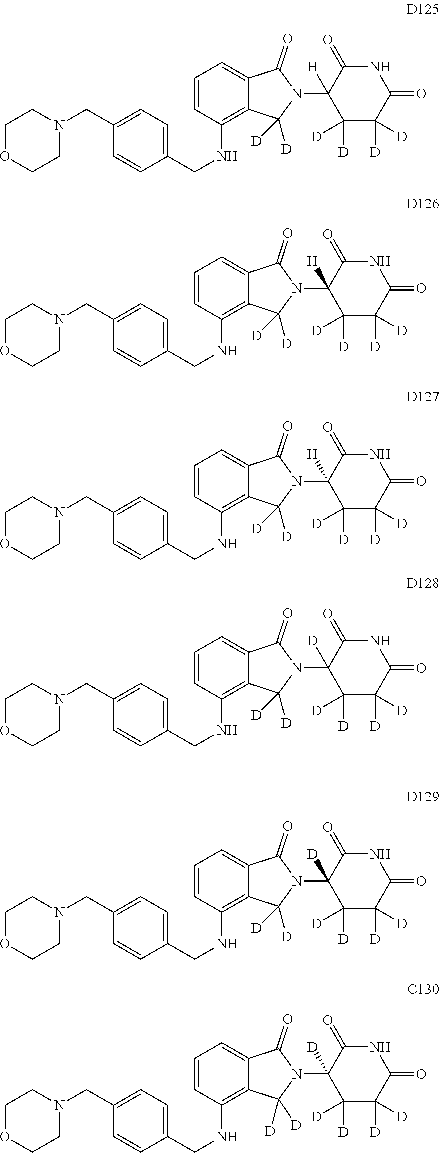

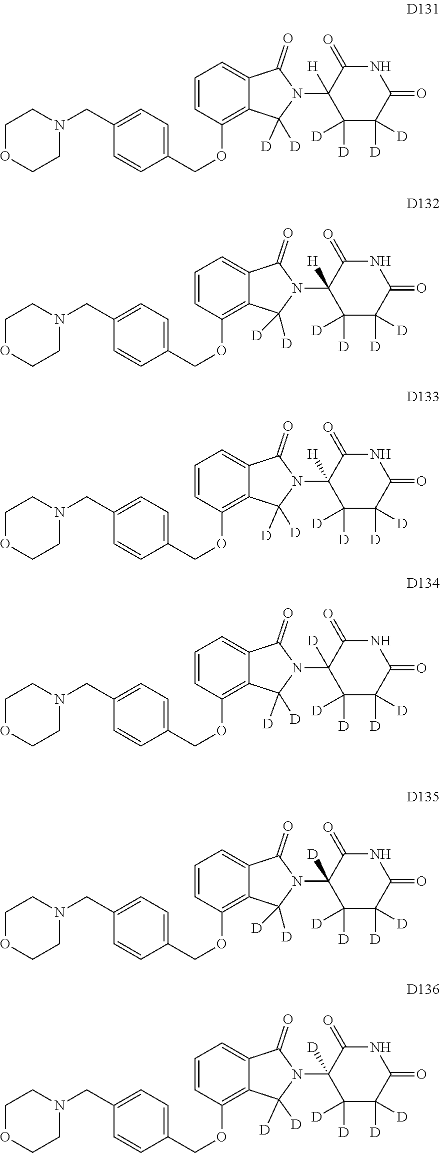

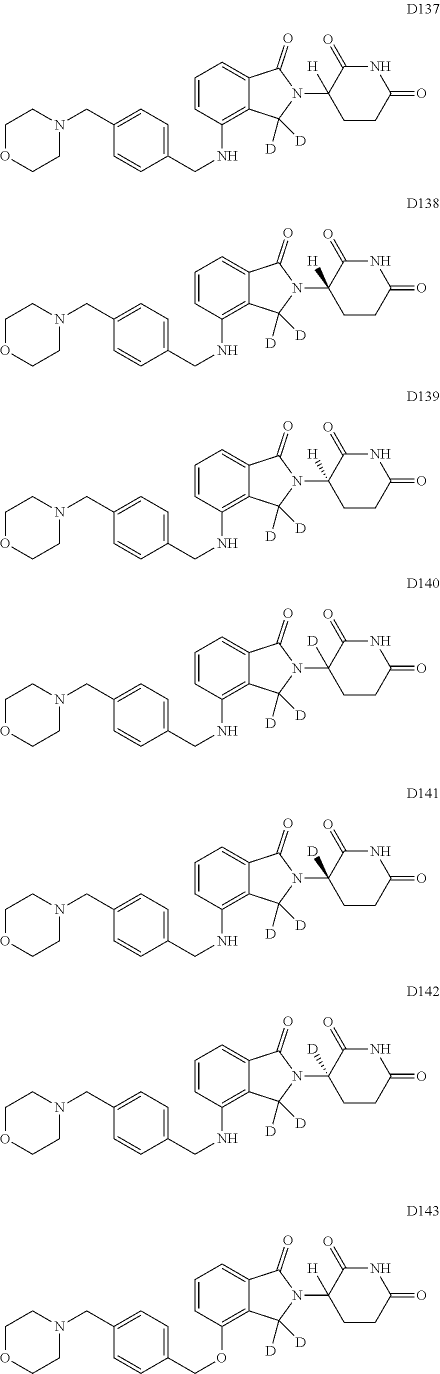

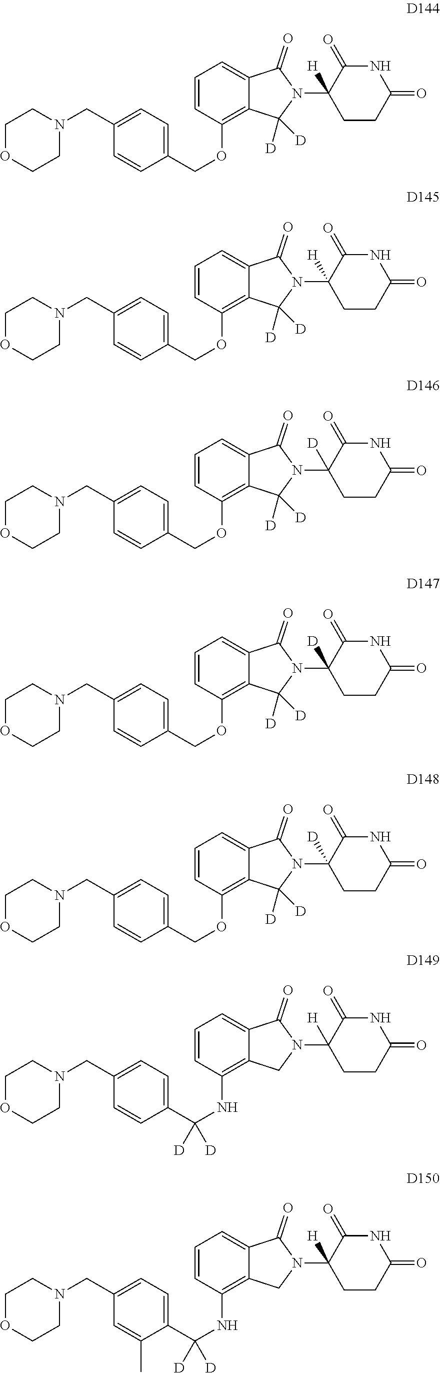

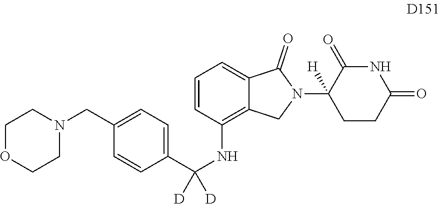 [SMILES: [2H]C([2H])(Nc1cccc2c1CN([C@@H]1CCC(=O)NC1=O)C2=O)c1ccc(CN2CCOCC2)cc1]

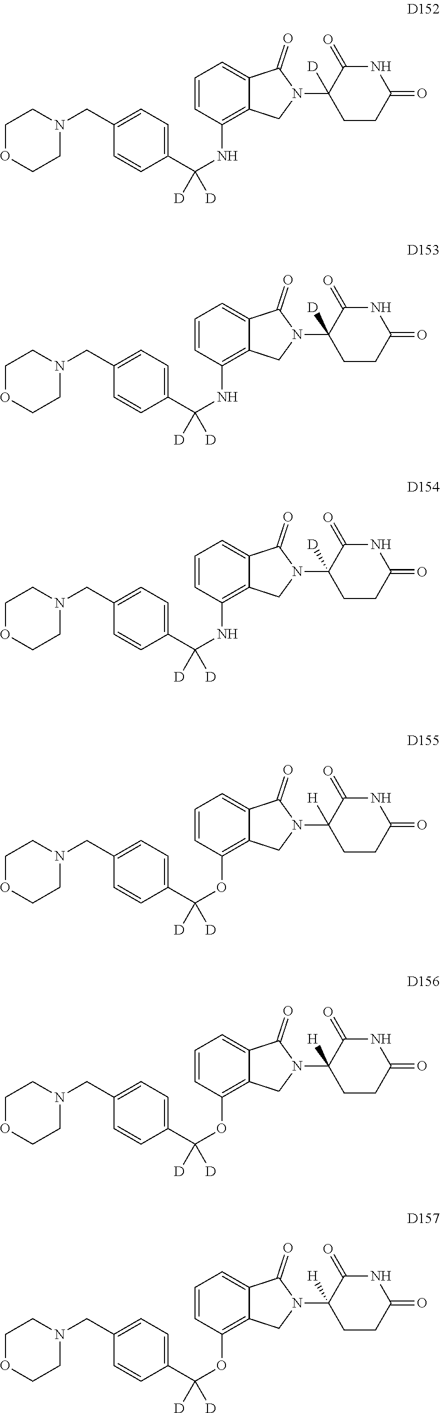 [SMILES: [2H]C([2H])(Nc1cccc2c1CN(C1([2H])CCC(=O)NC1=O)C2=O)c1ccc(CN2CCOCC2)cc1 ; [2H]C([2H])(Nc1cccc2c1CN([C@@]1([2H])CCC(=O)NC1=O)C2=O)c1ccc(CN2CCOCC2)cc1 ; [2H]C([2H])(Nc1cccc2c1CN([C@]1([2H])CCC(=O)NC1=O)C2=O)c1ccc(CN2CCOCC2)cc1 ; [2H]C([2H])(Oc1cccc2c1CN(C1CCC(=O)NC1=O)C2=O)c1ccc(CN2CCOCC2)cc1 ; [2H]C([2H])(Oc1cccc2c1CN([C@H]1CCC(=O)NC1=O)C2=O)c1ccc(CN2CCOCC2)cc1 ; [2H]C([2H])(Oc1cccc2c1CN([C@@H]1CCC(=O)NC1=O)C2=O)c1ccc(CN2CCOCC2)cc1]

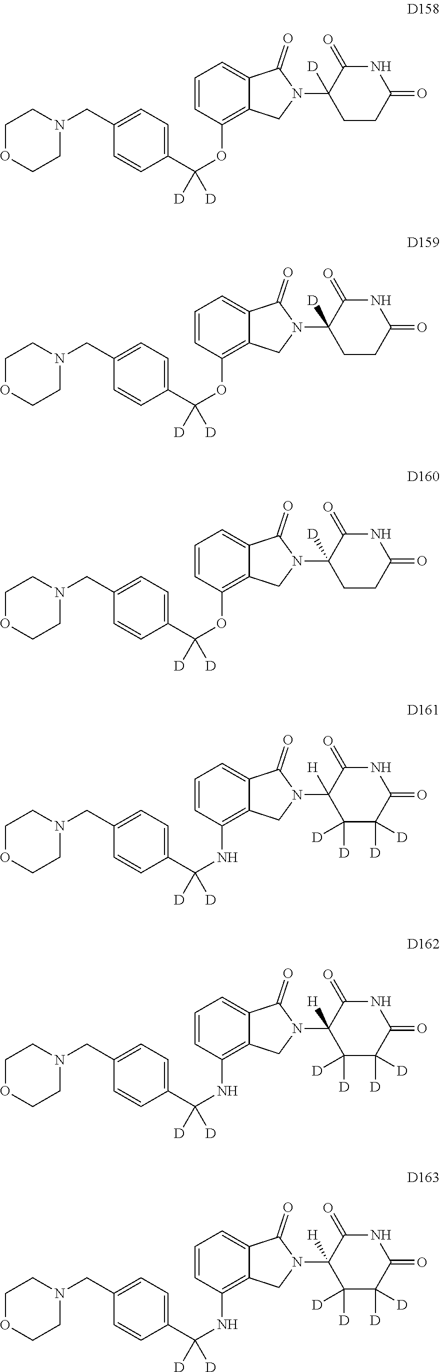

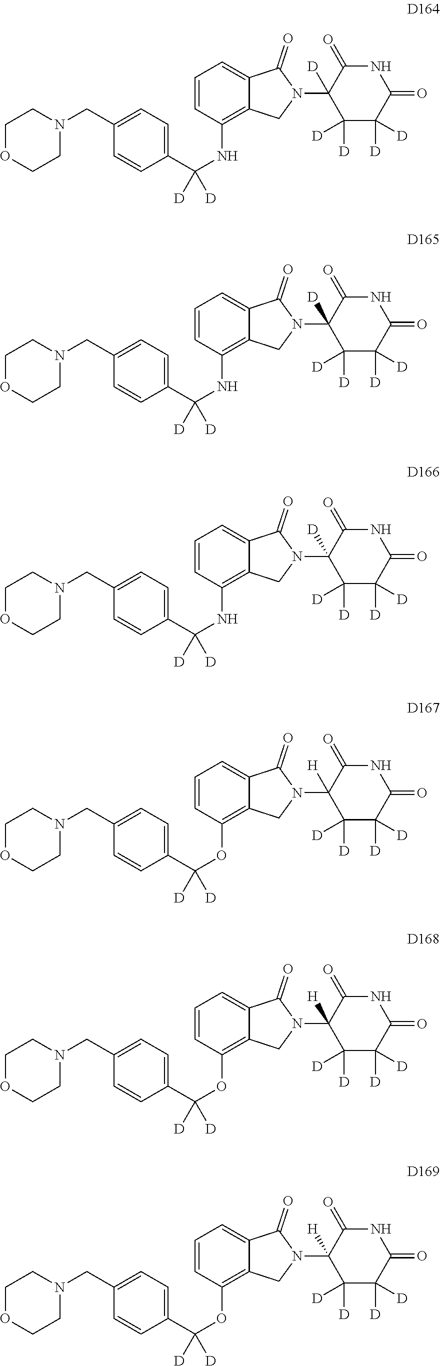

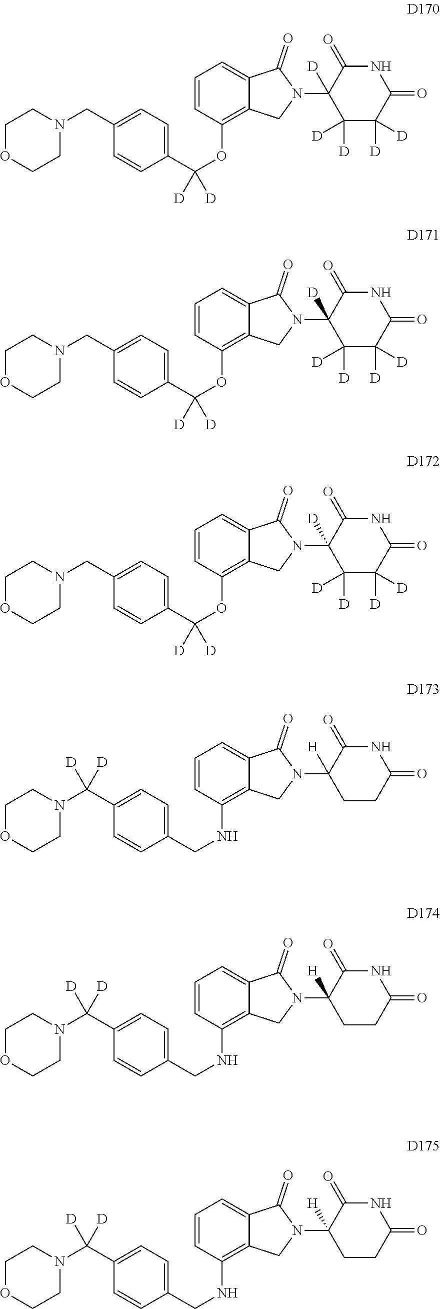

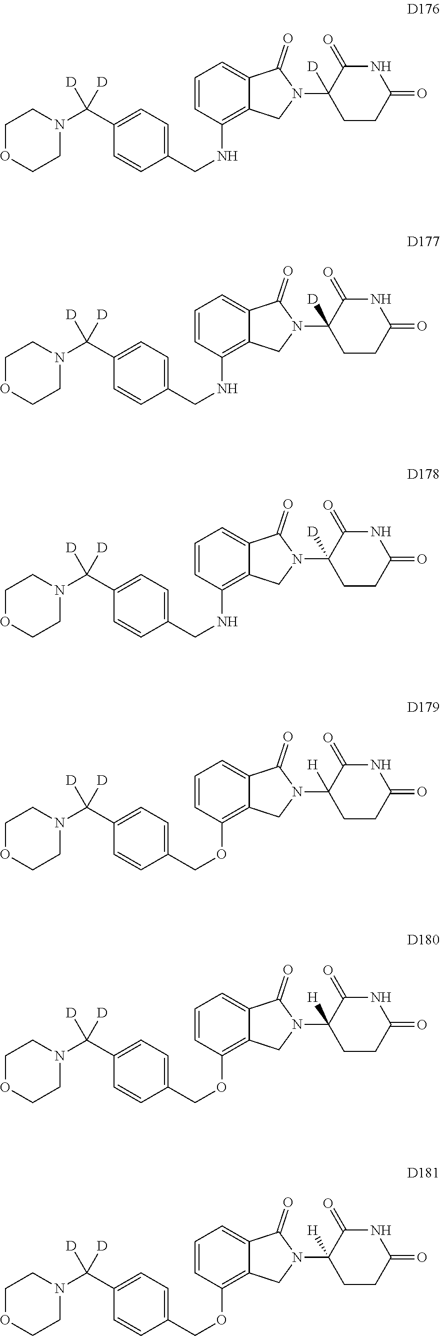 [SMILES: [2H]C1(N2Cc3c(NCc4ccc(C([2H])([2H])N5CCOCC5)cc4)cccc3C2=O)CCC(=O)NC1=O ; [2H]C([2H])(c1ccc(CNc2cccc3c2CN([C@@]2([2H])CCC(=O)NC2=O)C3=O)cc1)N1CCOCC1 ; [2H]C([2H])(c1ccc(CNc2cccc3c2CN([C@]2([2H])CCC(=O)NC2=O)C3=O)cc1)N1CCOCC1 ; [2H]C([2H])(c1ccc(COc2cccc3c2CN(C2CCC(=O)NC2=O)C3=O)cc1)N1CCOCC1 ; [2H]C([2H])(c1ccc(COc2cccc3c2CN([C@H]2CCC(=O)NC2=O)C3=O)cc1)N1CCOCC1 ; [2H]C([2H])(c1ccc(COc2cccc3c2CN([C@@H]2CCC(=O)NC2=O)C3=O)cc1)N1CCOCC1]

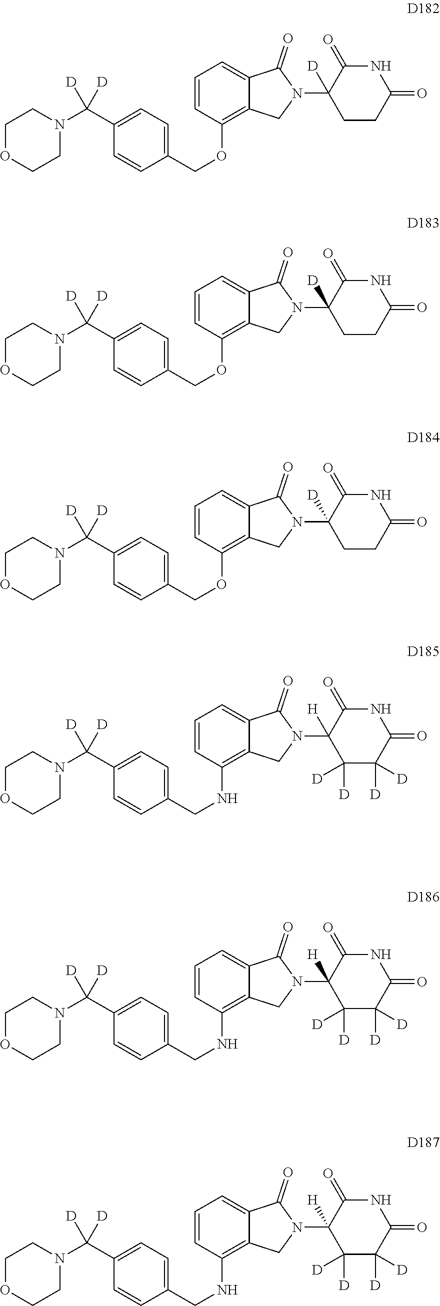

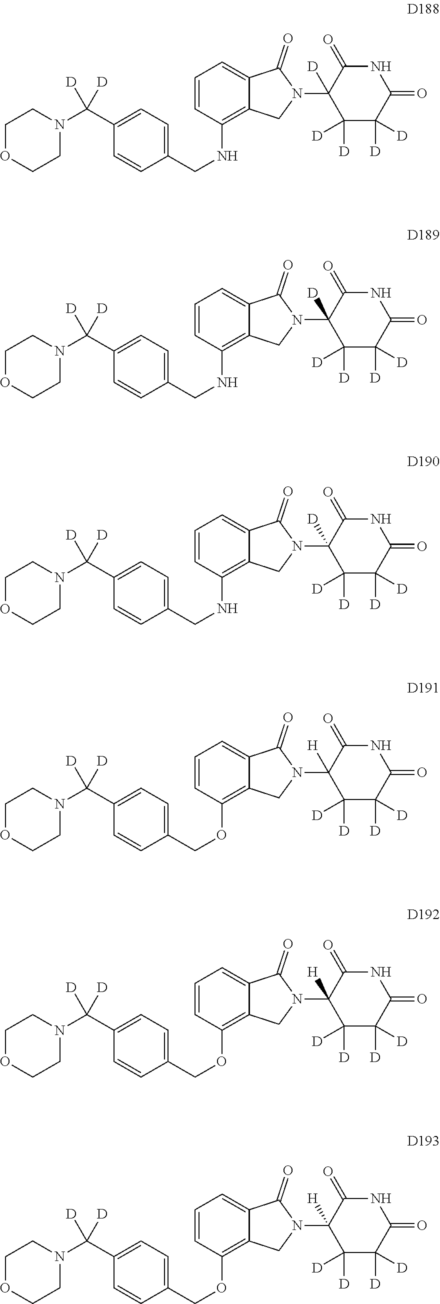

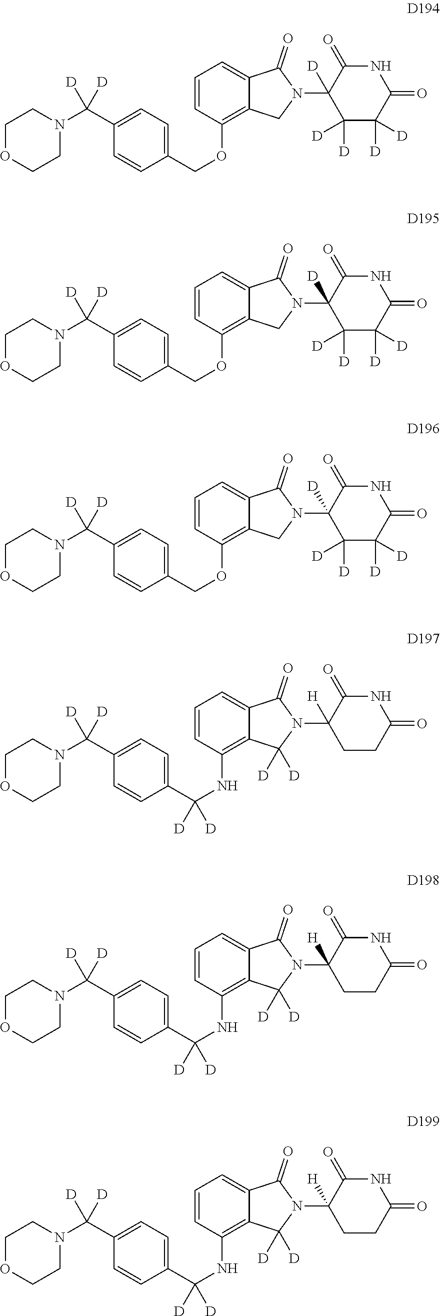

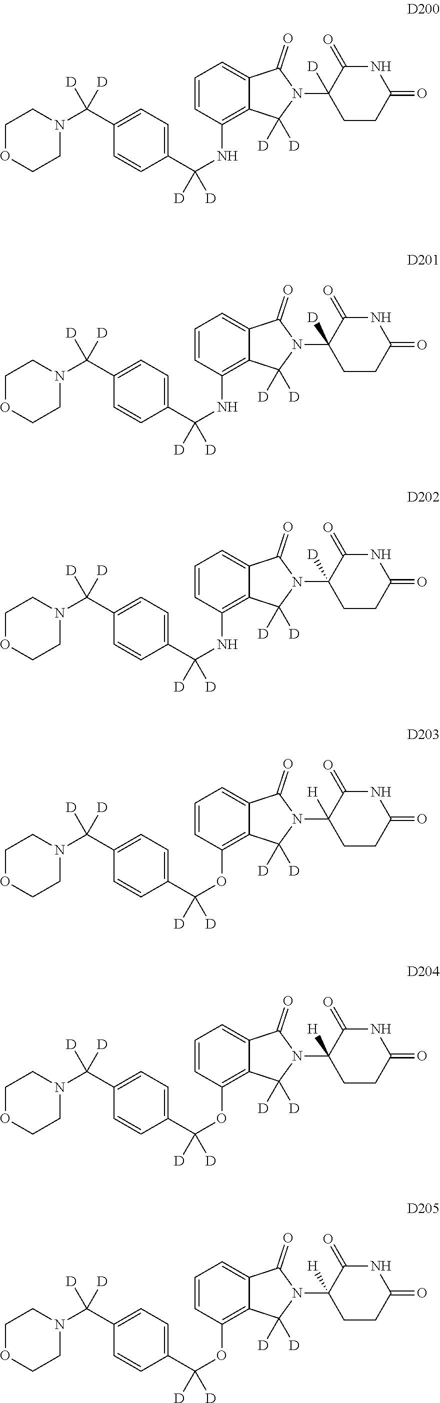

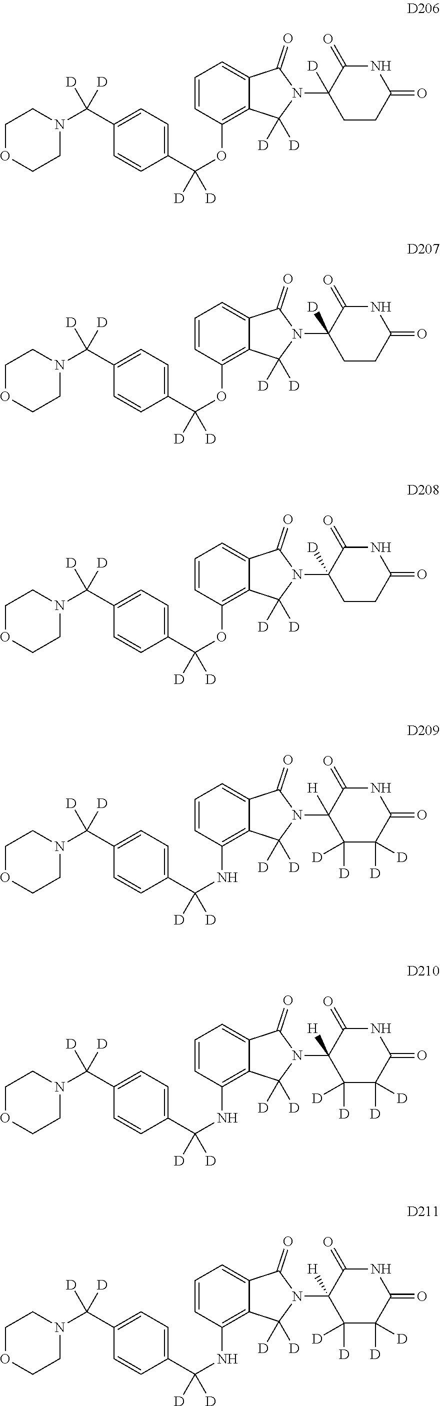

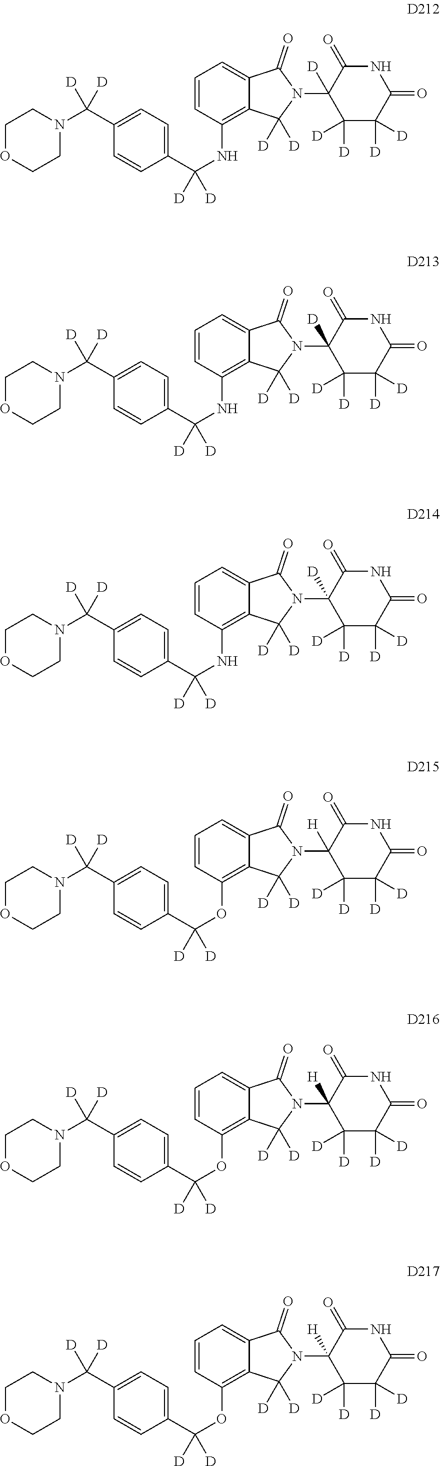

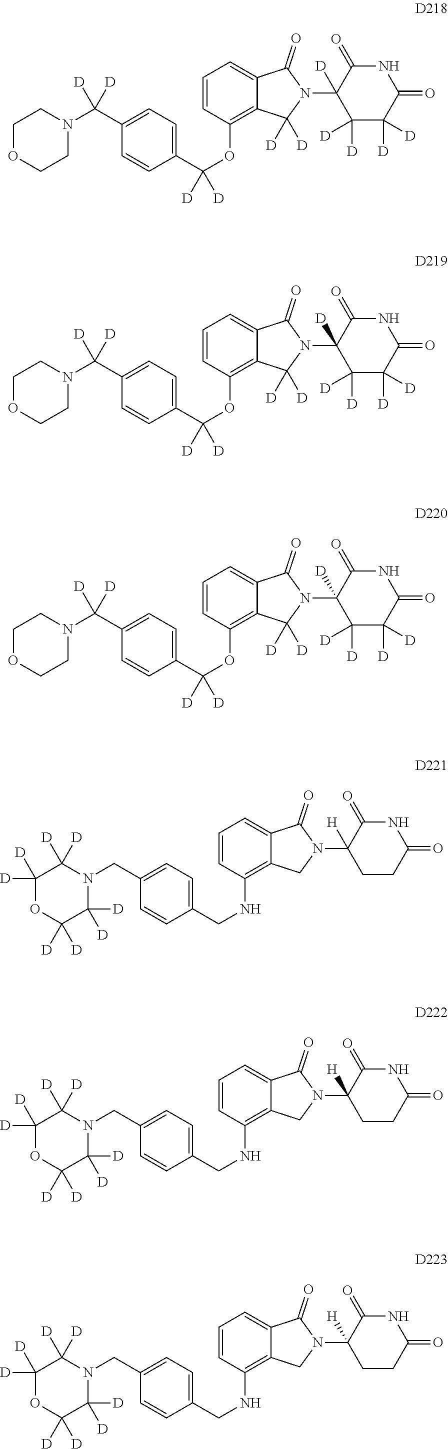

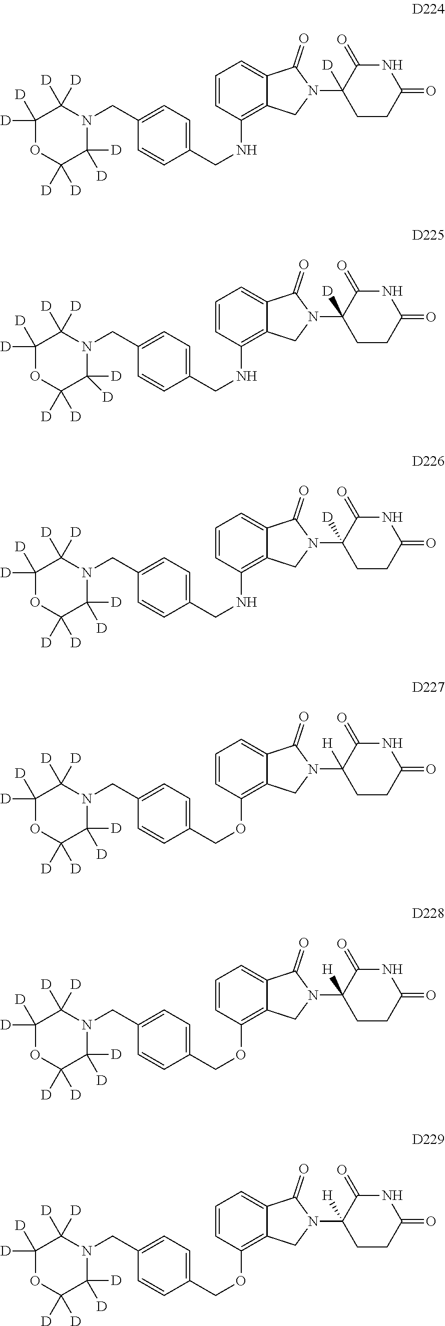

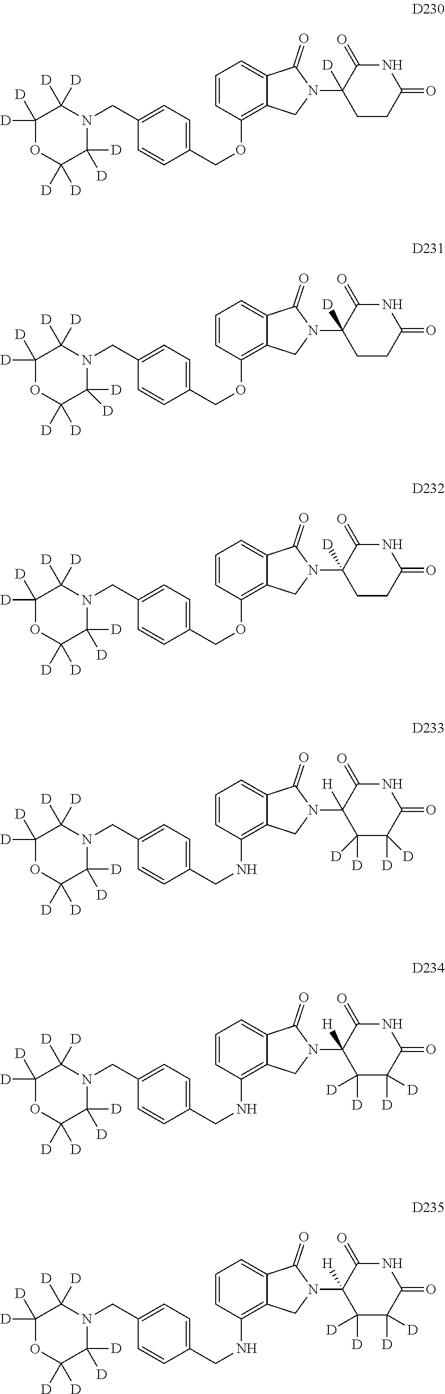

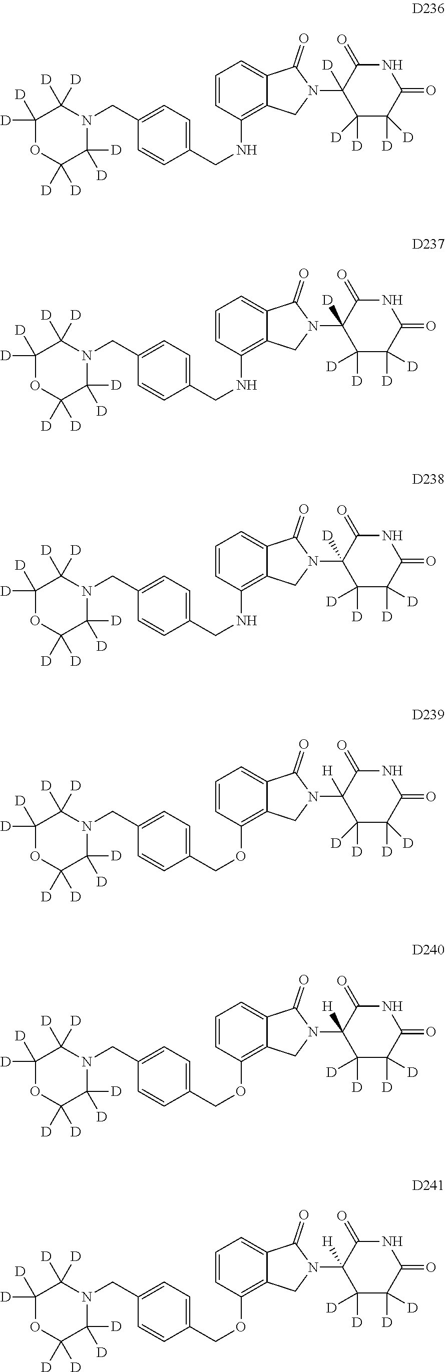

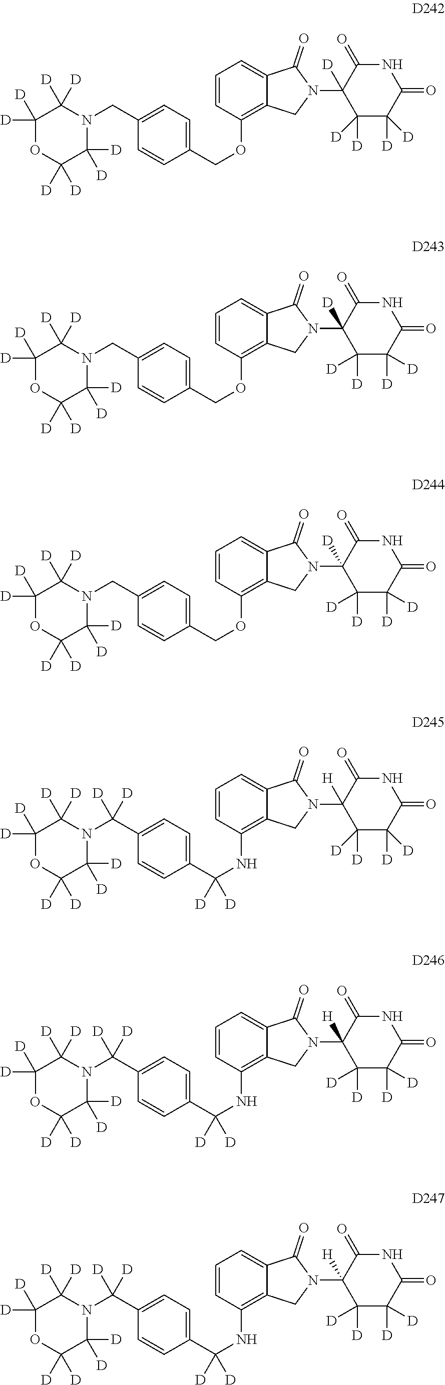

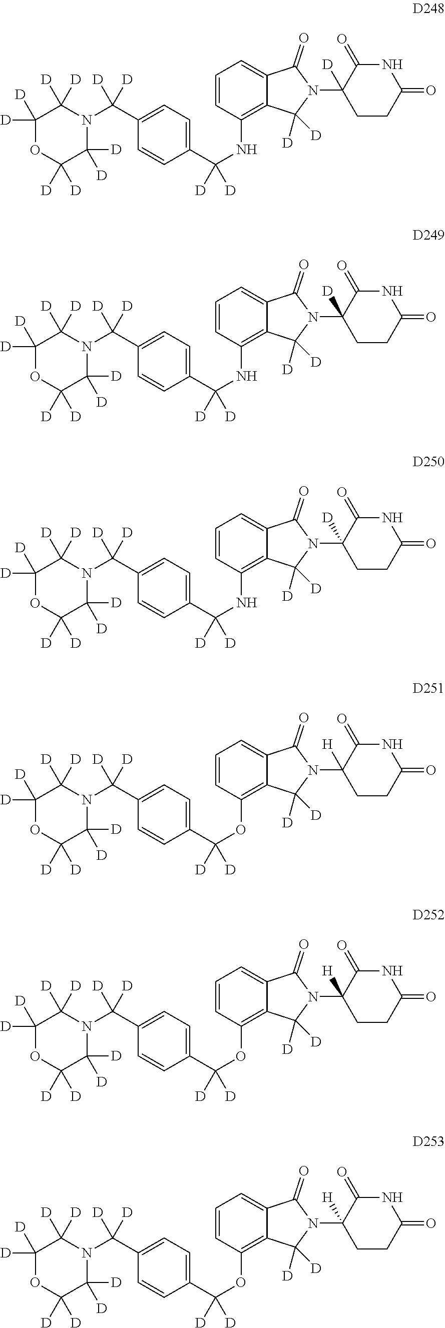

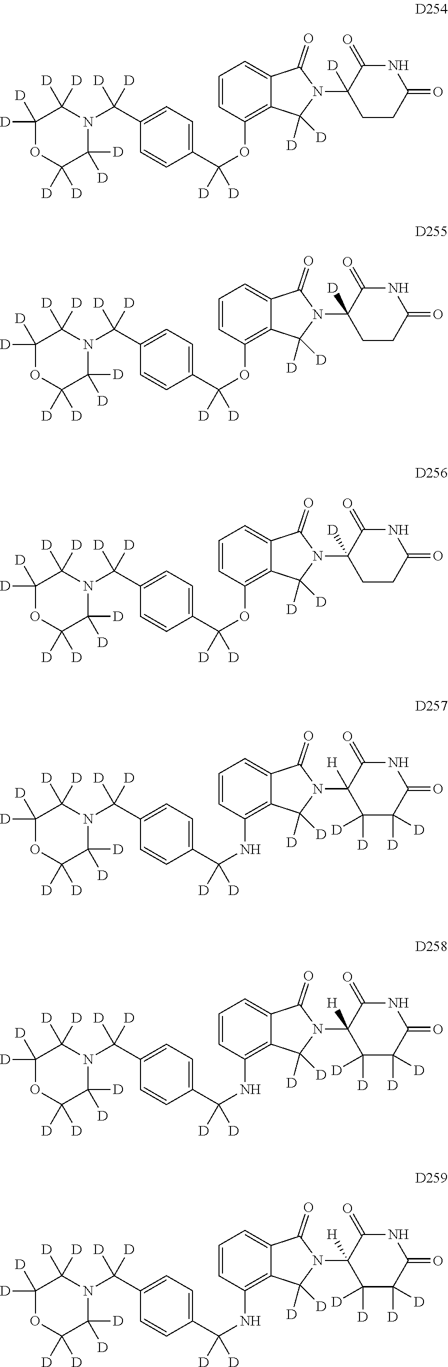

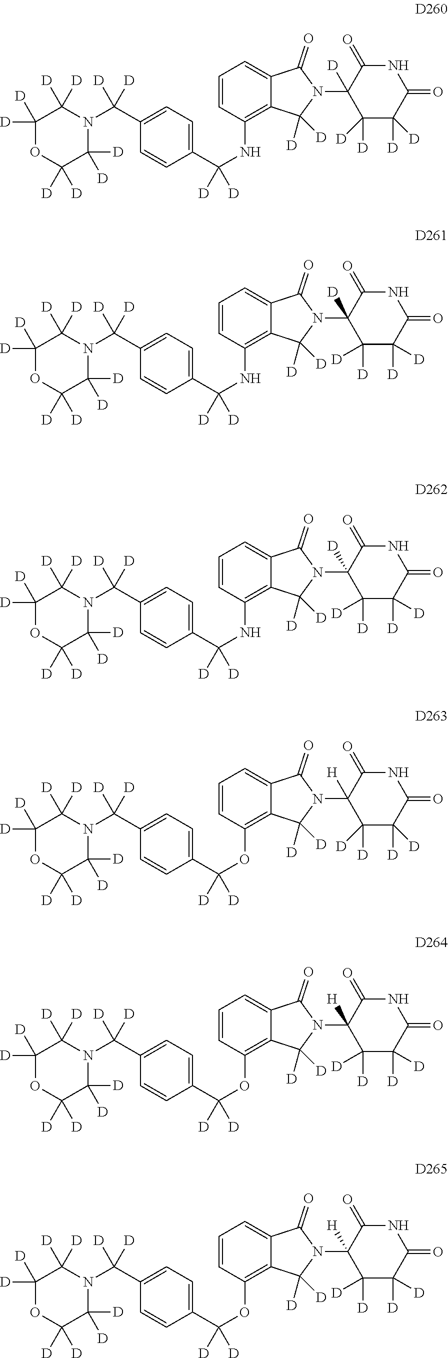

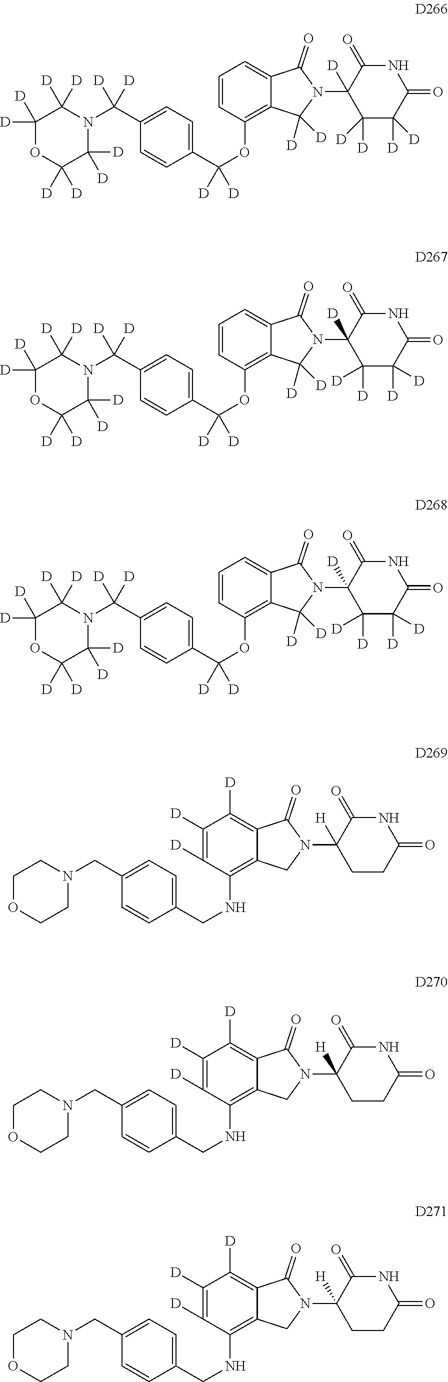

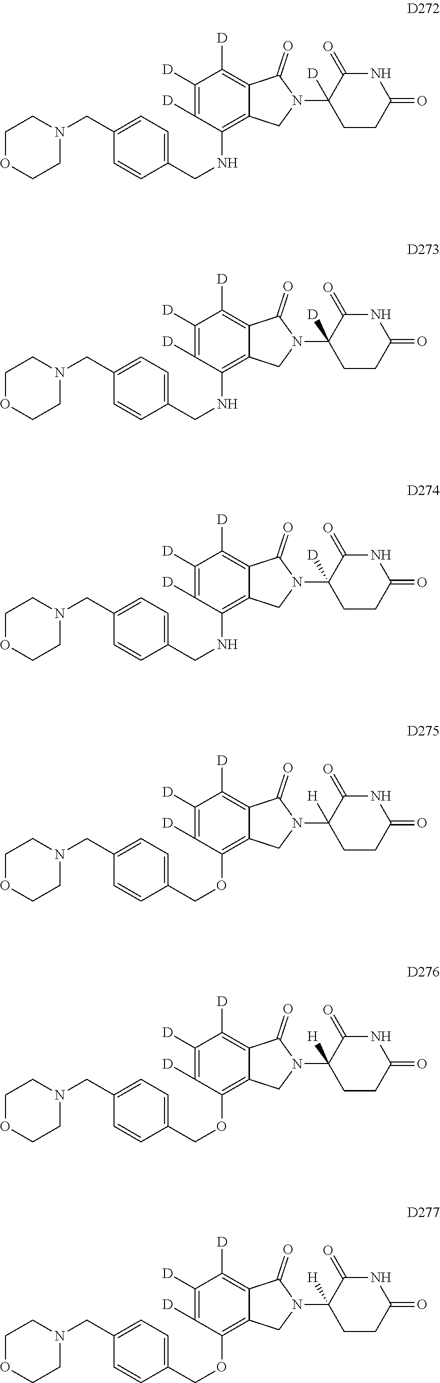 [SMILES: [2H]c1c([2H])c(NCc2ccc(CN3CCOCC3)cc2)c2c(c1[2H])C(=O)N(C1([2H])CCC(=O)NC1=O)C2 ; [2H]c1c([2H])c(NCc2ccc(CN3CCOCC3)cc2)c2c(c1[2H])C(=O)N([C@@]1([2H])CCC(=O)NC1=O)C2 ; [2H]c1c([2H])c(NCc2ccc(CN3CCOCC3)cc2)c2c(c1[2H])C(=O)N([C@]1([2H])CCC(=O)NC1=O)C2 ; [2H]c1c([2H])c(OCc2ccc(CN3CCOCC3)cc2)c2c(c1[2H])C(=O)N(C1CCC(=O)NC1=O)C2 ; [2H]c1c([2H])c(OCc2ccc(CN3CCOCC3)cc2)c2c(c1[2H])C(=O)N([C@H]1CCC(=O)NC1=O)C2 ; [2H]c1c([2H])c(OCc2ccc(CN3CCOCC3)cc2)c2c(c1[2H])C(=O)N([C@@H]1CCC(=O)NC1=O)C2]

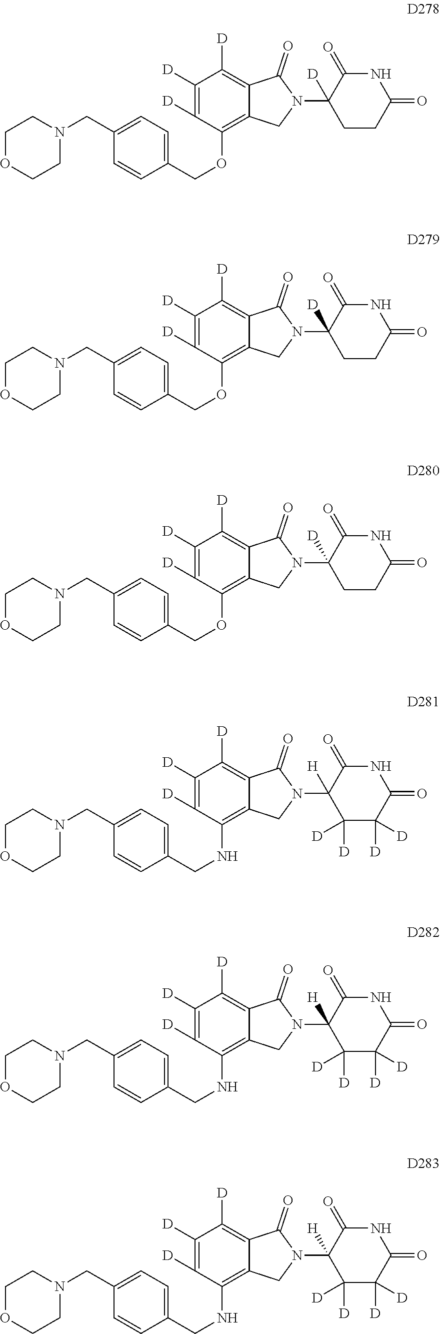

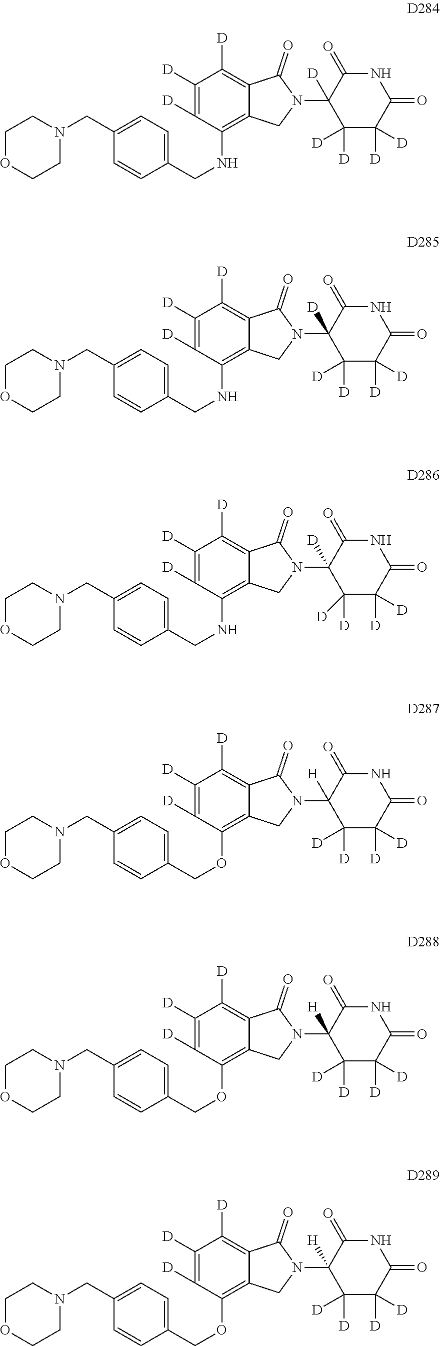

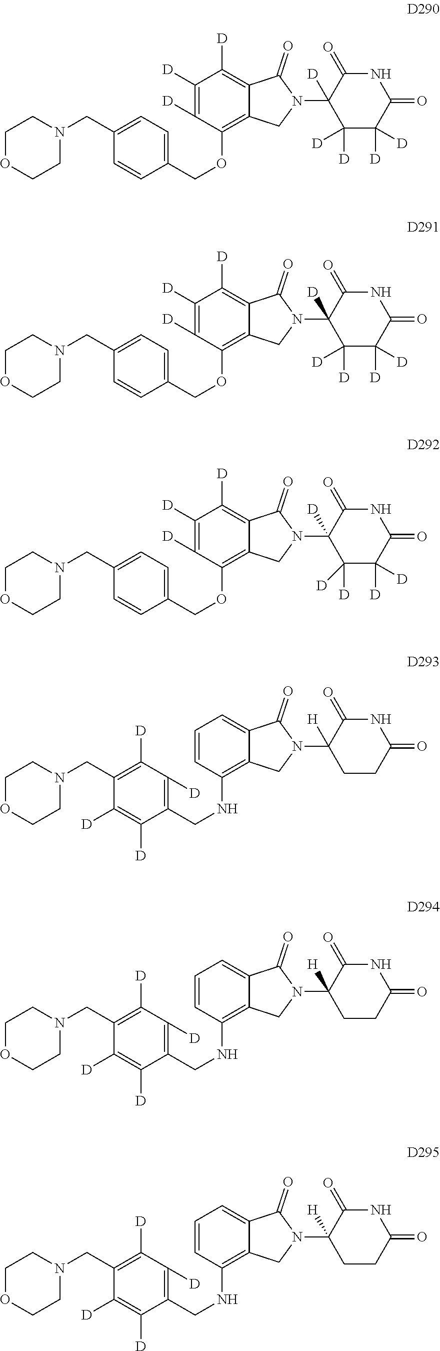

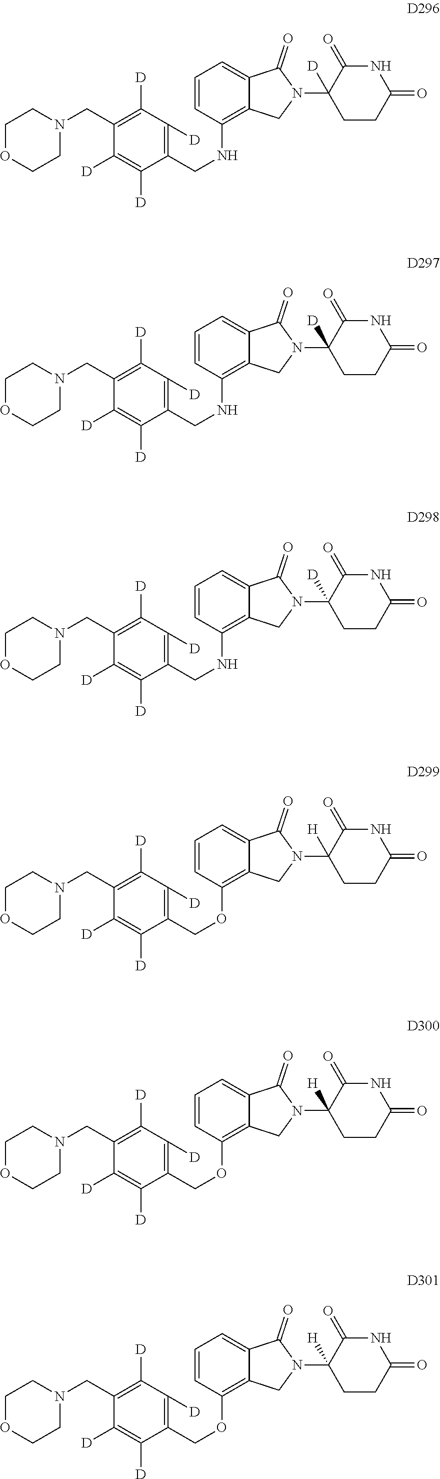 [SMILES: [2H]c1c([2H])c(CN2CCOCC2)c([2H])c([2H])c1CNc1cccc2c1CN(C1([2H])CCC(=O)NC1=O)C2=O ; [2H]c1c([2H])c(CN2CCOCC2)c([2H])c([2H])c1CNc1cccc2c1CN([C@@]1([2H])CCC(=O)NC1=O)C2=O ; [2H]c1c([2H])c(CN2CCOCC2)c([2H])c([2H])c1CNc1cccc2c1CN([C@]1([2H])CCC(=O)NC1=O)C2=O ; [2H]c1c([2H])c(CN2CCOCC2)c([2H])c([2H])c1COc1cccc2c1CN(C1CCC(=O)NC1=O)C2=O ; [2H]c1c([2H])c(CN2CCOCC2)c([2H])c([2H])c1COc1cccc2c1CN([C@H]1CCC(=O)NC1=O)C2=O ; [2H]c1c([2H])c(CN2CCOCC2)c([2H])c([2H])c1COc1cccc2c1CN([C@@H]1CCC(=O)NC1=O)C2=O]

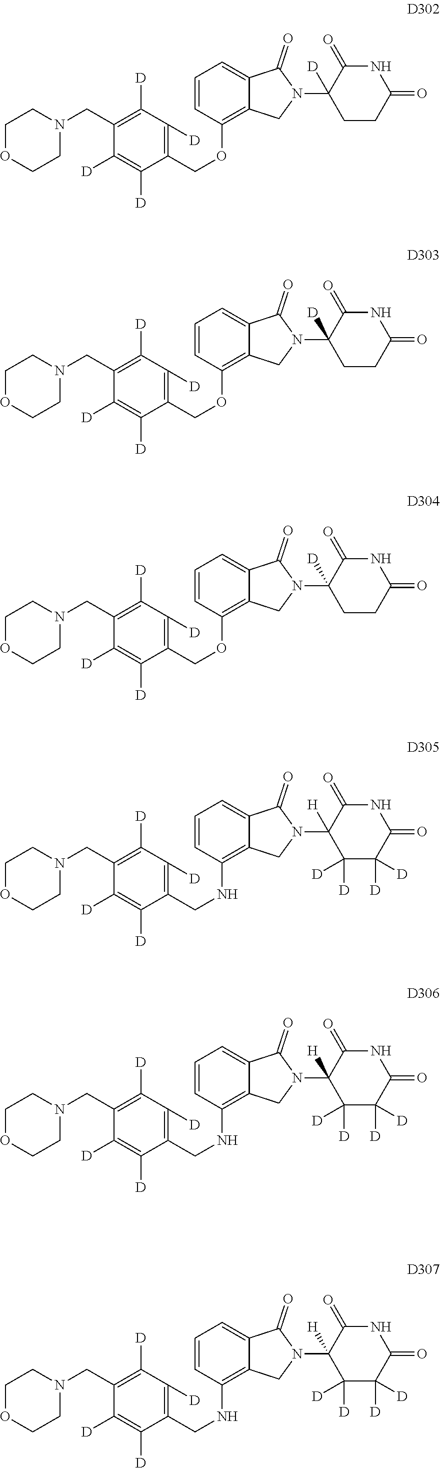

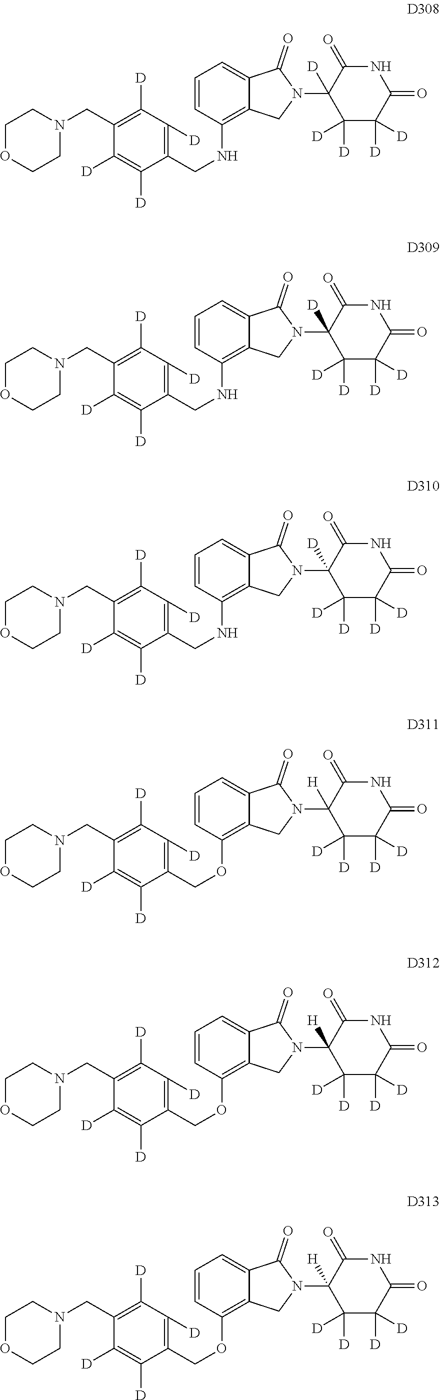

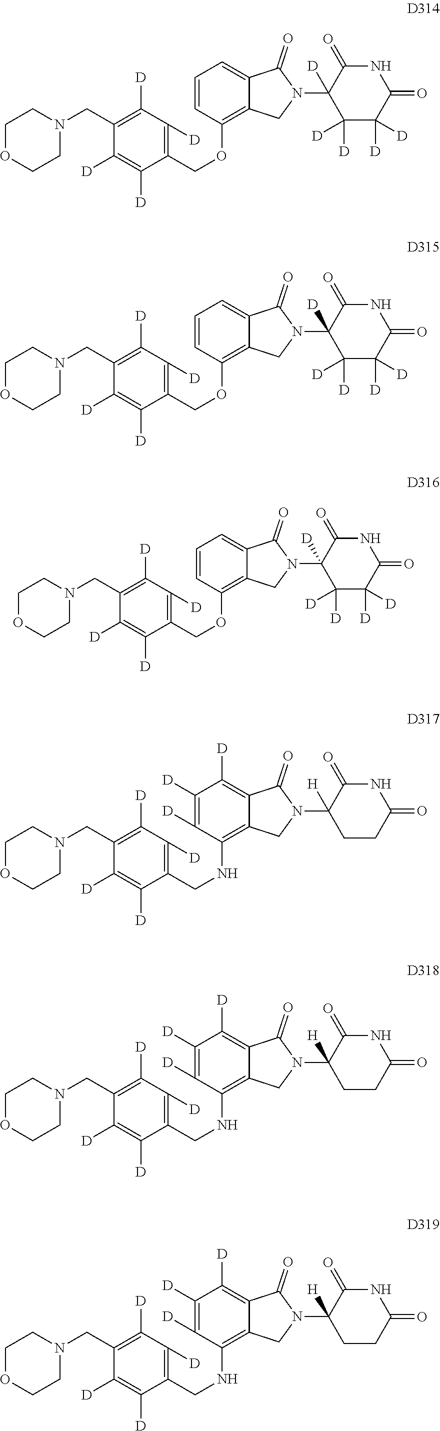

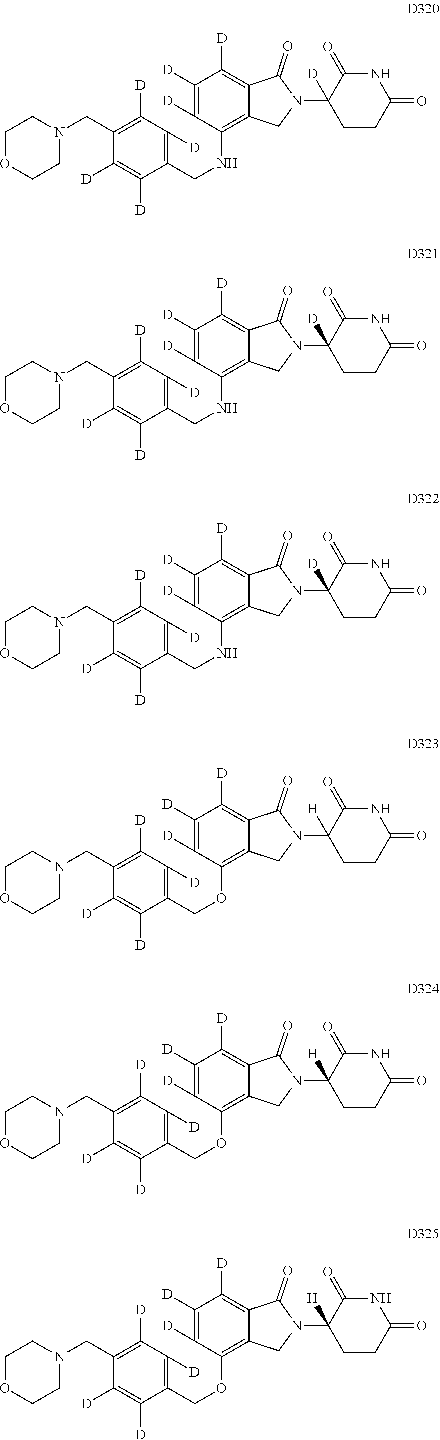

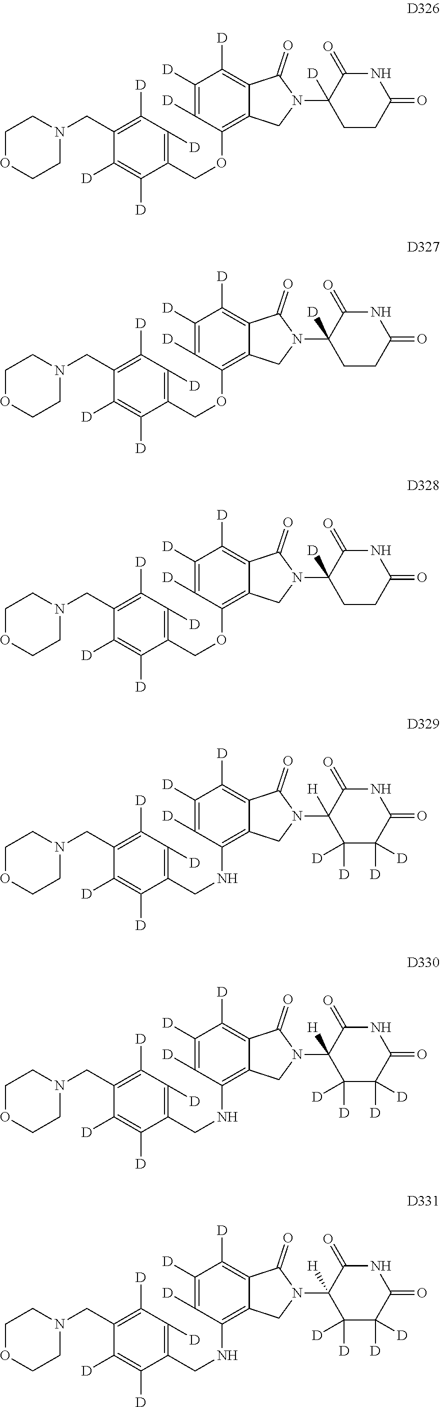

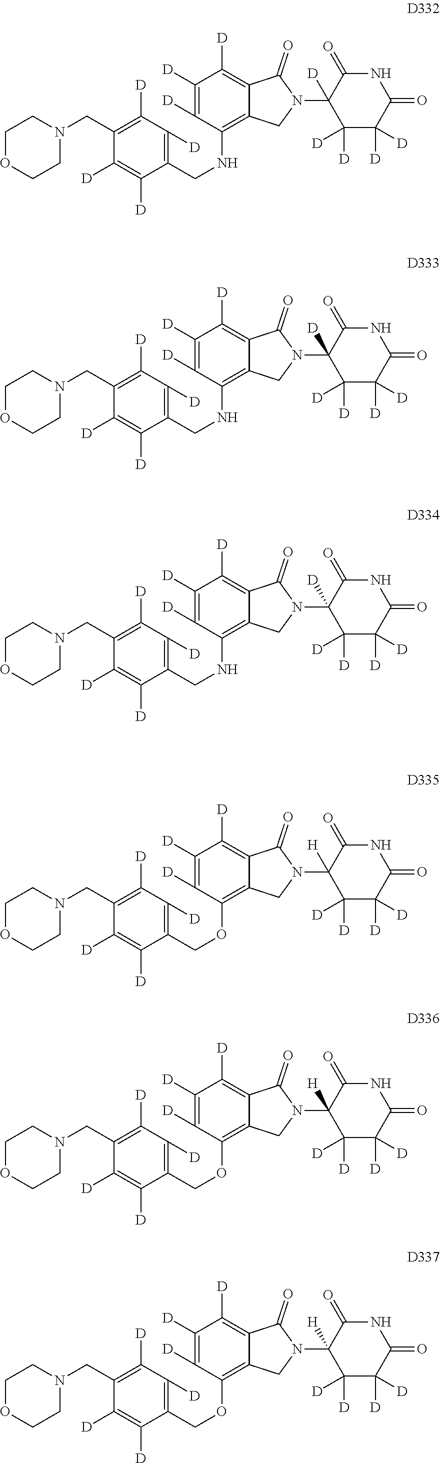

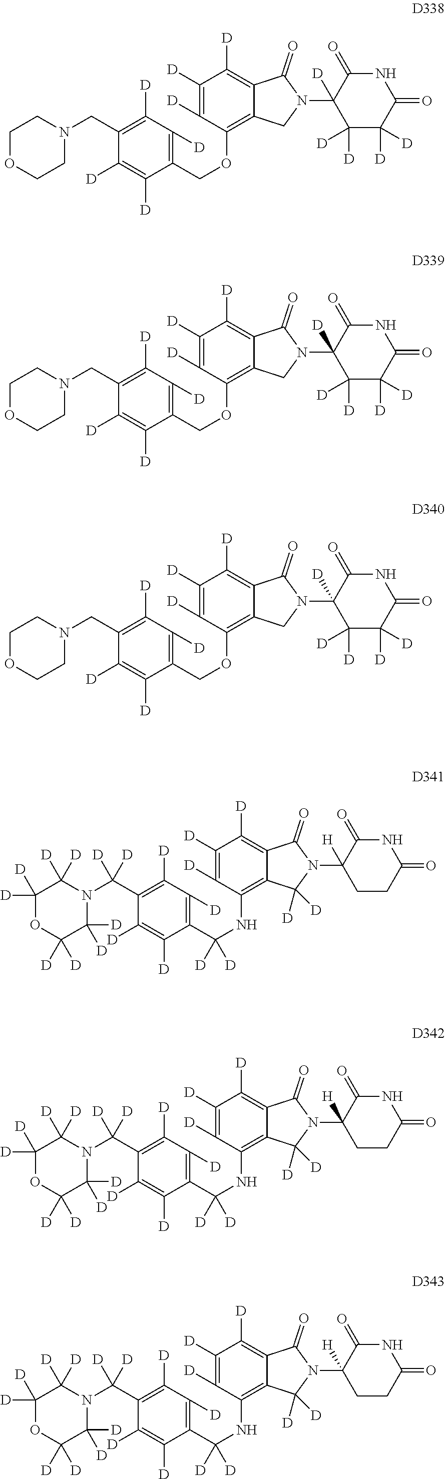

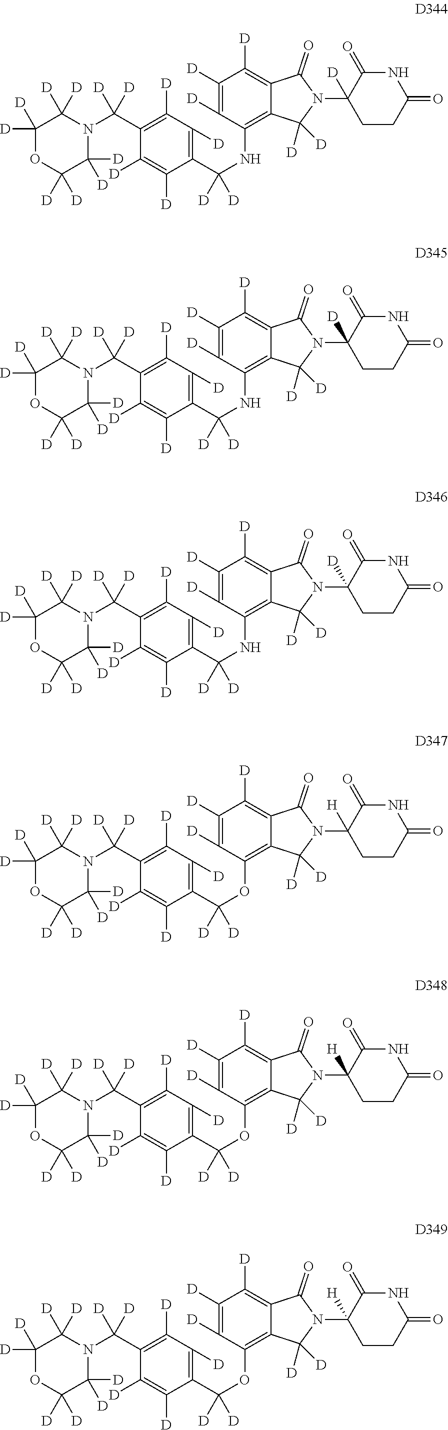

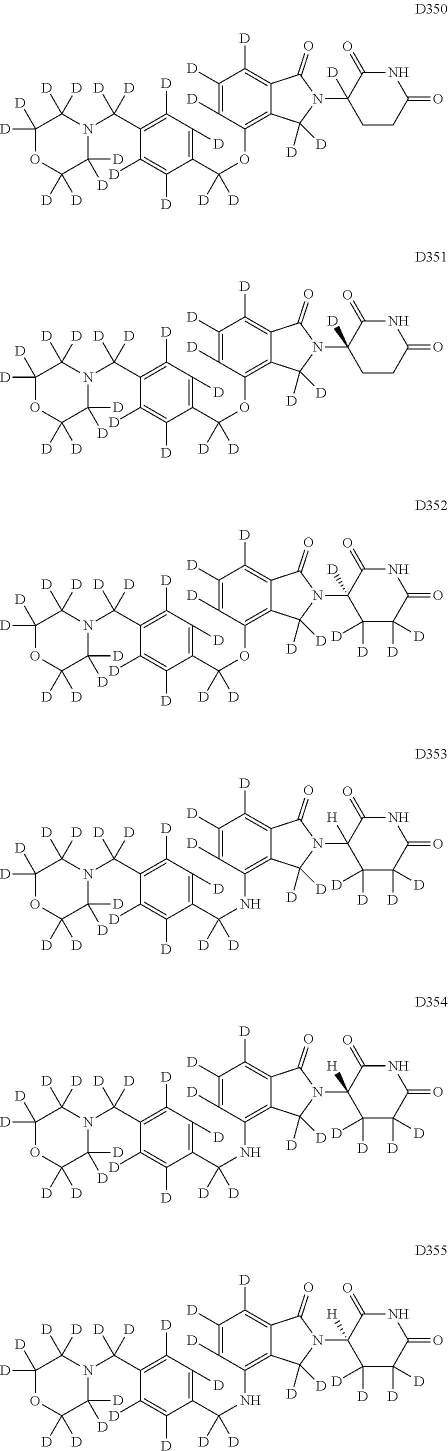

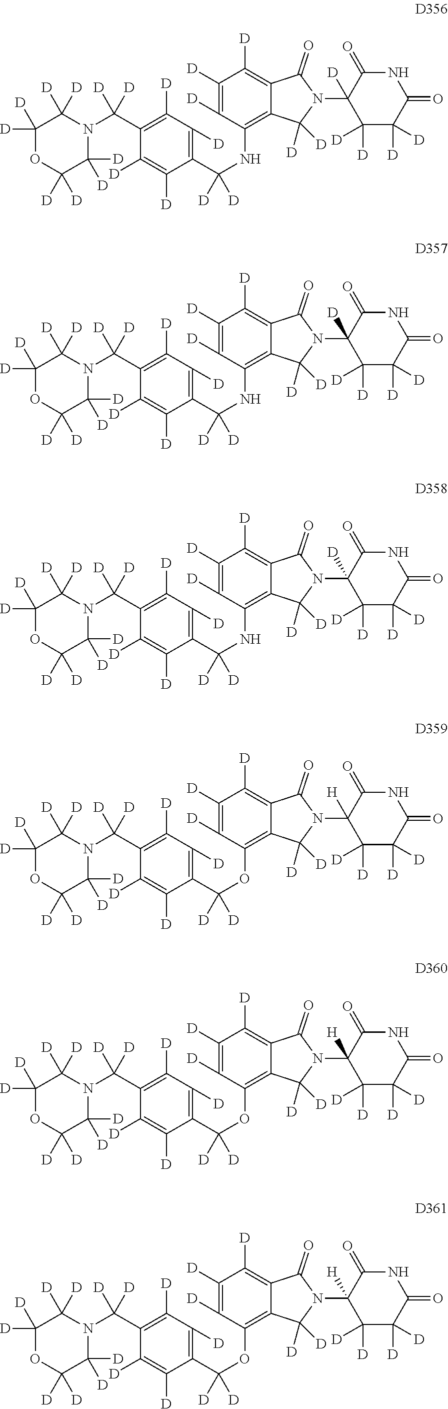

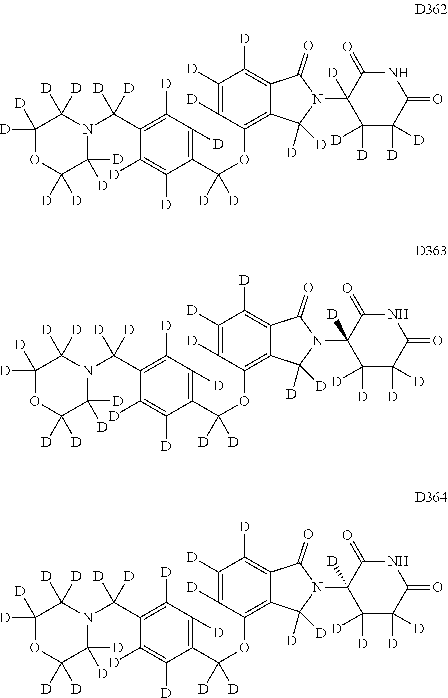

The benzoheterocyclic compound as shown in formula (I) of the present invention is more preferably any one of the following compounds: B001, B002, B003, B004, B005, B006, F001, F002, F003, F004, F005, F006, K001, K002, K003, K004, K005, K006, D101, D102, D103, D104, D105, D106, D107, D108, D109, D110, D111 or D112. The benzoheterocyclic compound as shown in formula (I) of the present invention is most preferably any one of the following compounds: B001, B002, B003, B004, B005, B006, F001, K001, D101, D107 or D108.

In some embodiments of the present invention, the androgen receptor pathway modulator is preferably Enzalutamide, ARN-509, Galeterone, ODM-201, ORM-15341, Enzalutamide and Galeterone, Enzalutamide and Abiraterone acetate, Enzalutamide and Abiraterone, Enzalutamide and ODM-201, Enzalutamide and ORM-15341, ARN-509 and Galeterone, ARN-509 and Abiraterone acetate, ARN-509 and Abirateron, ARN-509 and ODM-201, ARN-509 and ORM-15341, ODM-201 and Galeterone, ODM-201 and Abiraterone acetate, ODM-201 and Abiraterone, ORM-15341 and Galeterone, ORM-15341 and Abiraterone acetate, or ORM-15341 and Abiraterone.

In some embodiments of the present invention, the androgen receptor pathway modulator is more preferably Enzalutamide, ARN-509, Galeterone, ODM-201, Enzalutamide and Galeterone, Enzalutamide and Abiraterone acetate, ARN-509 and Galeterone, ARN-509 and Abiraterone acetate, ODM-201 and Enzalutamide, ODM-201 and ARN-509, ODM-201 and Galeterone, ODM-201 and Abiraterone, or ODM-201 and Abiraterone acetate.

In some embodiments of the present invention, in the pharmaceutical composition, the combination of the benzoheterocyclic compound as shown in the formula (I) and the androgen receptor pathway modulator is preferably that: the benzoheterocyclic compound as shown in the formula (I) is selected form B001, B002, B003, B004, B005, B006, F001, K001, D101, D107 or D108; the androgen receptor pathway modulator is selected from Enzalutamide, ARN-509, Galeterone, ODM-201, Enzalutamide and Galeterone, Enzalutamide and Abiraterone acetate, ARN-509 and Galeterone, ARN-509 and Abiraterone acetate. In the pharmaceutical composition of the present invention, the combination of the benzoheterocyclic compound as shown in the formula (I) and the androgen receptor pathway modulator is more preferably that: B001 and Enzalutamide, K001 and Enzalutamide, D107 and Enzalutamide, B001 and ARN-509, K001 and ARN-509, D107 and ARN-509, D108 and ARN-509, B001 and Galeterone, K001 and Galeterone, D107 and Galeterone, D108 and Galeterone, B001 and ODM-201, K001 and ODM-201, D108 and ODM-201, F001 and ODM-201, B002 and Enzalutamide, B003 and Enzalutamide, B004 and Enzalutamide, B005 and Enzalutamide, B006 and Enzalutamide, B002 and ARN-509, B003 and ARN-509, B004 and ARN-509, B005 and ARN-509, B006 and ARN-509, D108 and Enzalutamide, B001 and Enzalutamide and ARN-509, B001 and Enzalutamide and Galeterone, B001 and Enzalutamide and Abiraterone acetate, B001 and ARN-509 and Abiraterone acetate, B001 and ARN-509 and Galeterone, B001 and Galeterone Abiraterone acetate, K001 and Enzalutamide and ARN-509, K001 and Enzalutamide and Galeterone, K001 and Enzalutamide and Abiraterone acetate, K001 and ARN-509 and Abiraterone acetate, K001 and ARN-509 and Galeterone, K001 and Galeterone and Abiraterone acetate, D101 and Enzalutamide and ARN-509, D101 and Enzalutamide and Galeterone, D101 and Enzalutamide and Abiraterone acetate, D101 and ARN-509 and Abiraterone acetate, D101 and ARN-509 and Galeterone, D101 and Galeterone and Abiraterone acetate, D108 and Enzalutamide and ARN-509, D108 and Enzalutamide and Galeterone, D108 and Enzalutamide and Abiraterone acetate, D108 and ARN-509 and Abiraterone acetate, D108 and ARN-509 and Galeterone, or D108 and Galeterone and Abiraterone acetate.

In some embodiments of the present invention, the pharmaceutical composition may further comprise a hormone compound which is preferably one or more of prednisone, dexamethasone, dehydroepiandrosterone, isoandrosterone and megestrol acetate. Therefore, the pharmaceutical composition comprises one or more of a benzoheterocyclic compound as shown in formula (I), a pharmaceutically acceptable salt thereof, a solvate thereof, a polymorph thereof, a co-crystal thereof, a stereoisomer thereof, an isotope compound thereof, a metabolite thereof or a prodrug thereof, an androgen receptor pathway modulator and a hormone compound. The combination of the androgen receptor pathway modulator and the hormone compound is preferably Galeterone and prednisone, prednisone and Abiraterone acetate, Enzalutamide and prednisone, ARN-509 and prednisone, Enzalutamide and dexamethasone, Enzalutamide and Galeterone and prednisone, Enzalutamide and Galeterone and dexamethasone, Enzalutamide and ODM-201 and prednisone, Enzalutamide and ODM-201 and dexamethasone, Enzalutamide and ORM-15341 and prednisone, Enzalutamide and ORM-15341 and dexamethasone, Enzalutamide and Abiraterone acetate and prednisone, Enzalutamide and Abiraterone acetate and dexamethasone, Enzalutamide and Abiraterone and prednisone, Enzalutamide and Abiraterone and dexamethasone, ARN-509 and dexamethasone, ARN-509 and Galeterone and prednisone, ARN-509 and Galeterone and dexamethasone, ARN-509 and ODM-201 and prednisone, ARN-509 and ODM-201 and dexamethasone, ARN-509 and ORM-15341 and prednisone, ARN-509 and ORM-15341 and dexamethasone, ARN-509 and Abiraterone acetate and prednisone, ARN-509 and Abiraterone acetate and dexamethasone, ARN-509 and Abiraterone and prednisone, ARN-509 and Abiraterone and dexamethasone, ODM-201 and prednisone, ODM-201 and dexamethasone, ODM-201 and Galeterone and prednisone, ODM-201 and Galeterone and dexamethasone, ODM-201 and Abiraterone acetate and prednisone, ODM-201 and Abiraterone acetate and dexamethasone, ODM-201 and Abiraterone and prednisone, ODM-201 and Abiraterone and dexamethasone, ORM-15341 and prednisone, ORM-15341 and dexamethasone, ORM-15341 and Galeterone and prednisone, ORM-15341 and Galeterone and dexamethasone, ORM-15341 and Abiraterone acetate and prednisone, ORM-15341 and Abiraterone acetate and dexamethasone, ORM-15341 and Abiraterone and prednisone, ORM-15341 and Abiraterone and dexamethasone, Galeterone and dexamethasone, Galeterone and Abiraterone acetate and prednisone, Galeterone and Abiraterone acetate and dexamethasone, Galeterone and Abiraterone and prednisone, or Galeterone and Abiraterone and dexamethasone.

In some more preferred embodiments of the invention, in the pharmaceutical composition, the combination of the androgen receptor pathway modulator and the hormone compound is more preferably Galeterone and prednisone, prednisone and Abiraterone acetate, Enzalutamide and prednisone, ARN-509 and prednisone, Enzalutamide and Galeterone and prednisone, Enzalutamide and Abiraterone acetate and prednisone, ARN-509 and Galeterone and prednisone, ARN-509 and Abiraterone acetate and prednisone, ODM-201 and prednisone, ODM-201 and Galeterone and prednisone, ODM-201 and Abiraterone acetate and prednisone, ODM-201 and Abiraterone and prednisone, Enzalutamide and ODM-201 and prednisone, or ARN-509 and ODM-201 and prednisone.

In the pharmaceutical composition, the combination of the benzoheterocyclic compound as shown in formula (I), the androgen receptor pathway modulator and the hormone compound is more preferably that: the benzoheterocyclic compound as shown in formula (I) is selected form B001, K001, D101, D107 or D108; the combination of the androgen receptor pathway modulator and the hormone compound is selected from Galeterone and prednisone, prednisone and Abiraterone acetate, Enzalutamide and prednisone, ARN-509 and prednisone, Enzalutamide and Galeterone and prednisone, Enzalutamide and Abiraterone acetate and prednisone, ARN-509 and Galeterone and prednisone, ARN-509 and Abiraterone acetate and prednisone, ODM-201 and prednisone, ODM-201 and Galeterone and prednisone, ODM-201 and Abiraterone acetate and prednisone, ODM-201 and Abiraterone and prednisone, Enzalutamide and ODM-201 and prednisone, or ARN-509 and ODM-201 and prednisone.

In another aspect, the present invention provides the benzoheterocyclic compound as shown in formula (I), the pharmaceutically acceptable salt thereof, the solvate thereof, the polymorph thereof, the co-crystal thereof, the stereoisomer thereof, the isotope compound thereof, the metabolite thereof or the prodrug thereof for use in manufacturing a medicament for the prevention and/or treatment of prostate cancer in combination with an androgen receptor pathway modulator;

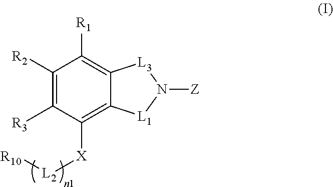

wherein, the androgen receptor pathway modulator, n1, L₁, L₃, L₂, X, R₁, R₂, R₃, R₁₀ and Z are defined as described above.

In some preferred embodiments of the present invention, in the use, the benzoheterocyclic compound as shown in the formula (I) is selected from B001, B002, B003, B004, B005, B006, F001, K001, D101, D107 or D108; the androgen receptor pathway modulator is selected from Enzalutamide, ARN-509, Galeterone, ODM-201, Enzalutamide and Galeterone, Enzalutamide and Abiraterone acetate, ARN-509 and Galeterone, ARN-509 and Abiraterone acetate, ODM-201 and Enzalutamide; ODM-201 and ARN-509, ODM-201 and Galeterone, ODM-201 and Abiraterone, or ODM-201 and Abiraterone acetate.

Some embodiments of the present invention also provides the benzoheterocyclic compound as shown in formula (I), the pharmaceutically acceptable salt thereof, the solvate thereof, the polymorph thereof, the co-crystal thereof, the stereoisomer thereof, the isotope compound thereof, the metabolite thereof or the prodrug thereof for use in manufacturing a medicament for the prevention and/or treatment of prostate cancer in combination with an androgen receptor pathway modulator and a hormone compound; the hormone compound is preferably one or more of prednisone, dexamethasone, dehydroepiandrosterone, isoandrosterone and megestrol acetate. In some preferred embodiments of the present invention, in the use, the combination of the androgen receptor pathway modulator and the hormone compound is preferably Galeterone and prednisone, prednisone and Abiraterone acetate, Enzalutamide and prednisone, ARN-509 and prednisone, Enzalutamide and dexamethasone, Enzalutamide and Galeterone and prednisone, Enzalutamide and Galeterone and dexamethasone, Enzalutamide and ODM-201 and prednisone, Enzalutamide and ODM-201 and dexamethasone, Enzalutamide and ORM-15341 and prednisone, Enzalutamide and ORM-15341 and dexamethasone, Enzalutamide and Abiraterone acetate and prednisone, Enzalutamide and Abiraterone acetate and dexamethasone, Enzalutamide and Abiraterone and prednisone, Enzalutamide and Abiraterone and dexamethasone, ARN-509 and dexamethasone, ARN-509 and Galeterone and prednisone, ARN-509 and Galeterone and dexamethasone, ARN-509 and ODM-201 and prednisone, ARN-509 and ODM-201 and dexamethasone, ARN-509 and ORM-15341 and prednisone, ARN-509 and ORM-15341 and dexamethasone, ARN-509 and Abiraterone acetate and prednisone, ARN-509 and Abiraterone acetate and dexamethasone, ARN-509 and Abiraterone and prednisone, ARN-509 and Abiraterone and dexamethasone, ODM-201 and prednisone, ODM-201 and dexamethasone, ODM-201 and Galeterone and prednisone, ODM-201 and Galeterone and dexamethasone, ODM-201 and Abiraterone acetate and prednisone, ODM-201 and Abiraterone acetate and dexamethasone, ODM-201 and Abiraterone and prednisone, ODM-201 and Abiraterone and dexamethasone, ORM-15341 and prednisone, ORM-15341 and dexamethasone, ORM-15341 and Galeterone and prednisone, ORM-15341 and Galeterone and dexamethasone, ORM-15341 and Abiraterone acetate and prednisone, ORM-15341 and Abiraterone acetate and dexamethasone, ORM-15341 and Abiraterone and prednisone, ORM-15341 and Abiraterone and dexamethasone, Galeterone and dexamethasone, Galeterone and Abiraterone acetate and prednisone, Galeterone and Abiraterone acetate and dexamethasone, Galeterone and Abiraterone and prednisone, or Galeterone and Abiraterone and dexamethasone.

In some more preferred embodiments of the present invention, in the use, the combination of the androgen receptor pathway modulator and the hormone compound is more preferably Galeterone and prednisone, prednisone and Abiraterone acetate, Enzalutamide and prednisone, ARN-509 and prednisone, Enzalutamide and Galeterone and prednisone, Enzalutamide and Abiraterone acetate and prednisone, ARN-509 and Galeterone and prednisone, ARN-509 and Abiraterone acetate and prednisone, ODM-201 and prednisone, ODM-201 and Galeterone and prednisone, ODM-201 and Abiraterone acetate and prednisone, ODM-201 and Abiraterone and prednisone, Enzalutamide and ODM-201 and prednisone, or ARN-509 and ODM-201 and prednisone.

In some more preferred embodiments of the present invention, in the use, the benzoheterocyclic compound as shown in formula (I) is selected from B001, K001, D101, D107 or D108; the combination of the androgen receptor pathway modulator and the hormone compound is selected from Galeterone and prednisone, prednisone and Abiraterone acetate, Enzalutamide and prednisone, ARN-509 and prednisone, Enzalutamide and Galeterone and prednisone, Enzalutamide and Abiraterone acetate and prednisone, ARN-509 and Galeterone and prednisone, ARN-509 and Abiraterone acetate and prednisone, ODM-201 and prednisone, ODM-201 and Galeterone and prednisone, ODM-201 and Abiraterone acetate and prednisone, ODM-201 and Abiraterone and prednisone, or Enzalutamide and ODM-201 and prednisone, or ARN-509 and ODM-201 and prednisone.

In another aspect, the present invention provides a method of prevention and/or treatment of prostate cancer, comprising administration of a therapeutically effective amount of the benzoheterocyclic compound of the formula (I), the pharmaceutically acceptable salt thereof, the solvate thereof, the polymorph thereof, the co-crystal thereof, the stereoisomer thereof, the isotope compound thereof, the metabolite thereof or the prodrug thereof and the androgen receptor pathway modulator to the patients in need;

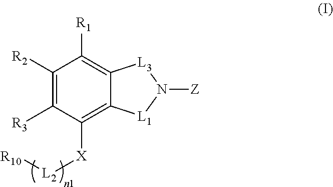

wherein, the androgen receptor pathway modulator, n1, L₁, L₃, L₂, X, R₁, R₂, R₃, R₁₀ and Z are defined as described above.

In some preferred embodiments of the present invention, in the method of prevention and/or treatment of prostate cancer, the benzoheterocyclic compound as shown in formula (I) is selected from B001, B002, B003, B004, B005, B006, F001, K001, D101, D107 or D108; the androgen receptor pathway modulator is selected from Enzalutamide, ARN-509, Galeterone, ODM-201, Enzalutamide and Galeterone, Enzalutamide and Abiraterone acetate, ARN-509 and Galeterone, ARN-509 and Abiraterone acetate, ODM-201 and Enzalutamide, ODM-201 and ARN-509, ODM-201 and Galeterone, ODM-201 and Abiraterone, ODM-201 and Abiraterone acetate.

In some embodiments of the present invention, preferably, the method of prevention and/or treatment of prostate cancer preferably comprises administration of a therapeutically effective amount of the benzoheterocyclic compound of the formula (I), the pharmaceutically acceptable salt thereof, the solvate thereof, the polymorph thereof, the co-crystal thereof, the stereoisomer thereof, the isotope compound thereof, the metabolite thereof or the prodrug thereof and the androgen receptor pathway modulator and the hormone compound to the patients in need; the hormone compound is preferably one or more of prednisone, dexamethasone, dehydroepiandrosterone, isoandrosterone and megestrol acetate. In the method of prevention and/or treatment of prostate cancer, the combination of the androgen receptor pathway modulator and the hormone compound is preferably Galeterone and prednisone, prednisone and Abiraterone acetate, Enzalutamide and prednisone, ARN-509 and prednisone, Enzalutamide and dexamethasone, Enzalutamide and Galeterone and prednisone, Enzalutamide and Galeterone and dexamethasone, Enzalutamide and ODM-201 and prednisone, Enzalutamide and ODM-201 and dexamethasone, Enzalutamide and ORM-15341 and prednisone, Enzalutamide and ORM-15341 and dexamethasone, Enzalutamide and Abiraterone acetate and prednisone, Enzalutamide and Abiraterone acetate and dexamethasone, Enzalutamide and Abiraterone and prednisone, Enzalutamide and Abiraterone and dexamethasone, ARN-509 and dexamethasone, ARN-509 and Galeterone and prednisone, ARN-509 and Galeterone and dexamethasone, ARN-509 and ODM-201 and prednisone, ARN-509 and ODM-201 and dexamethasone, ARN-509 and ORM-15341 and prednisone, ARN-509 and ORM-15341 and dexamethasone, ARN-509 and Abiraterone acetate and prednisone, ARN-509 and Abiraterone acetate and dexamethasone, ARN-509 and Abiraterone and prednisone, ARN-509 and Abiraterone and dexamethasone, ODM-201 and prednisone, ODM-201 and dexamethasone, ODM-201 and Galeterone and prednisone, ODM-201 and Galeterone and dexamethasone, ODM-201 and Abiraterone acetate and prednisone, ODM-201 and Abiraterone acetate and dexamethasone, ODM-201 and Abiraterone and prednisone, ODM-201 and Abiraterone and dexamethasone, ORM-15341 and prednisone, ORM-15341 and dexamethasone, ORM-15341 and Galeterone and prednisone, ORM-15341 and Galeterone and dexamethasone, ORM-15341 and Abiraterone acetate and prednisone, ORM-15341 and Abiraterone acetate and dexamethasone, ORM-15341 and Abiraterone and prednisone, ORM-15341 and Abiraterone and dexamethasone, Galeterone and dexamethasone, Galeterone and Abiraterone acetate and prednisone, Galeterone and Abiraterone acetate and dexamethasone, Galeterone and Abiraterone and prednisone, or Galeterone and Abiraterone and dexamethasone.

In some more preferred embodiments of the present invention, in the method of prevention and/or treatment of prostate cancer, the combination of the androgen receptor pathway modulator and the hormone compound is more preferably Galeterone and prednisone, prednisone and Abiraterone acetate, Enzalutamide and prednisone, ARN-509 and prednisone, Enzalutamide and Galeterone and prednisone, Enzalutamide and Abiraterone acetate and prednisone, ARN-509 and Galeterone and prednisone, ARN-509 and Abiraterone acetate and prednisone, ODM-201 and prednisone, ODM-201 and Galeterone and prednisone, ODM-201 and Abiraterone acetate and prednisone, ODM-201 and Abiraterone and prednisone, Enzalutamide and ODM-201 and prednisone, or ARN-509 and ODM-201 and prednisone.

In the method of prevention and/or treatment of prostate cancer, the combination of the benzoheterocyclic compound as shown in the formula (I), the androgen receptor pathway modulator and the hormone compound is more preferably that: the benzoheterocyclic compound as shown in the formula (I) is selected from B001, K001, D101, D107 or D108; the combination of the androgen receptor pathway modulator and the hormone compound is selected from Galeterone and prednisone, prednisone and Abiraterone acetate, Enzalutamide and prednisone, ARN-509 and prednisone, Enzalutamide and Galeterone and prednisone, Enzalutamide and Abiraterone acetate and prednisone, ARN-509 and Galeterone and prednisone, ARN-509 and Abiraterone acetate and prednisone, ODM-201 and prednisone, ODM-201 and Galeterone and prednisone, ODM-201 and Abiraterone acetate and prednisone, ODM-201 and Abiraterone and prednisone, or Enzalutamide and ODM-201 and prednisone, or ARN-509 and ODM-201 and prednisone.

In the pharmaceutical composition, the use or the method of prevention and/or treatment of prostate cancer of the present invention, the mole ratio of the benzoheterocyclic compound as shown in the formula (I) and the androgen receptor pathway modulator, which may be selected in accordance with the conventional art, is preferably 1:0.0001-1:50, more preferably 1:0.0005-1:30 (1:0.0005, 1:0014, 1:0.004, 1:0.013, 1:0.04, 1:0.12, 1:0.33, 1:1, 1:3, 1:9, 1:27), even more preferably 1:0.1-1:10.

In the pharmaceutical composition, the use or the method of prevention and/or treatment of prostate cancer of the present invention, the amount of the benzoheterocyclic compound as shown in the formula (I) and the androgen receptor pathway modulator is not particularly limited, which may be selected in accordance with the conventional art, for example, the amount of the benzoheterocyclic compound as shown in the formula (I) may be 0.01-300 μM, preferably 0.05-200 μM, more preferably 0.4-100 μM (for example 100.00 μM, 33.33 μM, 11.11 μM, 3.70 μM, 1.23 μM, 0.41 μM); the amount of the androgen receptor pathway modulator may be 0.01-100 μM, preferably 0.05-50 μM, more preferably 0.05-30 μM, even more preferably 0.05-12 μM (for example 11.11 μM, 3.70 μM, 1.23 μM, 0.41 μM, 0.14 μM, 0.05 μM), particularly preferably 0.1-10 μM.

In the pharmaceutical composition, the use or the method of prevention and/or treatment of prostate cancer of the present invention, when further comprising the hormonal compound, the amount of the hormonal compound is not particularly limited if the interaction between the benzoheterocyclic compound as shown in the formula (I) and the androgen receptor pathway modulator as described above is not affected; the mole ratio of the hormonal compound and the androgen receptor pathway modulator is preferably 1:0.01-1:100, more preferably 1:0.1-1:10 (for example 1:0.1, 1:1, 2:1, 1:10).

In the pharmaceutical composition, the use or the method of prevention and/or treatment of prostate cancer of the present invention, when further comprising the hormonal compound, the amount of the hormonal compound is not particularly limited if the interaction between the benzoheterocyclic compound as shown in the formula (I) and the androgen receptor pathway modulator as described above is not affected; the amount of the hormonal compound and the androgen receptor pathway modulator may be independently 0.01-100 μM, preferably 0.05-50 μM, more preferably 0.05-30 μM, even more preferably 0.05-12 μM (for example 11.11 μM, 10 μM, 5 μM, 3.70 μM, 2 μM, 1.23 μM, 1 μM, 0.41 μM, 0.14 μM, 0.1 μM, 0.05 μM and so on), particularly preferably 0.1-10 μM.

The pharmaceutical composition of the present invention may be formulated into any form for administration, including injection (intravenous), mucosal, oral administration (solid and liquid preparation), inhalation, ocular administration, rectal administration, topical or parenteral (infusion, injection, implantation, subcutaneous, vein, artery, intramuscular) administration. The pharmaceutical composition of the present invention can also be a controlled release or delayed release preparation. Examples of solid oral preparations include but not limited to powder, capsule, caplet, soft capsule, pill and tablet. Examples of liquid preparations for oral or mucosal administration include but not limited to suspension, emulsion, elixir and solution. Examples of preparations for topical administration include but not limited to emulsion, gel, ointment, cream, patch, paste, foam, lotion, drops or serum preparation. Examples of preparations for parenteral administration include but not limited to injection solution, dry preparation which can be dissolved or suspended in a pharmaceutically acceptable carrier, injection suspension and injection emulsion. Examples of other suitable preparations of the pharmaceutical composition, include but not limited to eye drops and other ophthalmic preparations; aerosol, such as nasal spray or inhalation; liquid dosage forms suitable for parenteral administration; suppository and pastille.

The pharmaceutical composition of the present invention may further comprises a pharmaceutically acceptable excipient, such as those widely used in drug manufacture field. The excipient is mainly used to provide a safe, stable and functionalized pharmaceutical composition, and can also provide a process which makes the active ingredients dissolved at a desired rate after the subject receives administration or promotes the effective absorption of the active ingredients after the subject is administered with the composition. The excipient can be an inert filler, or provide a certain function, such as stabilizing the overall pH value of the composition or preventing the degradation of the active ingredients of the composition. The pharmaceutically acceptable excipient may comprise one or more of the following excipients: binder, suspending agent, emulsifier, diluent, filler, granulating agent, adhesive, disintegrating agent, lubricant, anti-adhesive agent, glidant, wetting agent, gelling agent, absorption retarder, dissolution inhibitor, reinforcing agent, adsorbent, buffer, chelating agent, preservative, colorant, flavoring agent and sweetening agent. The pharmaceutically acceptable carrier can take a variety of forms depending on the form of preparation desired. For example, for liquid oral preparation, the suitable carriers and additives include water, glycols, oils, alcohols, flavoring agent, preservative, colorant, etc. As another illustrative example, for solid oral preparation, suitable carriers and additives include starch, sugar, diluent, granulation agent, lubricant, adhesive, disintegrating agent, etc. The pharmaceutically acceptable carriers or excipients usually should be non-toxic. The pharmaceutical composition of the present invention may comprise one or more suitable carrier(s)/excipient(s). The amount and type of the excipient vary depending on the requirements. A person skilled in the art can easily determine the appropriate carrier(s)/excipient(s) to be added to the pharmaceutical composition of the present invention based on the contents disclosed herein.

The pharmaceutical composition of the present invention can be prepared according to the disclosure using any method known to people skilled in the art. For example, the pharmaceutical composition of the present invention can be prepared by mixing one or more of the benzoheterocyclic compound as shown in formula (I), the pharmaceutically acceptable salt thereof, the solvate thereof, the polymorph thereof, the co-crystal thereof, the stereoisomer thereof, the isotope compound thereof, the metabolite thereof and the prodrug thereof as well as the androgen receptor pathway modulator with the pharmaceutically acceptable carrier and, or by mixing one or more of the benzoheterocyclic compound as shown in formula (I), the pharmaceutically acceptable salt thereof, the solvate thereof, the polymorph thereof, the co-crystal thereof, the stereoisomer thereof, the isotope compound thereof, the metabolite thereof and the prodrug thereof, the androgen receptor pathway modulator as well as the hormonal compound with the pharmaceutically acceptable carrier, according to conventional drug compounding technologies, which include but not limited to conventional mixing, dissolving, granulating, emulsifying, grinding, encapsulating, embedding or lyophilization.

In some embodiments, the pharmaceutical composition of the present invention relates to a controlled release preparation. As used herein, “controlled release preparation” refers to a preparation, wherein the therapeutic active ingredient of the pharmaceutical composition has a controlled release rate, or a specific delay to control the release site of the therapeutic active ingredient in the subject administered with the pharmaceutical composition. One controlled release preparation may comprise a controlled release agent, such as a sustained release agent (sustained release or delayed release) and a delayed release agent (delayed release).

As used herein, the terms “sustained release” and “delayed release” refer to prolonging the release of the therapeutic active ingredient from the pharmaceutical composition. As used herein, the term “delayed release” refers to that the therapeutic active ingredient releases from the pharmaceutical composition at a specific site or in a desired environment when the composition reaches the desired environment in the subject who has received administration or after a specific period of time since the subject receives administration.

As used herein, the terms “sustained release agent” and “delayed release agent” refer to a compound or an additive which controls the releasing of the therapeutic active ingredient from the composition, so as to make the release gradual and prolong the time of release. The sustained or delayed release agent may make the therapeutic active ingredient released within a specific period of time after the composition was administered to a subject.

The “controlled release” from the pharmaceutical composition of the present invention can be achieved by a variety of conditions, including but not limited to pH, temperature, enzymes, water, or other physiological conditions or compounds. The pharmaceutical composition of the present invention may further comprise an enteric coating which controls the release of the active ingredient in the pharmaceutical composition, making it released gradually and continuously from the composition in a desired period of time, so that the active ingredient can play a therapeutic or preventive role for an extended period of time. The controlled release pharmaceutical composition may further comprise one or more of other therapeutic agents or medicaments as disclosed below.

One skilled in the art may be familiar with those appropriate controlled release preparations, sustained and delayed release agents based on the disclosed contents. Unrestrictive examples of the controlled release agents which can be incorporated into the pharmaceutical composition of the present invention in order to provide a controlled release composition include polymers (such as hydroxypropyl methyl cellulose), gel, permeable membrane, particle, liposome, microsphere and the combination thereof. Any composition described herein may be suitable for the controlled release preparation, such as tablet, capsule, soft capsule and caplet.

In the present invention, the term “active ingredient” refers to the active ingredient in the pharmaceutical composition of the present invention, that is, one or more of the compound as shown in formula (I), the pharmaceutically acceptable salt thereof, the solvate thereof, the polymorph thereof, the co-crystal thereof, the stereoisomer thereof, the isotope compound thereof, the metabolite thereof and the prodrug thereof and the androgen receptor pathway modulator, or one or more of the compound as shown in formula (I), the pharmaceutically acceptable salt thereof, the solvate thereof, the polymorph thereof, the co-crystal thereof, the stereoisomer thereof, the isotope compound thereof, the metabolite thereof and the prodrug thereof, the androgen receptor pathway modulator and the hormonal compound.

When the pharmaceutical composition of the present invention is administered to a subject for the purpose of treating or preventing a disease, disorder or condition, the active ingredient in the pharmaceutical composition may be administered by the same route or by a different route. The route of administration may be any route described herein, including but not limited to oral, inhalation, injection, ophthalmic, mucosal, rectal, emulsion, liposome, long-acting implant or sustained controlled release process. The specific route of administration will depend on the therapeutic agent itself and the preparation, as well as the disease, disorder or condition to be prevented or treated. According to the present disclosure, the skill level of an ordinary person skilled in the art is sufficient to determine the route of administration of other therapeutic agents. The active ingredient in the pharmaceutical composition of the present invention may be administered to the subject within a period of time (administration period) followed by a period of no administration of the compound (non-administration period). The administration period and non-administration period can be repeated for desired times. The desired length and times of the administration period or non-administration period will depend on the type and/or severity of the disease, disorder or condition being treated or prevented, as well as the sex, age, weight, and other parameters (e.g. the individual subject's biological, physical, and physiological status, etc.) of the individual subject. Each of the active ingredients in the pharmaceutical composition of the present invention may be administered simultaneously to the subject in a period of time and may also be administered to the subject sequentially in a period of time. According to the present disclosure, the skill level of an ordinary person skilled in the art is sufficient to determine the appropriate length and times of administration period and/or non-administration period.

The therapeutic method in the present invention comprises administering the pharmaceutical composition to a subject by any suitable processes, such as injection, mucosal, oral, inhalation, ocular, rectal, long-acting implant, liposome, emulsion or sustained release process.

One skilled in that art will understand that the therapeutically or prophylactically effective amount of the pharmaceutical composition of the present invention may vary with factors, for a specific subject, such as age, diet, health, etc., the symptom or disease to be treated or prevented, the severity of the disorder or condition, and the complications and types, and the preparations used etc. According to the disclosures in present invention, one skilled in the art can easily determine the desired therapeutically or prophylactically effective amount administered to the subject, so as to induce the desired biological or medical response in the subject.

The combined use of each of the active ingredients in the pharmaceutical composition according to the present invention may play a synergistic effect in the treatment or prevention of any disease, disorder or condition.

In any of the methods described herein, the pharmaceutical composition according to the present invention may be used alone or in combination with ultrasound therapy, radiation therapy (referred to as radiotherapy) or radioimmunotherapy etc., and may also be used in combination with one or more of other pharmacologically active therapeutic agents (hereinafter referred to as “other therapeutic agents”). The amount and type of other therapeutic agents will depend on the disease, disorder or condition to be treated or prevented; the severity of the disease, disorder or condition; factors of the subject administrated with the composition, such as age, weight, physical conditions, etc.; the route of administration, and so on. According to the embodiments of the present invention, the other therapeutic agent may be a natural, semi-synthetic or synthetic compound. In another embodiment, the other therapeutic agent may be a small molecule, such as a synthetic organic or inorganic molecule; or a larger molecule or biomolecule, such as a protein or nucleic acid with pharmacological activity. In another embodiment, the other therapeutic agent may be one or more of a chemotherapeutant, an antiangiogenic drug (also known as an angiogenesis inhibitor), an immunomodulatory agent, an immunotherapeutic agent, a monoclonal antibody, a polyclonal antibody, and a kinase inhibitor.

The chemotherapeutant (chemotherapeutic agent), is a chemically synthesized drug. Currently, the chemotherapeutant is the main drug in the treatment of cancer and some autoimmune diseases, what commonly used are: epirubicin, doxorubicin, daunorubicin, mitomycin, fluorouracil deoxynucleotides and so on.

The antiangiogenic drug inhibits angiogenesis by inhibiting pro-angiogenic growth factor, growth factor receptor or signaling pathway downstream etc., so as to inhibit the growth and metastasis of the tumors, and it mainly includes vascular endothelial growth inhibitor, receptor tyrosine kinase inhibitor, PI3K/AKT/mTOR pathway inhibitor, recombinant fusion protein (e.g. aflibercept) acting on VEGF-A, VEGF-B and placental growth factor, recombinant human endostatin and so on.

The immunomodulatory agent is a drug which can enhance, promote and regulate immune functions and have a certain effect on immune dysfunction, some secondary immunodeficiency diseases and some malignant tumors. In accordance with the functions of the immunomodulatory agent, the immunomodulatory agent is mainly divided into immunosuppressant and immunopotentiator. The former is used for anti-inflammatory, anti-autoimmune reactions, anti-allergy, anti-transplant rejection and anti-tumor, and the latter is for anti-infection, anti-allergy, and anti-tumor. Various kinds of drugs belong to immunosuppressant, including antimetabolic drugs (cyclosporin A, azathioprine, cyclophosphamide, methotrexate, mycophenolate, tacrolimus, mizoribine etc.), glucocorticoid, monoclonal antibody (anti-TNF-alpha/receptor, anti-IFN-γ, and anti-CD25 monoclonal antibody, etc.), cytokines IFN-β, IL-10 and TGF-↑, chemicals (leflunomide and 5-HT3 receptor antagonist), non-steroid anti-inflammatory drugs, nucleic acids, statins anti-lipid drugs, HMG coenzyme A reductase inhibitor, plants (Tripterygium wilfordii, extract of Cordyceps sinensis FTY720, artemisinin and Parviline etc.) and other biological products (cholera toxin B subunit, sNTB-A-Fc fusion protein, CMV-IkappaBa carrier inhibitor and B7-HI inhibitor etc.). There are also various kinds of immunopotentiators, including cytokines (interferon α, interferon γ, thymic peptide, Thymopentin, G-CSF/GM-CSF, IL-2, IL-12, recombinant human erythropoietin, epidermal growth factor, chemokine intercellular adhesion molecule-1, vascular cell adhesion molecule-1, P-selectin, and other intercellular adhesion molecules, etc.), biological products [IVIG, transfer factor, immune ribonucleic acid, bacteria and its extract (Bacillus Calmette Guerin and its extract, defatted and deactivated mycobacterium vaccine, other bacterial extracts, low calcium response V or V antigen LcrV, vibrio cholerae products Lot and mycobacterium etc.)], plant drugs (polysaccharides, saponins and other plant ingredients), chemicals (Levamisole, Tagamet, Pidotimod, NS-398 Imiquimod, Propagermanium and liposome etc.), micronutrients (vitamin A/C/D, trace elements iron, zinc, selenium) and others (macrolide antibiotics, aminophylline).

Immunotherapy refers to the modulation of the immune response of a subject to produce the desired therapeutic effect, the immunotherapeutic agent refers to a drug that when administered to a subject modulates the immune system of the subject so as to be sufficient to ultimately reduce the symptoms associated with an adverse immune response or ultimately reduce the symptoms caused by the increase of the required immune response.

The monoclonal antibody refers to a highly uniform antibody, produced by a single B cell clone, targeting only a specific epitope.

The polyclonal antibody refers to different antibodies produced by using an antigen immune receptor that contains multiple antigenic determinants to stimulate multiple B cell clones in the body, targeting multiple antigenic epitopes.

In biochemistry, kinases are enzymes that transfer phosphate groups from high-energy donor molecules (such as ATP) to specific target molecules (substrates); and this process is called phosphorylation; the kinase inhibitor refers to a class of molecules that may bind with kinases and reduce their activity.

Hormones are a class of chemicals that are produced by certain tissues of a normal body, and then diffuse into the blood, and are transported to other tissues in the body by blood circulation to exert special physiological functions. Hormonal compounds include synthetic or natural hormonal chemicals.

Examples of the other therapeutic agents suitable for the present invention include, but not limited to: obinutuzumab (Gazyva®), nivolumab (Opdivo®), pembrolizumab (Keytruda®), elotuzumab, anti Her2/neu antibody (e.g. trastuzumab (trade name: Herceptin®) and pertuzumab (trade name: Omnitarg™); abciximab (trade name: ReoPro®), rituximab (trade name: Mabthera®), basiliximab (trade name: Simulect®), palivizumab (trade name: Synagis®), infliximab (trade name: Remicade®), Trastuzumab (trade name: Herceptin®), alemtuzumab (trade name: Campath®), ibritumomab tiuxetan (trade name: Zevalin®), adalimumab (trade name: Humira®), omalizumab (trade name: Xolair®), tositumomab-I-131 (trade name: Bexxar®), cetuximab (trade name: Erbitux®), natalizumab (trade name: Tysabri®), tocilizumab (trade name: Actemra®), panitumumab (trade name: Vectibix®), ranibizumab (trade name: Lucentis®), eculizumab (trade name: Soliris®), certolizumab pegol (trade name: Cimzia®), golimumab (trade name: Simponi®), canakinumab (trade name: Ilaris®), ustekinumab (trade name: Stelara®), ofatumumab (trade name: Arzerra®), denosumab (trade name: Prolia®), motavizumab (trade name: Numax®), edrecolomab (trade name: Panorex®), raxibacumab (trade name: ABThrax®), belimumab (trade name: Benlysta®), ipilimumab (trade name: Yervoy®), brentuximab vedotin (trade name: Adcetris®), pertuzumab (trade name: Perjeta® or Omnitar™), ado-Trastuzumab emtansine (trade name: Adcyla®), anti-CD40 monoclonal antibody, anti-TNF-α antibody and VEGFR antibody (e.g., bevacizumab (trade name: Avastin™); Akt inhibitor; ALK inhibitor; AMPK inhibitor; antisense oligonucleotide; alkylating chemotherapeutic agent, such as nitrogen mustards (e.g., Cyclophosphamide), Mechlorethamine, HN2 (trade name: Mustardgen), Uramustine, uracil mustard, Melphalan, Chlorambucil, Ifosfamide and Bendamustine; Nitrosoureas (e.g., Carmustine), Lomustine and Streptozocin; alkyl sulfonate (e.g., Busulfan); and aziridines such as Thiotepa; platinum-based chemotherapeutants (e.g., Cisplatin, Carboplatin, Nedaplatin, Oxaliplatin, Satraplatin and Triplatin tetranitrate, Procarbazine, Altretamine, Dacarbazine, Mitozolomide and Temozolomide; APC inhibitor; apoptosis gene regulator; apoptosis regulator; ATM/ATR inhibitor; aurora kinase inhibitor; Axl inhibitor; Bcl-2 inhibitor; BCR/ABL antagonist; bFGF inhibitor; BTK inhibitor; casein kinase inhibitor (ICOS); cysteine proteinase inhibitor; CAR-T; CDK inhibitor such as palbociclib; ChK inhibitor; c-Kit inhibitor; c-Met inhibitor; EGFR inhibitor; c-Myc inhibitor; C-RET inhibitor; CSF-1R inhibitor; cytokine; DNA-PK inhibitor; dynein inhibitor; EGF receptor inhibitor; EGFR inhibitor; EGFR/ERBB inhibitor; liver protein receptor inhibitor; ERK inhibitor; estrogen agonist; estrogen antagonist; FAK inhibitor; FGFR inhibitor; FLT3 inhibitor; GF receptor antagonist; glutathione inhibitor; GSK-3 inhibitor; heat shock protein-90 inhibitor (e.g., 17-AAG); hemopoietic growth factor; HDAC inhibitor; androgen receptor pathway regulators other than the aforementioned androgen receptor pathway regulators, HER2 inhibitor; HIF inhibitor; histone deacetylase inhibitor (e.g. SAHA and LAQ 824); HSP inhibitor; IAP inhibitor; IGF-1R inhibitor; IkB kinase inhibitor; Insulin like growth factor-1 receptor inhibitor; integrin inhibitor; interferon agonist; interferon; interleukin; JAK inhibitor; JNK inhibitor; leukaemia inhibitory factor; leukocyte α interferon; lysophosphatidate acyltransferase inhibitor; matrilysin inhibitor; matrix metallo-proteinase inhibitor; Mdm2 inhibitor; MEK inhibitor; MIF inhibitor; mTOR inhibitor; oligonucleotide; P13K inhibitor (e.g., wortmannin); p38 MAPK inhibitor; p53 inhibitor; PAK inhibitor; PARP inhibitor; PDGFR inhibitor; PDK-1 inhibitor; PD-1 inhibitor; PDL-1 inhibitor; phosphatase inhibitor; Pim inhibitor; PKC inhibitor; PLK inhibitor; immunomodulatory agent based on protein A; protein kinase C inhibitor; protein tyrosine phosphatase inhibitor; purine nucleoside phosphorylase inhibitor; RacGTPase inhibitor; Raf inhibitor; Ras famesyl protein transferase inhibitor; Ras inhibitor; Ras-GAP inhibitor; ROCK inhibitor; S6 kinase inhibitor; signal transduction inhibitor; deacetylase inhibitor; Src inhibitor; STAT inhibitor; survivin inhibitor; Syk inhibitor; telomerase inhibitor; TNF-α inhibitor; topoisomerase inhibitor; Trk inhibitor; tyrosine kinase inhibitor; urokinase receptor antagonist; vascular endothelial growth factor receptor kinase inhibitor (e.g. PTK787); VDA inhibitor; VEGFR inhibitor (e.g. flk-1 specific kinase inhibitor, SU5416 and ptk787/zk222584); Wee1 inhibitor; and Wnt signaling pathway inhibitor.

Other specific therapeutic agents suitable for the present invention include, but are not limited to: acivicin; aclarubicin; acodazole hydrochloride; acronine; acylfulvene; adecypenol; adozelesin; aldesleukin; altretamine; ambamustine; ambomycin; ametantrone acetate; amidox; amifostine; aminolevulinic acid; amrubicin; amsacrine; anagrelide; anastrozole; andrographolide; antarelix; anthramycin; anti-dorsalizing morphogenetic protein-1; antineoplaston; aphidicolin glycinate; apurinic acid; ara-CDP-DL-PTBA; asparaginase; asperlin; aspirin; asulacrine; atamestane; atrimustine; axinastatin 1; axinastatin 2; axinastatin 3; azacitidine; azasetron; azatoxin; azatyrosine; azetepa; azotomycin; balanol; batimastat; benzochlorins; benzodepa; benzoylstaurosPorine; beta lactam derivatives; β-alethine; betaclamycin B; betulinic acid; bicalutamide; bisantrene hydrochloride; bisaziridinylspermine; bisnafide dimesylate; bistratene A; bizelesin; bleomycin sulfate; bortezomib; gemcitabine; brequinar sodium; bretlate; bropirimine; budotitane; busulfan; buthionine sulfoximine; cactinomycin; calcipotriol; calphostin C; calusterone; camptothecin derivatives; capecitabine; caracemide; carbetimer; carboplatin; carboxamide-amino-triazole; carboxyamidotriazole; carboxyamidotriazole; carmustine; carubicin hydrochloride; carzelesin; castanospermine; cecropin B; cedefingol; celecoxib; cetrorelix; chlorambucil; chlorins; chloroquinoxaline sulfonamide; cicaprost; cirolemycin; cisplatin; cis-porphyrin; cladribine; clomifene analogues; clotrimazole; collismycin A; collismycin B; combretastatin A4; combretastatin derivatives; conagenin; crambescidin 816; crisnatol mesylate; crisnatol; cryptophycin 8; cryptophycin A analogues; curacin A; cyclopentanthraquinones; cyclophosphamide; cycloplatam; cyclosporin; cypemycin; cytarabine ocfosfate; cytarabine; cytostatin; dacarbazine; dacliximab; dactinomycin; daunorubicin hydrochloride; decitabine; dehydrodidemnin B; deslorelin; dexamethasone; dexifosfamide; ormaplatin; dex (ormaplatin); Dextrazoxane; dexverapamil; dezaguanine mesylate; dezaguanine; diaziquone; didemnin B; didox; diethylnorspermine; dihydro-5-azacytidine; 9-dihydrotaxol; dioxamycin; diphenyl spiromustine; docetaxel; docosanol; dolasetron; doxifluridine; doxorubicin hydrochloride; doxorubicin; doxycycline; droloxifene citrate; droloxifene; dromostanolone propionate; dronabinol; duazomycin; duocarmycin SA; ebselen; ecomustine; edatrexate; edelfosine; edrecolomab; eflomithine hydrochloride; eflomithine; elemene; elotuzumab; elsamitrucin; emitefur; enloplatin; enpromate; epipropidine; epirubicin hydrochloride; epirubicin; epristeride; erbitux; erbulozole; esorubicin hydrochloride; estramustine derivatives; estramustine phosphate sodium; estramustine; etanercept; etanidazole; etoposide phosphate; etoposide; etoprine; exemestane; fadrozole hydrochloride; fadrozole; fazarabine; fenretinide; filgrastim; finasteride; flavopiridol; flezelastine; floxuridine; fluasterone; fludarabine phosphate; fludarabine; fluorocitabine; fluorodaunorunicin hydrochloride; fluorouracil; forfenimex; formestane; fosquidone; fostriecin sodium; fostriecin; fotemustine; gadolinium texaphyrin; gallium nitrate; galocitabine; ganirelix; gemcitabine hydrochloride; gemcitabine; hepsulfam; heregulin; hexamethylene bisacetamide; hydroxyurea; hypericin; ibandronic acid; ibrutinib; idarubicin hydrochloride; idarubicin; idoxifene; idramantone; ifosfamide; ilmofosine; ilomastat; imatinib (trade name: Gleevec®); imiquimod; immunostimulant peptides; iobenguane; iododoxorubicin; 4-ipomeanol; iproplatin; irinotecan hydrochloride; irinotecan; iroplact; irsogladine; isobengazole; isohomohalicondrin B; itasetron; jasplakinolide; kahalalide F; lamellarin-N triacetate; lanreotide acetate; lanreotide; lapatinib, trade name: Tykerb®; leinamycin; lenograstim; lentinan sulfate; leptolstatin; letrozole; leuprolide acetate; leuprolide+estrogen+progesterone; leuprorelin; levamisole; liarozole hydrochloride; liarozole; lipophilic disaccharide peptide; lipophilic platinum analogues; lissoclinamide 7; lobaplatin; lombricine; lometrexol sodium; lometrexol; lomustine; lonidamine; losoxantrone hydrochloride; losoxantrone; loxoribine; lurtotecan; lutetium texaphyrin; lysofylline; lytic peptides; maitansine; Amannostatin A; marimastat; masoprocol; maspin; maytansine; mechlorethamine hydrochloride; megestrol acetate; melengestrol acetate; melphalan; menogaril; merbarone; mercaptopurine; meterelin; methioninase; methotrexate sodium; methotrexate; metoclopramide; metoprine; meturedepa; mifepristone; miltefosine; mirimostim; mitindomide; mitocarcin; mitocromin; mitogillin; mitoguazone; mitolactol; mitomalcin; mitomycin derivatives; mitomycin; mitonafide; mitosper; mitotane; mitotoxin fibroblast growth factor-saporinmitotoxin; mitoxantrone hydrochloride; mitoxantrone; mofarotene; molgramostim; mopidamol; mycaperoxide B; mycophenolic acid; myriaporone; N-acetyldinaline; nafarelin; nagrestip; naloxone+pentazocine; napavin; naphterpin; nartograstim; nedaplatin; nemorubicin; neridronic acid; nilutamide; nisamycin; nitrullyn; nivolumab (Opdivo®); nocodazole; nogalamycin; O6-benzylguanine; oblimersen (trade name: Genasense®; octreotide; okicenone; onapristone; ondansetron; oracin; ormaplatin; osaterone; oxaliplatin; oxaunomycin; oxisuran; paclitaxel; paclitaxel derivatives; palauamine; palbociclib; palmitoylrhizoxin; pamidronic acid; panaxytriol; panomifene; panobinostat; parabactin; pazelliptine; pegaspargase; peldesine; peliomycin; pembrolizumab (Keytruda®); pentamustine; pentosan polysulfate sodium; pentostatin; pentrozole; peplomycin sulfate; perflubron; perfosfamide; perillyl alcohol; phenazinomycin; phenylacetate; picibanil; pilocarpine hydrochloride; pipobroman; piposulfan; pirarubicin; piritrexim; piroxantrone hydrochloride; placetin A; placetin B; platinum complex; plicamycin; plomestane; porfimer sodium; porfiromycin; prednimustine; procarbazine hydrochloride; propyl bis-acridone; prostaglandin J2; puromycin hydrochloride; puromycin; purpurins; pyrazofurin; pyrazoloacridine; raltitrexed; ramosetron; rapamycin; rapamycin derivatives (e.g., everolimus); merilimus; olcorolimus; ridaforolimus; sirolimus; temsirolimu (sirolimus, trade name: Torisel); umirolimus and zotarolimus; retelliptine demethylated; rhenium Re 186 etidronate; rhizoxin; riboprine; ribozymes; RII retinamide; rohitukine; romurtide; roquinimex; rubiginone B1; ruboxyl; safingol hydrochloride; safingol; saintopin; SarCNU; sarcophytol A; sargramostim; Sdi 1 mimetics; semaxanib; semustine; simtrazene; sizofuran; sobuzoxane; sodium borocaptate; sodium phenylacetate; solverol; somatomedin binding protein; sonermin; sparfosate sodium; sparfosate; sparsomycin; spicamycin D; spirogermanium hydrochloride; spiromustine; spiroplatin; splenopentin; spongistatin 1; squalamine; stipiamide; streptonigrin; streptozocin; sulfinosine; sulofenur; suradista; suramin; swainsonine; talisomycin; tallimustine; tamoxifen methiodide; tauromustine; taxotere; taxotere; tecogalan sodium; tegafur; tellurapyrylium; teloxantrone hydrochloride; temoporfin; teniposide; teroxirone; teroxirone; tetrachlorodecaoxide; tetrazomine; thaliblastine; thiamiprine; thiocoraline; thioguanine; thiotepa; thrombopoietin mimetics; thrombopoietin; thymalfasin; thymotrinan; tiazofurin; tin ethyl etiopurpurin; tirapazamine; titanocene bichloride; topsentin; toremifene citrate; toremifene; trestolone acetate; tretinoin; triacetyluridine; triciribine phosphate; triciribine; trimetrexate glucoronate; trimetrexate; triptorelin; tropisetron; tubulozole hydrochloride; turosteride; tyrphostins; ubenimex; uracil mustard; uredepa; vapreotide; variolin B; velaresol; veramine; verdins; verteporfin; vinblastine sulfate; vincristine sulfate; vindesine sulfate; vindesine; vinepidine sulfate; vinglycinate sulfate; vinleurosine sulfate; vinorelbine tartrate; vinorelbine; vinrosidine sulfate; vinxaltine; vinzolidine sulfate; vitamin; vitaxin; vorozole; zanoterone; zeniplatin; zinostatin; 5-ethynyluracil and zorubicin hydrochloride.

In a preferred embodiment, the other therapeutic agent is selected from one or more of daratumumab, elotuzumab, palbociclib, panobinostat, nivolumab, pembrolizumab, pemetrexed, topotecan, doxorubicin, bortezomib, gemcitabine, dacarbazine, biaxin, vincristine, azacitidine, CAR-T, rituximab, trastuzumab, PD-1 inhibitor, PD-L1 inhibitor, HDAC inhibitor, androgen receptor pathway regulators other than the aforementioned androgen receptor pathway regulators, docetaxel, clofarabine injection, Ublituximab, romidepsin, BTK inhibitor, erythropoietin, eltrombopag, minocycline and melphalan.

The present invention also provides the application of the pharmaceutical composition as described above for manufacturing a medicament for prevention or treatment of prostate cancer. The prostate cancer is preferably castration-resistant prostate cancer.

The pharmaceutical composition of the present invention may be administered to a subject for the treatment of the disease, disorder or condition of prostate cancer.

In one embodiment of the invention, the active ingredients in the pharmaceutical composition are administered to a subject simultaneously. In another embodiment, the active ingredients in the pharmaceutical composition are administered in a sequential order. In another embodiment, the active ingredients in the pharmaceutical composition are administered separately. The androgen receptor pathway modulator and/or the hormonal compound may be administered before, simultaneously with or after the administration of one or more of the compound as shown in formula (I), the pharmaceutically acceptable salt thereof, the solvate thereof, the polymorph thereof, the co-crystal thereof, the stereoisomer thereof, the isotope compound thereof, the metabolite thereof or the prodrug thereof.

In some embodiments, the therapeutically or prophylactically effective amount of the compound (herein referred as to one or more of the benzoheterocyclic compound as shown in formula (I), the pharmaceutically acceptable salt thereof, the solvate thereof, the crystalline form thereof, the co-crystal thereof, the stereoisomer thereof, the isotope compound thereof, the metabolite thereof or the prodrug thereof, the androgen receptor pathway modulator, or the hormonal compound) administered to each subject is from about 0.005 to about 1000 mg/day, from about 0.01 to about 500 mg/day, from about 0.01 to about 250 mg/day, from about 0.01 to about 100 mg/day, from about 0.1 to about 100 mg/day, from about 0.5 to about 100 mg/day, from about 1 to about 100 mg/day, from about 0.01 to about 50 mg/day, from about 0.1 to about 50 mg/day, from about 0.5 to about 50 mg/day, from about 1 to about 50 mg/day, from about 0.02 to about 25 mg/day, or from about 0.05 to about 10 mg/day.

In some embodiments, the therapeutically or prophylactically effective amount (herein referred as to the therapeutically or prophylactically effective amount of one or more of the benzoheterocyclic compound as shown in formula (I), the pharmaceutically acceptable salt thereof, the solvate thereof, the polymorph thereof, the co-crystal thereof, the stereoisomer thereof, the isotope compound thereof, the metabolite thereof or the prodrug thereof, the androgen receptor pathway modulator, or the hormonal compound) is about 0.01, about 0.05, about 0.1, about 0.2, about 0.3, about 0.4, about 0.5, about 0.6, about 0.8, about 1, about 2, about 5, about 10, about 15, about 20, about 25, about 30, about 40, about 45, about 50, About 60, about 70, about 80, about 90, about 100, about 150, about 200, about 250, about 300, about 350, about 400, about 450, about 500, about 550, about 600, about 650, about 700 About 750, about 800, about 850, about 900, or about 1000 mg/day/subject.

In another embodiment, the therapeutically or prophylactically effective amount of the androgen receptor pathway modulator or the hormone compound in the pharmaceutical composition of the present invention may be lower than the effective amount when the compound as shown in formula (I), the pharmaceutically acceptable salt thereof, the solvate thereof, the polymorph thereof, the co-crystal thereof, the stereoisomer thereof, the isotope compound thereof, the metabolite thereof or the prodrug thereof of the present invention is not administered.

In the present invention, the amount of the compound administered, the therapeutically or prophylactically effective amount, the dosage, the starting dosage and the like are all referred to the amount of a specific compound, for example, a specific heterocyclic compound as shown in formula (I), a pharmaceutically acceptable salt thereof, a solvate thereof, a polymorph thereof, a co-crystal thereof, a stereoisomer thereof, an isotope compound thereof, a metabolite thereof or a prodrug thereof, a specific androgen receptor pathway modulator or a specific hormone, rather than a combination of multiple compounds.

In the present invention, the therapeutically or prophylactically effective amount of the androgen receptor pathway modulator or the hormone compound in the method is well known to people skilled in the art, and the guidance for administration can be found in the patents and published patent applications cited herein, and Wells et al, eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000) and other medical literatures, and the contents of the literatures are incorporated herein in entirety. However, an ordinary person skilled in the art is well-qualified to determine the optimal dose range of the other therapeutic agents.

The term “androgen receptor pathway modulator” in the present invention comprises androgen inhibitor, androgen receptor inhibitor, androgen biosynthesis inhibitor and other drugs that affect the androgen receptor pathway.

As used herein, when referring to a specific salt, composition, and excipient etc. as “pharmaceutical acceptable”, it means that the salt, the composition, the excipient etc. are generally non-toxic, safe, and suitable for use in a subject, preferably a mammalian subject, more preferably a human subject.

The term “pharmaceutically acceptable salt” herein refers to a pharmaceutically acceptable organic or inorganic salt. Examples of the salt include but are not limited to: sulfate, citrate, acetate, oxalate, chloride, bromide, iodide, nitrate, hydrosulfate, phosphate, acid phosphate, isonicotinic acid salt, lactate, salicylic acid salt, acid citrate, tartrate, oleate, tannate, pantothenate, bitartrate, ascorbate, succinate, maleate, gentisinate, fumarate, gluconate, glucuronate, saccharate, formate, benzoate, glutamate, methane sulfonate, ethane sulfonate, benzene sulfonate, p-toluene sulfonate, and embonate (i.e. 1-1-methylene-bis(2-hydroxy-3-naphthoate)). The compounds of the present invention may form pharmaceutically acceptable salts with various amino acids. Suitable alkali salts include but are not limited to, aluminum salt, calcium salt, lithium salt, magnesium salt, potassium salt, sodium salt, zinc salt, bismuth salt and diethanolamine salt. For a review of the pharmaceutically acceptable salts, see Handbook of Pharmaceutical Salts: Properties, Selection, and Use (P. Heinrich Stahl and Camille G. Wermuth, ed., Wiley-VCH, 2002)

As used herein, the term “metabolite” refers to an active substance produced by changes in chemical structure that a drug molecule undergoes in vivo, the active substance is generally a derivative of the aforementioned drug molecule, and can also be chemically modified.

As used herein, the term “polymorph” refers to one or more crystal structures formed by the different arrangement of molecules in the lattice space when crystallized.

As used herein, the term “co-crystal” refers to a multi-component system comprising one or more API (active pharmaceutical ingredient) molecules and one or more object (or ligand) molecules. In the co-crystal, API molecules and object (or ligand) molecules exist as solids at room temperature when they are used as their pure form alone (in order to distinguish co-crystal from solvate or hydrate). From this particular definition, salts in which significant or complete proton exchange occurs between API molecules and guest molecules are excluded. In the co-crystal, API and ligands interact through hydrogen bonds and other possible non-covalent interactions. It is noted that the co-crystal itself may form solvates, including hydrates. The object (or ligand) refers to other physiologically acceptable acids, bases or non-ionic compounds.

As used herein, the term “solvate” refers to a crystal form of the compound as shown in formula (I), the pharmaceutically acceptable salt, the polymorph, the co-crystal, the stereoisomer, the isotopic compound, the metabolite or the prodrug thereof, which further comprises one or more solvent molecule(s) incorporated into the crystal structure. The solvate may include a stoichiometric amount or a non-stoichiometric amount of solvent, and the solvent molecule in the solvent may exist in an ordered or non-ordered arrangement. The solvate containing a non-stoichiometric amount of solvent molecules may be obtained by the loss of at least one solvent molecule (but not all) from the solvate. In a particular embodiment, a solvate refers to a hydrate, which means the crystal of the compound further comprises water molecules, with water molecules as the solvent.

As used herein, the term “prodrug” refers to a derivative of the compound comprising a biologically reactive functional group such that the biological reactive functional group can be cleaved from the compound or react in other ways to give the compound under biological conditions (in vivo or in vitro). Usually, the prodrug is inactive, or at least has lower activity than the compound itself, such that the compound exhibit its activity until it is cleaved from the biologically reactive functional group. The biologically reactive functional group can be hydrolyzed or oxidized under biological conditions to give the compound. For instance, the prodrug may contain a biologically hydrolysable group. Examples of the biologically hydrolysable group include, but are not limited to: a biologically hydrolysable phosphate, a biologically hydrolysable ester, a biologically hydrolysable amide, a biologically hydrolysable carbonic ester, a biologically hydrolysable carbamate and a biologically hydrolysable ureide. For a review of the prodrug, see, for example, J. Rautio et al., Nature Reviews Drug Discovery (2008) 7, 255-270 and Prodrugs: Challenges and Rewards (V. Stella et al. ed., Springer, 2007).

The compound as shown in formula (I), the pharmaceutically acceptable salt, the solvate, the polymorph, the eutectic, the stereoisomer, the isotopic compound, the metabolite or the prodrug thereof in the pharmaceutical composition of the present invention, can contain one or more asymmetric centers (“stereoisomer”). As used herein, the term “stereoisomer” refers to all stereoisomers including enantiomers, diastereoisomers, epimers, endo-exo isomers, atropisomers, regioisomers, cis- and trans-isomers. The “stereoisomer” herein also includes “pure stereoisomer” and “enriched stereoisomer” or “racemic isomer” of the various aforementioned stereoisomers. These stereoisomers can be prepared according to an asymmetric synthesis process, or separated, purified and enriched by a chiral separation process (including but not limited to thin layer chromatography, rotating chromatography, column chromatography, gas chromatography, high pressure liquid chromatography, etc.), and can also be obtained through chiral separation by means of bonding (chemical binding etc.) or salifying (physical binding etc.) with other chiral compound(s). The term “pure stereoisomer” herein refers to a stereoisomer of the compound with the mass content of no less than 95% relative to other stereoisomers of the compound. The term “enriched stereoisomer” herein refers to a stereoisomer of the compound with the mass content of no less than 50% relative to other stereoisomers of the compound. The term “racemic isomer” herein refers to a stereoisomer of the compound with the mass content equal to that of other stereoisomers of the compound.

As used herein, D represents deuterium-enriched hydrogen, and H represents non-deuterium-enriched hydrogen. “Deuterium-enriched” compound means that abundance of deuterium at any relevant site in the compound as shown in formula (I), a pharmaceutically acceptable salt thereof, a solvate thereof, a polymorph thereof, a co-crystal thereof, a stereoisomer thereof, an isotope compound thereof, a metabolite thereof or a prodrug thereof is greater than its natural abundance at that site (0.0156%). So, in the “deuterium-enriched” compounds, the abundance of deuterium at any of its related sites may be in the range of 0.0156% to 100%. An example of a process for obtaining deuterium-enriched compounds is to exchange hydrogen with deuterium or to synthesize the compound from deuterium-enriched starting material.

Based on the general knowledge in the art, the symbol H may be omitted in the non-deuterium-enriched site. “Non-deuterium enriched” refers to hydrogen in nature, i.e., in the form of isotopic mixture of H (hydrogen or protium), D (²H or deuterium) and T (³H or tritium).

The term “isotopic compound” used herein refers to the compound as shown in formula (I), the pharmaceutically acceptable salt, the solvate, the polymorph, the co-crystal, the stereoisomer, the isotopic compound, the metabolite or the prodrug thereof containing one or more atomic isotope(s) with natural or non-natural abundance. Atomic isotopes with non-natural abundance include, but are not limited to: deuterium (²H or D), tritium (³H or T), iodine-125 (¹²⁵I) phosphorus-32 (³²P), carbon-13 (¹³C) or carbon-14 (¹⁴C). The aforementioned isotopic compound can also be used as a therapeutic or diagnostic agent (i.e., internal developing agent) or a research tool. All the isotopic variants of the compound of the present invention, whether radioactive or not, are included in the scope of the present invention.

The term “isotope enriched” used herein refers to the compound as shown in formula (I), the pharmaceutically acceptable salt, the solvate, the polymorph, the co-crystal, the stereoisomer, the isotopic compound, the metabolite or the prodrug thereof containing one or more atomic isotope(s) with non-natural abundance. The term “isotope enriched” also refers to the compound as shown in formula (I), the pharmaceutically acceptable salt, the solvate, the polymorph, the co-crystal, the stereoisomer, the isotopic compound, the metabolite or the prodrug thereof containing at least one isotopic atom with non-natural abundance.

As used herein, the term “subject” refers to any animal to be treated or treated with the compound or the composition according to the embodiments of the present invention, preferably mammal, and most preferably human. The term “mammal” used herein includes any mammal. Examples of mammals include but are not limited to cattle, horse, sheep, pig, cat, dog, mouse, rat, rabbit, guinea pig, monkey, human and the like, most preferably human

In an embodiment, the terms “treat” and “treating” refers to an improvement, prevention or reversal of a disease or condition or at least one of identifiable symptoms thereof, such as treating cancer by reducing or stabilizing the symptoms of the cancer or the condition. In another embodiment, “treat” or “treating” refers to an improvement, prevention or reversal of at least one measurable body parameter of a disease or condition which is being treated, but may not be identified in mammal. However, in another embodiment, the term “treat” or “treating” refers to slowing the progression of a disease or condition, in physical, such as stabilizing identifiable symptoms, or in physiological, such as stabilizing physical parameters, or in both. In another embodiment, the term “treat” or “treating” refers to delaying the development of a disease or symptom.

In some embodiments, the pharmaceutical composition is administered for a prevention purpose. As used herein, “prevent” or “preventing” refers to a reduction in a risk of obtaining a given disease or condition. In a preferred embodiment, the designated pharmaceutical composition is administered for a prevention purpose to a subject, such as a subject with family history or tendency of cancer or autoimmune disease.

As used herein, “therapeutically effective amount” refers to an amount of the compound or the composition (which is sought by researchers, veterinarians, physicians, or other clinicians) that can cause a biological or medical response in a tissue system, an animal or a person, which may include relieving symptoms of the disease or symptom which is being treated. In a preferred embodiment, the therapeutically effective amount is an amount which is enough to effectively treat, improve or prevent cancer, condition or undesirable angiogenesis.

The term “prophylactically effective amount” refers to an amount of the active compound or medicament (sought by researchers, veterinarians, physicians or other clinicians) that can inhibit the development of a disease in a subject. A prophylactically effective amount of the compound refers to an amount of the therapeutic agent used alone or in combination with other active compound, which can provide a therapeutic benefit for treating or preventing the disease, condition or disorder.

Each preferred conditions aforementioned can be combined randomly without departing from the common knowledge in the art thereby forming various preferred embodiments of the present invention.

Unless otherwise specified, the singular form of the term used herein, “a” or “an”, also includes a plural meaning.

Unless otherwise specified, the term “or” or “and” used herein refers to “and/or”.

Various publications, articles, and patents are cited or described herein. The citation or description of these references or the incorporation in their entirety or the discussion about them intends to illustrate the background of the present invention, but not to mean that the contents thereof form a part of the prior art of the present invention.

Unless defined otherwise, all technical and scientific terms used herein have the same meaning as commonly understood by an ordinary person skilled in the art to which this invention belongs. Otherwise, the meaning of certain terms used herein has the meaning set forth in this description.

In the present invention, the structures of the androgen receptor pathway modulator and the hormone compound are as follows:

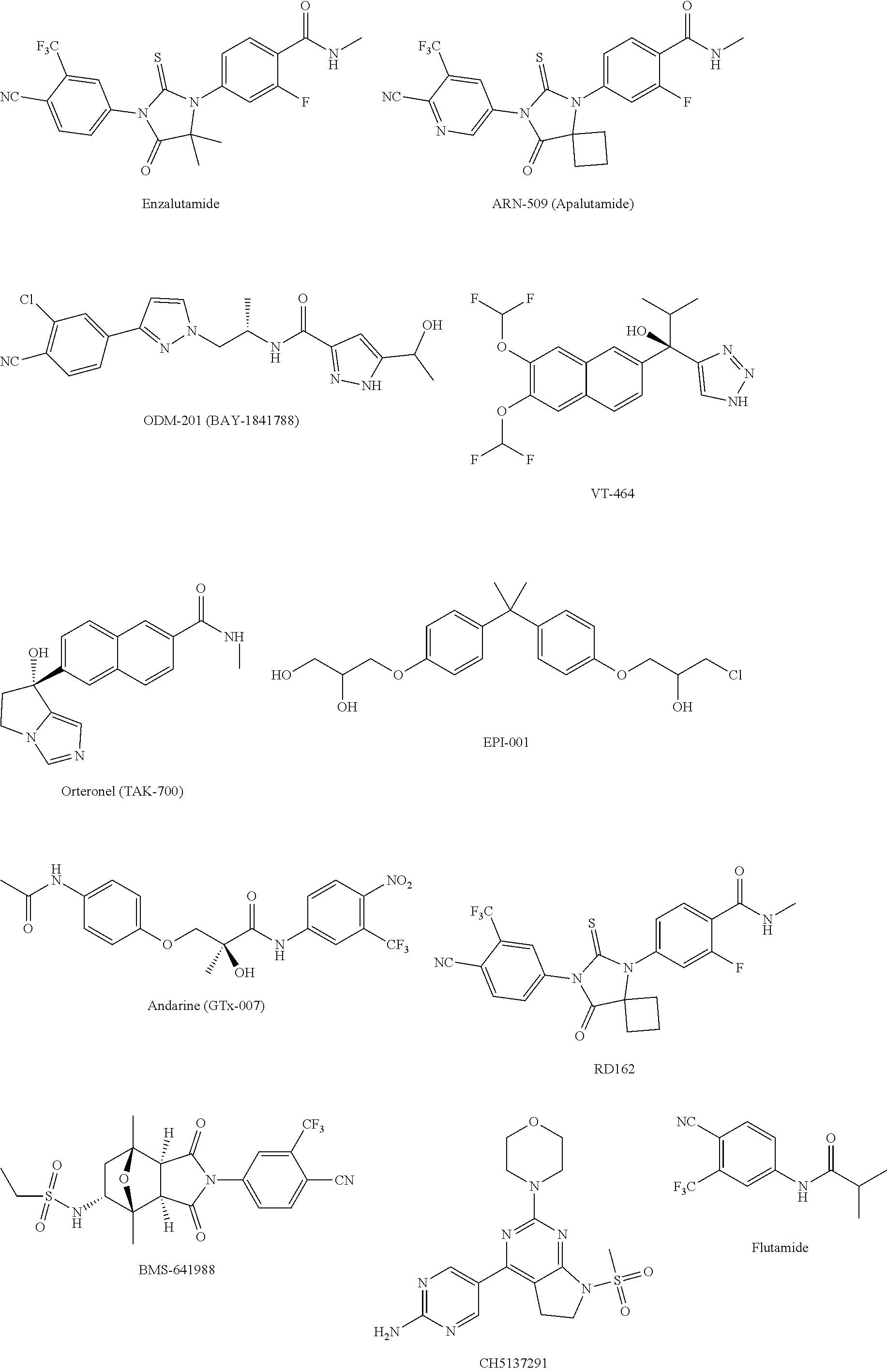

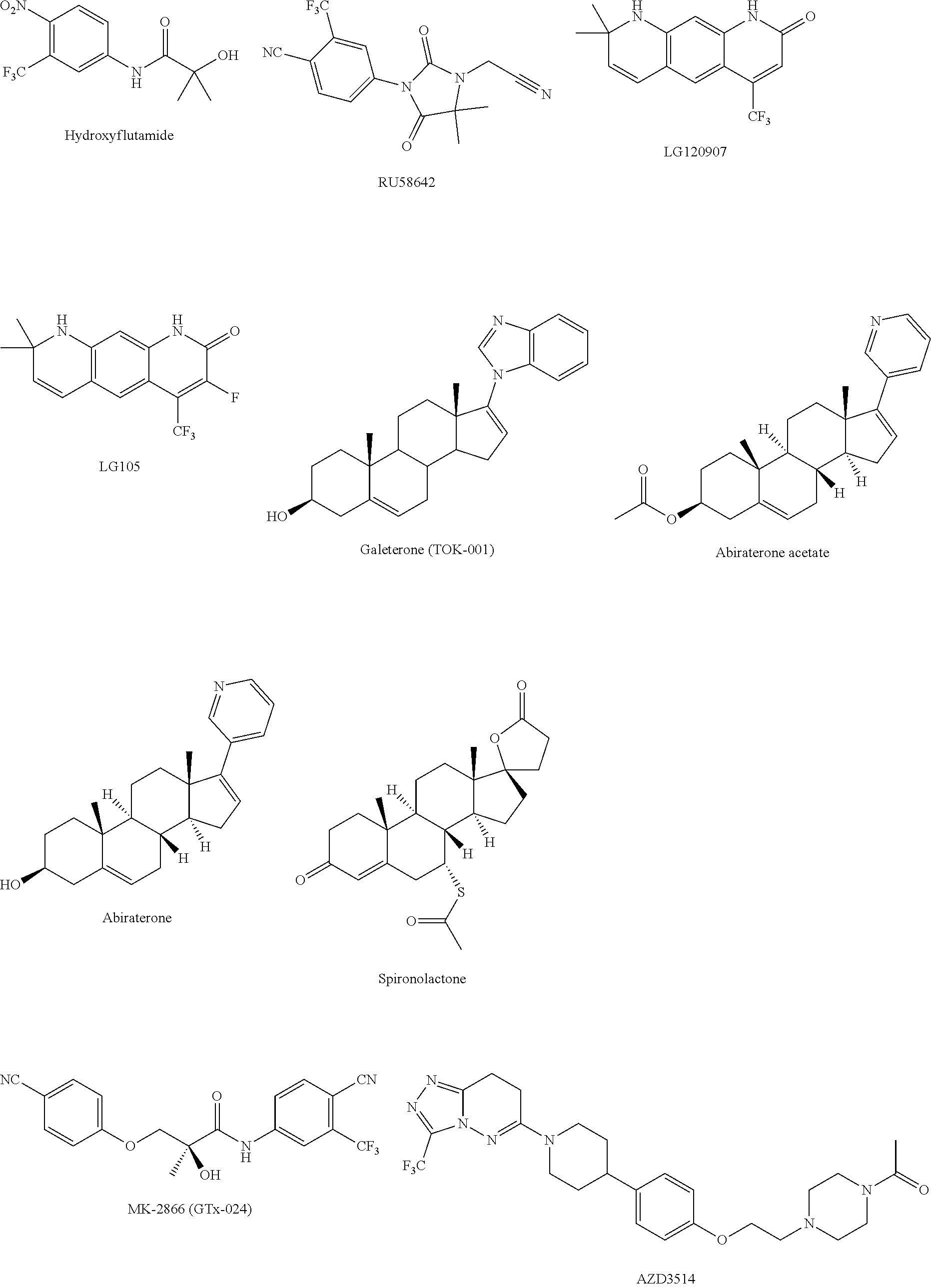

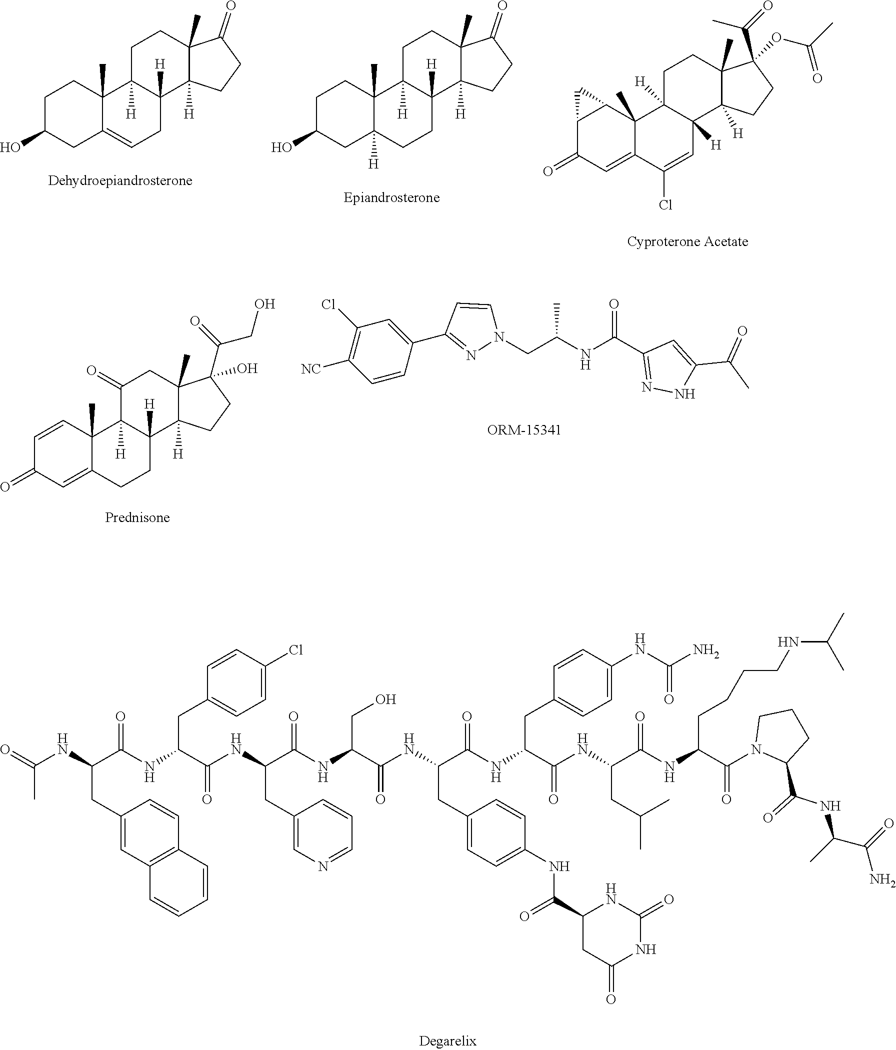

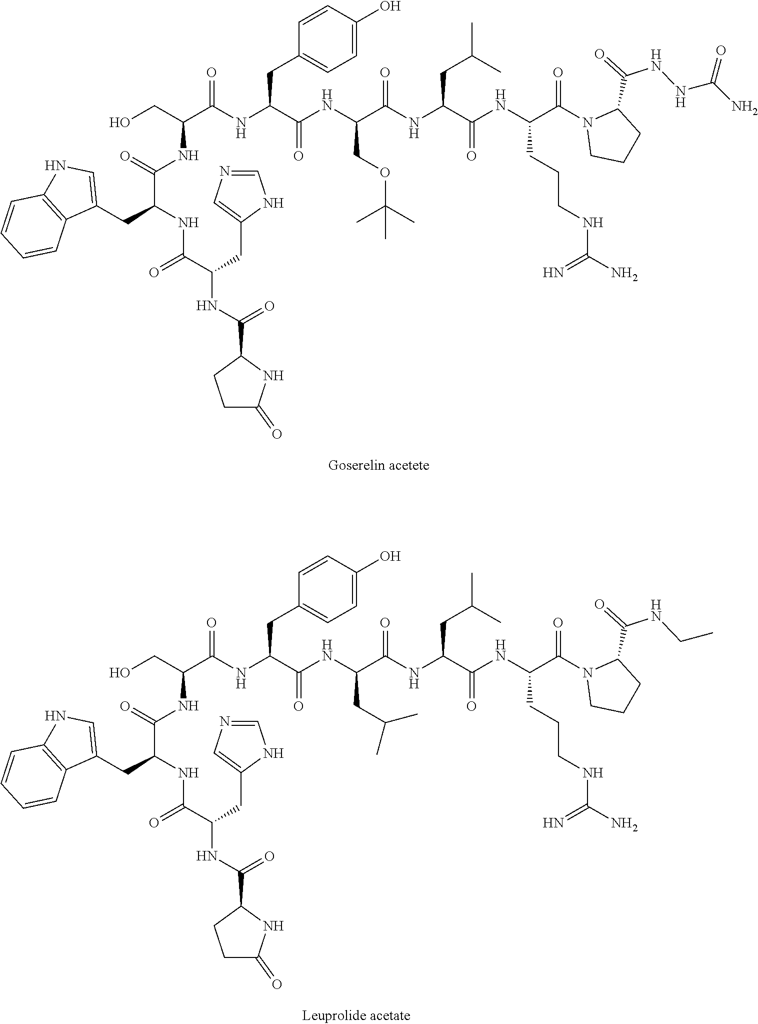 [SMILES: CC(C)C[C@H](NC(=O)[C@@H](COC(C)(C)C)NC(=O)[C@H](Cc1ccc(O)cc1)NC(=O)[C@H](CO)NC(=O)[C@H](Cc1c[nH]c2ccccc12)NC(=O)[C@H](Cc1cnc[nH]1)NC(=O)[C@@H]1CCC(=O)N1)C(=O)N[C@@H](CCCNC(=N)N)C(=O)N1CCC[C@H]1C(=O)NNC(N)=O ; CCNC(=O)[C@@H]1CCCN1C(=O)[C@H](CCCNC(=N)N)NC(=O)[C@H](CC(C)C)NC(=O)[C@@H](CC(C)C)NC(=O)[C@H](Cc1ccc(O)cc1)NC(=O)[C@H](CO)NC(=O)[C@H](Cc1c[nH]c2ccccc12)NC(=O)[C@H](Cc1cnc[nH]1)NC(=O)[C@@H]1CCC(=O)N1]

The reagents used in the present invention are all commercially available. The compound as shown as formula (I) and the androgen receptor pathway modulator in the present invention may be obtained commercially or prepared by people skilled in the art according to synthetic methods well known in the art, or readily synthesized according to the published literatures or patents, such as WO9803502, WO2010056344, WO2012079022, WO2012015986, WO2011100380, WO2014116573, WO2008039489, WO2014110558, WO2014039421, WO2006124118 and so on. The entire contents of the above patents are incorporated herein by reference.

Each preferred conditions aforementioned can be combined randomly without departing from the common knowledge in the art thereby forming various preferred embodiments of the present invention.

The positive effect of the present invention is that the pharmaceutical composition of the present invention can inhibit the growth of prostate cancer cells more effectively.

### DETAILED DESCRIPTION OF THE EMBODIMENTS

### Effect Embodiment 1 CTG Cell Proliferation Assay

In vitro test of the inhibition effect of the compound in combination with androgen receptor pathway modulators and the like on the prostate cancer cell proliferation.

Inhibition effect of the compounds such as B001, K001, D101, D107, D108, F001, B002, B003, B004, B005, B006 and the like alone or in combination with androgen receptor pathway modulators on the prostate cancer cell proliferation were tested on Vcap cells (androgen receptor (+) prostate cancer cells) (ATCC, catalogue number CRL-2876) The specific experimental operation was as follows: 5×10³ Vcap cells per well were inoculated into 96-well plates with transparent bottom and white wall (Corning, catalogue number CLS3903) containing the specific medium, and were cultured in a 37° C., 5% CO₂ incubator for 24 hours. The tested compounds and the androgen receptor pathway modulators were prepared to a 150 mM stocking solution with DMSO (Sigma, catalogue number 276855), diluted with culture medium to the desired concentrations (the final concentration of DMSO is 0.2%), and then added to each well, 2 wells/concentration, followed by being incubated in a 37° C., 5% CO₂ incubator for 5 days. The tested compounds were used alone or in combination with other androgen receptor pathway modulators respectively. The combination drugs were: Enzalutamide (Kangpu Biopharmaceuticals, Ltd.), ARN-509 (Selleck, catalogue number S2840), Abiraterone acetate (Selleck, catalogue number S2246), Galeterone (Selleck, catalogue number S2803), ODM-201 (Kangpu Biopharmaceuticals, Ltd.) or Prednisone (Selleck, catalogue number S1622). The concentration setting of each drug was shown in the following tables of experimental results. After that, 100 μl of CellTiter-Glo® cell viability assay reagent (Promega, catalogue number G7570) was added to each well and mixed well on a vibrator for 10 minutes to induce cell lysis. The 96-well plate was placed at room temperature for 10 minutes, so as to stabilize its luminescence signal. A white bottom membrane was pasted on the bottom of the plate and the plate was tested using EnSpire. The data was processed by Graphpad/Prism and Calcusyn software to calculate the average cell proliferation inhibition rate or survival rate for each compound or the synergism index of the drug combination, and the specific experimental results were shown in Tables 1-10.

TABLE 1
Vcap cell proliferation inhibition rate: the combination of B001 and Enzalutamide

| | | B001 (μM) | | | | | | |
|---|---|---|---|---|---|---|---|---|
| | | 100.00 | 33.33 | 11.11 | 3.70 | 1.23 | 0.41 | 0.00 |
| Enzalutamide | 11.11 | 59.3% | 59.0% | 56.9% | 55.4% | 54.3% | 52.5% | 36.7% |
| (μM) | 3.70 | 60.1% | 61.3% | 61.1% | 59.6% | 57.6% | 55.1% | 37.4% |
| | 1.23 | 57.2% | 56.7% | 58.9% | 57.1% | 54.8% | 52.6% | 28.7% |
| | 0.41 | 44.4% | 52.0% | 52.6% | 50.2% | 50.7% | 48.1% | 28.1% |
| | 0.14 | 34.1% | 39.4% | 41.2% | 39.8% | 38.6% | 37.9% | 25.0% |
| | 0.05 | 27.4% | 32.1% | 31.6% | 30.0% | 30.8% | 30.2% | 17.2% |
| | 0.00 | 19.6% | 22.0% | 20.9% | 21.4% | 20.7% | 18.7% | 6.5% |

Noes of table 1: The cell proliferation inhibition rate (%) was measured after processing the Vcap cells with different concentrations of B001 (0.41-100 μM) and Enzalutamide (0.05-11.11 μM) alone or in combination for 5 days. The effect of drug combination was outstanding, for example, the inhibition rate on Vcap cells was 20.7% when B001 was used alone (concentration of 1.23 μM), the inhibition rate on Vcap cells was 28.7% when Enzalutamide was used alone (concentration of 1.23 μM), and the inhibition rate on Vcap cells was 54.8% when the two were combined (1.23 μM B001 and 1.23 μM Enzalutamide).

TABLE 2
Synergism index of the combination of B001 and Enzalutamide
(carrying out synergism analysis on the experimental data of the
drug combination in table 1)

| | | B001 (μM) | | | | | |
|---|---|---|---|---|---|---|---|
| | | 100.00 | 33.33 | 11.11 | 3.70 | 1.23 | 0.41 |
| Enzalutamide | 11.11 | 0.012 | 0.012 | 0.02 | 0.028 | 0.035 | 0.053 |
| (μM) | 3.70 | 0.003 | 0.002 | 0.003 | 0.004 | 0.006 | 0.01 |
| | 1.23 | 0.002 | 0.002 | 0.001 | 0.002 | 0.004 | 0.006 |
| | 0.41 | 0.012 | 0.002 | 0.002 | 0.003 | 0.003 | 0.005 |
| | 0.14 | 0.043 | 0.012 | 0.008 | 0.011 | 0.015 | 0.017 |
| | 0.05 | 0.088 | 0.025 | 0.029 | 0.044 | 0.036 | 0.042 |

Notes of the synergism index of drug combination: <0.1: very strong synergism; 0.1-0.3: strong synergism; 0.3-0.7: synergism; 0.7-0.85: mild synergism; 0.85-0.90: slight synergism; 0.90-1.10: approximately additive action; 1.10-1.20: slight antagonism; 1.20-1.45: mild antagonism; 1.45-3.3: antagonism; 3.3-10: strong antagonism; >10: very strong antagonism.

Notes of table 2: The synergism analysis was carried out on the experimental data of the drug combination of B001 and Enzalutamide in table 1 to give the data in Table 2 which showed a strong synergism when the two drugs are used in combination.

TABLE 3
Survival rate of Vcap cells: tested compound alone, tested compound
in combination with Enzalutamide or ARN-509

| | Tested compound | | | | | | | | |
|---|---|---|---|---|---|---|---|---|---|
| | | | | In combination | | | In combination | | |
| | | | | with 1 μM | | | with 1 μM | | |
| | Tested compound alone | | | Enzalutamide | | | ARN-509 | | |
| | Concentration of the tested compound | | | | | | | | |
| | 10 μM | 1.0 μM | 0.1 μM | 10 μM | 1.0 μM | 0.1 μM | 10 μM | 1.0 μM | 0.1 μM |
| Enzalutamide | 52.3% | 54.0% | 74.5% | | | | | | |
| ARN-509 | 48.9% | 52.2% | 81.8% | | | | | | |
| B001 | 81.9% | 82.8% | 92.0% | 33.7% | 35.6% | 40.3% | 33.5% | 34.7% | 42.0% |
| K001 | 61.4% | 65.8% | 85.1% | 35.1% | 36.4% | 42.4% | 33.5% | 36.0% | 43.6% |
| D107 | 69.3% | 77.2% | 85.3% | 32.3% | 35.0% | 37.3% | 34.2% | 36.3% | 37.0% |

Notes of table 3: The effect of the drug combination was outstanding, for example, the cell survival rates were 82.8%, 65.8%, 77.2%, 54.0% and 52.2% when B001, K001, D107, enzalutamide and ARN-509 were used alone respectively at 1.0 μM. The cell survival rates were 35.6%, 36.4% and 35.0% when 1.0 μM B001, K001, or D107 was used in combination with 1.0 μM enzalutamide respectively. The cell survival rates were 34.7%, 36.0% and or 36.3% when 1.0 μM B001, K001, or D107 was used in combination with 1.0 μM ARN-509 respectively.

TABLE 4
Survival rate of Vcap cells: tested compound alone, tested compound
in combination with Galeterone or Abiraterone acetate

| | Tested compounds | | | | | | | | |
|---|---|---|---|---|---|---|---|---|---|
| | | | | In combination with | | | In combination with 1 μM | | |
| | Tested compounds alone | | | 1 μM Galeterone | | | Abiraterone acetate | | |
| | Concentration of the tested compounds | | | | | | | | |
| | 10 μM | 1 μM | 0.1 μM | 10 μM | 1 μM | 0.1 μM | 10 μM | 1 μM | 0.1 μM |
| Enzalutamide | 52.3% | 54.0% | 74.5% | 50.9% | 52.4% | 59.5% | 51.7% | 50.7% | 69.7% |
| B001 | 81.9% | 82.8% | 92.0% | 42.8% | 43.3% | 51.1% | 62.9% | 68.0% | 78.5% |
| K001 | 61.4% | 65.8% | 85.1% | 39.4% | 41.3% | 49.1% | 48.1% | 53.1% | 70.4% |
| D107 | 69.3% | 77.2% | 85.3% | 40.0% | 42.4% | 43.9% | 59.6% | 61.7% | 66.0% |
| Galeterone | 19.3% | 62.8% | 95.9% | / | / | / | / | / | / |
| Abiraterone | 31.4% | 91.7% | 106.3% | / | / | / | / | / | / |
| acetate | | | | | | | | | |

Notes of table 4: The effect of the drug combination was outstanding, for example, the cell survival rates were 82.8%, 65.8%, 77.2%, 62.8% and 91.7% when B001, K001, D107, Galeterone and Abiraterone acetate were used alone respectively at 1.0 μM. The cell survival rates were 43.3%, 41.3% and 42.4% when 1.0 μM B001, K001, or D107 was used in combination with 1.0 μM Galeterone respectively. The cell survival rates were 68.0%, 53.1% and 61.7% when 1.0 μM B001, K001, or D107 was used in combination with 1.0 μM Abiraterone acetate respectively.

TABLE 5
Survival rate of Vcap cells: tested compound alone, tested compound
in combination with ODM-201

| | Tested compounds | | | | | |
|---|---|---|---|---|---|---|
| | | | | In combination with | | |
| | Tested compounds alone | | | 1 μM ODM-201 | | |
| | Concentration of the tested compound | | | | | |
| | 10 μM | 1.0 μM | 0.1 μM | 10 μM | 1.0 μM | 0.1 μM |
| ODM- | 75.4% | 69.0% | 90.7% | | | |
| 201 | | | | | | |
| K001 | 51.0% | 59.7% | 79.2% | 35.2% | 40.4% | 53.5% |
| B001 | 72.3% | 75.3% | 87.7% | 37.5% | 41.4% | 52.7% |
| D108 | 58.5% | 67.3% | 73.0% | 32.8% | 40.1% | 42.7% |
| F001 | 55.7% | 59.9% | 79.7% | 37.3% | 40.9% | 49.4% |

Notes of table 5: The effect of the drugs combination was outstanding, for example, the cell survival rates were 69.0%, 59.7%, 75.3%, 67.3% and 59.9% respectively when ODM-201, K001, B001, D108, and F001 were used alone at 1.0 μM. The cell survival rates were 40.4%, 41.4%, 40.1% and 40.9% when 1.0 μM K001, B001, D108 or F001 was used in combination with 1.0 μM ODM-201 respectively.

TABLE 6
Survival rate of Vcap cells: B001 in combination with Enzalutamide,
ARN-509, Prednisone, Galeterone, or Abiraterone acetate respectively

| | | | | | | | | | | Abiraterone | Cell |
|---|---|---|---|---|---|---|---|---|---|---|---|
| Drug | B001 | Enzalutamide | | | ARN-509 | | | Prednisone | Galeterone | acetate | survival |
| combination | 1.0 μM | 10 μM | 1.0 μM | 0.1 μM | 10 μM | 1.0 μM | 0.1 μM | 1.0 μM | 1.0 μM | 1.0 μM | rate |
| 2-drug combination | | v | | | | | | v | | | 51.8% |
| | | | v | | | | | v | | | 54.5% |
| | | | | v | | | | v | | | 84.6% |
| | | v | | | | | | | v | | 50.9% |
| | | | v | | | | | | v | | 52.4% |
| | | | | v | | | | | v | | 59.5% |
| | | v | | | | | | | | v | 51.7% |
| | | | v | | | | | | | v | 50.7% |
| | | | | v | | | | | | v | 69.7% |
| | | | | | v | | | v | | | 47.4% |
| | | | | | | v | | v | | | 54.3% |
| | | | | | | | v | v | | | 79.6% |
| | | | | | v | | | | v | | 46.5% |
| | | | | | | v | | | v | | 50.8% |
| | | | | | | | v | | v | | 61.0% |
| | | | | | v | | | | | v | 46.6% |
| | | | | | | v | | | | v | 50.5% |
| | | | | | | | v | | | v | 68.9% |
| 3-drug combination | | v | | | | | | v | v | | 52.6% |
| | | | v | | | | | v | v | | 53.8% |
| | | | | v | | | | v | v | | 55.4% |
| | | v | | | | | | v | | v | 47.6% |
| | | | v | | | | | v | | v | 49.2% |
| | | | | v | | | | v | | v | 68.4% |
| | | | | | v | | | v | v | | 47.5% |
| | | | | | | v | | v | v | | 49.4% |
| | | | | | | | v | v | v | | 59.7% |
| | | | | | v | | | v | | v | 43.7% |
| | | | | | | v | | v | | v | 47.4% |
| | | | | | | | v | v | | v | 70.0% |
| | v | | | | | | | v | v | | 42.3% |
| | v | | | | | | | v | | v | 60.5% |
| | v | v | | | | | | | v | | 33.2% |
| | v | | v | | | | | | v | | 34.1% |
| | v | | | v | | | | | v | | 36.7% |
| | v | v | | | | | | | | v | 31.7% |
| | v | | v | | | | | | | v | 34.8% |
| | v | | | v | | | | | | v | 44.1% |
| | v | v | | | | | | v | | | 33.3% |
| | v | | v | | | | | v | | | 33.5% |
| | v | | | v | | | | v | | | 55.6% |
| | v | | | | v | | | | v | | 29.0% |
| | v | | | | | v | | | v | | 30.8% |
| | v | | | | | | v | | v | | 40.8% |
| | v | | | | v | | | | | v | 29.7% |
| | v | | | | | v | | | | v | 31.0% |
| | v | | | | | | v | | | v | 41.1% |
| | v | | | | v | | | v | | | 32.4% |
| | v | | | | | v | | v | | | 30.2% |
| | v | | | | | | v | v | | | 52.4% |
| 4-drug combination | v | v | | | | | | v | v | | 37.4% |
| | v | | v | | | | | v | v | | 33.4% |
| | v | | | v | | | | v | v | | 36.6% |
| | v | v | | | | | | v | | v | 36.8% |
| | v | | v | | | | | v | | v | 34.7% |
| | v | | | v | | | | v | | v | 42.2% |
| | v | | | | v | | | v | v | | 32.3% |
| | v | | | | | v | | v | v | | 32.9% |
| | v | | | | | | v | v | v | | 34.1% |
| | v | | | | v | | | v | | v | 31.1% |
| | v | | | | | v | | v | | v | 31.9% |
| | v | | | | | | v | v | | v | 42.4% |

“V” refers to the components contained in the combination. The blank indicates the component was not contained. The same below.

Notes of table 6: The effect of the drug combination was outstanding, for example, the cell survival rate was 50.7% when 1.0 μM Enzalutamide was used in combination with 1.0 μM Abiraterone acetate. The cell survival rate decreased to 34.8% when 1.0 μM Enzalutamide was used in combination with 1.0 μM Abiraterone acetate and 1.0 μM B001. The cell survival rate was 49.2% when 1.0 μM Enzalutamide was used in combination with 1.0 μM Prednisone and 1.0 μM Abiraterone acetate. The cell survival rate decreased to 34.7% when 1.0 μM Enzalutamide was used in combination with 1.0 μM Prednisone and 1.0 μM Abiraterone acetate and 1.0 μM B001.

TABLE 7
Survival rate of Vcap cells: K001 in combination with Enzalutamide,
ARN-509, Prednisone, Galeterone, or Abiraterone acetate respectively

| | | | | | | | | | | Abiraterone | Cell |
|---|---|---|---|---|---|---|---|---|---|---|---|
| Drug | K001 | Enzalutamide | | | ARN-509 | | | Prednisone | Galeterone | acetate | survival |
| combination | 1.0 μM | 10 μM | 1.0 μM | 0.1 μM | 10 μM | 1.0 μM | 0.1 μM | 1.0 μM | 1.0 μM | 1.0 μM | rate |
| 2-drug | | v | | | | | | v | | | 51.8% |
| combination | | | v | | | | | v | | | 54.5% |
| | | | | v | | | | v | | | 84.6% |
| | | v | | | | | | | v | | 50.9% |
| | | | v | | | | | | v | | 52.4% |
| | | | | v | | | | | v | | 59.5% |
| | | v | | | | | | | | v | 51.7% |
| | | | v | | | | | | | v | 50.7% |
| | | | | v | | | | | | v | 69.7% |
| | | | | | v | | | v | | | 47.4% |
| | | | | | | v | | v | | | 54.3% |
| | | | | | | | v | v | | | 79.6% |
| | | | | | v | | | | v | | 46.5% |
| | | | | | | v | | | v | | 50.8% |
| | | | | | | | v | | v | | 61.0% |
| | | | | | v | | | | | v | 46.6% |
| | | | | | | v | | | | v | 50.5% |
| | | | | | | | v | | | v | 68.9% |
| 3-drug combination | | v | | | | | | v | v | | 53.6% |
| | | | v | | | | | v | v | | 50.5% |
| | | | | v | | | | v | v | | 55.5% |
| | | v | | | | | | v | | v | 49.2% |
| | | | v | | | | | v | | v | 48.0% |
| | | | | v | | | | v | | v | 67.1% |
| | | | | | v | | | v | v | | 49.3% |
| | | | | | | v | | v | v | | 48.1% |
| | | | | | | | v | v | v | | 61.0% |
| | | | | | v | | | v | | v | 46.9% |
| | | | | | | v | | v | | v | 46.1% |
| | | | | | | | v | v | | v | 73.3% |
| | v | | | | | | | v | v | | 42.5% |
| | v | | | | | | | v | | v | 52.4% |
| | v | v | | | | | | | v | | 32.6% |
| | v | | v | | | | | | v | | 32.8% |
| | v | | | v | | | | | v | | 40.2% |
| | v | v | | | | | | | | v | 33.0% |
| | v | | v | | | | | | | v | 32.0% |
| | v | | | v | | | | | | v | 44.2% |
| | v | v | | | | | | v | | | 33.7% |
| | v | | v | | | | | v | | | 33.0% |
| | v | | | v | | | | v | | | 47.7% |
| | v | | | | v | | | | v | | 31.1% |
| | v | | | | | v | | | v | | 31.2% |
| | v | | | | | | v | | v | | 35.9% |
| | v | | | | v | | | | | v | 33.4% |
| | v | | | | | v | | | | v | 30.5% |
| | v | | | | | | v | | | v | 38.5% |
| | v | | | | v | | | v | | | 34.1% |
| | v | | | | | v | | v | | | 31.7% |
| | v | | | | | | v | v | | | 43.7% |
| 4-drug combination | v | v | | | | | | v | v | | 36.7% |
| | v | | v | | | | | v | v | | 34.6% |
| | v | | | v | | | | v | v | | 36.8% |
| | v | v | | | | | | v | | v | 34.6% |
| | v | | v | | | | | v | | v | 35.0% |
| | v | | | v | | | | v | | v | 42.9% |
| | v | | | | v | | | v | v | | 31.7% |
| | v | | | | | v | | v | v | | 35.3% |
| | v | | | | | | v | v | v | | 37.3% |
| | v | | | | v | | | v | | v | 30.2% |
| | v | | | | | v | | v | | v | 33.4% |
| | v | | | | | | v | v | | v | 39.0% |

Notes of table 7: the effect of the drug combination was outstanding, for example, the cell survival rate was 50.4% when 1.0 μM Enzalutamide was used in combination with 1.0 μM Abiraterone acetate. The cell survival rate decreased to 32.0% when 1.0 μM Enzalutamide was used in combination with 1.0 μM Abiraterone acetate and 1.0 μM K001. The cell survival rate was 48.0% when 1.0 μM Enzalutamide was used in combination with 1.0 μM Prednisone and 1.0 μM Abiraterone acetate. The cell survival rate decreased to 35.0% when 1.0 μM Enzalutamide was used in combination with 1.0 μM Prednisone, 1.0 μM Abiraterone acetate and 1.0 μM K001.

TABLE 8
Survival rate of Vcap cells: D101 in combination with Enzalutamide,
ARN-509, Prednisone, Galeterone, or Abiraterone acetate respectively

| | | | | | | | | | | Abiraterone | Cell |
|---|---|---|---|---|---|---|---|---|---|---|---|
| Drug | D101 | Enzalutamide | | | ARN-509 | | | Prednisone | Galeterone | acetate | survival |
| combination | 1.0 μM | 10 μM | 1.0 μM | 0.1 μM | 10 μM | 1.0 μM | 0.1 μM | 1.0 μM | 1.0 μM | 1.0 μM | rate |
| 2-drug combination | | v | | | | | | v | | | 51.8% |
| | | | v | | | | | v | | | 54.5% |
| | | | | v | | | | v | | | 84.6% |
| | | v | | | | | | | v | | 50.9% |
| | | | v | | | | | | v | | 52.4% |
| | | | | v | | | | | v | | 59.5% |
| | | v | | | | | | | | v | 51.7% |
| | | | v | | | | | | | v | 50.7% |
| | | | | v | | | | | | v | 69.7% |
| | | | | | v | | | v | | | 47.4% |
| | | | | | | v | | v | | | 54.3% |
| | | | | | | | v | v | | | 79.6% |
| | | | | | v | | | | v | | 46.5% |
| | | | | | | v | | | v | | 50.8% |
| | | | | | | | v | | v | | 61.0% |
| | | | | | v | | | | | v | 46.6% |
| | | | | | | v | | | | v | 50.5% |
| | | | | | | | v | | | v | 68.9% |
| 3-rug combination | | v | | | | | | v | v | | 56.3% |
| | | | v | | | | | v | v | | 54.3% |
| | | | | v | | | | v | v | | 57.0% |
| | | v | | | | | | v | | v | 52.8% |
| | | | v | | | | | v | | v | 53.0% |
| | | | | v | | | | v | | v | 73.6% |
| | | | | | v | | | v | v | | 50.6% |
| | | | | | | v | | v | v | | 53.6% |
| | | | | | | | v | v | v | | 58.1% |
| | | | | | v | | | | | v | 47.1% |
| | | | | | | v | | | | v | 50.5% |
| | | | | | | | v | | | v | 75.1% |
| | v | | | | | | | v | v | | 43.6% |
| | v | | | | | | | v | | v | 59.2% |
| | v | v | | | | | | | v | | 33.2% |
| | v | | v | | | | | | v | | 33.1% |
| | v | | | v | | | | | v | | 39.3% |
| | v | v | | | | | | | | v | 34.1% |
| | v | | v | | | | | | | v | 33.9% |
| | v | | | v | | | | | | v | 50.1% |
| | v | v | | | | | | v | | | 36.0% |
| | v | | v | | | | | v | | | 34.8% |
| | v | | | v | | | | v | | | 55.0% |
| | v | | | | v | | | | v | | 30.3% |
| | v | | | | | v | | | v | | 32.8% |
| | v | | | | | | v | | v | | 37.1% |
| | v | | | | v | | | | | v | 31.6% |
| | v | | | | | v | | | | v | 32.5% |
| | v | | | | | | v | | | v | 41.9% |
| | v | | | | v | | | v | | | 33.9% |
| | v | | | | | v | | v | | | 34.0% |
| | v | | | | | | v | v | | | 49.2% |
| 4- | v | v | | | | | | v | v | | 36.5% |
| rug | v | | v | | | | | v | v | | 33.9% |
| comb | v | | | v | | | | v | v | | 38.4% |
| | v | v | | | | | | v | | v | 36.1% |
| | v | | v | | | | | v | | v | 33.8% |
| | v | | | v | | | | v | | v | 44.7% |
| | v | | | | v | | | v | v | | 30.9% |
| | v | | | | | v | | v | v | | 31.9% |
| | v | | | | | | v | v | v | | 34.1% |
| | v | | | | v | | | v | | v | 29.5% |
| | v | | | | | v | | v | | v | 31.2% |
| | v | | | | | | v | v | | v | 43.9% |

Notes of table 8: The effect of the drug combination was outstanding, for example, the cell survival rate was 50.7% when 1.0 μM Enzalutamide was used in combination with 1.0 μM Abiraterone acetate. The cell survival rate decreased to 33.9% when 1.0 μM Enzalutamide was used in combination with 1.0 μM Abiraterone acetate and 1.0 μM D101. The cell survival rate was 53.0% when 1.0 μM Enzalutamide was used in combination with 1.0 μM Prednisone and 1.0 μM Abiraterone acetate. The cell survival rate decreased to 33.8% when 1.0 μM Enzalutamide was used in combination with 1.0 μM Prednisone, 1.0 μM Abiraterone acetate and 1.0 μM D101.

TABLE 9
Survival rate of Vcap cells: Survival rate of Vcap cells: tested
compound alone, tested compound in combination with Enzalutamide or ARN-509

| | Tested compound | | | | | | | | |
|---|---|---|---|---|---|---|---|---|---|
| | | | | In combination with 1 μM | | | In combination with 1 μM | | |
| | Tested compounds alone | | | Enzalutamide | | | ARN-509 | | |
| | Concentration of the tested compound | | | | | | | | |
| | 10 μM | 1.0 μM | 0.1 μM | 10 μM | 1.0 μM | 0.1 μM | 10 μM | 1.0 μM | 0.1 μM |
| B001 | 69.1% | 74.8% | 83.9% | 38.4% | 42.3% | 52.1% | 40.9% | 42.6% | 50.9% |
| B002 | 75.9% | 79.8% | 85.6% | 43.1% | 44.7% | 53.9% | 44.3% | 44.7% | 52.5% |
| B003 | 80.4% | 81.3% | 83.7% | 44.2% | 45.4% | 53.5% | 42.4% | 46.5% | 54.0% |
| B004 | 88.0% | 88.4% | 94.1% | 51.1% | 50.8% | 60.4% | 48.0% | 51.2% | 59.1% |
| B005 | 83.5% | 85.0% | 91.1% | 46.5% | 48.4% | 56.5% | 46.2% | 47.6% | 53.4% |
| B006 | 85.7% | 89.7% | 91.7% | 48.3% | 50.5% | 61.2% | 45.0% | 51.7% | 63.2% |
| Enzalutamide | 58.4% | 65.7% | 85.9% | / | / | / | / | / | / |
| ARN-509 | 61.9% | 70.4% | 95.9% | / | / | / | / | / | / |

Notes of table 9: The effect of the drugs combination was outstanding, for example, the cell survival rates were 65.7% and 70.4% when Enzalutamide and ARN-509 were used alone respectively. The cell survival rates were 42.3%, 44.7%, 45.4%, 50.8%, 48.4% and 50.5% when 1.0 μM Enzalutamide was used in combination with 1.0 μM B001, B002, B003, B004, B005 or B006 respectively. The cell survival rates were 42.6%, 44.7%, 46.5%, 51.2%, 47.6% and 51.7% when 1.0 μM Enzalutamide was used in combination with 1.0 μM B001, B002, B003, B004, B005 or B006 respectively.

TABLE 10
Comparision of survival rate of Vcap cells: tested compound alone,
tested compound in combination with B001 or K001

| | Tested compound | | | | | | | | |
|---|---|---|---|---|---|---|---|---|---|
| | | | | In combination with | | | In combination with | | |
| | Tested compound alone | | | 1 μM B001 | | | 1 μM K001 | | |
| | Concentration of the tested compound | | | | | | | | |
| | 10 μM | 1.0 μM | 0.1 μM | 10 μM | 1.0 μM | 0.1 μM | 10 μM | 1.0 μM | 0.1 μM |
| B001 | 69.1% | 74.7% | 83.9% | / | / | / | / | / | / |
| K001 | 66.1% | 73.8% | 91.2% | / | / | / | / | / | / |
| Prednisone | 99.4% | 99.7% | 99.1% | 73.8% | 78.7% | 87.6% | 68.0% | 74.2% | 91.5% |
| Abiraterone | 81.9% | 92.4% | 98.5% | 66.3% | 74.9% | 78.9% | / | / | / |
| bicalutamide | 92.0% | 94.0% | 99.2% | 67.1% | 69.0% | 77.1% | 61.5% | 64.1% | 78.7% |
| MK-2866 | 69.4% | 77.4% | 76.5% | 57.8% | 65.6% | 64.8% | / | / | / |
| ARN-509 | 61.9% | 70.4% | 95.9% | 40.9% | 42.6% | 50.9% | 41.8% | 45.1% | 54.1% |
| Dehydroepiandrosterone | 100.4% | 102.4% | 101.2% | 85.9% | 90.6% | 90.7% | / | / | / |
| Epiandrosterone | 90.4% | 82.0% | 87.3% | 79.7% | 71.0% | 77.5% | / | / | / |
| Abiraterone acetate | 60.5% | 97.0% | 96.1% | 66.9% | 70.5% | 79.8% | / | / | / |

### Effect Embodiment 2 PSA Inhibition Rate Experiment

The purpose of this experiment was to test the change of the secretion level of PSA (Prostate antigen) in the supernatant of VCap cells processed by the tested compounds in combination with Enzalutamide for 5 days.

VCap cells were processed by 0.5 μM Enzalutamide alone, and by different concentrations of 3 tested compounds in combination with 0.5 μM Enzalutamide for 5 days respectively, then the PSA level of each treatment group was tested by electrochemiluminescence immunoassay.

Experimental Materials and Methods

1. Cell Line

| | | | Processing |
|---|---|---|---|
| Type of tumor | Name of cell | Medium | time (days) |
| prostate cancer | VCap | DMEM + 10% FBS | 5 |

The cell culture conditions were: 37° C., 5% CO₂ and 95% humidity.

2. Reagents

1) DMEM medium (Thermo scientific, product number: SH30243.01)

2) FBS (Fetal Bovine Serum) (Gibco, product number: 10099-141)

3) 0.25% trypsin-EDTA (Gibco, product number: 25200-072)

4) DMSO (Sigma, product number: D2650)

5) Prostate specific antigen reagent (Roche, product number: 04641655190) (provided by Taicang First People's Hospital)

3. Equipments

1) Carbon dioxide incubator: SANYO Electric Co., Ltd. (Japan). (Equipment ID: TAINC0490)

2) Microscope: Chongguang XDS-1B, Chongqing Guangdian Corp. (Chongqing, P.R.China). (Equipment ID: TAMIC0130)

3) Refrigerator: Haier Z16TXZ (China). (Equipment ID: TAREF0490)

4) Electronic Balance: Mettlertoledo AL104. (Shanghai, China). (Equipment ID: TBBAL0560)

5) Automatic Electrochemical Immunoassay Analyzer: Roche Cobas e601 (Taicang First People's Hospital)

4. Secretion Inhibition Rate of the Tested Compounds on VCap Cell PSA

Cell Inoculation

Cells were collected in exponential growth phase for viable cell count. The cell suspension was adjusted to 4.17×10e⁴/ml with the medium mentioned above. 120n1 of cell suspension was added to each well of a 96-well cell culture plateau final concentration of cells was 5000 cells/well. The cells were incubated overnight in a 37° C., 5% CO₂ incubator.

Dosing Treatment

10 mM stocking solution was prepared by dissolving each tested compound in DMSO. A series of 4x serial gradient dilutions were prepared with the stocking solution and DMSO, followed by being diluted with medium to be 10-fold dilutions respectively, and a 10-fold solution of Enzalutamide was prepared meanwhile. Enzalutamide and the equivalent volume of corresponding solution of the tested compound were added to each well for each cell line respectively, and a duplicate well was set for each drug concentration. The final concentrations of Enzalutamide and the tested compound used in the test are shown in Table 11. The final concentration of DMSO per well was 0.2%. The cells were incubated for 5 days in a 37° C., 5% CO₂ incubator.

Detection

After 5 days' drug treatment, the cell supernatant of each well was collected, and centrifuged at 2000 r/min for 5 minutes, then transferred to a clean EP tube for PSA detection.

5. Data Analysis

Calculation formula of PSA inhibition rate: (1-(Vₛₐₘₚₗₑ/V_{DMSO}))×100%. Wherein, Vₛₐₘₚₗₑ is the PSA reading of the drug treatment group, and V_{DMSO} is the average value of PSA of the solvent control group.

TABLE 11
PSA inhibition rate(%): 0.5 μM Enzalutamide used alone, and in
combination with different concentrations of the tested compound

| | Concentration of the tested compound (μM) | | | | | | | |
|---|---|---|---|---|---|---|---|---|
| | 100 | 25 | 6.25 | 1.56 | 0.39 | 0.10 | 0.02 | 0.01 |
| Enzalutamide (0.5 μM) + K001 | 37.3% | 50% | 55.2% | 52.7% | 52.7% | 55% | 53% | 50% |
| Enzalutamide (0.5 μM) + B001 | 65.4% | 63% | 63.2% | 63.9% | 59.7% | 50% | 46.7% | 43.6% |
| Enzalutamide (0.5 μM) + D108 | 69.2% | 60.3% | 61.6% | 57.9% | 57.9% | 57.2% | 56.8% | 56.1% |

PSA inhibition rate was 30.7% when Enzalutamide (0.5 μM) was used alone

Notes of Table 11: The PSA inhibition rate was 30.7% when 0.5 μM Enzalutamide was used alone. The PSA inhibition rates were 55%, 50% and 57.2% when 0.5 μM Enzalutamide was used in combination with 0.1 μM K001, B001 and D108 respectively. It can be seen that the effect of drug combination was significantly enhanced compared with Enzalutamide used alone.

Although the specific embodiments of the present invention are described above, it will be understood by people skilled in the art that these are just examples. Many changes and modifications can be made to these embodiments without departing from the principle and essence of the present invention. Therefore, the protection scope of the present invention is defined by the claims attached.

What is claimed is:

1. A pharmaceutical composition, comprising one selected from the group consisting of a benzoheterocyclic compound as shown in formula (I), a pharmaceutically acceptable salt thereof, a solvate thereof, a polymorph thereof, a co-crystal thereof, a stereoisomer thereof, an isotope compound thereof, a metabolite thereof and a prodrug thereof, and an androgen receptor pathway modulator; 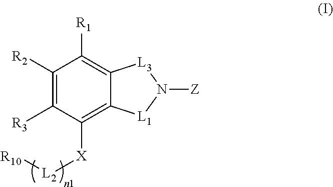
in formula (I), n1 is 0 or 1;
L₁ and L₃ are independently 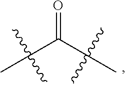 [SMILES: *C(*)=O]
CH₂, CHD, CD₂, 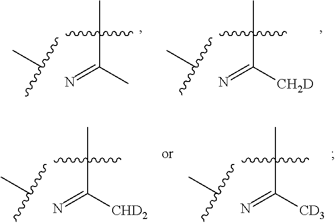
L₂ is CD₂, CHD or CH₂;
X is NH, ND or O;
R₁, R₂ and R₃ are independently H or D;
Z is 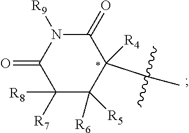
wherein, R₄ is H, D, CH₃, CH₂D, CHD₂ or CD₃; R₅, R₆, R₇, R₈ and R₉ are independently H or D; the carbon marked with * is an asymmetric center;
R₁₀ is H, D or 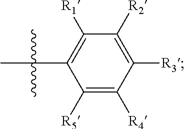
wherein, R₁′, R₂′, R₃′, R₄′ and R₅′ are independently selected form H, D, or a substituted or unsubstituted (C₁-C₁₂) alkyl;
the substituent on the substituted (C₁-C₁₂)alkyl is selected from the group consisting of D, (C₂-C₂₀)heterocycloalkyl, deuterated(C₂-C₂₀)heterocycloalkyl, (C₂-C₂₀)heterocycloalkyl substituted by (C₁-C₁₂)alkyl and (C₂-C₂₀)heterocycloalkyl substituted by deuterated(C₁-C₁₂) alkyl;
when there are a plurality of substituents on the substituted (C₁-C₁₂)alkyl, the substituents are the same or different;
the heteroatom contained in the (C₂-C₂₀)heterocycloalkyl which is referred in the (C₂-C₂₀)heterocycloalkyl, deuterated(C₂-C₂₀)heterocycloalkyl, (C₂-C₂₀)heterocycloalkyl substituted by (C₁-C₁₂)alkyl and (C₂-C₂₀)heterocycloalkyl substituted by deuterated(C₁-C₁₂)alkyl is selected from the group consisting of O, N and S;
when n1 is 0, X is NH or ND, L₁ is 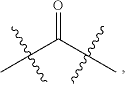 [SMILES: *C(*)=O]
CH₂, CHD or CD₂; and L₃ is 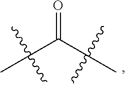
in formula (I), R₁₀ is H or D;
when n1 is 0, X is NH or ND, L₁ is 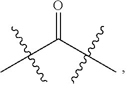 [SMILES: *C(*)=O]
and L₃ is 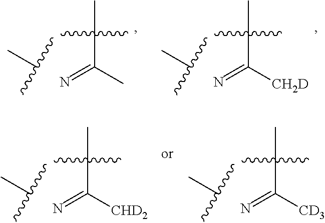
in formula (I), R₁₀ is H or D;
when n1 is 1, L₁ is CH₂, CHD or CD₂, L₃ is 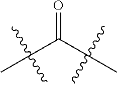 [SMILES: *C(*)=O]
in formula (I), R₁₀ is 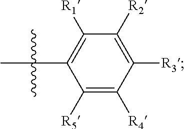
D represents a deuterium-enriched hydrogen, and H represents a non-deuterium-enriched hydrogen;
the androgen receptor pathway modulator is selected from the group consisting of Enzalutamide, ARN-509, ODM-201, VT-464, Orteronel, EPI-001, Andarine, RD162, BMS-641988, CH5137291, Flutamide, Hydroxyl Flutamide, RU58642, LG120907, LG105, Galeterone, Spironolactone, MK-2866, AZD3514, Cyproterone acetate, ORM-15341, Bicalutamide, Nilutamide, Degarelix, Goserelin acetate, Leuprolide acetate, Abiraterone and Abiraterone acetate;
when the androgen receptor pathway modulator is selected from one of the above compounds, the androgen receptor pathway modulator is not Bicalutamide, Nilutamide, Leuprolide acetate, Abiraterone or Abiraterone Acetate.

2. The pharmaceutical composition according to claim 1, wherein, the androgen receptor pathway modulator is Enzalutamide, ARN-509, Galeterone, ODM-201, ORM-15341, Enzalutamide and Galeterone, Enzalutamide and Abiraterone acetate, Enzalutamide and Abiraterone, Enzalutamide and ODM-201, Enzalutamide and ORM-15341, ARN-509 and Galeterone, ARN-509 and Abiraterone acetate, ARN-509 and Abirateron, ARN-509 and ODM-201, ARN-509 and ORM-15341, ODM-201 and Galeterone, ODM-201 and Abiraterone acetate, ODM-201 and Abiraterone, ORM-15341 and Galeterone, ORM-15341 and Abiraterone acetate, or ORM-15341 and Abiraterone.

3. The pharmaceutical composition according to claim 1, wherein
in formula (I), the asymmetric center refers to an achiral carbon, a (S)-configured carbon, a (R)-configured carbon or a racemate;
and/or, in formula (I), Z is selected from the group consisting of 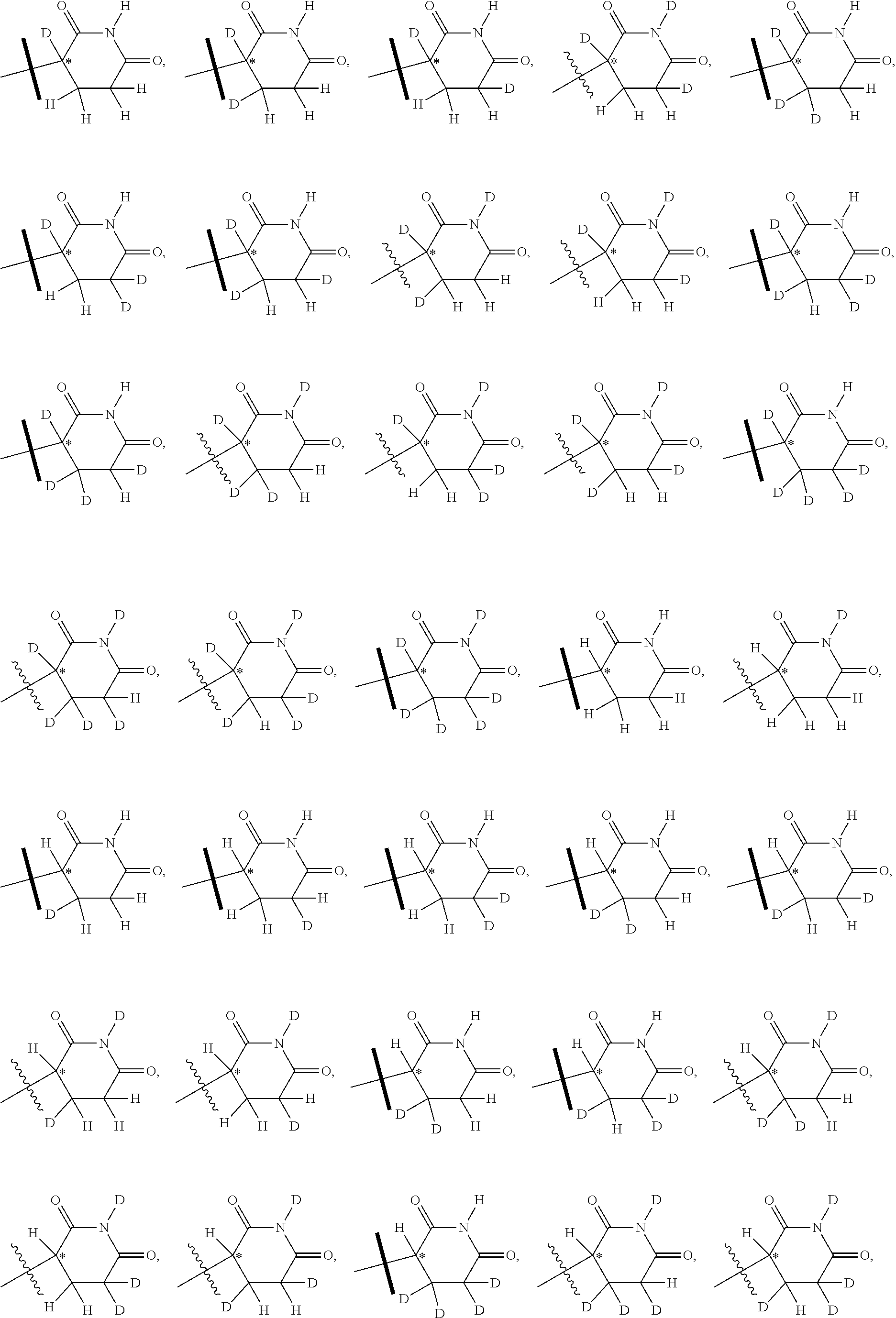 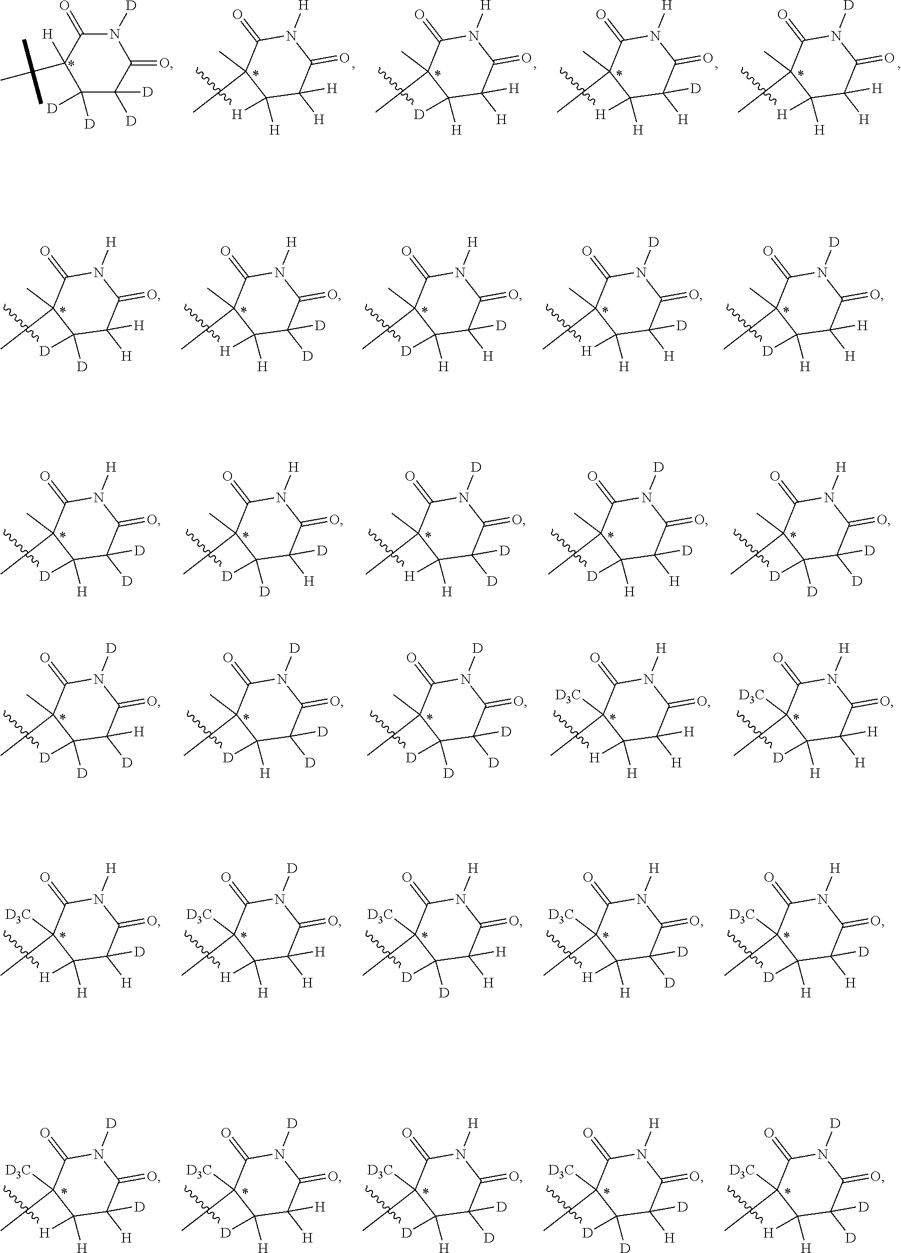 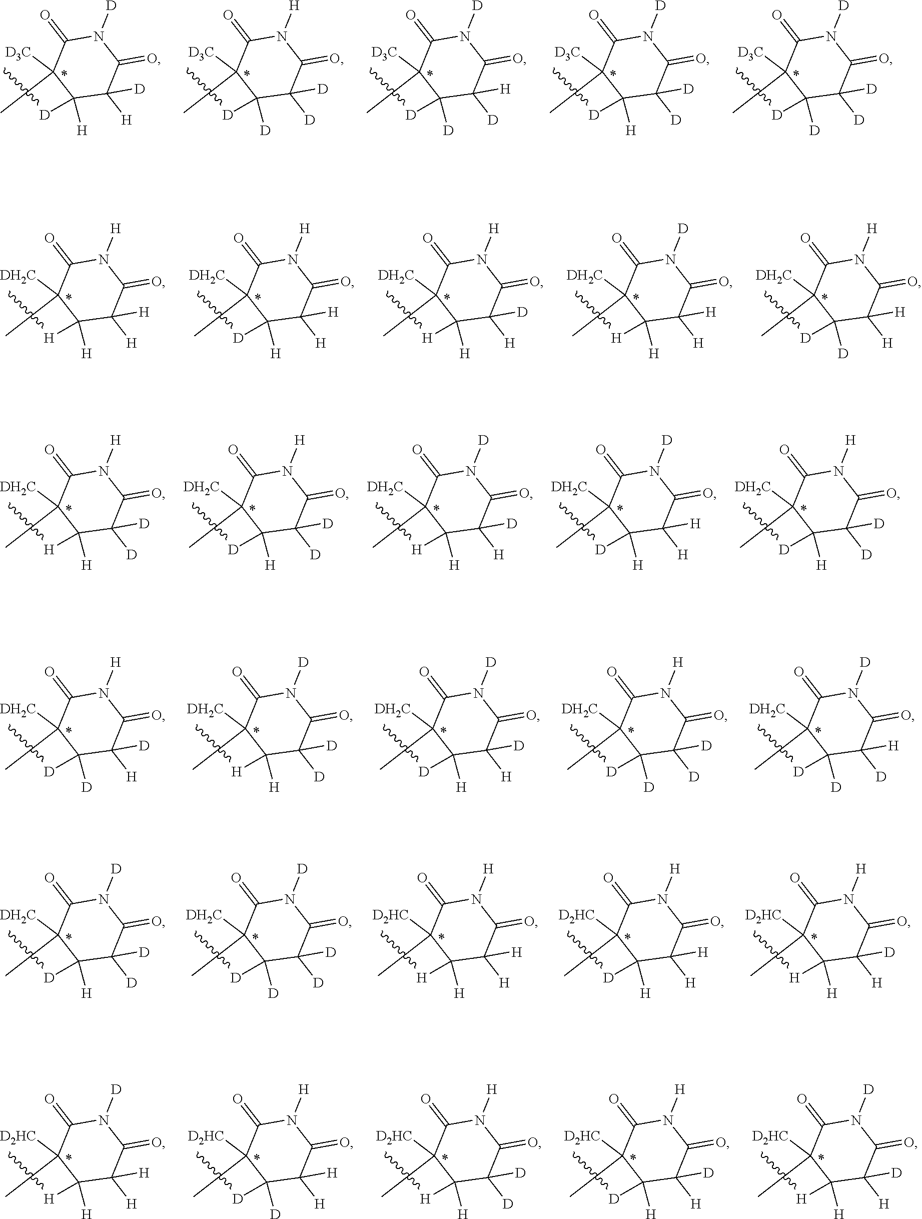 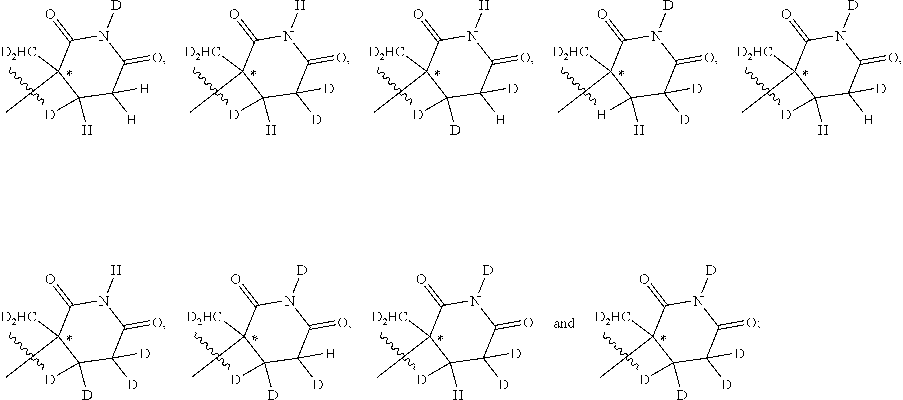
wherein, *, H and D are as defined in claim 1;
and/or, in formula (I), both the (C₂-C₂₀)heterocycloalkyl and the (C₂-C₂₀)heterocycloalkyl in the deuterated(C₂-C₂₀)heterocycloalkyl, the (C₂-C₂₀)heterocycloalkyl substituted by (C₁-C₁₂)alkyl or the (C₂-C₂₀)heterocycloalkyl substituted by deuterated (C₁-C₁₂)alkyl refer to a (C₂-C₆)heterocycloalkyl having 1 to 2 heteroatom selected from N or O; the (C₂-C₆)heterocycloalkyl is preferably a morpholinyl; the (C₁-C₁₂)alkyl which is referred in the (C₂-C₂₀)heterocycloalkyl substituted by (C₁-C₁₂)alkyl or in the (C₂-C₂₀)heterocycloalkyl substituted by deuterated(C₁-C₁₂)alkyl is a (C₁-C₄)alkyl; the (C₁-C₄)alkyl is preferably a methyl, an ethyl, a n-propyl, an isopropyl, a n-butyl, an isobutyl or a tert-butyl;
and/or, in formula (I), the substituted or unsubstituted (C₁-C₁₂)alkyl is a substituted or unsubstituted (C₁-C₄)alkyl; the substituted or unsubstituted (C₁-C₄)alkyl is preferably a substituted or unsubstituted methyl, a substituted or unsubstituted ethyl, a substituted or unsubstituted n-propyl, a substituted or unsubstituted isopropyl, a substituted or unsubstituted n-butyl, a substituted or unsubstituted isobutyl, or a substituted or unsubstituted tert-butyl; the substituted (C₁-C₄)alkyl is preferably 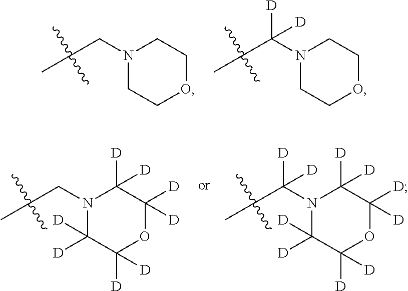
and/or, in formula (I), the

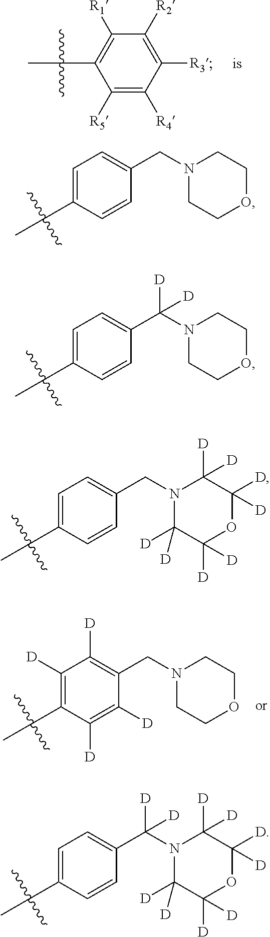

4. The pharmaceutical composition according to claim 1, wherein the benzoheterocyclic compound as shown in formula (I) is selected from the group consisting of

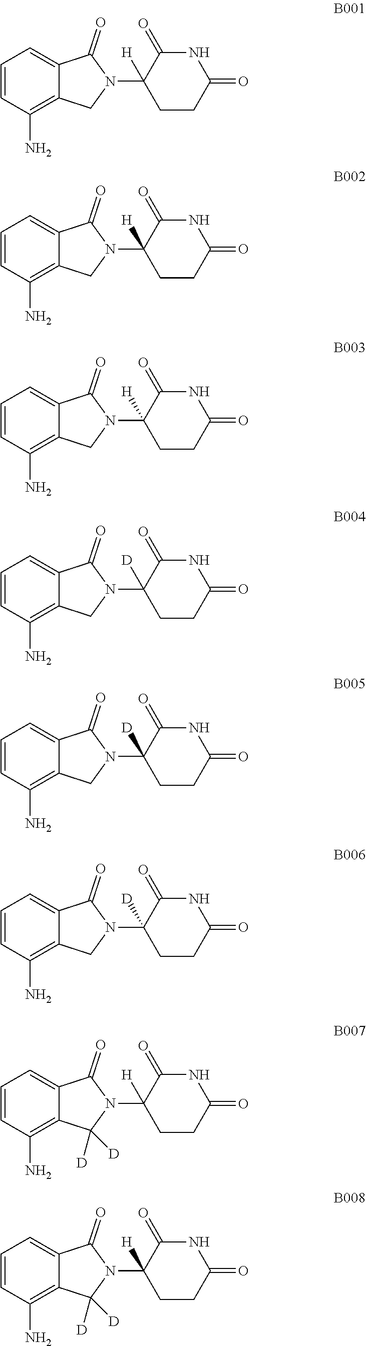

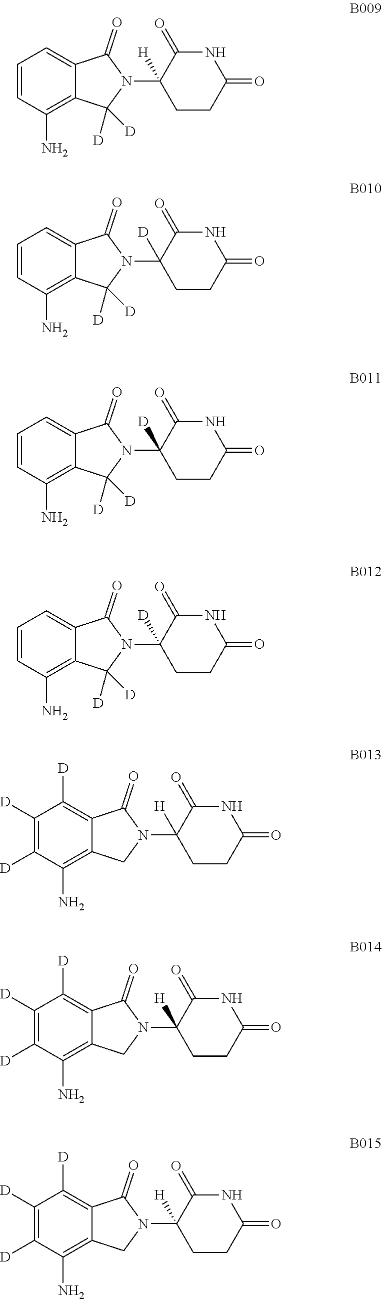

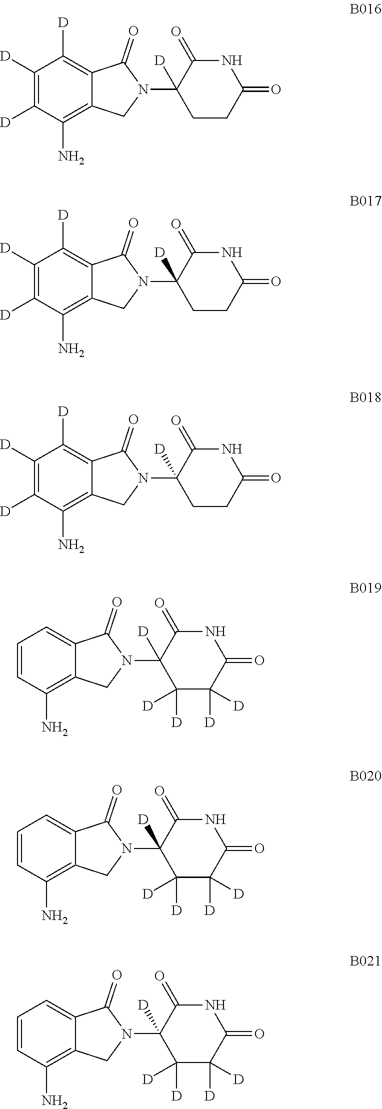 [SMILES: [2H]c1c([2H])c(N)c2c(c1[2H])C(=O)N(C1([2H])CCC(=O)NC1=O)C2 ; [2H]c1c([2H])c(N)c2c(c1[2H])C(=O)N([C@@]1([2H])CCC(=O)NC1=O)C2 ; [2H]c1c([2H])c(N)c2c(c1[2H])C(=O)N([C@]1([2H])CCC(=O)NC1=O)C2 ; [2H]C1([2H])C(=O)NC(=O)C([2H])(N2Cc3c(N)cccc3C2=O)C1([2H])[2H] ; [2H]C1([2H])C(=O)NC(=O)[C@@]([2H])(N2Cc3c(N)cccc3C2=O)C1([2H])[2H] ; [2H]C1([2H])C(=O)NC(=O)[C@]([2H])(N2Cc3c(N)cccc3C2=O)C1([2H])[2H]]

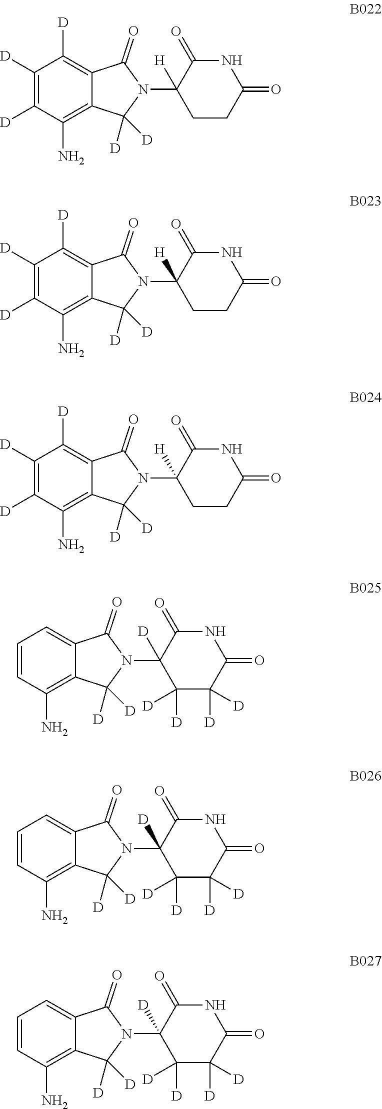 [SMILES: [2H]c1c([2H])c(N)c2c(c1[2H])C(=O)N(C1CCC(=O)NC1=O)C2([2H])[2H] ; [2H]c1c([2H])c(N)c2c(c1[2H])C(=O)N([C@H]1CCC(=O)NC1=O)C2([2H])[2H] ; [2H]c1c([2H])c(N)c2c(c1[2H])C(=O)N([C@@H]1CCC(=O)NC1=O)C2([2H])[2H] ; [2H]C1([2H])c2c(N)cccc2C(=O)N1C1([2H])C(=O)NC(=O)C([2H])([2H])C1([2H])[2H] ; [2H]C1([2H])c2c(N)cccc2C(=O)N1[C@]1([2H])C(=O)NC(=O)C([2H])([2H])C1([2H])[2H] ; [2H]C1([2H])c2c(N)cccc2C(=O)N1[C@@]1([2H])C(=O)NC(=O)C([2H])([2H])C1([2H])[2H]]

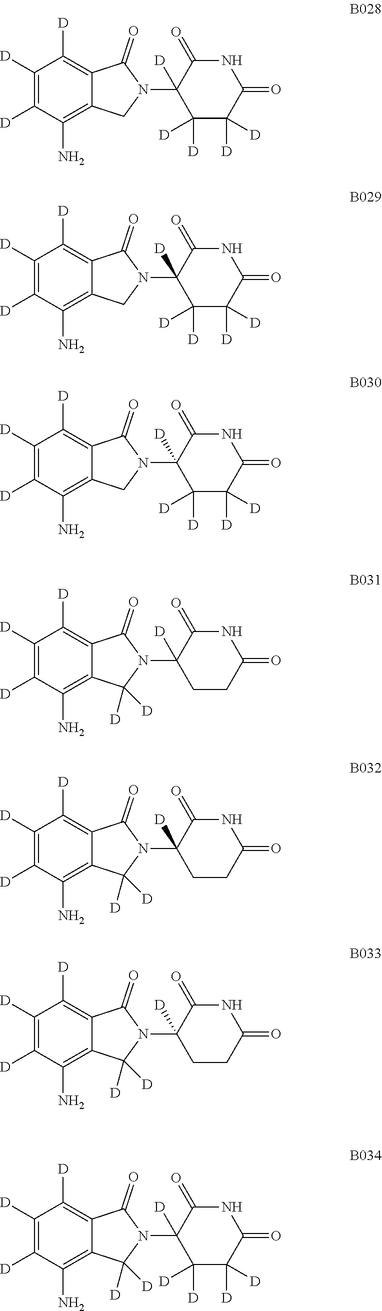

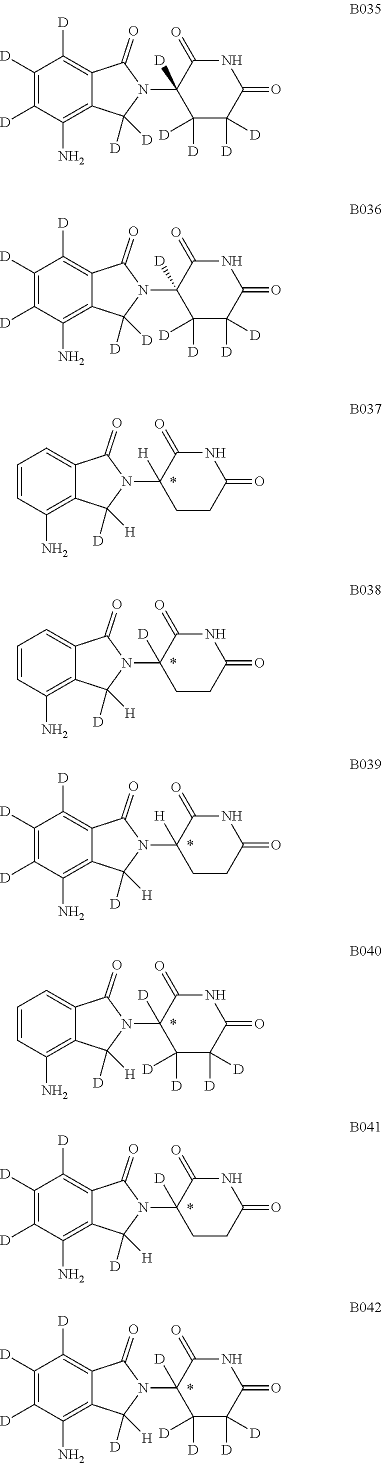

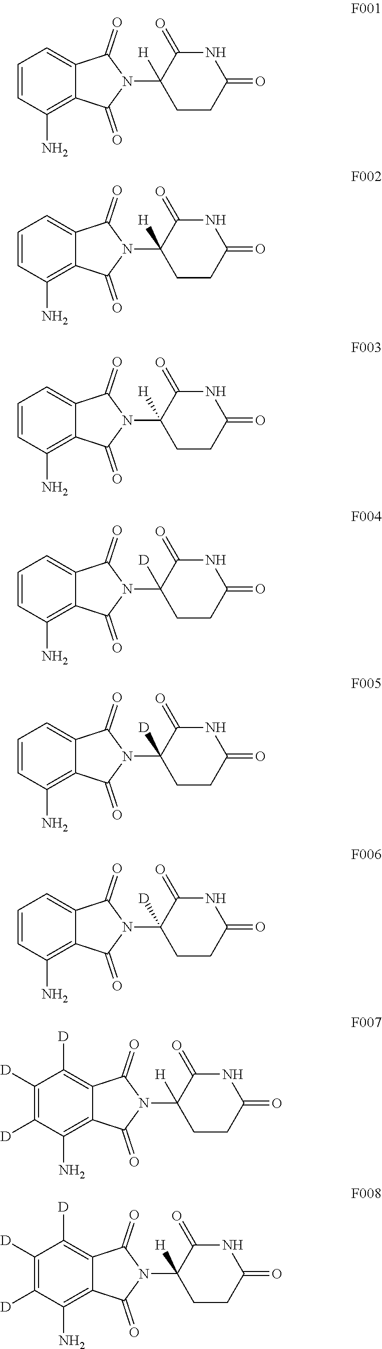

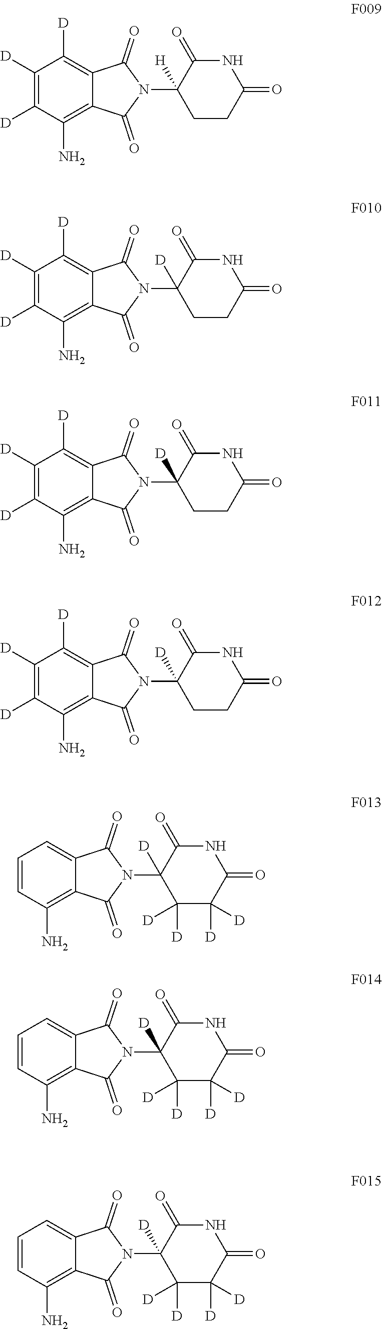

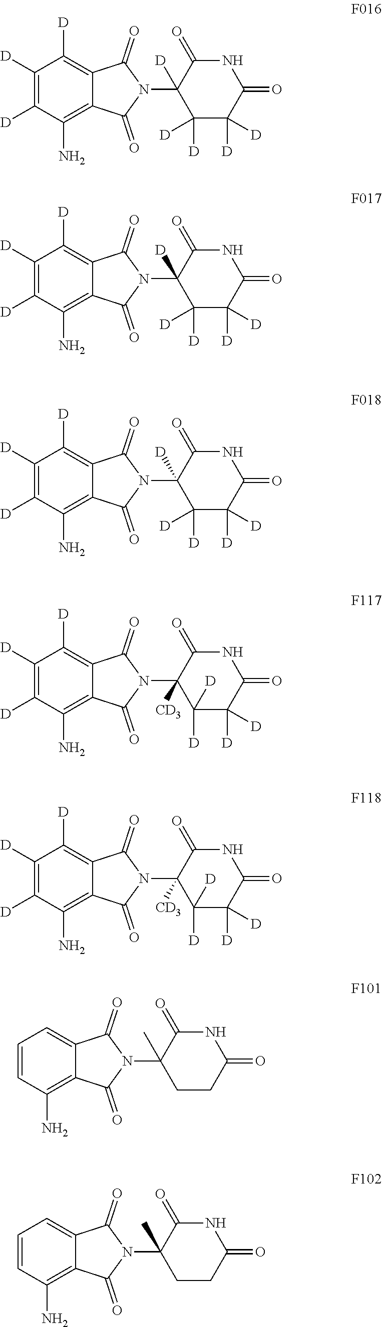

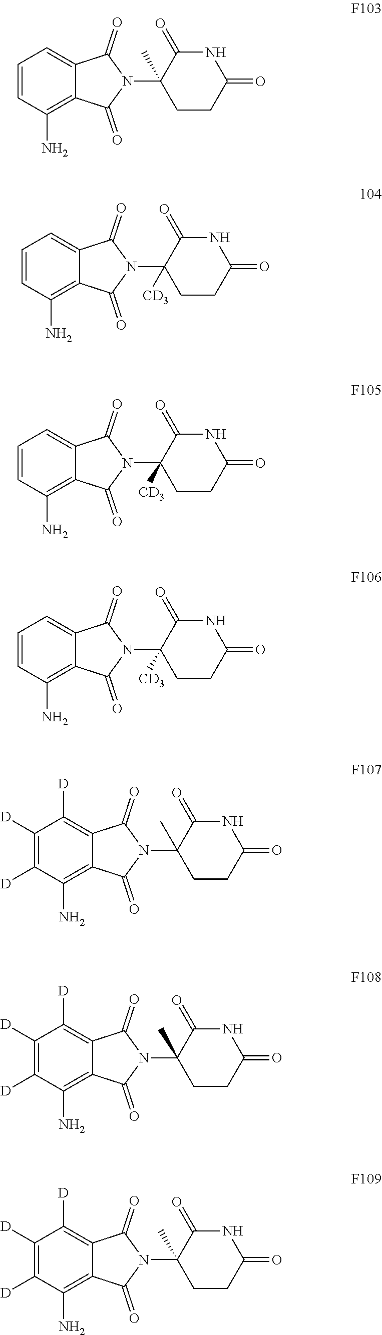

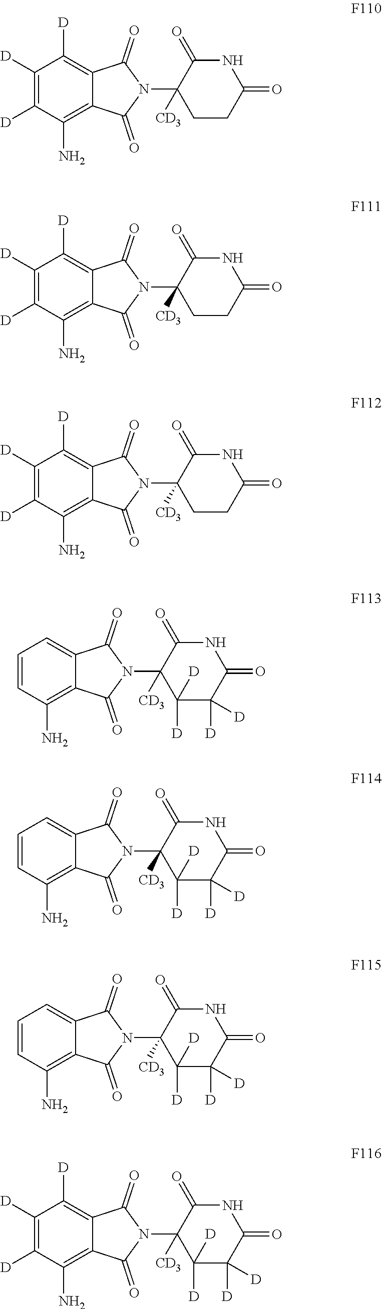

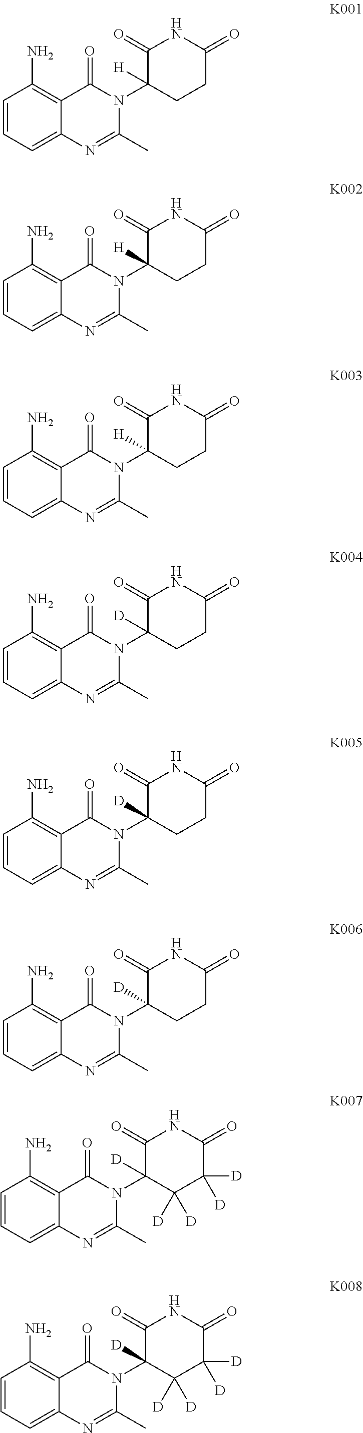

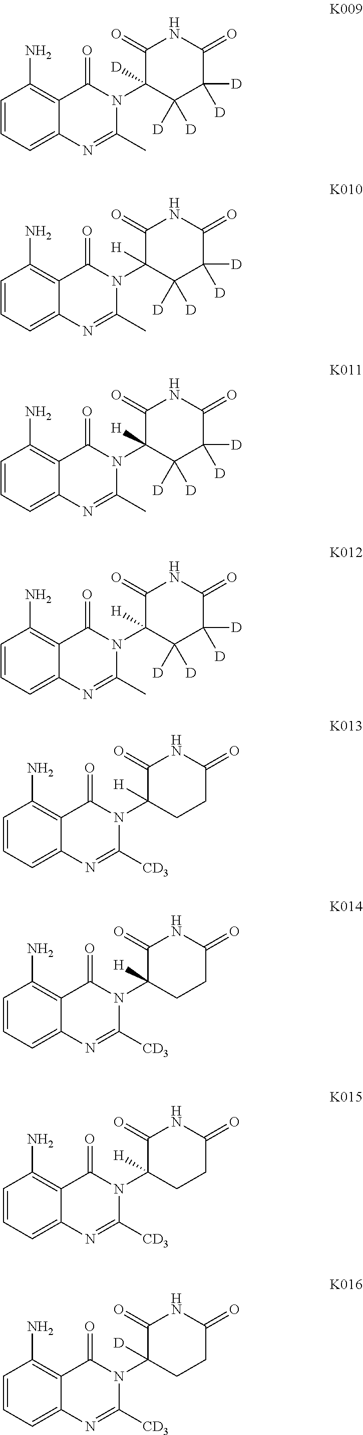

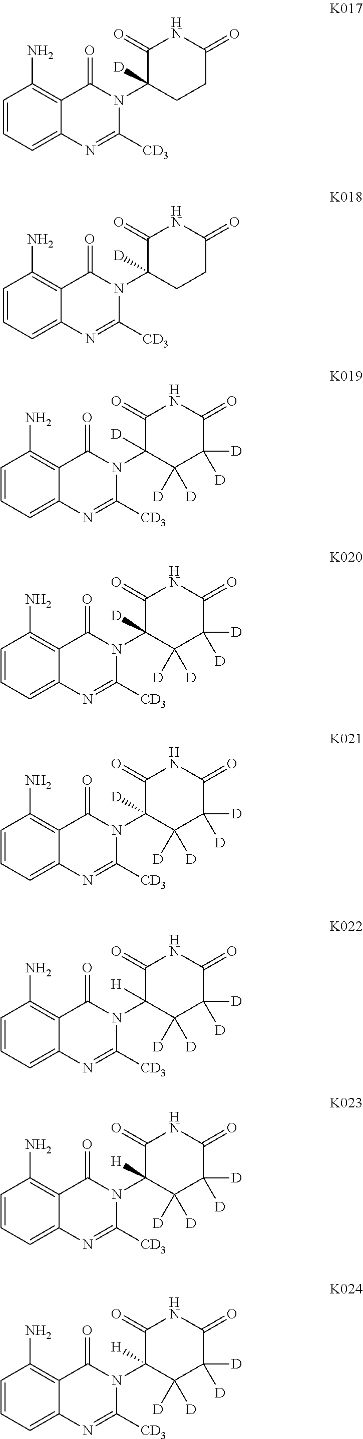

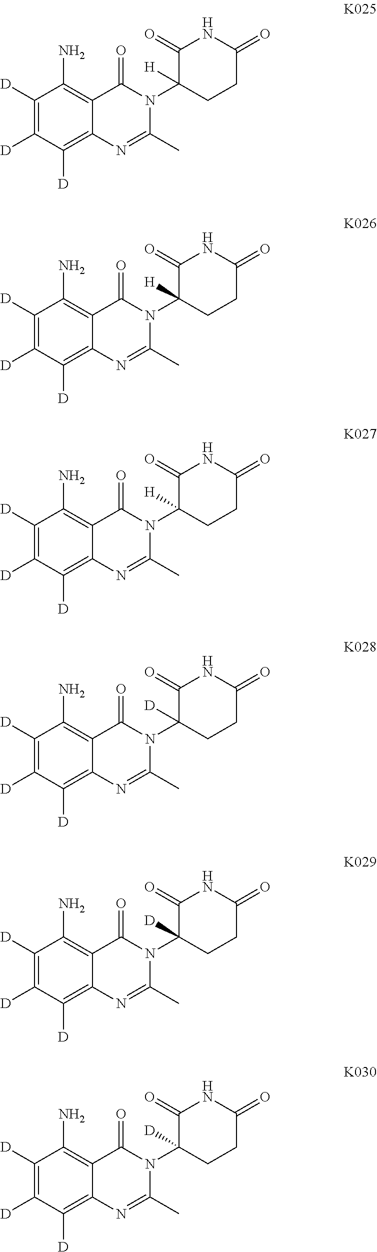 [SMILES: [2H]c1c([2H])c(N)c2c(=O)n(C3CCC(=O)NC3=O)c(C)nc2c1[2H] ; [2H]c1c([2H])c(N)c2c(=O)n([C@H]3CCC(=O)NC3=O)c(C)nc2c1[2H] ; [2H]c1c([2H])c(N)c2c(=O)n([C@@H]3CCC(=O)NC3=O)c(C)nc2c1[2H] ; [2H]c1c([2H])c(N)c2c(=O)n(C3([2H])CCC(=O)NC3=O)c(C)nc2c1[2H] ; [2H]c1c([2H])c(N)c2c(=O)n([C@@]3([2H])CCC(=O)NC3=O)c(C)nc2c1[2H] ; [2H]c1c([2H])c(N)c2c(=O)n([C@]3([2H])CCC(=O)NC3=O)c(C)nc2c1[2H]]

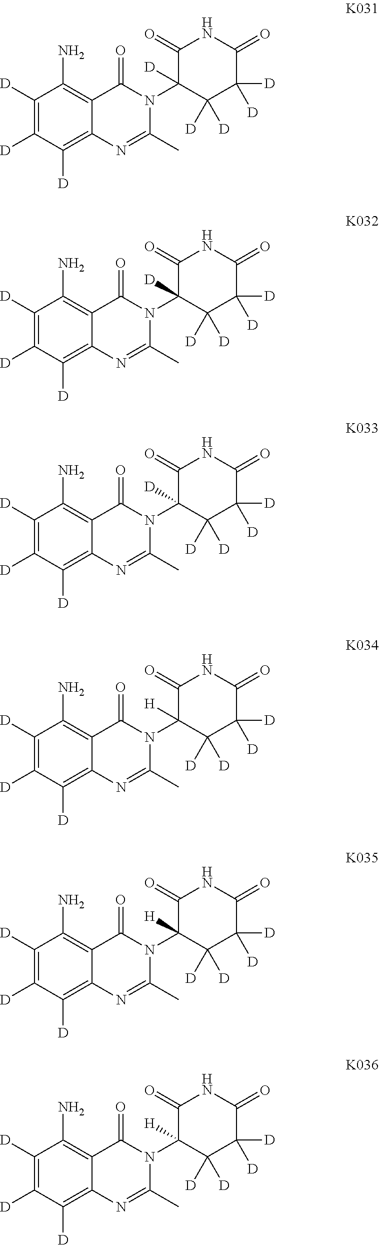

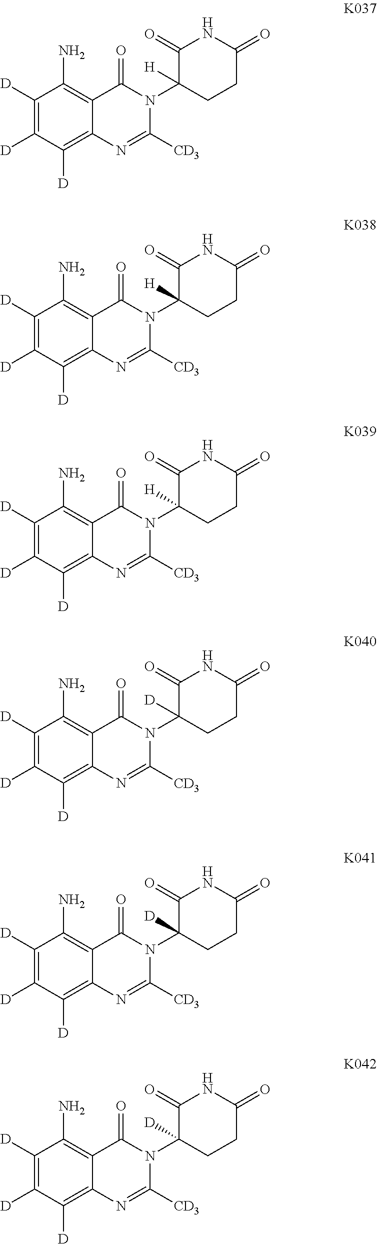 [SMILES: [2H]c1c([2H])c(N)c2c(=O)n(C3CCC(=O)NC3=O)c(C([2H])([2H])[2H])nc2c1[2H] ; [2H]c1c([2H])c(N)c2c(=O)n([C@H]3CCC(=O)NC3=O)c(C([2H])([2H])[2H])nc2c1[2H] ; [2H]c1c([2H])c(N)c2c(=O)n([C@@H]3CCC(=O)NC3=O)c(C([2H])([2H])[2H])nc2c1[2H] ; [2H]c1c([2H])c(N)c2c(=O)n(C3([2H])CCC(=O)NC3=O)c(C([2H])([2H])[2H])nc2c1[2H] ; [2H]c1c([2H])c(N)c2c(=O)n([C@@]3([2H])CCC(=O)NC3=O)c(C([2H])([2H])[2H])nc2c1[2H] ; [2H]c1c([2H])c(N)c2c(=O)n([C@]3([2H])CCC(=O)NC3=O)c(C([2H])([2H])[2H])nc2c1[2H]]

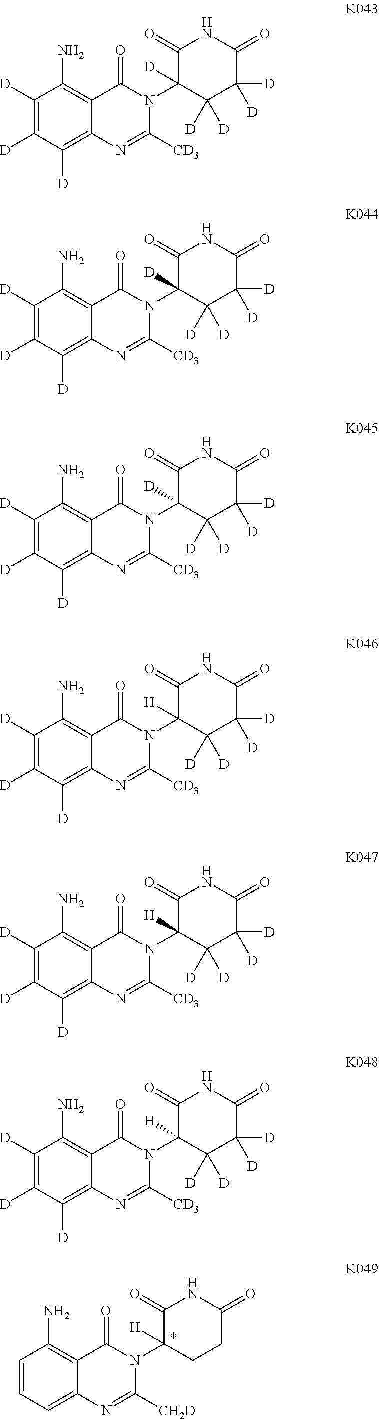

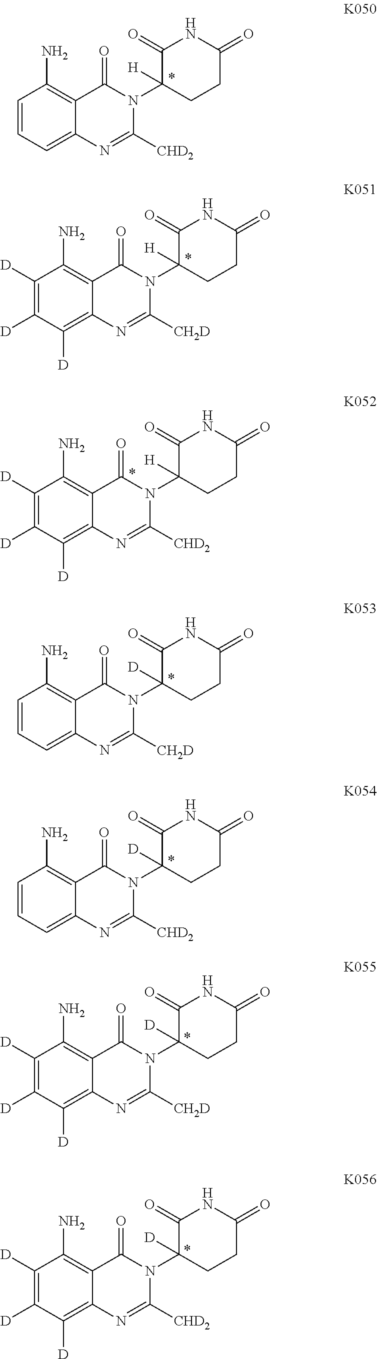

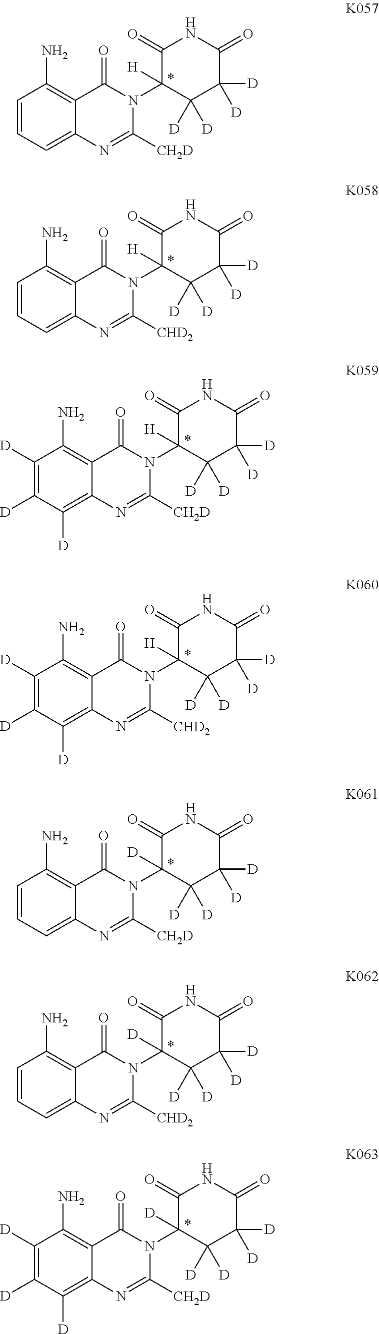

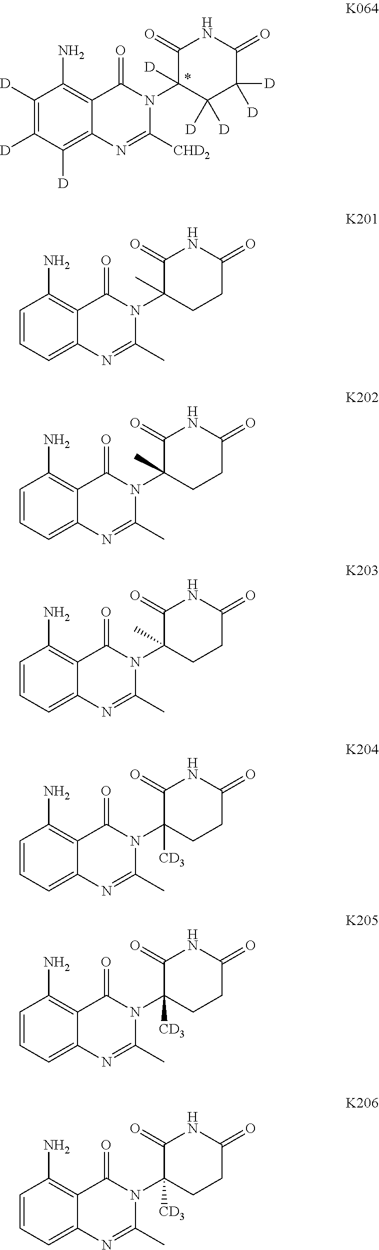

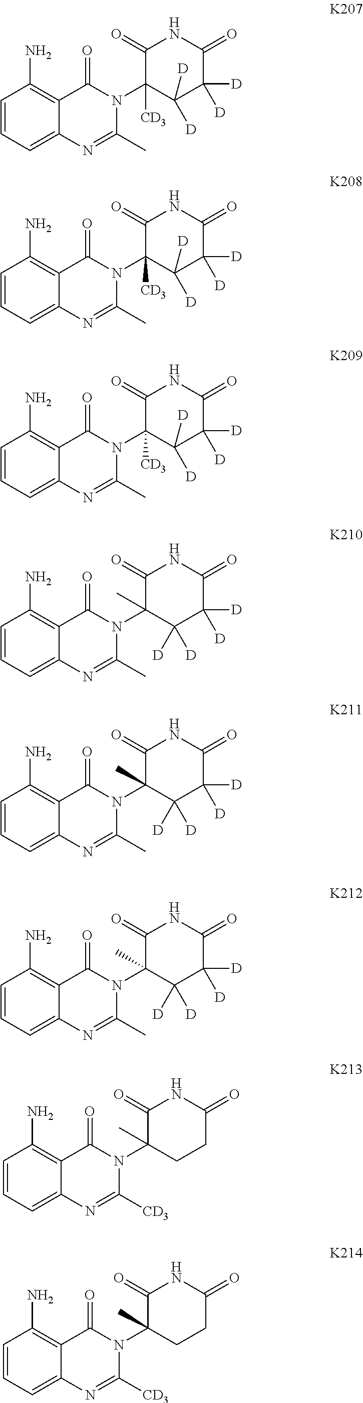

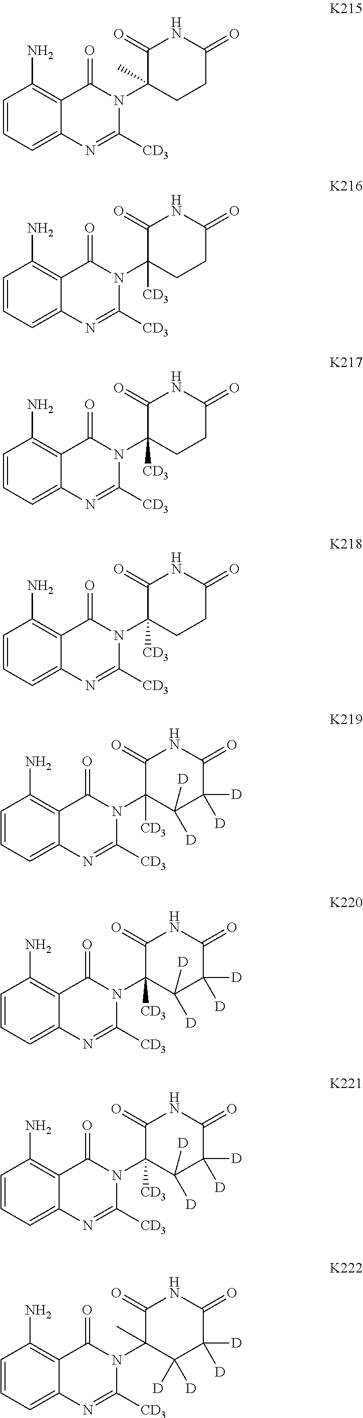

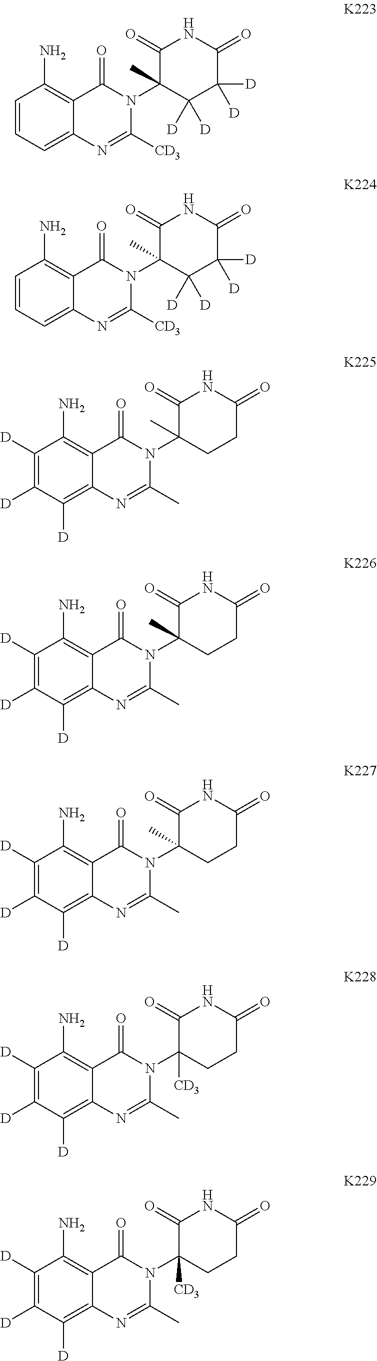

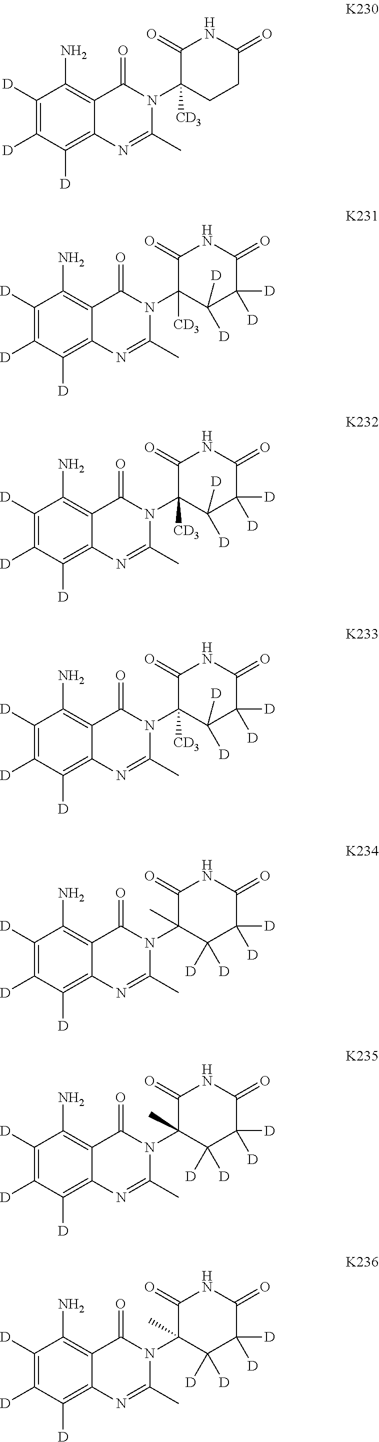

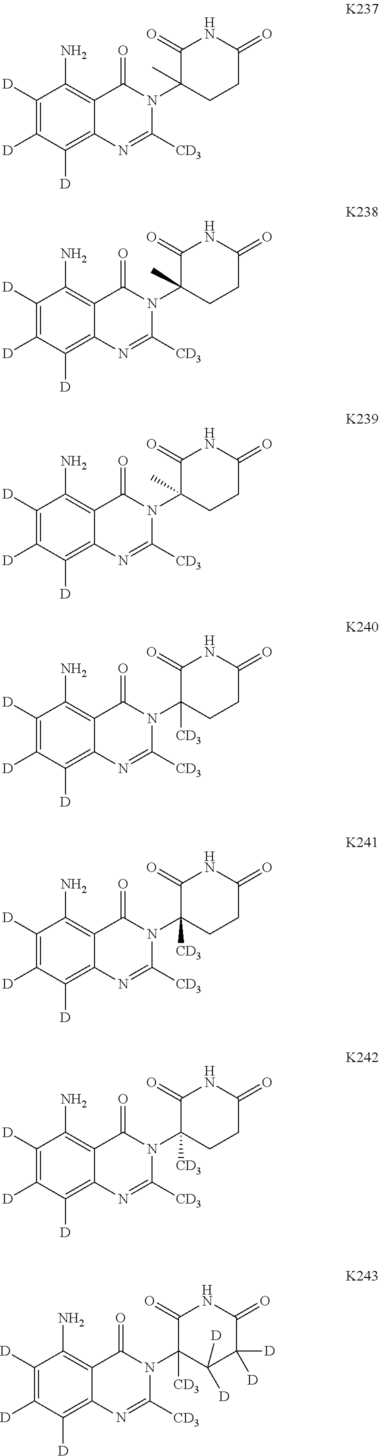

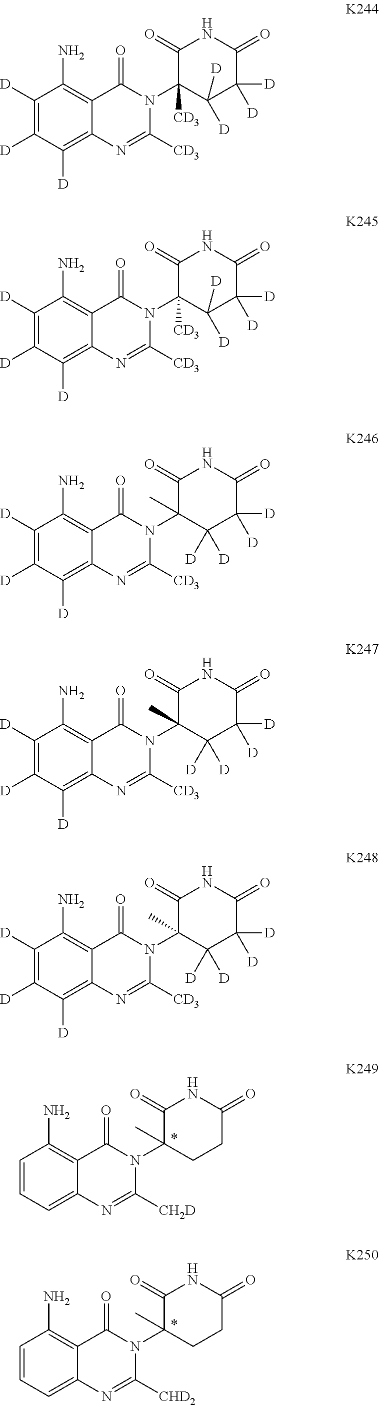

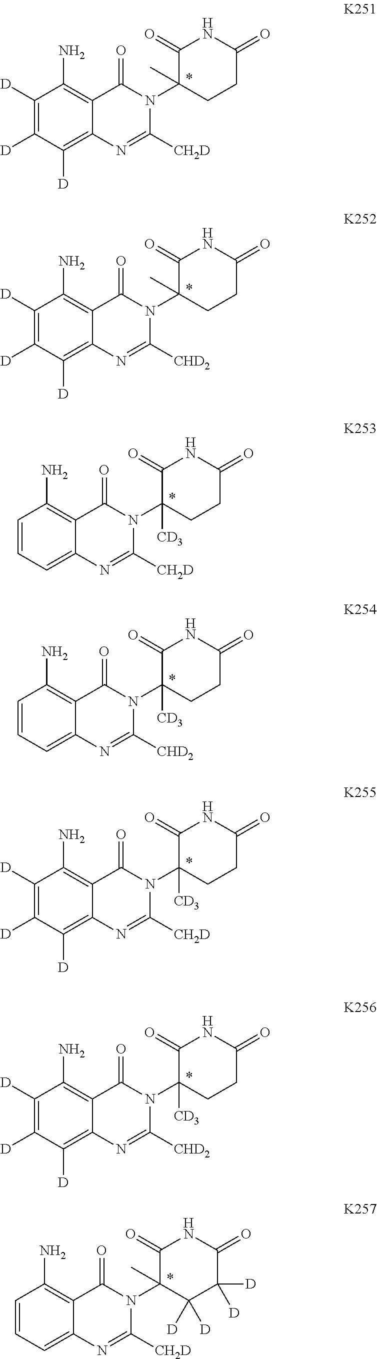

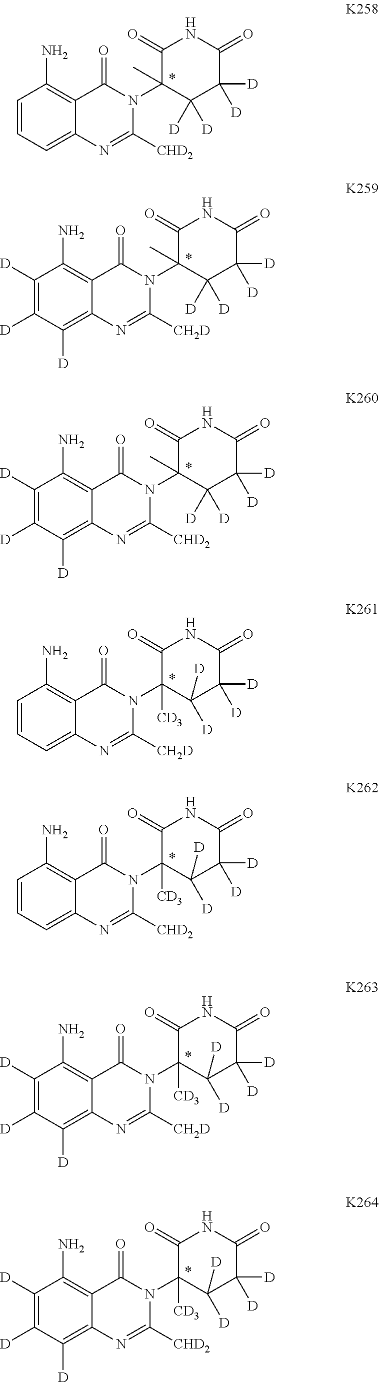

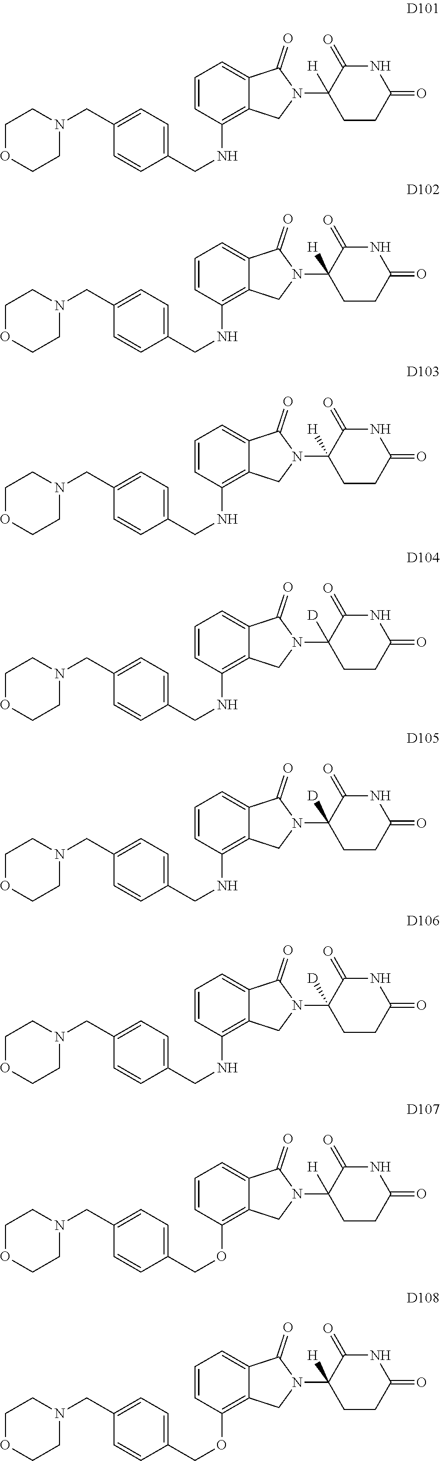

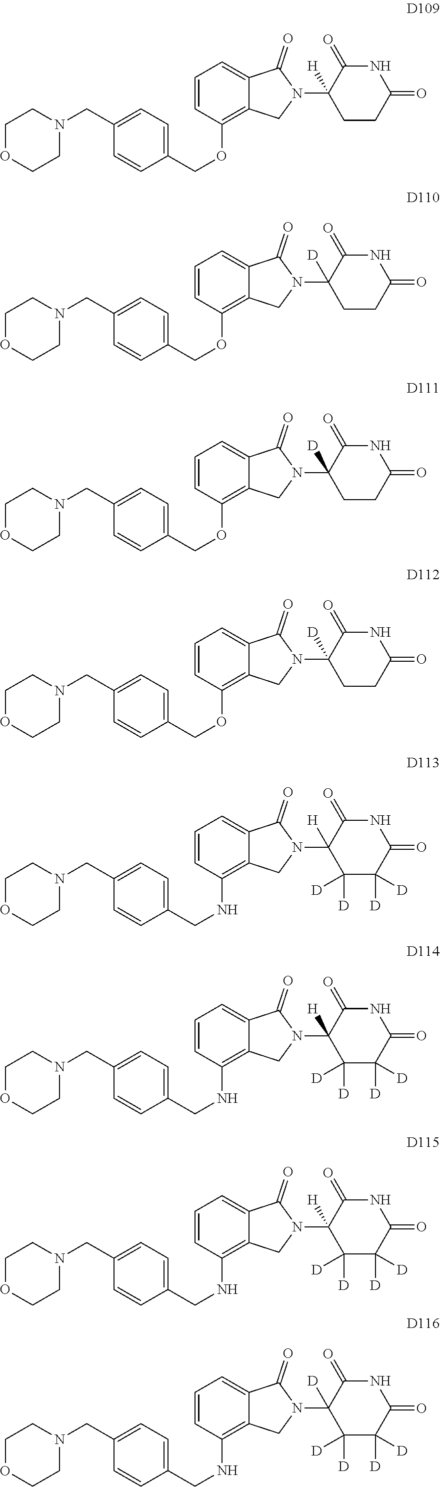

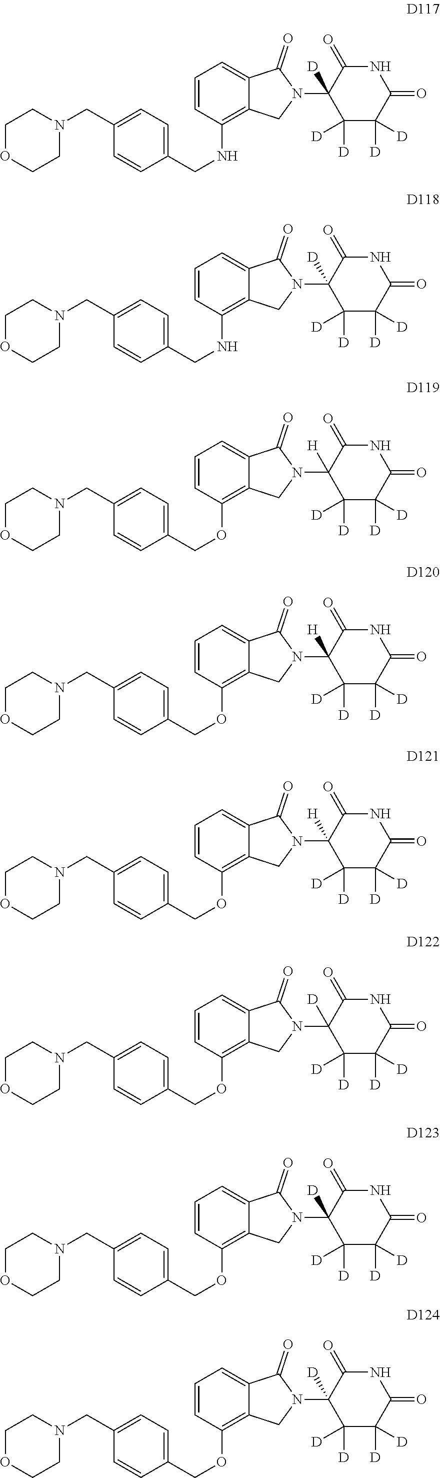

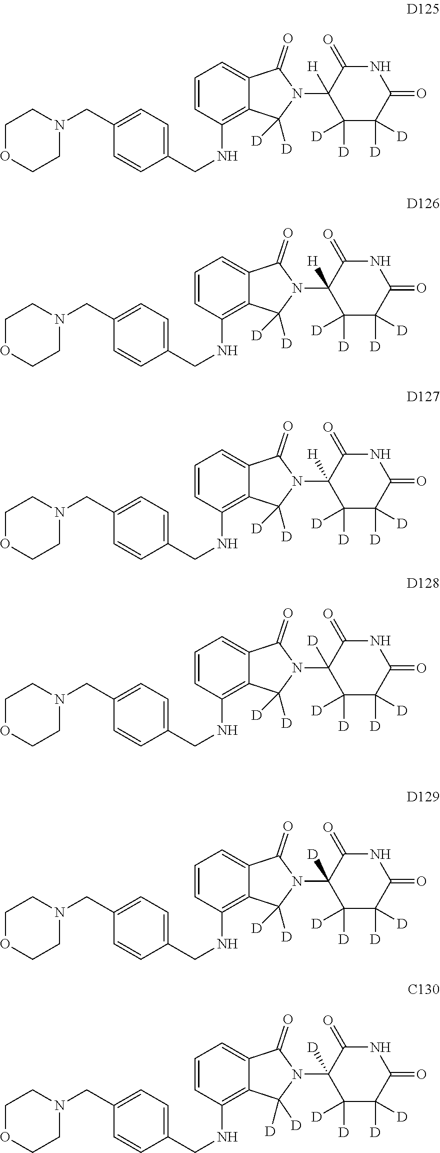

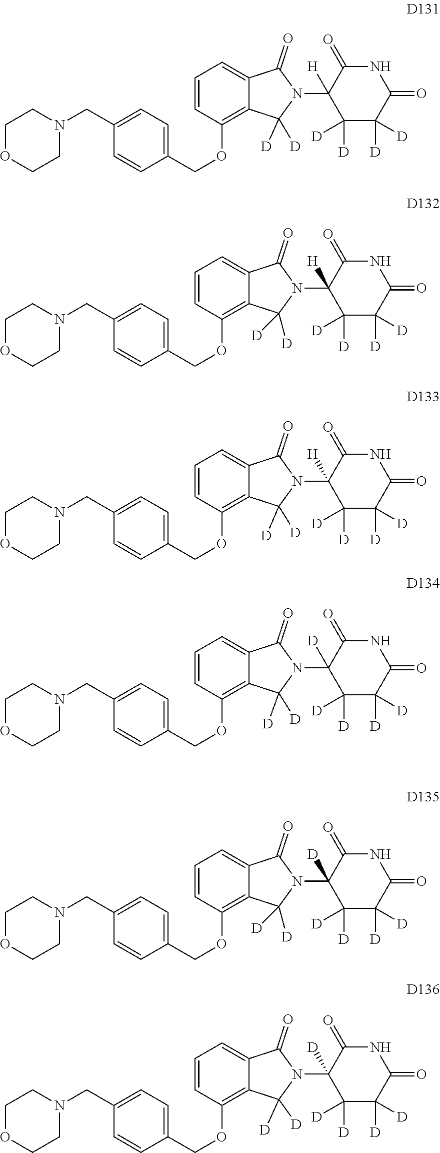

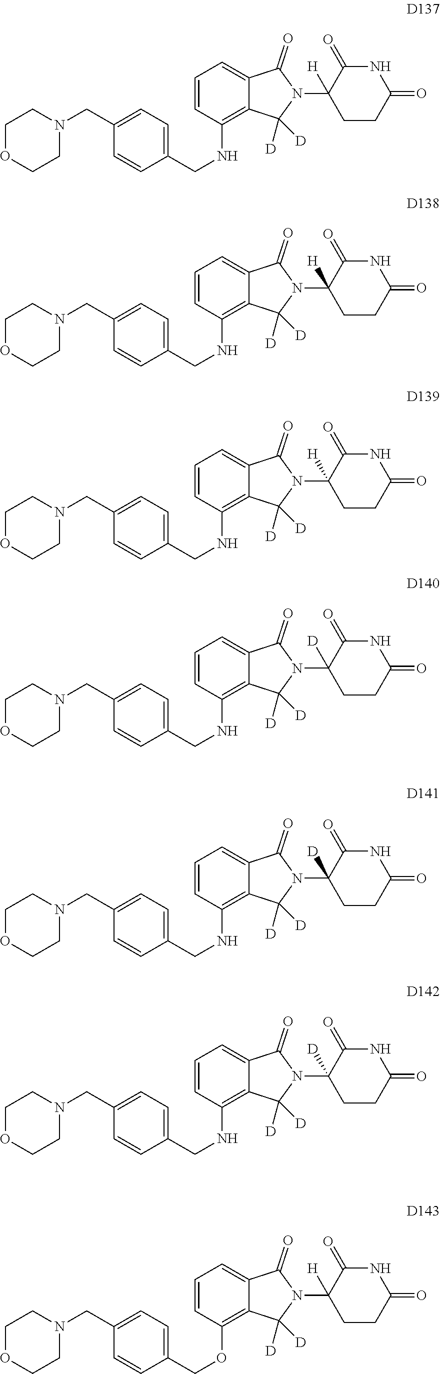

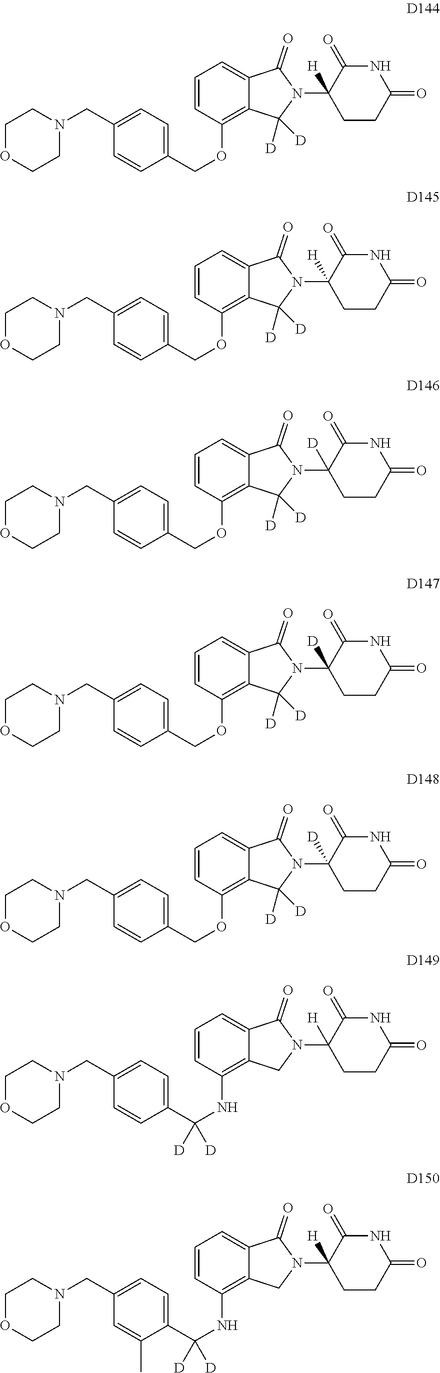

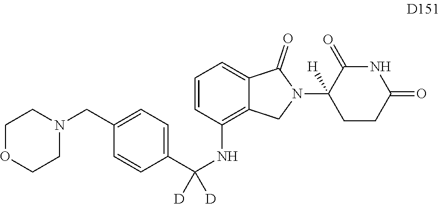 [SMILES: [2H]C([2H])(Nc1cccc2c1CN([C@@H]1CCC(=O)NC1=O)C2=O)c1ccc(CN2CCOCC2)cc1]

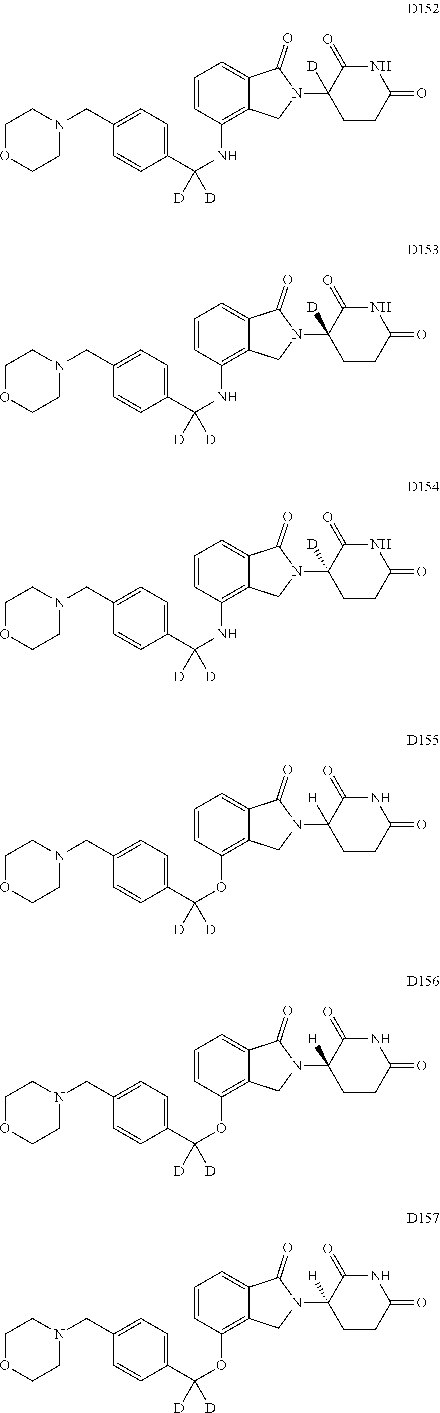 [SMILES: [2H]C([2H])(Nc1cccc2c1CN(C1([2H])CCC(=O)NC1=O)C2=O)c1ccc(CN2CCOCC2)cc1 ; [2H]C([2H])(Nc1cccc2c1CN([C@@]1([2H])CCC(=O)NC1=O)C2=O)c1ccc(CN2CCOCC2)cc1 ; [2H]C([2H])(Nc1cccc2c1CN([C@]1([2H])CCC(=O)NC1=O)C2=O)c1ccc(CN2CCOCC2)cc1 ; [2H]C([2H])(Oc1cccc2c1CN(C1CCC(=O)NC1=O)C2=O)c1ccc(CN2CCOCC2)cc1 ; [2H]C([2H])(Oc1cccc2c1CN([C@H]1CCC(=O)NC1=O)C2=O)c1ccc(CN2CCOCC2)cc1 ; [2H]C([2H])(Oc1cccc2c1CN([C@@H]1CCC(=O)NC1=O)C2=O)c1ccc(CN2CCOCC2)cc1]

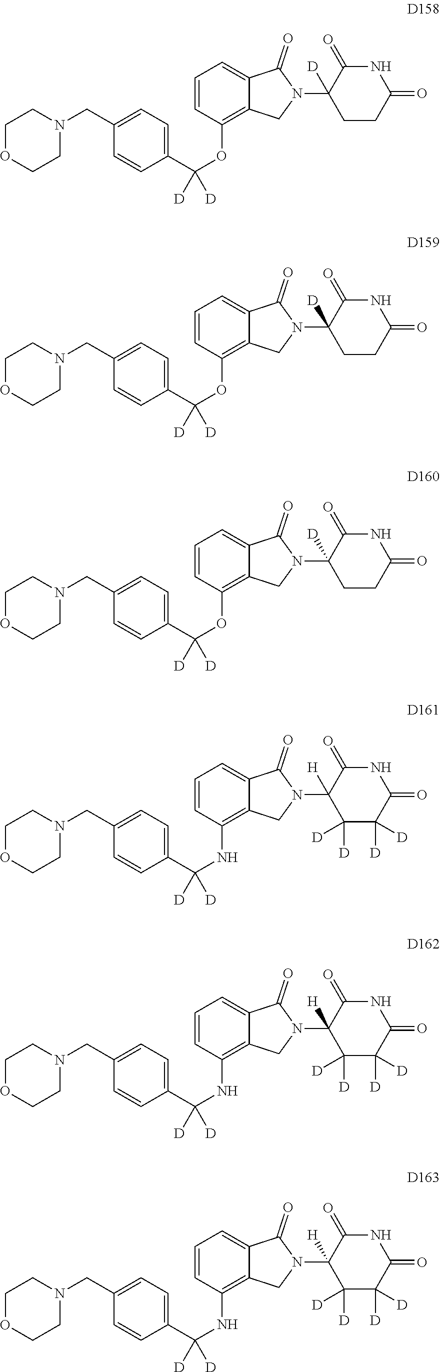

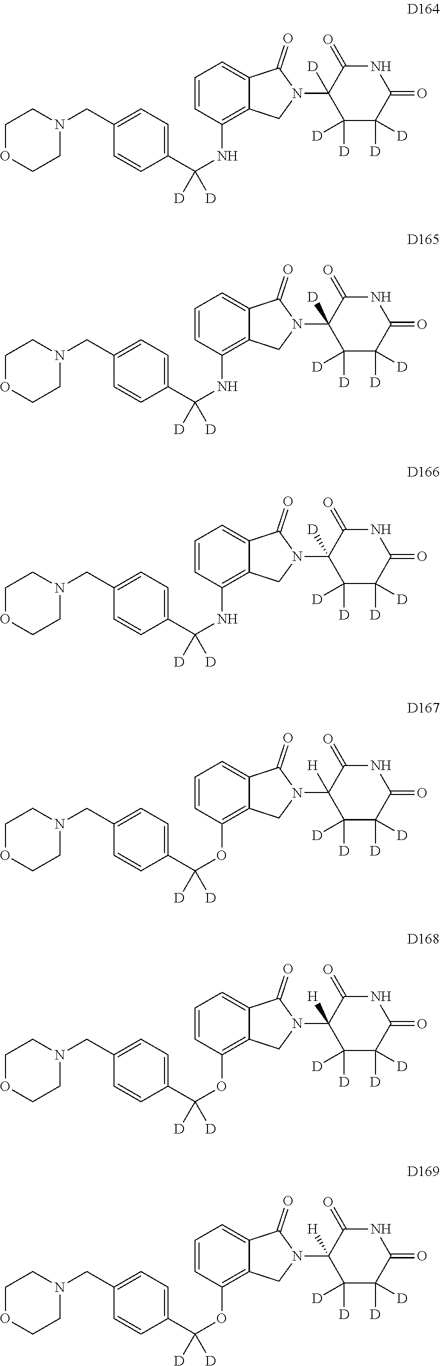

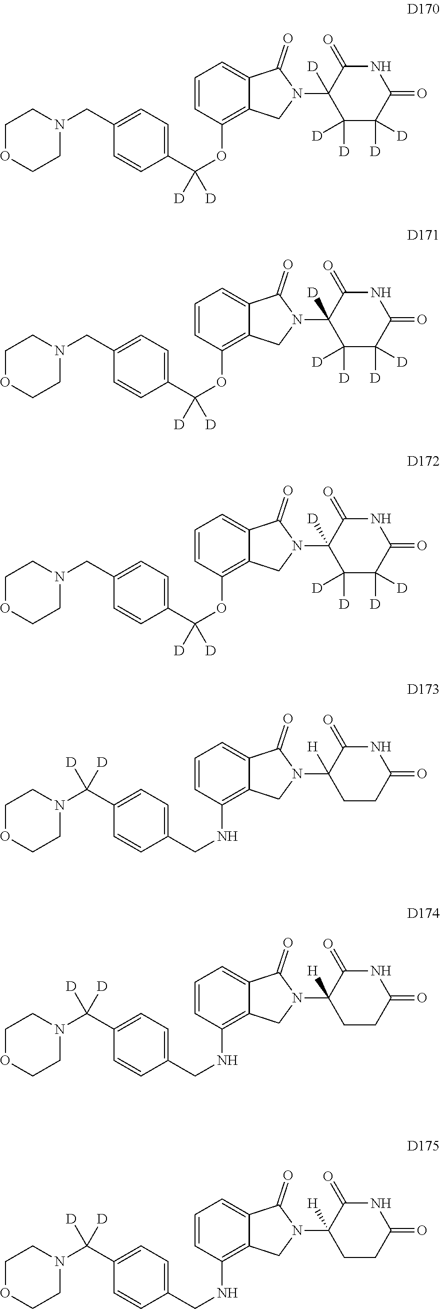

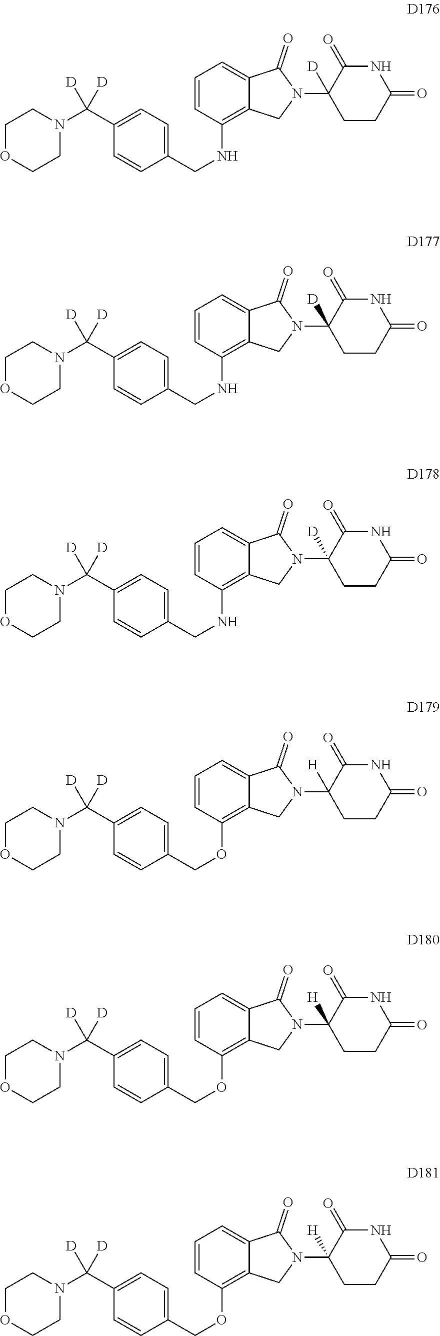 [SMILES: [2H]C1(N2Cc3c(NCc4ccc(C([2H])([2H])N5CCOCC5)cc4)cccc3C2=O)CCC(=O)NC1=O ; [2H]C([2H])(c1ccc(CNc2cccc3c2CN([C@@]2([2H])CCC(=O)NC2=O)C3=O)cc1)N1CCOCC1 ; [2H]C([2H])(c1ccc(CNc2cccc3c2CN([C@]2([2H])CCC(=O)NC2=O)C3=O)cc1)N1CCOCC1 ; [2H]C([2H])(c1ccc(COc2cccc3c2CN(C2CCC(=O)NC2=O)C3=O)cc1)N1CCOCC1 ; [2H]C([2H])(c1ccc(COc2cccc3c2CN([C@H]2CCC(=O)NC2=O)C3=O)cc1)N1CCOCC1 ; [2H]C([2H])(c1ccc(COc2cccc3c2CN([C@@H]2CCC(=O)NC2=O)C3=O)cc1)N1CCOCC1]

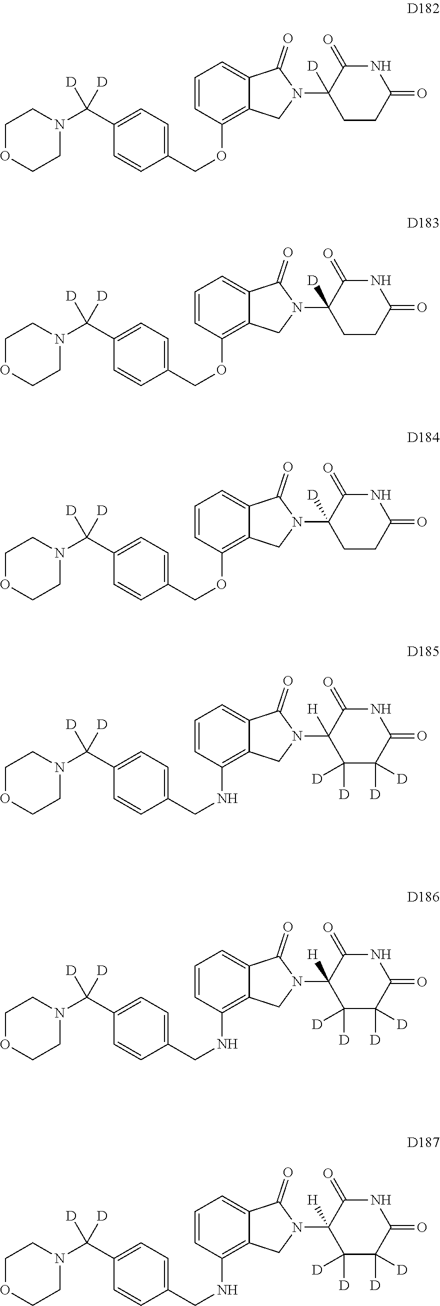

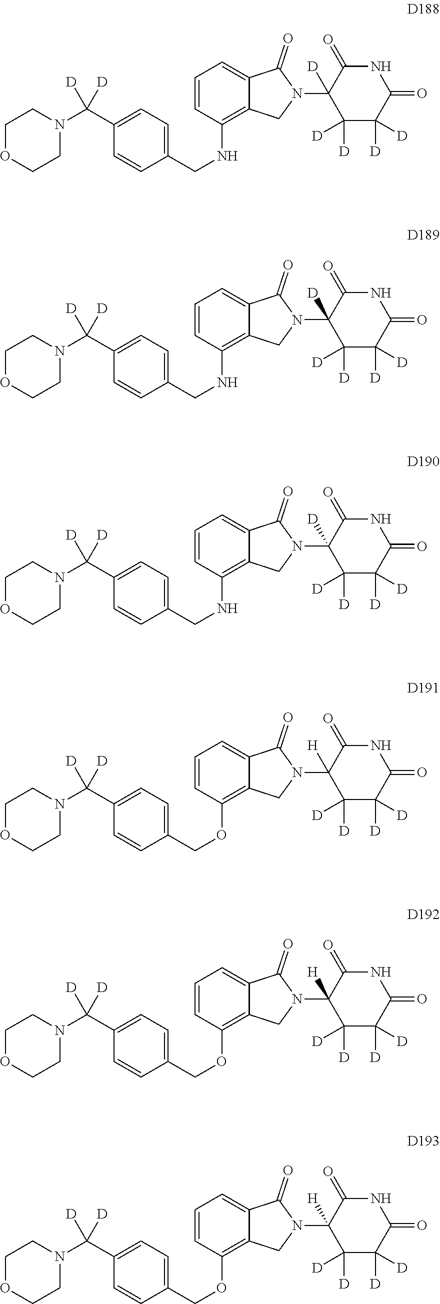

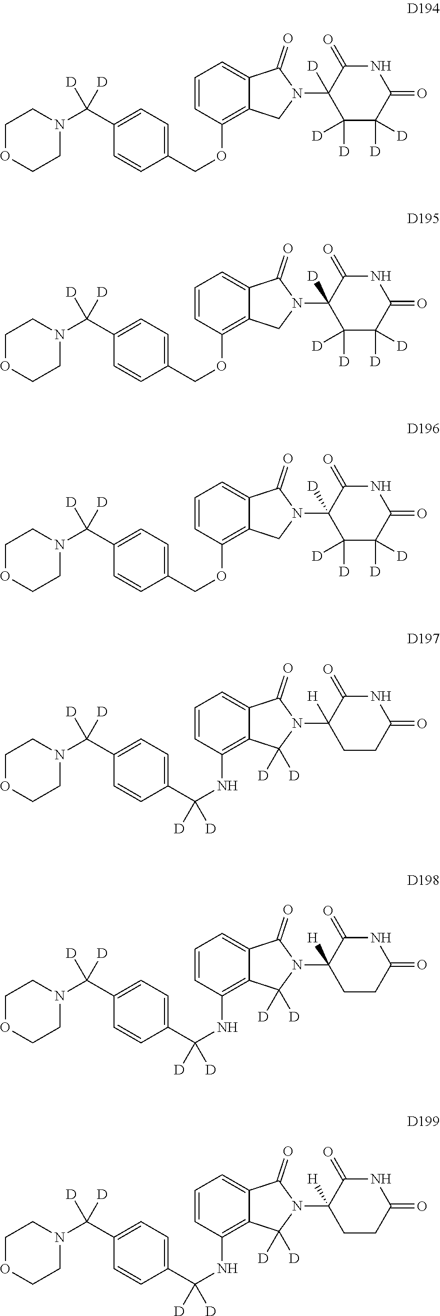

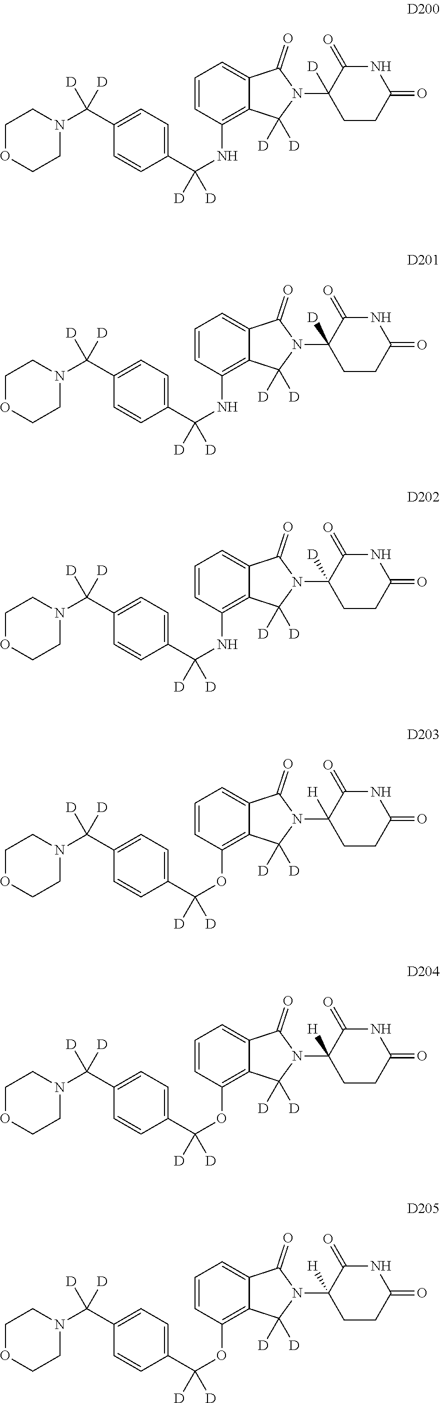

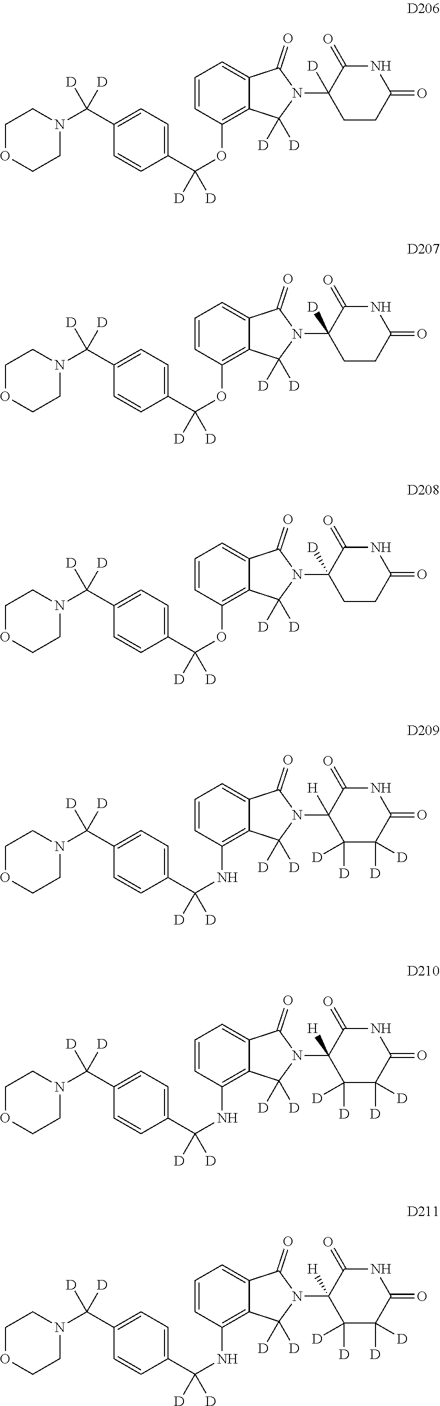

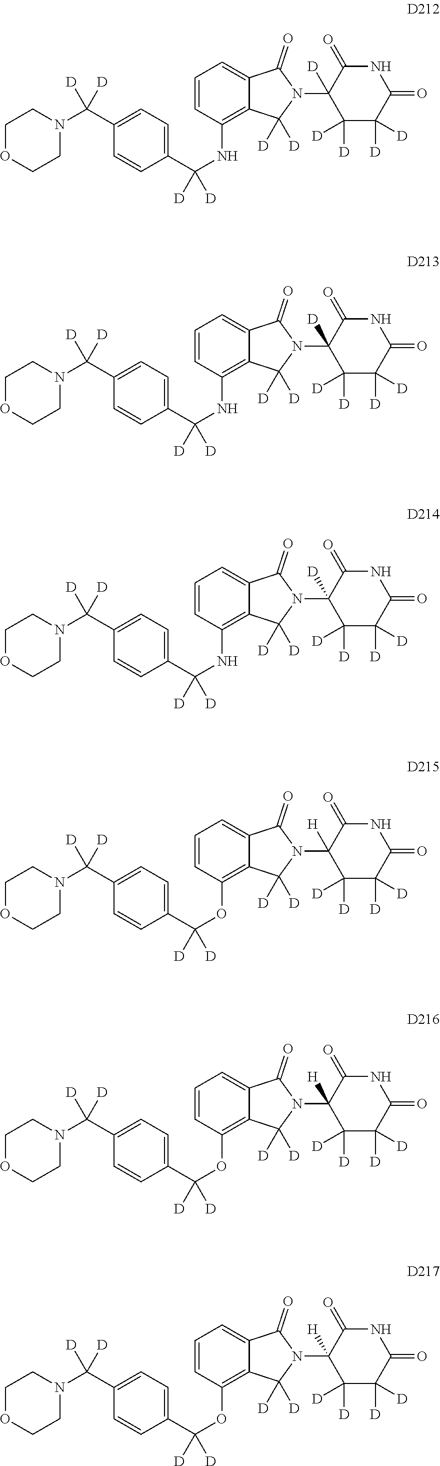

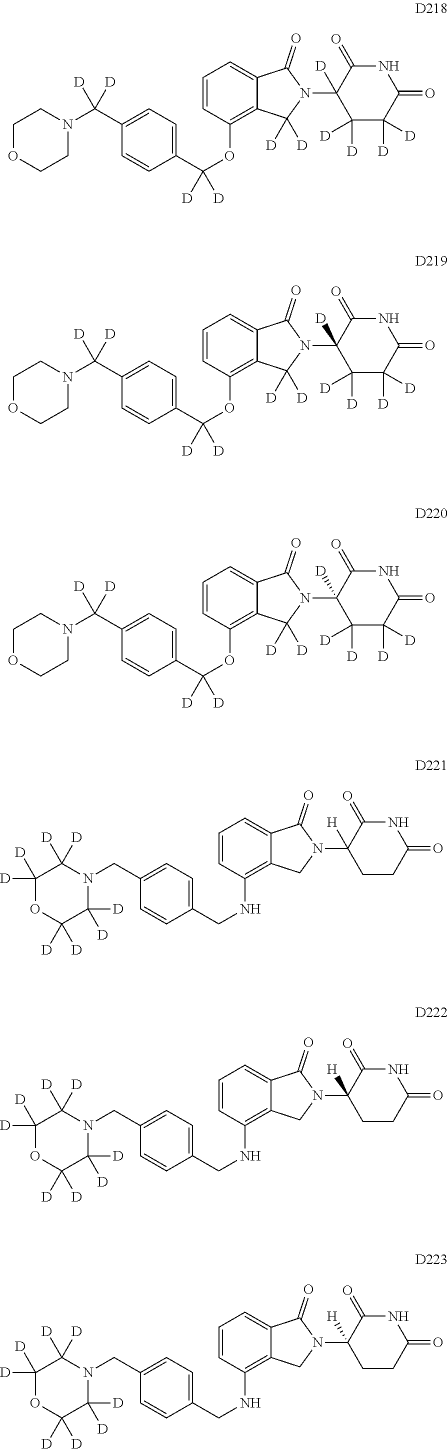

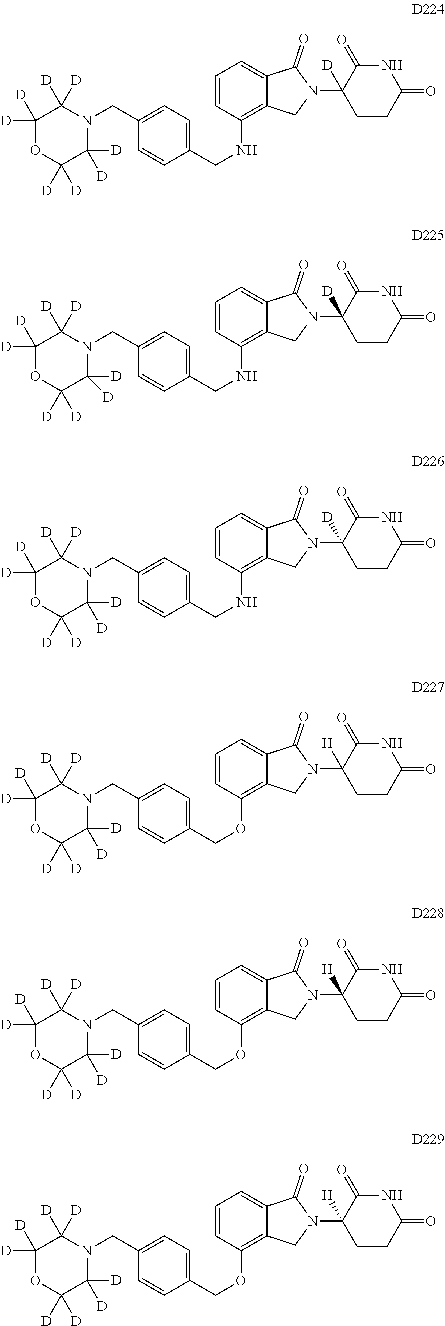

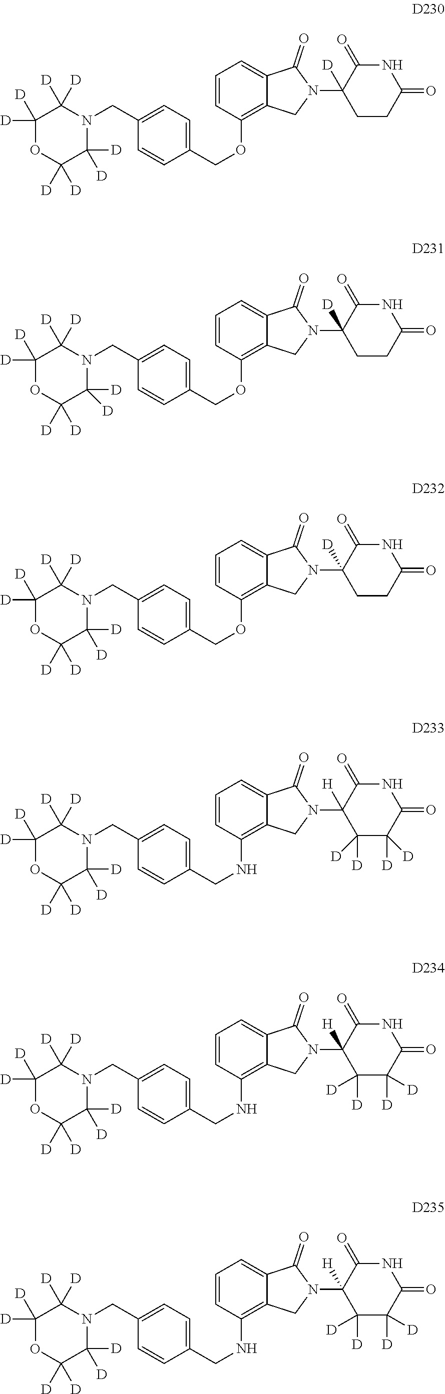

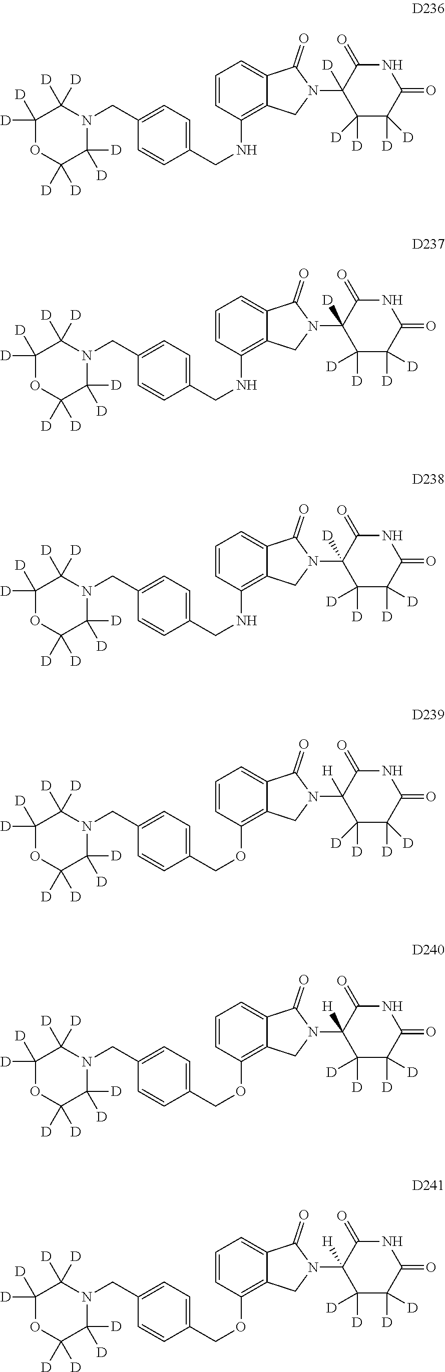

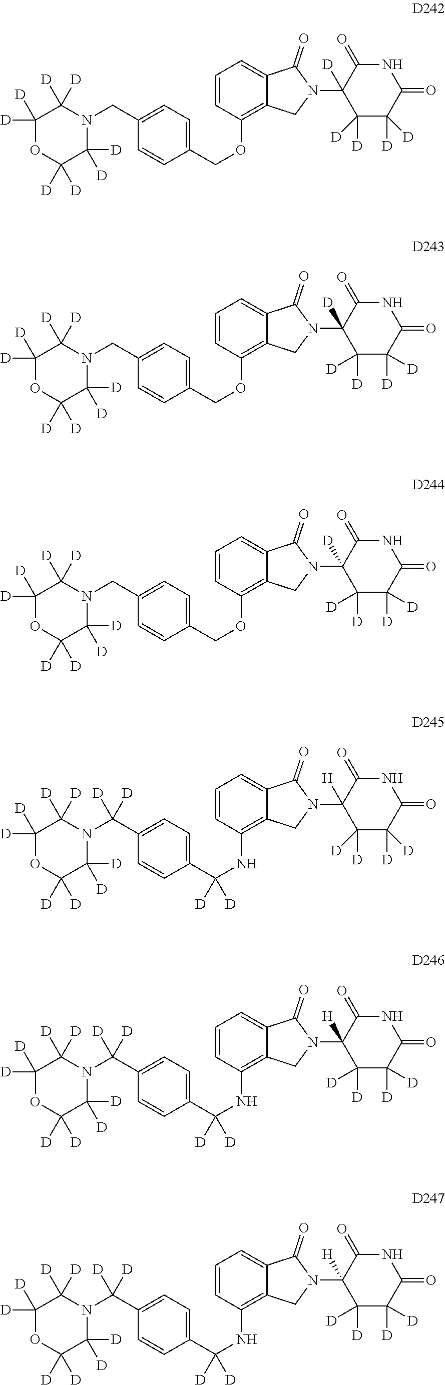

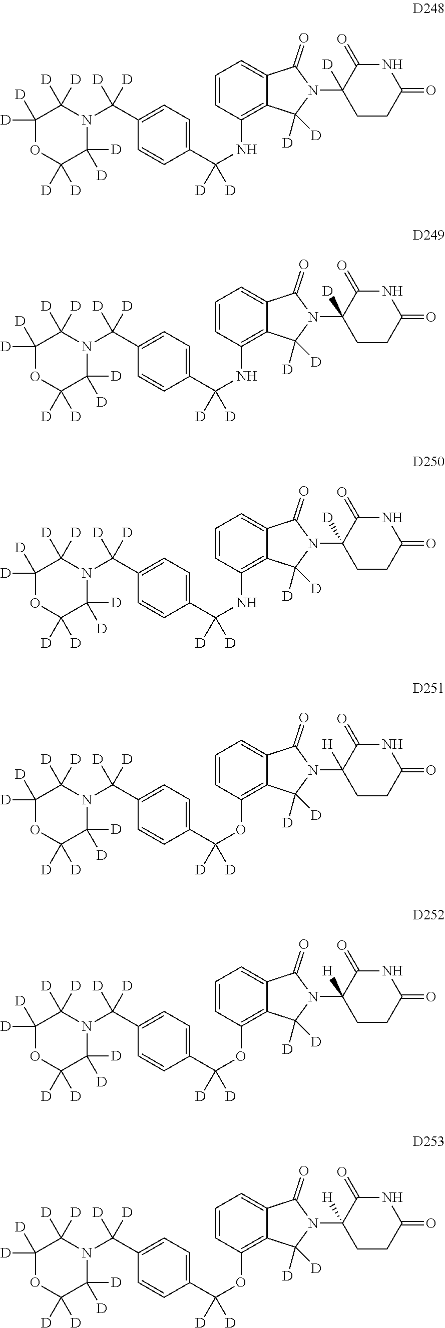

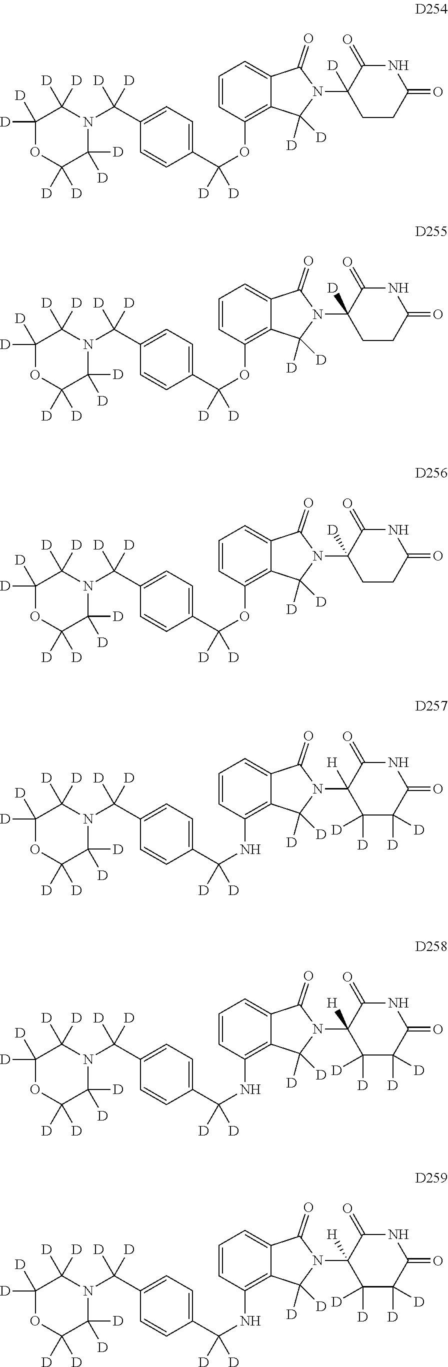

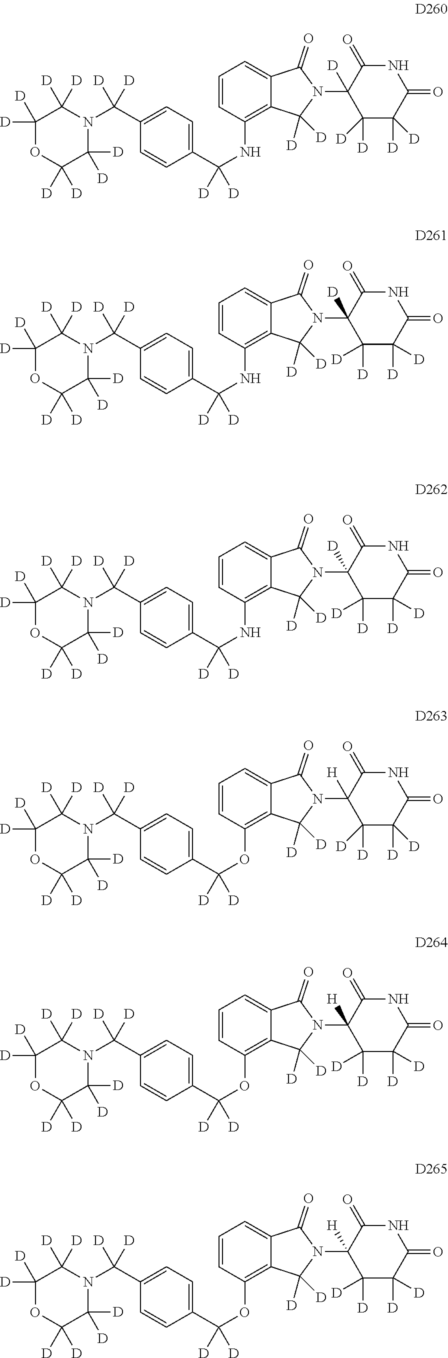

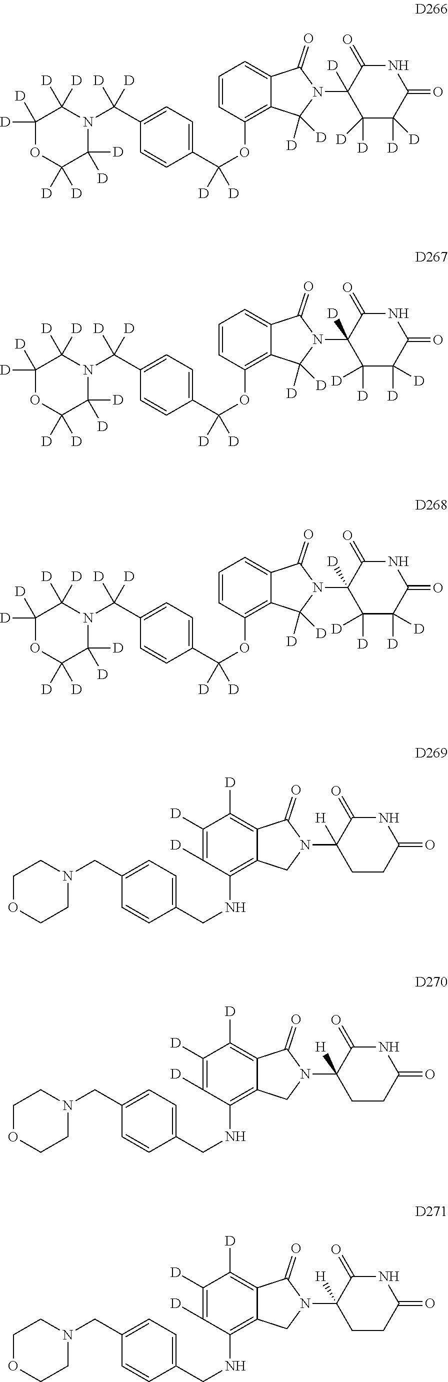

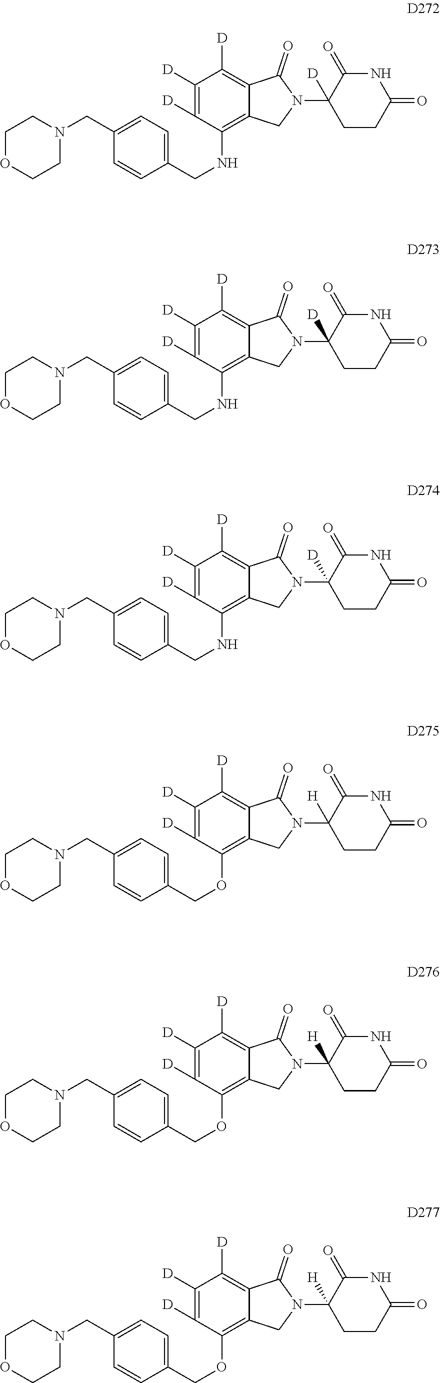 [SMILES: [2H]c1c([2H])c(NCc2ccc(CN3CCOCC3)cc2)c2c(c1[2H])C(=O)N(C1([2H])CCC(=O)NC1=O)C2 ; [2H]c1c([2H])c(NCc2ccc(CN3CCOCC3)cc2)c2c(c1[2H])C(=O)N([C@@]1([2H])CCC(=O)NC1=O)C2 ; [2H]c1c([2H])c(NCc2ccc(CN3CCOCC3)cc2)c2c(c1[2H])C(=O)N([C@]1([2H])CCC(=O)NC1=O)C2 ; [2H]c1c([2H])c(OCc2ccc(CN3CCOCC3)cc2)c2c(c1[2H])C(=O)N(C1CCC(=O)NC1=O)C2 ; [2H]c1c([2H])c(OCc2ccc(CN3CCOCC3)cc2)c2c(c1[2H])C(=O)N([C@H]1CCC(=O)NC1=O)C2 ; [2H]c1c([2H])c(OCc2ccc(CN3CCOCC3)cc2)c2c(c1[2H])C(=O)N([C@@H]1CCC(=O)NC1=O)C2]

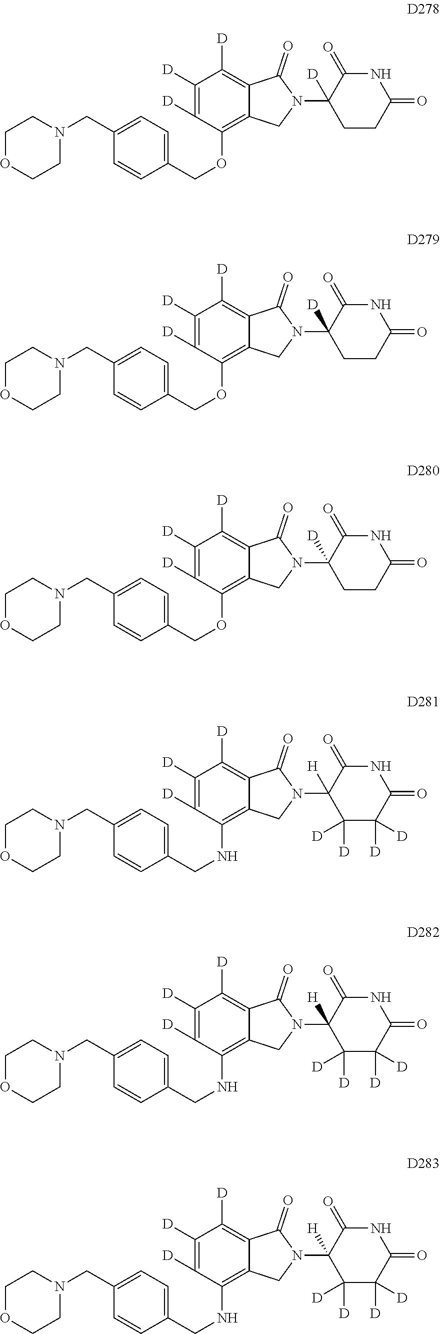

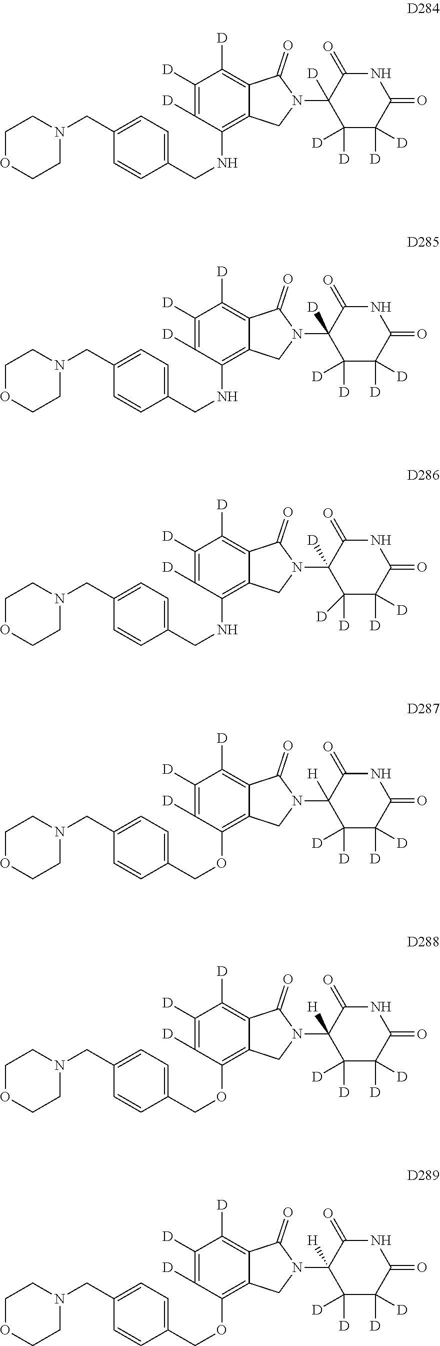

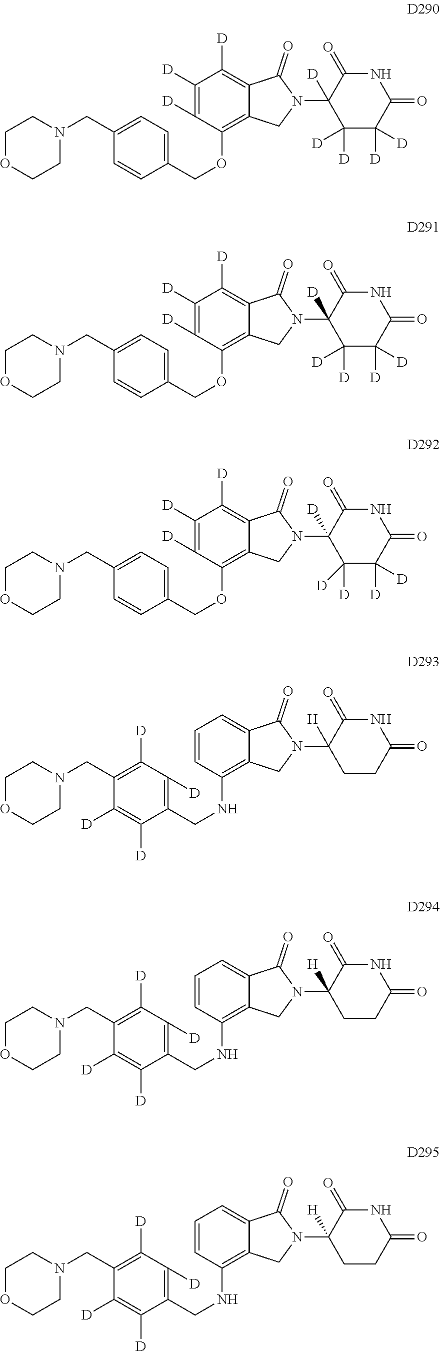

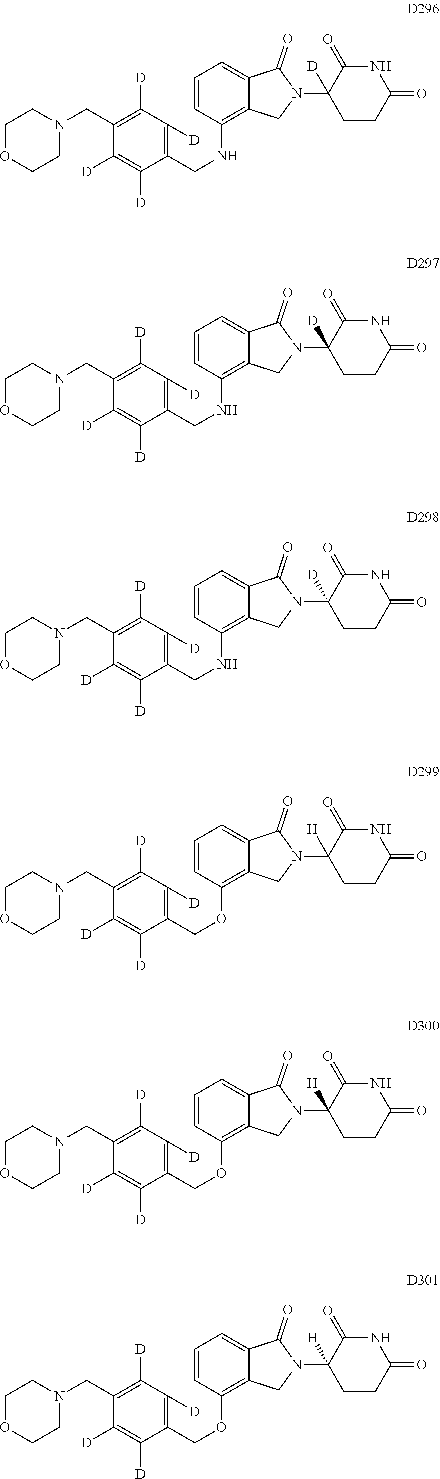 [SMILES: [2H]c1c([2H])c(CN2CCOCC2)c([2H])c([2H])c1CNc1cccc2c1CN(C1([2H])CCC(=O)NC1=O)C2=O ; [2H]c1c([2H])c(CN2CCOCC2)c([2H])c([2H])c1CNc1cccc2c1CN([C@@]1([2H])CCC(=O)NC1=O)C2=O ; [2H]c1c([2H])c(CN2CCOCC2)c([2H])c([2H])c1CNc1cccc2c1CN([C@]1([2H])CCC(=O)NC1=O)C2=O ; [2H]c1c([2H])c(CN2CCOCC2)c([2H])c([2H])c1COc1cccc2c1CN(C1CCC(=O)NC1=O)C2=O ; [2H]c1c([2H])c(CN2CCOCC2)c([2H])c([2H])c1COc1cccc2c1CN([C@H]1CCC(=O)NC1=O)C2=O ; [2H]c1c([2H])c(CN2CCOCC2)c([2H])c([2H])c1COc1cccc2c1CN([C@@H]1CCC(=O)NC1=O)C2=O]

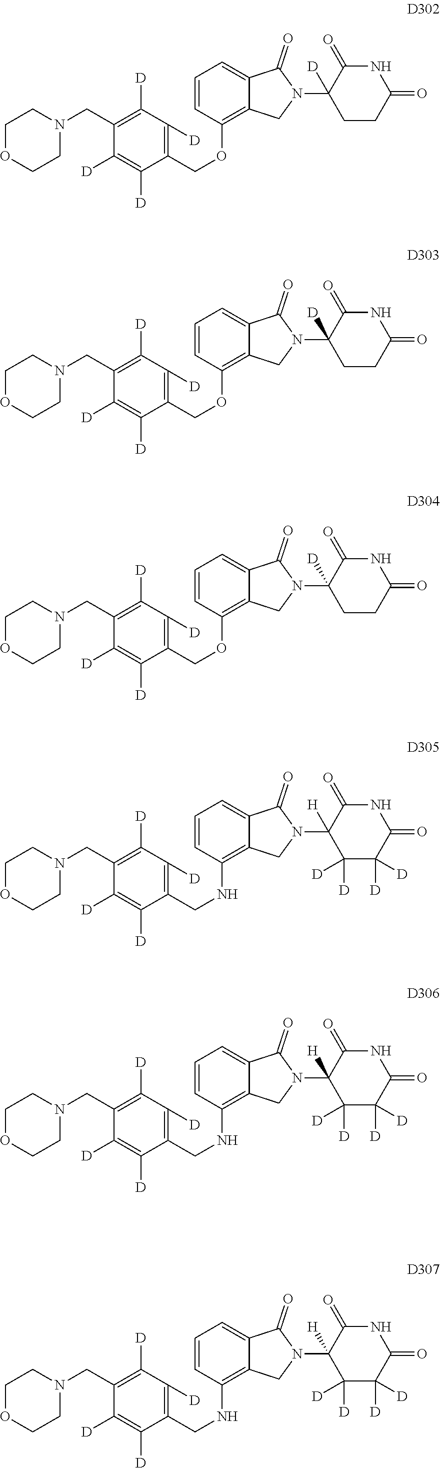

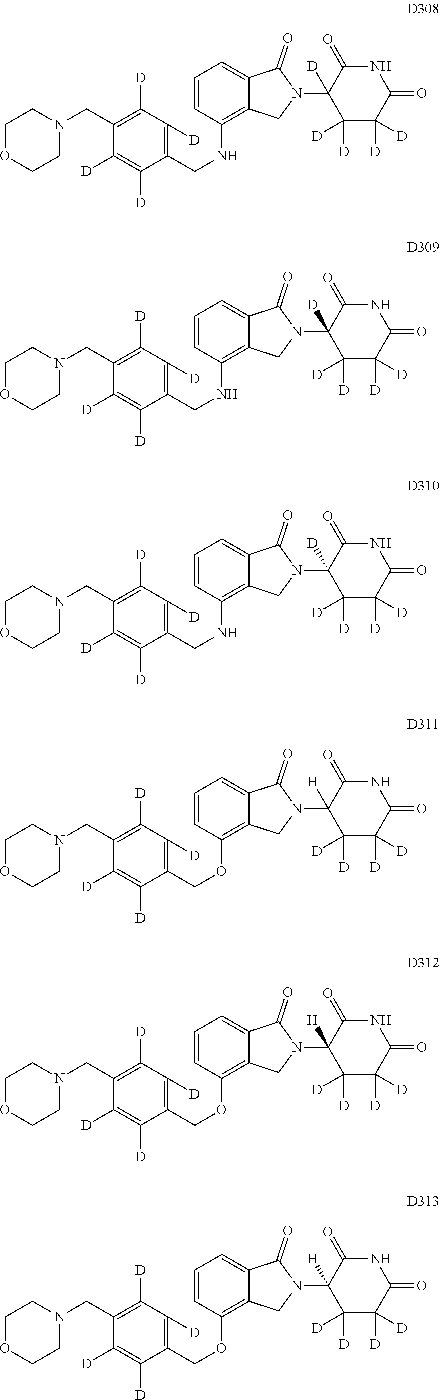

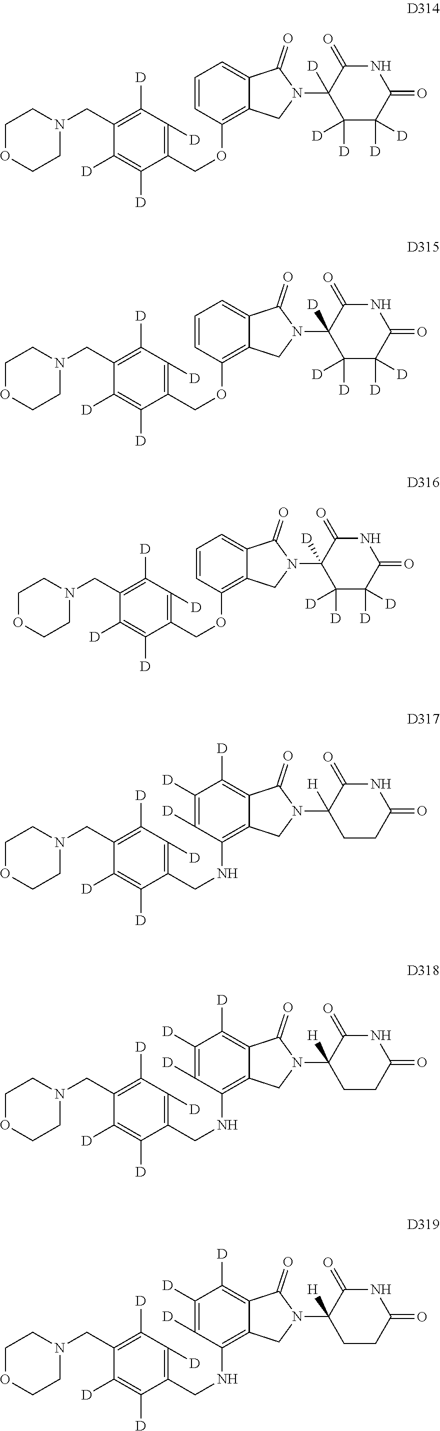

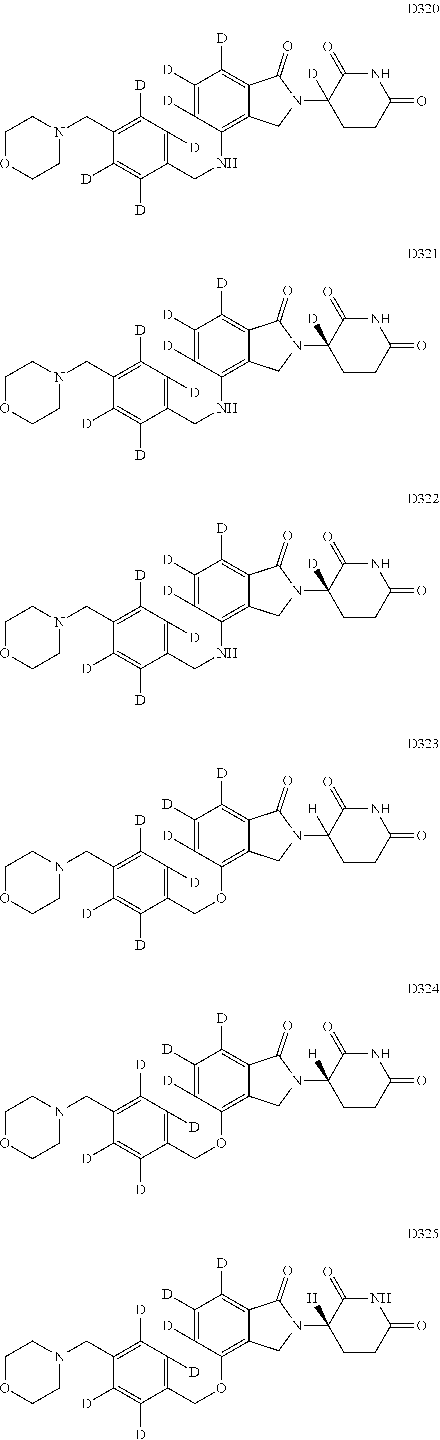

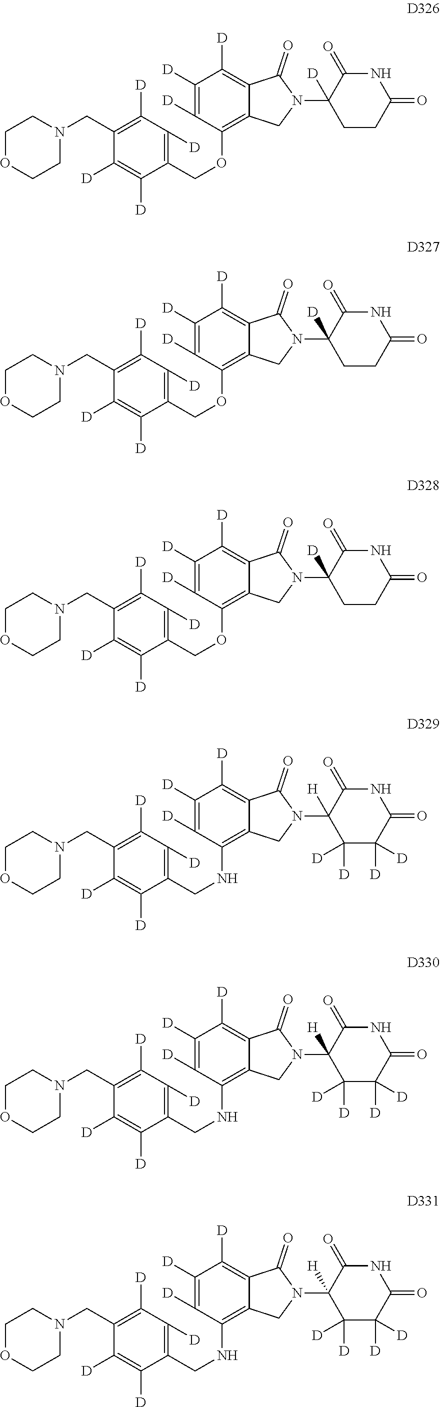

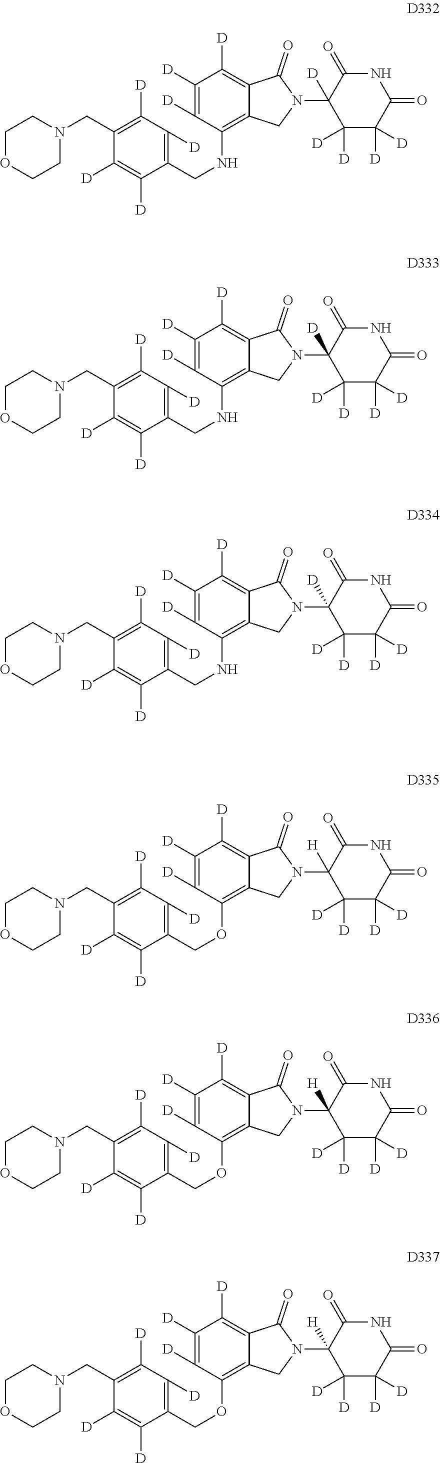

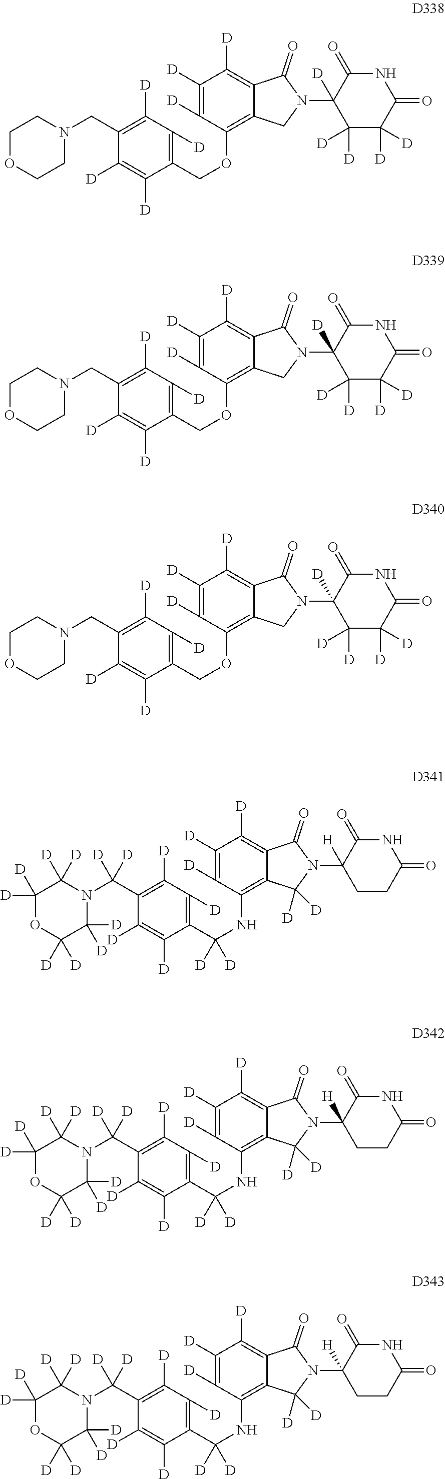

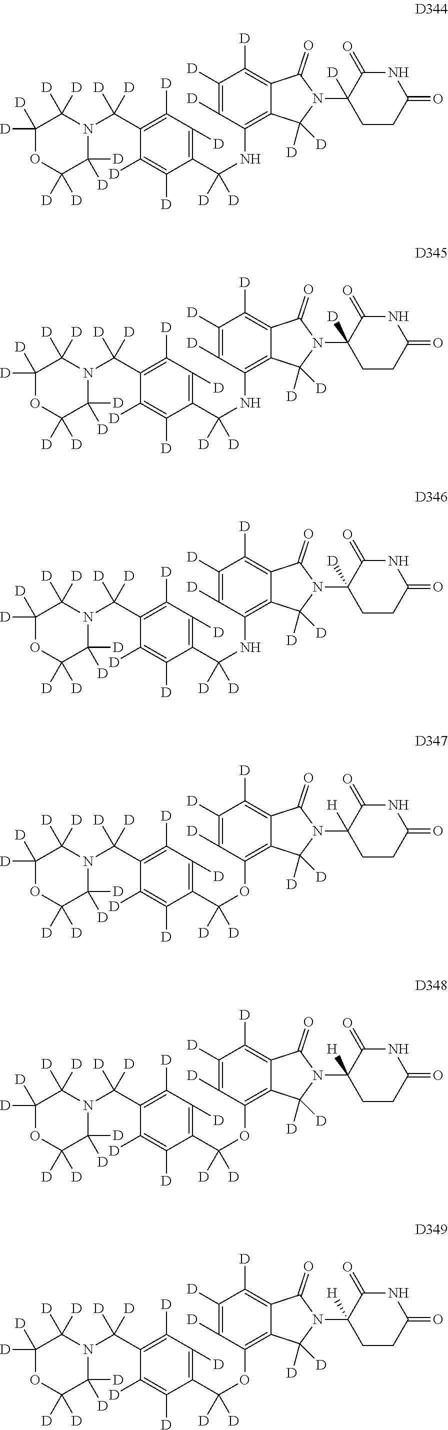

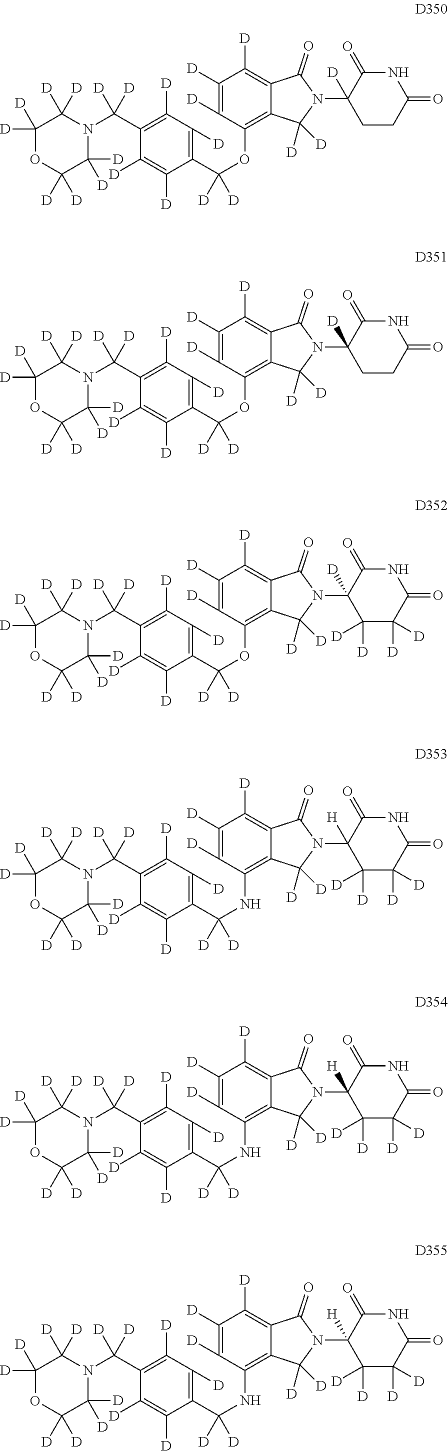

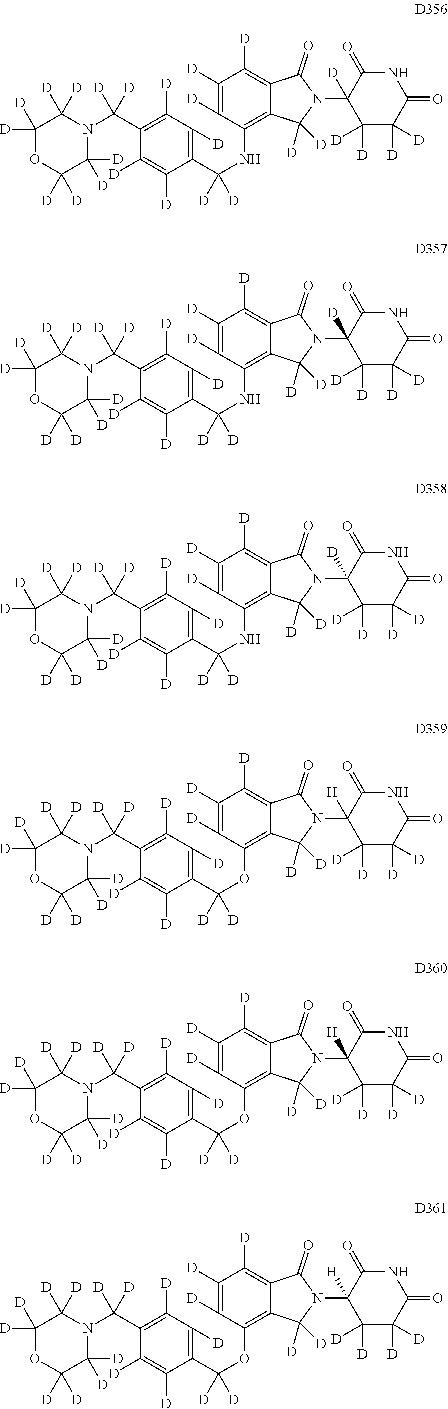

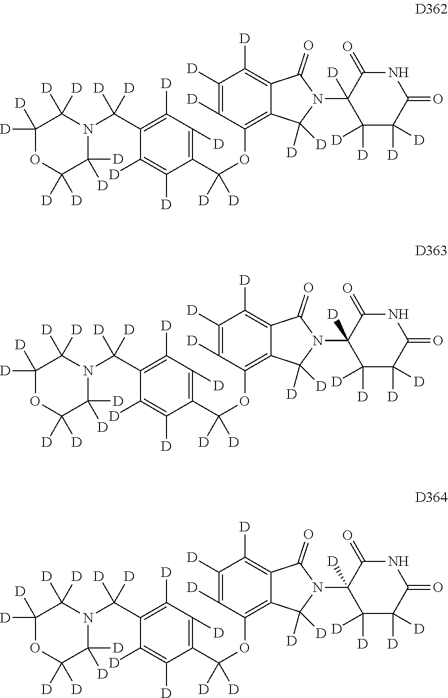

5. The pharmaceutical composition according to claim 4, wherein the benzoheterocyclic compound as shown in formula (I) is selected form B001, B002, B003, B004, B005, B006, F001, K001, D101, D107 or D108; the androgen receptor pathway modulator is selected from Enzalutamide, ARN-509, Galeterone, ODM-201, Enzalutamide and Galeterone, Enzalutamide and Abiraterone acetate, ARN-509 and Galeterone, ARN-509 and Abiraterone acetate, ODM-201 and Enzalutamide, ODM-201 and ARN-509, ODM-201 and Galeterone, ODM-201 and Abiraterone, or ODM-201 and Abiraterone acetate.

6. The pharmaceutical composition according to claim 1, wherein the pharmaceutical composition further comprises a hormone compound.

7. The pharmaceutical composition according to claim 6, wherein the hormone compound is selected from the group consisting of prednisone, dexamethasone, dehydroepiandrosterone, isoandrosterone and megestrol acetate.

8. The pharmaceutical composition according to claim 6, wherein the combination of the androgen receptor pathway modulator and the hormone compound is Galeterone and prednisone, prednisone and Abiraterone acetate, Enzalutamide and prednisone, ARN-509 and prednisone, Enzalutamide and dexamethasone, Enzalutamide and Galeterone and prednisone, Enzalutamide and Galeterone and dexamethasone, Enzalutamide and ODM-201 and prednisone, Enzalutamide and ODM-201 and dexamethasone, Enzalutamide and ORM-15341 and prednisone, Enzalutamide and ORM-15341 and dexamethasone, Enzalutamide and Abiraterone acetate and prednisone, Enzalutamide and Abiraterone acetate and dexamethasone, Enzalutamide and Abiraterone and prednisone, Enzalutamide and Abiraterone and dexamethasone, ARN-509 and dexamethasone, ARN-509 and Galeterone and prednisone, ARN-509 and Galeterone and dexamethasone, ARN-509 and ODM-201 and prednisone, ARN-509 and ODM-201 and dexamethasone, ARN-509 and ORM-15341 and prednisone, ARN-509 and ORM-15341 and dexamethasone, ARN-509 and Abiraterone acetate and prednisone, ARN-509 and Abiraterone acetate and dexamethasone, ARN-509 and Abiraterone and prednisone, ARN-509 and Abiraterone and dexamethasone, ODM-201 and prednisone, ODM-201 and dexamethasone, ODM-201 and Galeterone and prednisone, ODM-201 and Galeterone and dexamethasone, ODM-201 and Abiraterone acetate and prednisone, ODM-201 and Abiraterone acetate and dexamethasone, ODM-201 and Abiraterone and prednisone, ODM-201 and Abiraterone and dexamethasone, ORM-15341 and prednisone, ORM-15341 and dexamethasone, ORM-15341 and Galeterone and prednisone, ORM-15341 and Galeterone and dexamethasone, ORM-15341 and Abiraterone acetate and prednisone, ORM-15341 and Abiraterone acetate and dexamethasone, ORM-15341 and Abiraterone and prednisone, ORM-15341 and Abiraterone and dexamethasone, Galeterone and dexamethasone, Galeterone and Abiraterone acetate and prednisone, Galeterone and Abiraterone acetate and dexamethasone, Galeterone and Abiraterone and prednisone, or Galeterone and Abiraterone and dexamethasone.

9. The pharmaceutical composition according to claim 8, wherein the combination of the benzoheterocyclic compound as shown in formula (I), the androgen receptor pathway modulator and the hormone compound is that the benzoheterocyclic compound as shown in formula (I) is B001, K001, D101, D107 or D108; the combination of the androgen receptor pathway modulator and the hormone compound is Galeterone and prednisone, prednisone and Abiraterone acetate, Enzalutamide and prednisone, ARN-509 and prednisone, Enzalutamide and Galeterone and prednisone, Enzalutamide and Abiraterone acetate and prednisone, ARN-509 and Galeterone and prednisone, ARN-509 and Abiraterone acetate and prednisone, ODM-201 and prednisone, ODM-201 and Galeterone and prednisone, ODM-201 and Abiraterone acetate and prednisone, ODM-201 and Abiraterone and prednisone, Enzalutamide and ODM-201 and prednisone, or ARN-509 and ODM-201 and prednisone.

10. A process for the prevention and/or treatment of prostate cancer in a subject in need thereof, comprising: administering an effective amount of a benzoheterocyclic compound as shown in formula (I), a pharmaceutically acceptable salt, a solvate, a polymorph, a co-crystal, a stereoisomer, an isotope compound, a metabolite or a prodrug thereof together with an androgen receptor pathway modulator to the subject; 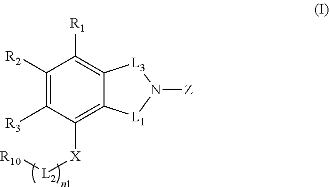
wherein, the androgen receptor pathway modulator, n1, L₁, L₃, L₂, X, R₁, R₂, R₃, R₁₀ and Z are as defined in claim 1.

11. A process for the prevention and/or treatment of prostate cancer in a subject in need thereof, comprising: administering an effective amount of a benzoheterocyclic compound as shown in formula (I), a pharmaceutically acceptable salt, a solvate, a polymorph, a co-crystal, a stereoisomer, an isotope compound, a metabolite or a prodrug thereof together with an androgen receptor pathway modulator and a hormone compound to the subject; 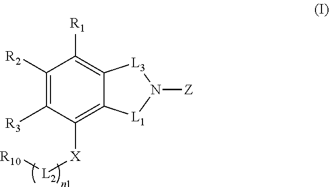
wherein, the androgen receptor pathway modulator, n1, L₁, L₃, L₂, X, R₁, R₂, R₃, R₁₀ and Z are as defined in claim 1; the hormone compound is as defined in claim 6.

12.-13. (canceled)

14. The method process according to claim 11, wherein the combination of the benzoheterocyclic compound as shown in formula (I), the androgen receptor pathway modulator and the hormone compound is that the benzoheterocyclic compound as shown in formula (I) is B001, K001, D101, D107 or D108; the combination of the androgen receptor pathway modulator and the hormone compound is Galeterone and prednisone, prednisone and Abiraterone acetate, Enzalutamide and prednisone, ARN-509 and prednisone, Enzalutamide and Galeterone and prednisone, Enzalutamide and Abiraterone acetate and prednisone, ARN-509 and Galeterone and prednisone, ARN-509 and Abiraterone acetate and prednisone, ODM-201 and prednisone, ODM-201 and Galeterone and prednisone, ODM-201 and Abiraterone acetate and prednisone, ODM-201 and Abiraterone and prednisone, Enzalutamide and ODM-201 and prednisone, or ARN-509 and ODM-201 and prednisone.
